(19) United States
(12) Patent Application Publication
(10) Pub. No.: US 2023/0066315 A1
(43) Pub. Date: Mar. 2, 2023

(54) COMPOUNDS AND USES THEREOF

(71) Applicant: Nuvation Bio Inc., New York, NY (US)

(72) Inventors: Son Minh PHAM, San Francisco, CA (US); Jayakanth KANKANALA, Plymouth, MN (US); Pradeep S. JADHAVAR, Greater Noida (IN); Baban Mohan MULIK, Noida (IN); Farha KHAN, New Delhi (IN); Sreekanth A. RAMACHANDRAN, Greater Noida (IN)

(21) Appl. No.: 17/423,772

(22) PCT Filed: Jan. 17, 2020
(86) PCT No.: PCT/US2020/014207

§ 371 Date: Jul. 16, 2021

Related U.S. Application Data
(60) Provisional application No. 62/794,525, filed on Jan. 18, 2019.

(51) Int. Cl. C07D 471/04; C07D 519/00

(52) U.S. Cl. CPC C07D 471/04; C07D 519/00

(57) ABSTRACT

Compounds as modulators of an adenosine receptor are provided. The compounds may find use as therapeutic agents for the treatment of diseases mediated through a G-protein-coupled receptor signaling pathway and may find particular use in oncology.

### CROSS REFERENCE TO RELATED APPLICATIONS

This application claims priority to U.S. Application Ser. No. 62/794,525, filed Jan. 18, 2019, which is hereby incorporated herein by reference in its entirety.

### FIELD OF THE INVENTION

This disclosure relates generally to therapeutics for treatment mediated through a G-protein-coupled receptor (GPCR) signaling pathway and, more particularly, to compounds that inhibit an adenosine receptor (such as an A_{2A} antagonist). The disclosure also provides pharmaceutically acceptable compositions comprising such compounds and methods of using the compounds or compositions in the treatment of a disease associated with a GPCR signaling pathway.

### BACKGROUND OF THE INVENTION

Adenosine receptors (ARs) are distributed throughout the body and are responsible for numerous biological functions. The seven trans-membrane G-protein-coupled receptors (GPCRs) have been divided into four different subtypes: A₁, A_{2A}, A_{2B}, and A₃. The A_{2A} and A_{2B} ARs stimulate activity of the adenylyl cyclase, inducing an increase of cAMP levels. A_{2A} ARs have a distinct tissue localization, different biochemical pathways, and specific pharmacological profiles.

Adenosine is one of the human body's most important neuromodulators in both the central and the peripheral nervous systems. Adenosine is released from tumor cells and its concentration in the extracellular fluid of tumors can reach immunosuppressive levels (Blay et al. (1997), Cancer Res., 57(13), pp. 2602-5). The extracellular fluid of solid carcinomas contains immunosuppressive concentrations of adenosine. Id. This increase in adenosine concentration is a result of increases in CD73 (ecto-5′-nucleotidase) and CD39 (nucleoside triphosphate dephosphorylase) enzymes, which are responsible for directly catabolizing ATP into adenosine. These upregulations are triggered by hypoxia and the generation of HIF-1α. High levels of adenosine around tumor cells act to regulate multiple immune cells (e.g., CD4⁺ T-cells and cytotoxic CD8⁺ T-cells) via activation of multiple adenosine receptor subtypes, but particularly A_{2A} receptors, resulting in the suppressing of pro-inflammatory activities and upregulation of anti-inflammatory molecules and immunoregulatory cells (Kumar et al. (2013), Adenosine as an endogenous immunoregulator in cancer pathogenesis: where to go? Purinergic Signal., 9(2), pp 145-65 and Sitkowsky et al., Hostile, hypoxia-A2-adenosinergic tumor biology as the next barrier to overcome for tumor immunologists. Cancer Immunol. Res. 2(7), pp 598-605; Ohta (2016), A Metabolic Immune Checkpoint: Adenosine in Tumor Microenvironment. Frontiers in Immunology., 7 article #109, pp 1-11). It was demonstrated that chimeric antigen receptor (CAR) T cells upregulate A2ARs upon antigen-specific stimulation in vitro and in vivo (Beavls (2017), Targeting the Adenosine 2A Receptor Enhances Chimeric Antigen Receptor T Cell Efficacy. J of Clin Invest. 127 (3): pp 929-941).

Survival of cancer cells is dependent on their ability to avoid attack by the immune system. In addition, tumor cells can overtake the immune system to facilitate tumor survival and metastasis. Adenosine, whose concentration increases within hypoxic regions of solid tumors, has been recognized as being able to interfere with the recognition of tumor cells by cytolytic effector cells of the immune system. (Tuite and Riss (2013). Recent developments in the pharmacological treatment of Parkinson's disease. Expert Opin. Investig. Drugs, 12(8) pp 1335-52, Popoli et al. (2002). Blockade of striatal adenosine A_{2A} receptor reduces, through a presynaptic mechanism, quinolinic acid-induced excitotoxicity: possible relevance to neuroprotective interventions in neurodegenerative diseases of the striatum, J. Neurosci, 22(5) pp. 1967-75, Gessi et al. (2011). Adenosine receptors and cancer. Biochim Biophys Acta, 1808(5), pp. 1400-12).

Although all adenosine receptors now have an increasing number of recognized biological roles in tumors, the A_{2A} and A₃ subtypes appear promising targets for therapeutic development. In particular, activation of A_{2A} receptors leads to immunosuppressive effects, which decreases anti-tumoral immunity and thereby encourages tumor growth.

The A_{2B} receptor is another potential target for therapeutic development. Autocrine/paracrine stimulation of A_{2B} expressed on tumor cells is believed to enhance their metastatic potential and A_{2B} blockade may reduce tumor metastasis in an immune-independent manner (Beavis et al. (2013). Blockade of A_{2A} receptors potently suppresses the metabolism of CD73⁺ Tumors. Proc. Natl. Acad. Sci., 110(36) pp. 14711-6). A_{2B} expression also correlates with relapse-free survival (RFS) in triple negative breast cancer suggesting that this pathway may be clinically relevant. A_{2B} blockade also has the potential to modulate the immunosuppressive properties of tumor-associated immune cells including dendritic cells and myeloid-derived suppressor cells (MDSCs) (Cekic et al. (2011). Adenosine A_{2B} receptor blockade slows growth of bladder and breast tumors. J. Immunol. 188(1), pp. 198-205; Sorrentino et al. (2015). Myeloid-derived suppressor cells contribute to A_{2B} adenosine receptor-induced VEGF production and angiogenesis in a mouse melanoma model. Oncotarget 6(29), pp. 27478-89; Iannone et al. (2013). Blockade of A_{2B} adenosine receptor reduces tumor growth and immune suppression mediated by myeloid-derived suppressor cells in a mouse model of melanoma. Neoplasia, 15(12), pp. 1400-9.

There remains a continuing need for new therapies for the treatment of diseases and disorders related to the adenosine signaling pathway.

### BRIEF SUMMARY OF THE INVENTION

In one aspect, provided is a compound of the formula (I):

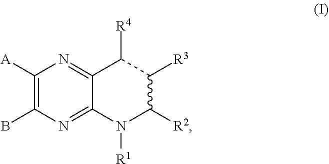

or a tautomer or isomer thereof, or a pharmaceutically acceptable salt of any of the foregoing, wherein A, B, R¹, R², R³ and R⁴ are as detailed herein.

In some embodiments, the compound of the formula (I), or a tautomer or isomer thereof, or a pharmaceutically acceptable salt of any of the foregoing, is of formula (II) or (III), or a tautomer or isomer thereof, or a pharmaceutically acceptable salt of any of the foregoing, as detailed herein. In some embodiments, provided is a compound of formula (II), or a tautomer thereof, or a salt of any of the foregoing. In some embodiments, provided is a compound of formula (III), or a tautomer thereof, or a salt of any of the foregoing.

In another aspect, provided is a method for any one or more of: (a) treating a disease, such as a proliferative disease, in an individual in need thereof; (b) enhancing an immune response in an individual in need thereof; (c) inhibiting tumor metastasis in an individual in need thereof; (d) modulating the activity of a G protein coupled receptor signaling pathway in an individual in need thereof; (e) modulating the activity of an adenosine receptor, such as an A_{2A} receptor, in an individual in need thereof; and (f) increasing the activity of a natural killer cell in an individual in need thereof, wherein the method comprises administering to the individual an effective amount of a compound of formula (I) or any related formula such as formula (II) or formula (III), or a tautomer or isomer thereof, or a pharmaceutically acceptable salt of any of the foregoing. In some embodiments, provided is a method for any one or more of: (a) treating a disease, such as a proliferative disease, in an individual in need thereof; (b) enhancing an immune response in an individual in need thereof; (c) inhibiting tumor metastasis in an individual in need thereof; (d) modulating the activity of a G protein coupled receptor signaling pathway in an individual in need thereof; (e) modulating the activity of an adenosine receptor, such as an A_{2A} receptor, in an individual in need thereof; and (f) increasing the activity of a natural killer cell in an individual in need thereof, wherein the method comprises administering to the individual an effective amount of a compound of formula (I) or any related formula such as formula (II) or formula (III), or a tautomer or isomer thereof, or a pharmaceutically acceptable salt of any of the foregoing. In one aspect, the compound of formula (I) or any related formula such as formula (II) or formula (III), or a tautomer or isomer thereof, or a pharmaceutically acceptable salt of any of the foregoing, is administered to the individual in combination with another therapeutic agent. In some embodiments, the compound of formula (I) or any related formula such as formula (II) or formula (III), or a tautomer or isomer thereof, or a pharmaceutically acceptable salt of any of the foregoing is administered to the individual in combination with another therapeutic agent.

Also provided are pharmaceutical compositions comprising (A) a compound detailed herein, such as a compound of formula (I) or any related formula such as formula (II) or formula (III), or a tautomer or isomer thereof, or a pharmaceutically acceptable salt of any of the foregoing and (B) a pharmaceutically acceptable carrier or excipient. In some embodiments, provided are pharmaceutical compositions comprising (A) a compound detailed herein, such as a compound of formula (I) or any related formula such as formula (II) or formula (III), or a tautomer or isomer thereof, or a or a salt of any of the foregoing, and (B) a pharmaceutically acceptable carrier or excipient. Kits comprising a compound detailed herein or a tautomer or isomer thereof, or a pharmaceutically acceptable salt of any of the foregoing and instructions for use are also provided. Kits comprising a compound detailed herein or a salt thereof and instructions for use are also provided. A compound detailed herein or a tautomer or isomer thereof, or a pharmaceutically acceptable salt of any of the foregoing is also provided for the manufacture of a medicament for the treatment of cancer. Compounds as detailed herein or a pharmaceutically acceptable salt thereof are also provided for the manufacture of a medicament for the treatment of cancer.

### DETAILED DESCRIPTION OF THE INVENTION

### Definitions

For use herein, unless clearly indicated otherwise, use of the terms “a”, “an” and the like refers to one or more.

“Alkenyl” as used herein refers to an unsaturated linear or branched univalent hydrocarbon chain or combination thereof, having at least one site of olefinic unsaturation (i.e., having at least one moiety of the formula C≡C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

The term “alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), isopropylene (—CH₂—C(H)(CH₃)—CH₂—) and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

The term “aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

The terms “cycloalkyl” or “carbocycle” are used interchangeably and refer to and include cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl or carbocycle groups can consist of one ring, such as cyclohexyl, or multiple rings, such as adamantyl, but excludes aryl groups. A cycloalkyl or carbocycle comprising more than one ring may be fused, spiro or bridged, or combinations thereof. A preferred cycloalkyl or carbocycle is a cyclic hydrocarbon having from 3 to 13 annular carbon atoms. A more preferred cycloalkyl or carbocycle is a cyclic hydrocarbon having from 3 to 8 annular carbon atoms (a “C₃-C₈ cycloalkyl”). Examples of cycloalkyl or carbocycle groups include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

The term “heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, and the like. Examples of heteroaryl groups also include, but are not limited to, pyridyl, pyrimidyl, thiophenyl, furanyl, thiazolyl, oxazolyl, isoxazolyl, thiophenyl, pyrrolyl, pyrazolyl, 1,3,4-oxadiazolyl, imidazolyl, isothiazolyl, triazolyl, 1,3,4-thiadiazolyl, tetrazolyl, benzofuranyl, benzothiophenyl, pyrazolopyridinyl, indazolyl, benzothiazolyl, benzooxazolyl or benzoimidazolyl and the like

In one variation, a heteroaryl containing at least one additional fused ring that is nonaromatic (e.g., cycloakyl or heterocyclyl) is attached to the parent structure at an annular atom of the additional ring. In another variation, a heteroaryl containing at least one additional ring that is nonaromatic (e.g., cycloakyl or heterocyclyl) is attached to the parent structure at an annular atom of the aromatic ring.

The term “heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl, cycloalkyl or heterocyclyl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

In one variation, a heterocyclyl containing at least one additional ring (such as a fused additional ring) that does not contain a heteroatom is attached to the parent structure at an annular atom of the additional ring. In another variation, a heterocyclyl containing at least one additional ring (such as a fused additional ring) that does not contain a heteroatom is attached to the parent structure at an annular atom of the ring containing a heteroatom.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Compounds

In one aspect, provided is a compound of the Formula (I):

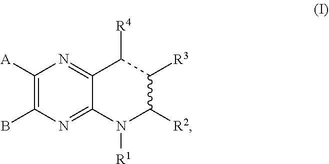

or a tautomer or isomer thereof, or a pharmaceutically acceptable salt of any of the foregoing, wherein:

R¹ is H or C₁-C₆ alkyl wherein the C₁-C₆ alkyl of R¹ is optionally substituted with oxo or R^{a};

R² and R⁴ are each independently H, R^{b} or oxo;

R³ is H or R^{c};

each R^{a}, R^{b}, and R^{c} is independently C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁸, —OC(O)R⁸, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁹S(O)R⁹, —NR⁹S(O)₂R⁹, —C(O)NR⁹S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁹, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁹S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{a}, R^{b}, and R^{c} is independently optionally substituted by halogen, oxo, —OR¹¹, —NR¹¹R¹², —C(O)R¹, —CN, —S(O)R¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen;

[symbol] is a single bond or a double bond, wherein when [symbol] is a double bond, R² is oxo;

[symbol] is a single bond or a double bond, wherein when [symbol] is a double bond, R⁴ is oxo; [symbol]
one of [symbol] and [symbol] is a double bond and the other is a single bond;

A is C₆-C₁₂ aryl, 5- to 10-membered heteroaryl, 9- to 10-membered carbocycle, or 9- to 10-membered heterocycle, wherein the C₆-C₁₂ aryl, 5- to 10-membered heteroaryl, 9- to 10-membered carbocycle, or 9- to 10-membered heterocycle of A is optionally further substituted with R⁶;

B is phenyl, 5- to 6-membered heteroaryl, 5- to 6-membered carbocycle, 5- to 6-membered heterocycle, or 9- to 10-membered heteroaryl, wherein the phenyl, 5- to 6-membered heteroaryl, 5- to 6-membered carbocycle, 5- to 6-membered heterocycle, or 9- to 10-membered heteroaryl of B is optionally further substituted with R¹;

each R⁶ and R⁷ is independently oxo, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁸, —OC(O)R⁸, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R⁶ and R⁷ is independently optionally substituted by halogen, oxo, —OR¹¹, —NR¹¹R¹², —C(O)R¹¹, —CN, —S(O)R¹¹, —S(O)₂R¹¹, —P(O)(OR¹¹))(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen,

each R⁸ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl, 3-6-membered heterocyclyl, —(C₁-C₃ alkylene)(C₃-C₆ cycloalkyl), —(C₁-C₃ alkylene)(C₆-C₁₄ aryl), —(C₁-C₃ alkylene)(5-6-membered heteroaryl), or —(C₁-C₃ alkylene)(3-6-membered heterocyclyl), wherein the C₁-C₆ alkyl, C₂-C₆ alkenyl, C₂-C₆ alkynyl, C₃-C₆ cycloalkyl, C₆-C₁₄ aryl, 5-6-membered heteroaryl, 3-6-membered heterocyclyl, —(C₁-C₃ alkylene)(C₃-C₆ cycloalkyl), —(C₁-C₃ alkylene)(C₆-C₁₄ aryl), —(C₁-C₃ alkylene)(5-6-membered heteroaryl), and —(C₁-C₃ alkylene)(3-6-membered heterocyclyl) of R⁸ are independently optionally substituted by halogen, oxo, —CN, —OR¹³, —NR¹³R¹⁴, —P(O)(OR¹³)(OR¹⁴), phenyl optionally substituted by halogen, or C₁-C₆ alkyl optionally substituted by halogen, —OH or oxo;

R⁹ and R¹⁰ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl, 3-6 membered heterocyclyl, —(C₁-C₃ alkylene)NR¹¹R¹², —(C₁-C₃ alkylene)(C₃-C₆ cycloalkyl), —(C₁-C₃ alkylene)(3-6-membered heterocyclyl), —(C₃-C₆ cycloalkylene)(5-6 membered heteroaryl), —(C₁-C₃ alkylene)(5-6-membered heteroaryl) or —(C₁-C₃ alkylene)(C aryl), wherein the C₁-C₆ alkyl, C₂-C₆ alkenyl, C₂-C₆ alkynyl, C₃-C₆ cycloalkyl, C₆-C₁₄ aryl, 5-6-membered heteroaryl, 3-6 membered heterocyclyl, —(C₁-C₃ alkylene)(C₃-C₆ cycloalkyl), —(C₁-C₃ alkylene)(3-6-membered heterocyclyl), —(C₃-C₆ cycloalkylene)(5-6 membered heteroaryl), —(C₁-C₃ alkylene)(5-6-membered heteroaryl) and —(C₁-C₃ alkylene)(C₆ aryl) of R⁹ and R¹⁰ are independently optionally substituted by halogen, oxo, —CN, —OR¹³, —NR¹³R¹⁴ or C₁-C₆ alkyl optionally substituted by halogen, —OH or oxo;
or R⁹ and R¹⁰ are taken together with the atom to which they attached to form a 3-6 membered heterocyclyl optionally substituted by halogen, oxo, —OR¹³, —NR¹³R¹⁴ or C₁-C₆ alkyl optionally substituted by halogen, oxo or —OH;

R¹¹ and R¹² are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹¹ and R¹² are taken together with the atom to which they attached to form a 3-6 membered heterocyclyl optionally substituted by halogen, oxo or C₁-C₆ alkyl optionally substituted by halogen or oxo; and

R¹³ and R¹⁴ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹³ and R¹⁴ are taken together with the atom to which they attached to form a 3-6 membered heterocyclyl optionally substituted by halogen, oxo or C₁-C₆ alkyl optionally substituted by oxo or halogen.

In some embodiments, provided is a compound of the Formula (I):

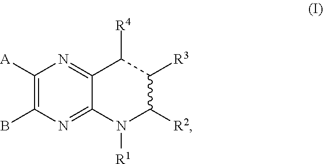

or a tautomer or isomer thereof, or a pharmaceutically acceptable salt of any of the foregoing, wherein:

R¹ is H or C₁-C₆ alkyl wherein the C₁-C₆ alkyl of R¹ is optionally substituted with oxo or R^{a};

R² and R⁴ are each independently H, R^{b} or oxo, provided that when R² is —NR⁹R¹⁰, then at least one of R⁹ and R¹⁰ is not H;

R³ is H or R^{c};

each R^{a}, R^{b}, and R^{c} is independently C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁸, —OC(O)R⁸, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁹S(O)R⁹, —NR⁹S(O)₂R⁹, —C(O)NR⁹S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁹S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{a}, R^{b}, and R^{c} is independently optionally substituted by halogen, oxo, —OR¹¹, —NR¹¹R¹², —C(O)R¹¹, —CN, —S(O)R¹¹, —S(O)₂R¹¹, —P(O)(OR¹))(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen;

[symbol] is a single bond or a double bond, wherein when [symbol] is a double bond, R² is oxo;

[symbol] is a single bond or a double bond, wherein when [symbol] is a double bond, R⁴ is oxo;
one of [symbol] and [symbol] is a double bond and the other is a single bond;

A is C₆-C₁₂ aryl, 5- to 10-membered heteroaryl, 9- to 10-membered carbocycle, or 9- to 10-membered heterocycle, wherein the C₆-C₁₂ aryl, 5- to 10-membered heteroaryl, 9- to 10-membered carbocycle, or 9- to 10-membered heterocycle of A is optionally further substituted with R⁶;

B is phenyl, 5- to 6-membered heteroaryl, 5- to 6-membered carbocycle, 5- to 6-membered heterocycle, or 9- to 10-membered heteroaryl, wherein the phenyl, 5- to 6-membered heteroaryl, 5- to 6-membered carbocycle, 5- to 6-membered heterocycle, or 9- to 10-membered heteroaryl of B is optionally further substituted with R¹;

each R⁶ and R⁷ is independently oxo, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R⁶ and R⁷ is independently optionally substituted by halogen, oxo, —OR¹¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen,
provided that when R¹ is C₁-C₆ alkyl, then R⁶ is oxo, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl);

each R⁸ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl, 3-6-membered heterocyclyl, —(C₁-C₃ alkylene)(C₃-C₆ cycloalkyl), —(C₁-C₃ alkylene)(C₆-C₁₄ aryl), —(C₁-C₃ alkylene)(5-6-membered heteroaryl), or —(C₁-C₃ alkylene)(3-6-membered heterocyclyl), wherein the C₁-C₆ alkyl, C₂-C₆ alkenyl, C₂-C₆ alkynyl, C₃-C₆ cycloalkyl, C₆-C₁₄ aryl, 5-6-membered heteroaryl, 3-6-membered heterocyclyl, —(C₁-C₃ alkylene)(C₃-C₆ cycloalkyl), —(C₁-C₃ alkylene)(C₆-C₁₄ aryl), —(C₁-C₃ alkylene)(5-6-membered heteroaryl), and —(C₁-C₃ alkylene)(3-6-membered heterocyclyl) of R⁸ are independently optionally substituted by halogen, oxo, —CN, —OR¹³, —NR¹³R¹⁴, —P(O)(OR¹³)(OR¹⁴), phenyl optionally substituted by halogen, or C₁-C₆ alkyl optionally substituted by halogen, —OH or oxo;

R⁹ and R¹⁰ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl, 3-6 membered heterocyclyl, —(C₁-C₃ alkylene)NR¹¹R¹², —(C₁-C₃ alkylene)(C₃-C₆ cycloalkyl), —(C₁-C₃ alkylene)(3-6-membered heterocyclyl), —(C₁-C₃ alkylene)(5-6-membered heteroaryl) or —(C₁-C₃ alkylene)(C aryl), wherein the C₁-C₆ alkyl, C₂-C₆ alkenyl, C₂-C₆ alkynyl, C₃-C₆ cycloalkyl, C₆-C₁₄ aryl, 5-6-membered heteroaryl, 3-6 membered heterocyclyl, —(C₁-C₃ alkylene)(C₃-C₆ cycloalkyl), —(C₁-C₃ alkylene)(3-6-membered heterocyclyl), —(C₁-C₃ alkylene)(5-6-membered heteroaryl) and —(C₁-C₃ alkylene)(C6 aryl) of R⁹ and R¹⁰ are independently optionally substituted by halogen, oxo, —CN, —OR¹³, —NR¹³R¹⁴ or C₁-C₆ alkyl optionally substituted by halogen, —OH or oxo;
or R⁹ and R¹⁰ are taken together with the atom to which they attached to form a 3-6 membered heterocyclyl optionally substituted by halogen, oxo, —OR¹³, —NR¹³R¹⁴ or C₁-C₆ alkyl optionally substituted by halogen, oxo or —OH;

R¹¹ and R¹² are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹ and R¹² are taken together with the atom to which they attached to form a 3-6 membered heterocyclyl optionally substituted by halogen, oxo or C₁-C₆ alkyl optionally substituted by halogen or oxo; and

R¹³ and R¹⁴ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹³ and R¹⁴ are taken together with the atom to which they attached to form a 3-6 membered heterocyclyl optionally substituted by halogen, oxo or C₁-C₆ alkyl optionally substituted by oxo or halogen.

In some variations of formula (I), or a tautomer or isomer thereof, or a pharmaceutically acceptable salt of any of the foregoing, the compound 3-phenyl-2-(quinolin-6-yl)pyrido[2,3-b]pyrazin-8(5H)-one, or a tautomer or isomer thereof, or a pharmaceutically acceptable salt of any of the foregoing, is excluded. Thus, in some variations is provided a compound of formula (I), or a tautomer or isomer thereof, or a pharmaceutically acceptable salt of any of the foregoing, provided the compound is not 3-phenyl-2-(quinolin-6-yl)pyrido[2,3-b]pyrazin-8(5H)-one, or a tautomer or isomer thereof, or a pharmaceutically acceptable salt of any of the foregoing. In some variations of formula (I), or a tautomer or isomer thereof, or a pharmaceutically acceptable salt of any of the foregoing, the compound 3-phenyl-2-(quinolin-6-yl)pyrido[2,3-b]pyrazin-8(5H)-one, or a tautomer or isomer thereof, or a pharmaceutically acceptable salt of any of the foregoing, is included. In some variations of formula (I), or a pharmaceutically acceptable salt thereof, the compound 3-phenyl-2-(quinolin-6-yl)pyrido[2,3-b]pyrazin-8(5H)-one, or a pharmaceutically acceptable salt thereof, is excluded. Thus, in some variations is provided a compound of formula (I), or a pharmaceutically acceptable salt thereof, provided the compound is not 3-phenyl-2-(quinolin-6-yl)pyrido[2,3-b]pyrazin-8(5H)-one, or a pharmaceutically acceptable salt thereof. In some variations of formula (I), or a pharmaceutically acceptable salt thereof, the compound 3-phenyl-2-(quinolin-6-yl)pyrido[2,3-b]pyrazin-8(5H)-one, or a pharmaceutically acceptable salt thereof, is included.

In some embodiments of the compound of Formula (I), when R² is oxo, R³ and R⁴ are H, and B is phenyl or phenyl substituted with (C₁-C₃ alkylene)NR⁹R¹⁰, then A is 5- to 10-membered heteroaryl, 9- to 10-membered carbocycle, or 9- to 10-membered heterocycle, wherein the 5- to 10-membered heteroaryl, 9- to 10-membered carbocycle, or 9- to 10-membered heterocycle of A is optionally further substituted with R⁶; and when R² is oxo, R³ and R⁴ are H, and A is phenyl or phenyl substituted with (C₁-C₃ alkylene)NR⁹R¹⁰, then B is 5- to 6-membered heteroaryl, 5- to 6-membered carbocycle, 5- to 6-membered heterocycle, or 9- to 10-membered heteroaryl, wherein the 5- to 6-membered heteroaryl, 5- to 6-membered carbocycle, 5- to 6-membered heterocycle, or 9- to 10-membered heteroaryl of B is optionally further substituted with R⁷. In some embodiments of the compound of Formula (I), when R² is oxo and B is phenyl or phenyl substituted with (C₁-C₃ alkylene)NR⁹R¹⁰, then A is 5- to 10-membered heteroaryl, 9- to 10-membered carbocycle, or 9- to 10-membered heterocycle, wherein the 5- to 10-membered heteroaryl, 9- to 10-membered carbocycle, or 9- to 10-membered heterocycle of A is optionally further substituted with R⁶; and when R² is oxo and A is phenyl or phenyl substituted with (C₁-C₃ alkylene)NR⁹R¹⁰, then B is 5- to 6-membered heteroaryl, 5- to 6-membered carbocycle, 5- to 6-membered heterocycle, or 9- to 10-membered heteroaryl, wherein the 5- to 6-membered heteroaryl, 5- to 6-membered carbocycle, 5- to 6-membered heterocycle, or 9- to 10-membered heteroaryl of B is optionally further substituted with R⁷.

In some embodiments of a compound of formula (I), the compound is other than the compounds in Table 1X, or a tautomer or isomer thereof, or a salt of any of the foregoing.

TABLE 1X

| Compound | |
|---|---|
| No. | Name |
| 1.1x | 2,3-diphenyl-3,4-dihydropyrido[2,3-b]pyrazin-6(2H)-one |
| 1.2x | 2-(4-(aminomethyl)phenyl)-3-phenyl-3,4- |
| | dihydropyrido[2,3-b]pyrazin-6(2H)-one |
| 1.3x | 3-(4-(aminomethyl)phenyl)-2-phenyl-3,4- |
| | dihydropyrido[2,3-b]pyrazin-6(2H)-one |

In some embodiments of the compound of Formula (I), A is selected from the group consisting of C₆-C₁₂ aryl and 5- to 10-membered heteroaryl, wherein the C₆-C₁₂ aryl and 5- to 10-membered heteroaryl of A is optionally further substituted with R⁶. In some embodiments of the compound of Formula (I), B is selected from the group consisting of phenyl and 5- to 6-membered heteroaryl, wherein the phenyl and 5- to 6-membered heteroaryl of B is optionally further substituted with R⁷. In some embodiments of the compound of Formula (I), A is selected from the group consisting of C₆-C₁₂ aryl and 5- to 10-membered heteroaryl, wherein the C₆-C₁₂ aryl and 5- to 10-membered heteroaryl of A is optionally further substituted with R⁶; and B is selected from the group consisting of phenyl and 5- to 6-membered heteroaryl, wherein the phenyl and 5- to 6-membered heteroaryl of B is optionally further substituted with R⁷.

In some embodiments of Formula (I), R^{a}, R^{b}, and R^{c} are independently C₁-C₆ alkyl, C₂-C₆ alkenyl, C₂-C₆ alkynyl, halogen, —CN, —OR⁸, —SR⁸, —NR⁹R¹⁰, —C(O)NR⁹R¹⁰, —NR⁸C(O)R⁹, —NR⁸C(O)NR⁹R¹⁰, —S(O)R⁸, —S(O)₂R⁸, —NR⁸S(O)R⁹, —NR⁸S(O)₂R⁹, —S(O)NR⁹R¹⁰, —S(O)₂NR⁹R¹⁰, C₃-C₆ cycloalkyl, 3-12-membered heterocyclyl, 5- to 10-membered heteroaryl or C₆-C₁₄ aryl.

In some embodiments of Formula (I), R^{a}, R^{b}, and R^{c} are independently C₁-C₆ alkyl, C₂-C₆ alkenyl, C₂-C₆ alkynyl, halogen, —CN, —OR⁸, —SR⁸, —NR⁹R¹⁰, —C(O)NR⁹R¹⁰, —NR⁸C(O)R⁹, C₃-C₆ cycloalkyl, 3-12-membered heterocyclyl, 5- to 10-membered heteroaryl or C₆-C₁₄ aryl.

In some embodiments of Formula (I), R^{a}, R^{b}, and R^{c} are independently C₁-C₆ alkyl, halogen, —CN, —OR⁸, —SR⁸ or —NR⁹R¹⁰.

In some embodiments of Formula (I), R^{a}, R^{b}, and R^{c} are independently —CH₃, halogen, —CN or —OCH₃.

In some embodiments of a compound of Formula (I), R² is H, oxo, C₁-C₆ alkyl, C₂-C₆ alkenyl, C₂-C₆ alkynyl, C₃-C₆ cycloalkyl, 3-12-membered heterocyclyl, 5- to 10-membered heteroaryl, C₆-C₁₄ aryl, halogen, —CN, —OR⁸, —C(O)NR⁹R¹⁰, —C(O)R⁸, —C(O)OR⁸, —OC(O)R⁸, —OC(O)NR⁹R¹⁰, —NR⁸C(O)R⁹, —NR⁸C(O)OR⁹, —NR⁸C(O)NR⁹R¹⁰, —S(O)₂R⁸, —NR⁸S(O)R⁹, —NR⁸S (O)₂R⁹, —C(O)NR⁸S(O)R⁹, —C(O)NR⁸S(O)₂R⁹, —S(O)NR⁹R¹⁰, —S(O)₂NR⁹R¹⁰, —(C₁-C₃ alkylene)CN, —(C₁-C₃ alkylene)SR⁸, —(C₁-C₃ alkylene)NR⁹R¹⁰, —(C₁-C₃ alkylene)C(O)R⁸, —(C₁-C₃ alkylene)C(O)NR⁹R¹⁰, —(C₁-C₃ alkylene)NR⁸C(O)R⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)(C₃-C₆ cycloalkyl), —S(O)R⁸, —(C₁-C₃ alkylene)OR⁸, or —(C₁-C₃ alkylene)(5-10-membered heteroaryl), each of which is optionally substituted by halogen, oxo, —OR¹¹, —NR¹¹R¹², —C(O)R¹¹, —CN, —S(O)R¹¹, —S(O)₂R¹¹, —P(O)(OR^{1I})(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR^{1I})(OR¹²), C₃-C₈ cycloalkyl, or C₁-C₆ alkyl optionally substituted by oxo, —OH or halogen. In some embodiments, R² is H. In some embodiments, R² is oxo.

In some embodiments of a compound of Formula (I), R² is selected from the group consisting of: H, oxo, methyl, phenyl, cyclopropyl, fluoro, chloro, bromo, —CN, methoxy,

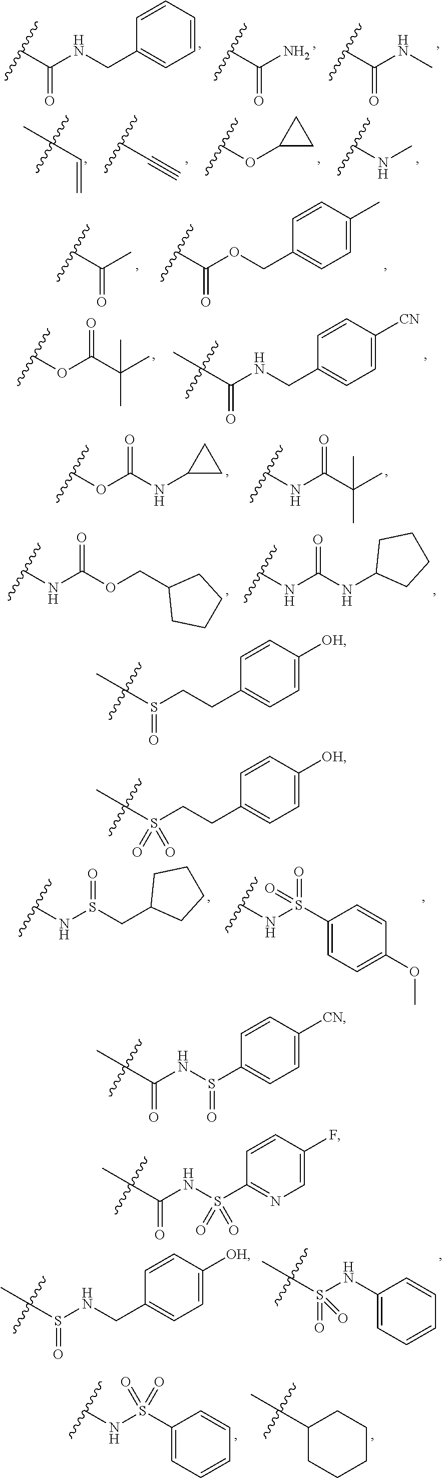

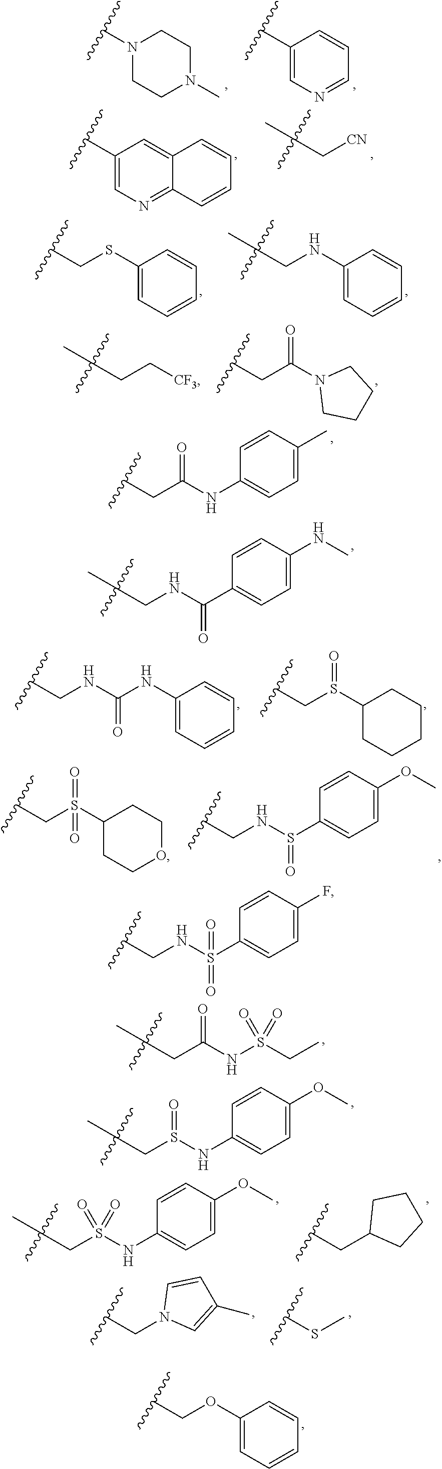

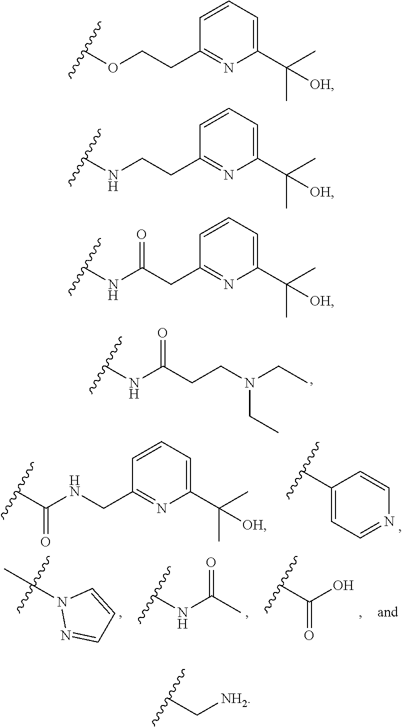

In some embodiments of a compound of Formula (I), R³ is H, —CN, halogen, C₁-C₆ alkyl, C₂-C₆ alkenyl, C₂-C₆ alkynyl, C₃-C₈ cycloalkyl, 3-12-membered heterocyclyl, 5- to 10-membered heteroaryl, C₆-C₁₄ aryl, —OR⁸, —C(O)NR⁹R¹⁰, —(C₁-C₃ alkylene)NR⁹R¹⁰, —NR⁹R¹⁰, —C(O)NR⁹R¹⁰, —NR⁸C(O)R⁹, —SR⁸, —C(O)R⁹, —OC(O)R⁸, —C(O)OR⁸, —OC(O)NR⁹R¹⁰, —NR⁸(O)OR⁹, —NR⁸(O)NR⁹R¹⁰, —S(O)R⁸, —S(O)₂R⁸, —NR⁸S(O)R⁹, —NR⁸S(O)₂R⁹, —C(O)NR⁸S(O)R⁹, —S(O)NR⁹R¹⁰, —S(O)₂NR⁹R¹⁰, —(C₁-C₃ alkylene)CN, —(C₁-C₃ alkylene)OR⁸, —(C₁-C₃ alkylene)SR⁸, —(C₁-C₃ alkylene) (3-12-membered heterocyclyl), —(C₁-C₃ alkylene(C₆-C₁₄ aryl), —(C₁-C₃ alkylene)C(O)R⁸, —(C₁-C₃ alkylene)C(O)NR⁹R¹⁰, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —(C₁-C₃ alkylene)NR⁹S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —C(O)NR⁹S(O)₂R⁹, or —(C₁-C₃ alkylene)NR⁸C(O)R⁹, each of which is optionally substituted by halogen, oxo, —OR¹¹, —NR¹¹R¹², —C(O)R¹¹, —CN, —S(O)R¹¹, —S(O)₂R¹¹, —P(O)(OR¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen. In some embodiments, R³ is H. In some embodiments, R³ is halogen. In some embodiments, R³ is C₁-C₆ alkyl. In some embodiments, R³ is —NR⁹R¹⁰.

In some embodiments of a compound of Formula (I), R³ is selected from the group consisting of: H, —CN, bromo, fluoro, chloro, methyl, cyclopropyl,

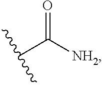 [SMILES: *C(N)=O]

hydroxy, methoxy,

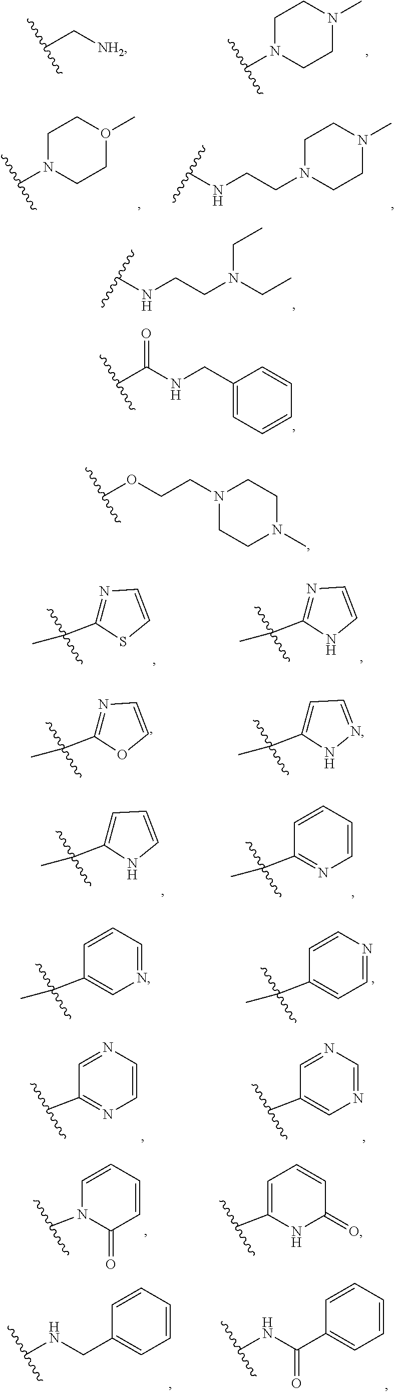

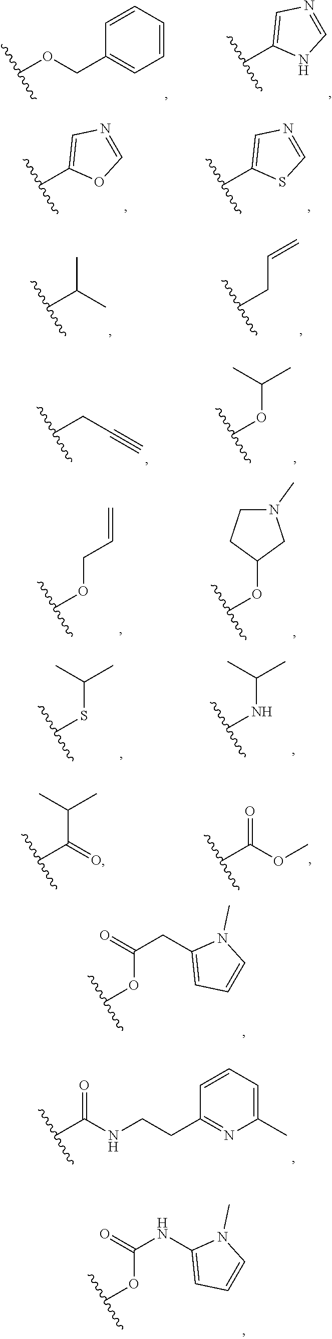

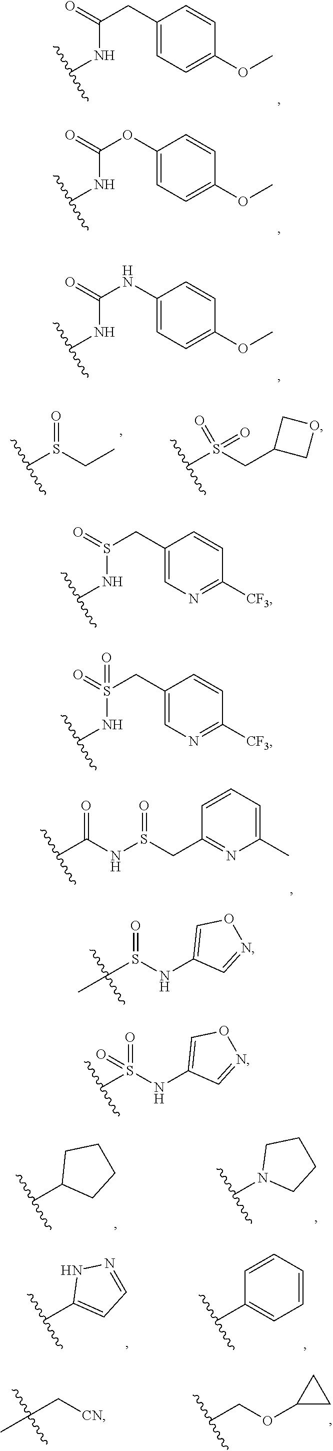

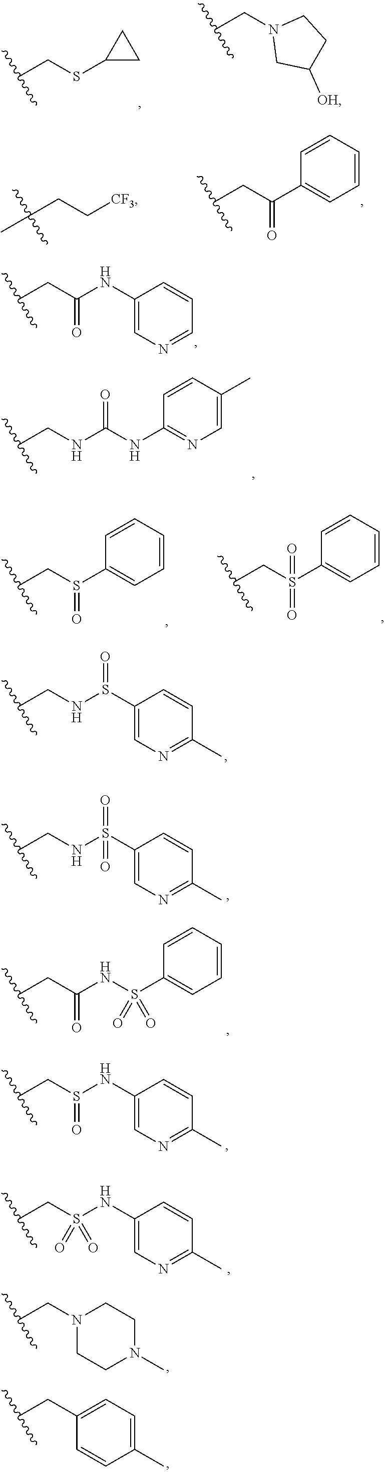

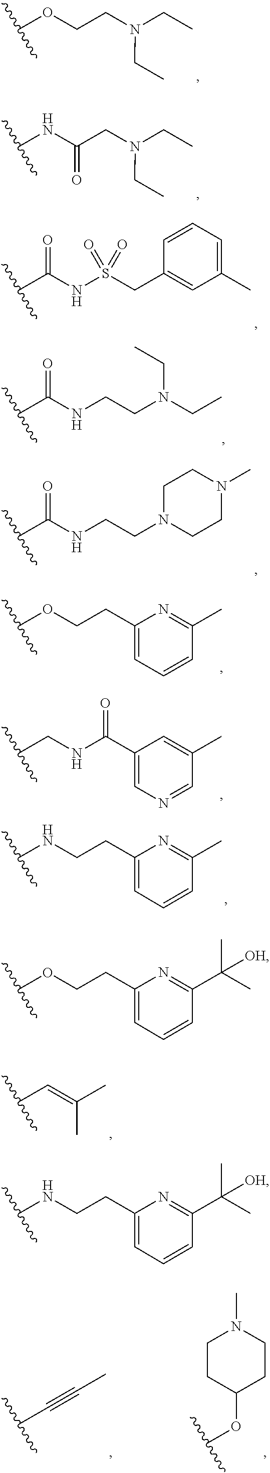

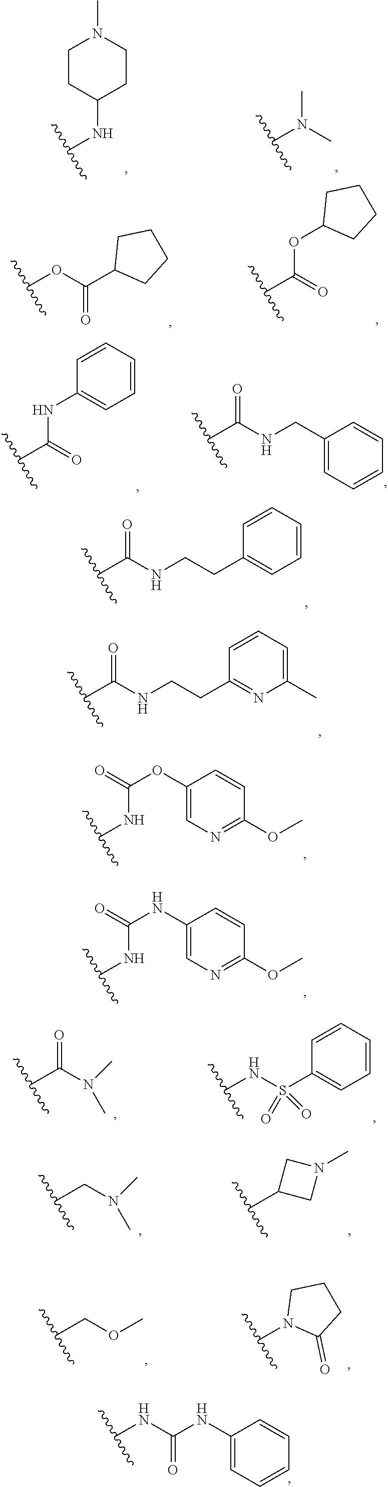

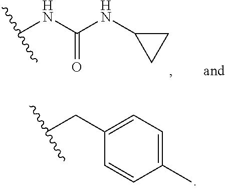

In some embodiments of the compound of Formula (I), R⁴ is H, oxo, C₁-C₆ alkyl, C₂-C₆ alkenyl, C₂-C₆ alkynyl, —CN, —NR⁹R¹⁰, halogen, 3-12-membered heterocyclyl, 5- to 10-membered heteroaryl, C₆-C₁₄ aryl, —C(O)NR⁹R¹⁰, —OR⁸, —SR⁸, —C(O)OR⁹, or —NR⁸C(O)R⁹, each of which is independently optionally substituted by halogen, oxo, —OR¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen. In some embodiments, R⁴ is H. In some embodiments, R⁴ is oxo. In some embodiments, R⁴ is C₁-C₆ alkyl. In some embodiments, R⁴ is —NR⁹R¹⁰. In some embodiments, R⁴ is —OR⁸.

In some embodiments of the compound of Formula (I), R⁴ is selected from the group consisting of: H, oxo, methyl, ethyl,

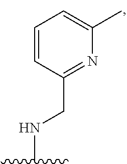 [SMILES: *NCc1cccc(C)n1]

—CN, phenyl, cyclopropyl, bromo, chloro, methoxy,

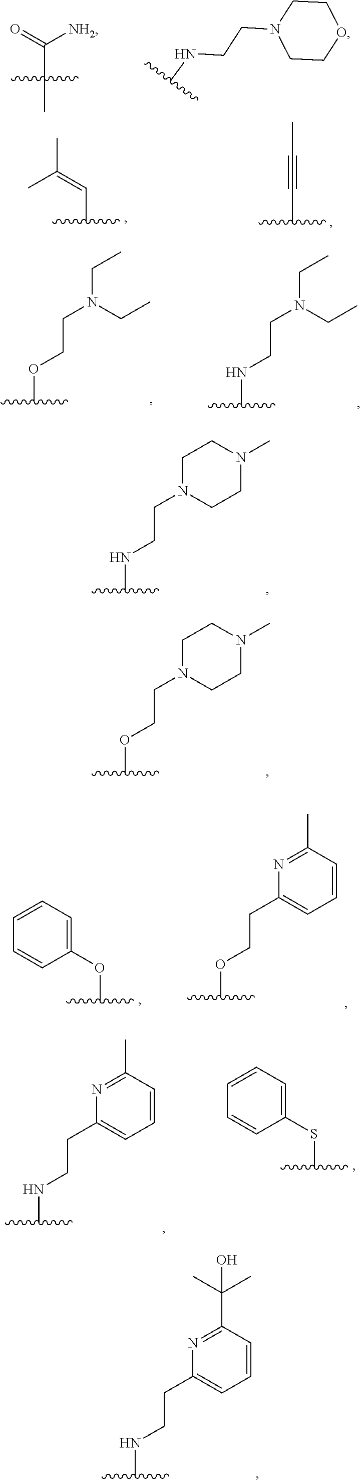

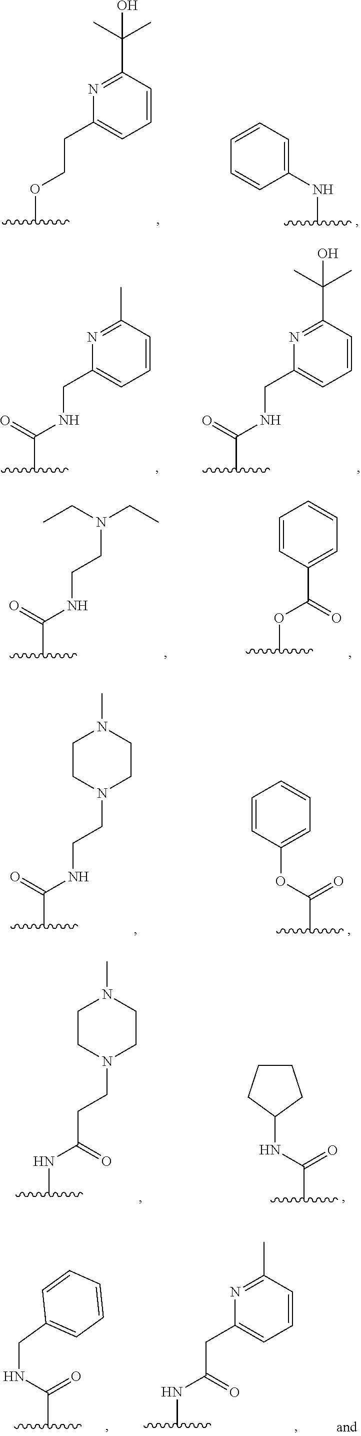

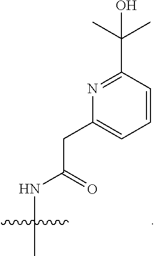 [SMILES: *NC(=O)Cc1cccc(C(C)(C)O)n1]

In some embodiments of the compound of Formula (I), R¹, R² and R³ are each H and R⁴ is oxo. In some embodiments of the compound of Formula (I), R¹, R³ and R⁴ are each H and R² is oxo.

In some embodiments, provided is a compound of Formula (II):

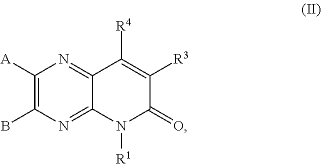

or a tautomer or isomer thereof, or a pharmaceutically acceptable salt of any of the foregoing, wherein A, B, R¹, R³ and R⁴ are as defined for Formula (I).

In some embodiments of a compound of Formula (II), at least one of R³ and R⁴ is not H. In some embodiments, at least one of R³ and R⁴ is C₁-C₆ alkyl, halogen, C₆-C₁₄ aryl, —CN, or —OR⁸. In some embodiments, R¹, R³ and R⁴ are each H.

In some embodiments, provided is a compound of Formula (III):

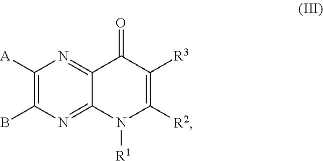

or a tautomer or isomer thereof, or a pharmaceutically acceptable salt of any of the foregoing, wherein A, B, R¹, R² and R³ are as defined for Formula (I).

In some embodiments of a compound of Formula (III), at least one of R² and R³ is not H. In some embodiments, at least one of R² and R³ is C₁-C₆ alkyl, halogen, C₆-C₁₄ aryl, —CN, or —OR⁸. In some embodiments, R¹, R² and R³ are each H.

In some embodiments of a compound of Formula (I), (II), or (III), R¹ is C₁-C₆ alkyl, such as methyl, ethyl, n-propyl, isopropyl, n-butyl, t-butyl, or isobutyl. In some embodiments of a compound of Formula (I), (II), or (III), R¹ is H or C₁-C₆ alkyl. In some embodiments of a compound of Formula (I), (II), or (III), R¹ is H or methyl. In some embodiments, R¹ is H.

In some embodiments of a compound of Formula (I) or (III), R² is H, C₁-C₆ alkyl, C₂-C₆ alkenyl, C₂-C₆ alkynyl, C₃-C₆ cycloalkyl, 3-12-membered heterocyclyl, 5- to 10-membered heteroaryl, C₆-C₁₄ aryl, halogen, —CN, —OR⁸, —C(O)NR⁹R¹⁰, —C(O)R⁹, —C(O)OR⁸, —OC(O)R⁸, —OC(O)NR⁹R¹⁰, —NR⁸C(O)R⁹, —NR⁸C(O)OR⁹, —NR⁸C(O)NR⁹R¹⁰, —S(O)₂R⁸, —NR⁸S(O)R⁹, —NR⁸S(O)₂R⁹, —C(O)NR⁸S(O)R⁹, —C(O)NR⁸S(O)₂R⁹, —S(O)NR⁹R¹⁰, —S(O)₂NR⁹R¹⁰, —(C₁-C₃ alkylene)CN, —(C₁-C₃ alkylene)SR⁸, —(C₁-C₃ alkylene)NR⁹R¹⁰, —(C₁-C₃ alkylene)C(O)R⁸, —(C₁-C₃ alkylene)C(O)NR⁹R¹⁰, —(C₁-C₃ alkylene)NR⁸C(O)R⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)(C₃-C₆ cycloalkyl), —S(O)R⁸, —(C₁-C₃ alkylene)OR⁸, or —(C₁-C₃ alkylene)(5-10-membered heteroaryl), each of which is optionally substituted by halogen, oxo, —OR¹¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen. In some embodiments, R² is H.

In some embodiments of a compound of Formula (I) or (III), R² is selected from the group consisting of: H, methyl, phenyl, cyclopropyl, fluoro, chloro, bromo, —CN, methoxy,

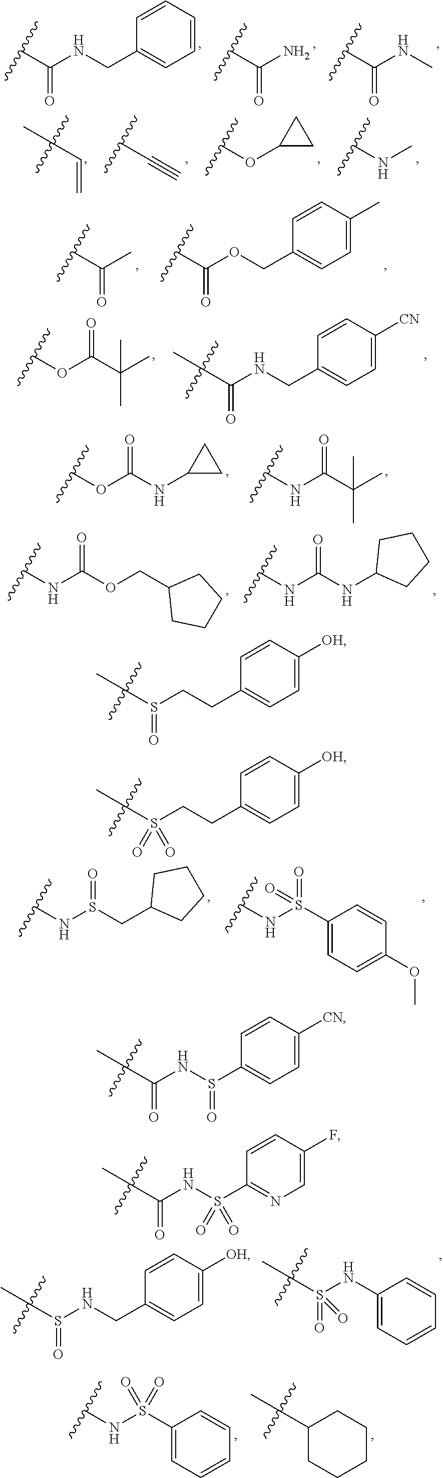

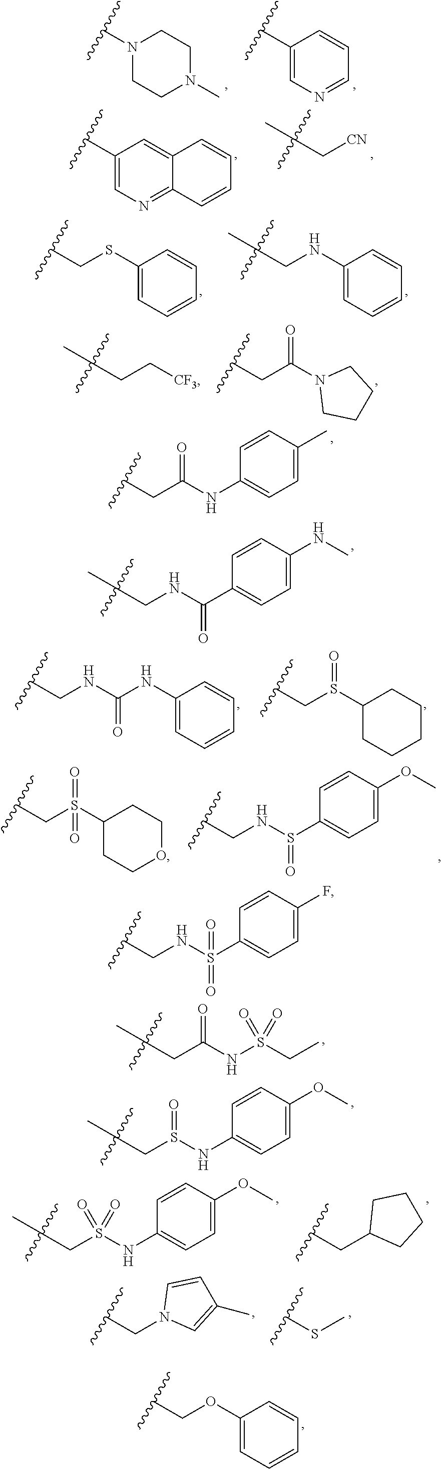

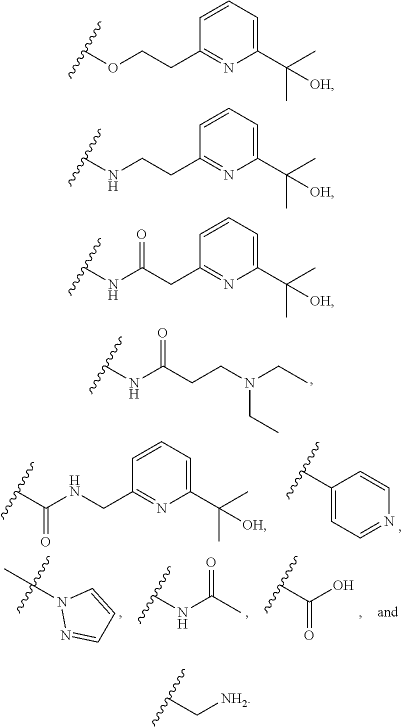

In some embodiments of a compound of Formula (I) or (II), R⁴ is H, C₁-C₆ alkyl, C₂-C₆ alkenyl, C₂-C₆ alkynyl, —CN, —NR⁹R¹⁰, halogen, 3-12-membered heterocyclyl, 5- to 10-membered heteroaryl, C₆-C₁₄ aryl, —C(O)NR⁹R¹⁰, —OR⁸, —SR⁸, —C(O)OR⁸, or —NR⁸C(O)R⁹, each of which is independently optionally substituted by halogen, oxo, —OR¹¹, —NR¹¹R¹², —C(O)R¹¹, C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen. In some embodiments, R¹⁴ is C₁-C₆ alkyl, C₂-C₆ alkenyl, C₂-C₈ alkynyl, halogen, —CN, —OR⁸, —SR⁸, —NR⁹R¹⁰, —C(O)NR⁹R¹⁰, —NR⁸C(O)R⁹, —NR⁸C(O)NR⁹R¹⁰, —S(O)R⁸, —S(O)₂R⁸, —NR⁸S(O)R⁹, —NR⁸S(O)₂R⁹, —S(O)NR⁹R¹⁰, —S(O)₂NR⁹R¹⁰, C₃-C₆ cycloalkyl, 3-12-membered heterocyclyl, 5- to 10-membered heteroaryl or C₆-C₁₄ aryl. In some embodiments of a compound of Formula (I), (II), or (III), R⁴ is C₁-C₈ alkyl, C₂-C₈ alkenyl, C₂-C₆ alkynyl, halogen, —CN, —OR⁸, —SR⁸, —NR⁹R¹⁰, —C(O)NR⁹R¹⁰, —NR⁸C(O)R⁹, C₃-C₆ cycloalkyl, 3-12-membered heterocyclyl, 5- to 10-membered heteroaryl or C₆-C₁₄ aryl. In some embodiments of a compound of Formula (I), (II), or (III), R⁴ is C₁-C₆ alkyl, halogen, —CN, —OR⁸, —SR⁸ or —NR⁹R¹⁰. In some embodiments of a compound of Formula (I), (II), or (III), R⁴ is —CH₃, halogen, —CN or —OCH₃. In some embodiments of a compound of Formula (I), (II), or (III), R⁴ is H or methyl. In some embodiments of a compound of Formula (I), (II), or (III), R⁴ is H, C₁-C₆ alkyl, halogen, —CN, or —OR⁸.

In some embodiments of a compound of formula (I) or (II), R⁴ is selected from the group consisting of: H, methyl, ethyl,

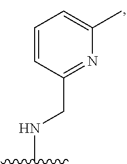 [SMILES: *NCc1cccc(C)n1]

—CN, phenyl, cyclopropyl, bromo, chloro, methoxy,

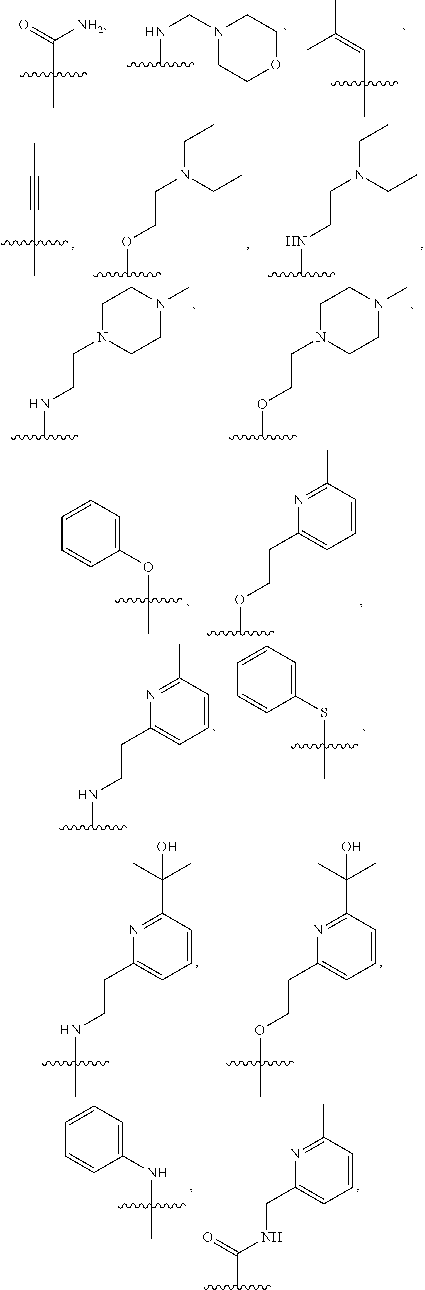

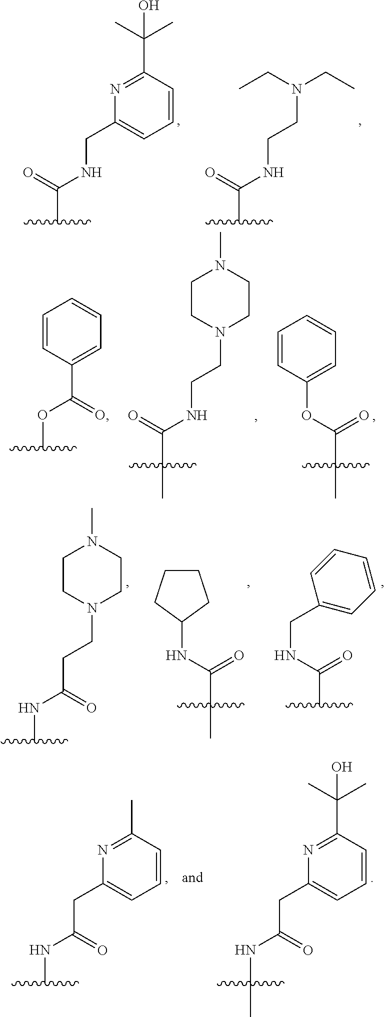

In some embodiments of a compound of Formula (I), (II), or (III), A is C₆-C₁₂ aryl optionally further substituted with R⁶. In some embodiments, A is C₆-C₁₂ aryl optionally substituted with R⁶, wherein each R⁶ is independently C₁-C₆ alkyl optionally substituted with halogen, halogen, —CN, —OR⁸, —NR⁹R¹⁰, or —NR⁹S(O)₂R⁹. In some embodiments, A is phenyl or naphthyl, optionally substituted with R⁶. In some embodiments, A is phenyl. In some embodiments, A is naphthyl. In some embodiments, A is phenyl or naphthyl, substituted with one or more groups selected from halogen, —CN, —OR⁸, —SR⁸, —NR⁹R¹⁰, —NO₂, —C(O)R¹, —C(O)OR⁸, —C(O)NR⁹R¹⁰, —NR⁹S(O)₂R⁹, —C(O)NR⁸S(O)₂R⁹, —OC(O)R⁹, —OC(O)NR⁹R¹⁰, —NR⁸C(O)R⁹, —NR⁸C(O)NR⁹R¹⁰, —S(O)R⁹, —S(O)₂R⁸, C₃-C₆ cycloalkyl and C₁-C₆ alkyl optionally substituted by halogen. In some embodiments, A is phenyl substituted with one or more groups selected from halogen, —CN, —OR⁸, —NR⁹R¹⁰, and —NR⁹S(O)₂R⁹. In some embodiments, A is phenyl, substituted with one or more groups selected from halogen, —CN, —OH, —OC₁-C₆ alkyl, —NH₂, —NO₂, C₃-C₆ cycloalkyl and C₁-C₆ alkyl optionally substituted by halogen. In some embodiments, A is phenyl, substituted with one or more groups selected from halogen, —OH, and C₁-C₆ alkyl.

In some embodiments of a compound of Formula (I), (II), or (III), A is 5- to 10-membered heteroaryl optionally further substituted with R⁶. In some embodiments, A is 5- to 10-membered heteroaryl optionally substituted with R⁶, wherein each R⁶ is independently C₁-C₆ alkyl optionally substituted with halogen, halogen, oxo, —CN, C₃-C₆ cycloalkyl, —OR⁸, or —(C₁-C₃ alkylene)(C6-C₁₄ aryl). In some embodiments, A is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl and imidazothiazolyl, each optionally substituted with R⁶. In one variation, the optional substitution with R⁶ provides a moiety that is unsubstituted. In one variation, the optional substitution with R⁶ provides a moiety that is substituted with 1-3 R⁶, which may be the same or different. In some embodiments, A is 5- to 10-membered heteroaryl optionally further substituted with one or more groups selected from halogen, —CN, —OR⁸, —SR⁸, —NR⁹R¹⁰, —NO₂, —C(O)R⁹, —C(O)OR⁸, —C(O)NR⁹R¹⁰, —C(O)NR⁸S(O)₂R⁹, —OC(O)R⁹, —OC(O)NR⁹R¹⁰, —NR⁸C(O)R⁹, —NR⁸C(O)NR⁹R¹⁰, —S(O)R⁹, —S(O)₂R⁸, C₃-C₆ cycloalkyl and C₁-C₆ alkyl optionally substituted by halogen. In some embodiments, A is 5- to 10-membered heteroaryl optionally further substituted with one or more groups selected from C₁-C₆ alkyl, halogen, —CN, —OH, and —OC₁-C₆ alkyl.

In some embodiments, A is a 10-membered heteroaryl optionally further substituted with R⁶, wherein the 10-membered heteroaryl is a 6/6-ring fused system (i.e., a ring system formed by fusing a 6-membered ring with a 6-membered ring). In some embodiments, A is a 9-membered heteroaryl, wherein the 9-membered heteroaryl is a 6/5-ring fused system (i.e., a ring system formed by fusing a 6-membered ring with a 5-membered ring). In some embodiments, the 6/5-ring fused system of A is attached to the rest of the compound via the 6-membered ring. In other embodiments, the 6/5-ring fused system of A is attached to the rest of the compound via the 5-membered ring.

In some embodiments, A is selected from the group consisting of:

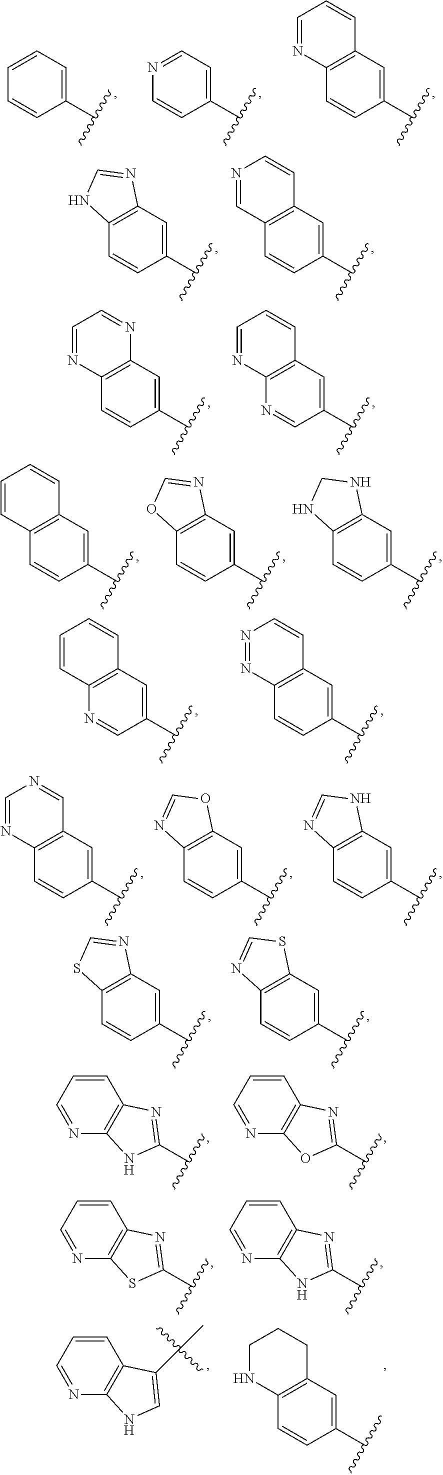

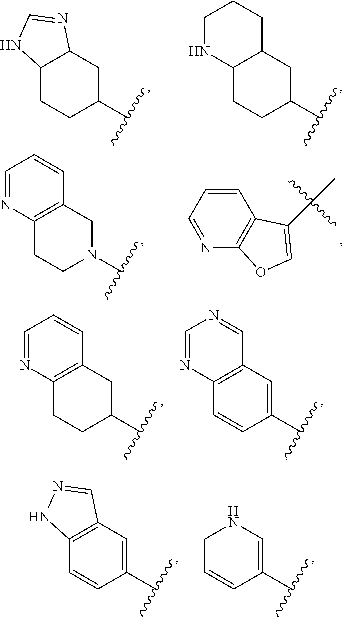

each optionally substituted with R⁶ and where the wavy line denotes attachment to the parent structure. In one variation, such groups are not further substituted with R⁶. In some of these embodiments, R⁶ is independently C₁-C₆ alkyl optionally substituted with halogen, halogen, oxo, C₃-C₆ cycloalkyl, —(C₁-C₃ alkylene)(C₆-C₁₄ aryl), —CN, —OR⁸, —NR⁹R¹⁰, or —NR⁹S(O)₂R⁹. In some of these embodiments, such groups are substituted with 1-3 R⁶, which may be the same or different.

In some embodiments of a compound of Formula (I), (II), or (III), A is a 9- to 10-membered carbocycle optionally further substituted with R⁶. In some embodiments, A is a 10-membered carbocycle, wherein the 10-membered carbocycle is a 6/6-ring fused system (i.e., a ring system formed by fusing a 6-membered ring with a 6-membered ring). In some embodiments, A is a 9-membered carbocycle, wherein the 9-membered carbocycle is a 6/5-ring fused system (i.e., a ring system formed by fusing a 6-membered ring with a 5-membered ring). In some embodiments, the 6/5-ring fused system of A is attached to the rest of the compound via the 6-membered ring. In other embodiments, the 6/5-ring fused system of A is attached to the rest of the compound via the 5-membered ring. In some embodiments, A is a fully saturated 9- to 10-membered carbocycle. In some embodiments, A is a partially saturated 9- to 10-membered carbocycle. In some embodiments of a compound of Formula (I), (II), or (III), A is selected from the group consisting of decahydronaphthalenyl, octahydroindenyl, 1,2,3,4-tetrahydronaphthalenyl, and 2,3-dihydroindenyl, each optionally substituted with R⁶. In some embodiments, A is a 9- to 10-membered carbocycle optionally further substituted with one or more groups selected from halogen, —CN, —OR⁸, —SR⁸, —NR⁹R¹⁰, —NO₂, —C(O)R⁹, —C(O)OR⁸, —C(O)NR⁹R¹⁰, —C(O)NR⁸S(O)₂R⁹, —OC(O)R⁸, —OC(O)NR⁹R¹⁰, —NR⁸C(O)R⁹, —NR⁸C(O)NR⁹R¹⁰, —S(O)R⁹, —S(O)₂R⁸, C₃-C₆ cycloalkyl and C₁-C₆ alkyl optionally substituted by halogen. In some embodiments, A is a 9- to 10-membered carbocycle optionally further substituted with one or more groups selected from C₁-C₆ alkyl, halogen, —CN, —OH, and —OC₁-C₆ alkyl.

In some embodiments, A is a 9- to 10-membered heterocycle optionally further substituted with R⁶. In some embodiments, A is a 10-membered heterocycle optionally further substituted with R⁶, wherein the 10-membered heterocycle is a 6/6-ring fused system (i.e., a ring system formed by fusing a 6-membered ring with a 6-membered ring). In some embodiments, A is a 9-membered heterocycle, wherein the 9-membered heterocycle is a 6/5-ring fused system (i.e., a ring system formed by fusing a 6-membered ring with a 5-membered ring). In some embodiments, the 6/5-ring fused system of A is attached to the rest of the compound via the 6-membered ring. In other embodiments, the 6/5-ring fused system of A is attached to the rest of the compound via the 5-membered ring. In some embodiments, A is a fully saturated 9- to 10-membered heterocycle. In some embodiments, A is a partially saturated 9- to 10-membered heterocycle. In some embodiments, A is selected from the group consisting of tetrahydroquinolinyl, tetrahydroisoquinolinyl, decahydroquinolinyl, decahydroisoquinolinyl, indolinyl, isoindolinyl, tetrahydronaphthyridinyl and hexahydrobenzoimidazolyl, each optionally further substituted with R⁶. In some embodiments, A is a 9- to 10-membered heterocycle optionally further substituted with one or more groups selected from halogen, —CN, —OR⁸, —SR⁸, —NR⁹R¹⁰, —NO₂, —C(O)R⁸, —C(O)OR⁸, —C(O)NR⁹R¹⁰, —C(O)NR⁸S(O)₂R⁹, —OC(O)R⁸, —OC(O)NR⁹R¹⁰, —NR⁸C(O)R⁹, —NR⁸C(O)NR⁹R¹⁰, —S(O)R⁹, —S(O)₂R⁸, C₃-C₆ cycloalkyl and C₁-C₆ alkyl optionally substituted by halogen. In some embodiments, A is a 9- to 10-membered heterocycle optionally further substituted with one or more groups selected from C₁-C₆ alkyl, halogen, —CN, —OH, and —OC₁-C₆ alkyl. In some embodiments, A is selected from the group consisting of

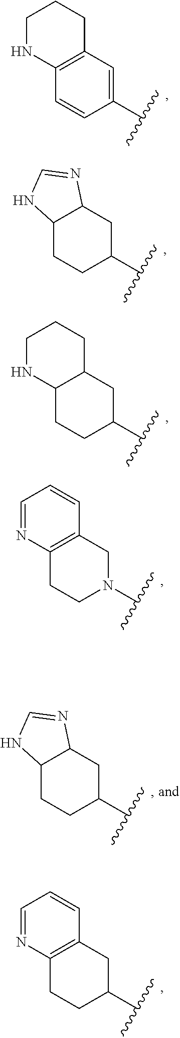 [SMILES: *c1ccc2c(c1)CCCN2 ; *C1CCC2NC=NC2C1 ; *C1CCC2NCCCC2C1 ; *N1CCc2ncccc2C1 ; *C1CCC2NC=NC2C1 ; *C1CCc2ncccc2C1]

each optionally substituted with R⁶. In some embodiments, such groups are not further substituted with R⁶. In some embodiments, such groups are further substituted with 1-3 R⁶, which may be the same or different. In some of these embodiments R⁶ is independently selected from the group consisting of halogen, —CN, —OR⁸, —SR⁸, —NR⁹R¹⁰, —NO₂, —C(O)R⁸, —C(O)OR⁹, —C(O)NR⁹R¹⁰, —C(O)NR⁸S(O)₂R⁹, —OC(O)R⁸, —OC(O)NR⁹R¹⁰, —NR⁸C(O)R⁹, —NR⁸C(O)NR⁹R¹⁰, —S(O)R⁸, —S(O)₂R⁸, C₃-C₆ cycloalkyl and C₁-C₆ alkyl optionally substituted by halogen. In some embodiments, R⁶ is independently selected from the group consisting of C₁-C₆ alkyl, halogen, —CN, —OH, and —OC₁-C₆ alkyl.

In some embodiments of a compound of Formula (I), (II), or (III), each R⁶ is independently selected from the group consisting of halogen, —CN, —OR⁸, —SR⁸, —NR⁹R¹⁰, —NO₂, —C(O)R⁸, —C(O)OR⁹, —C(O)NR⁹R¹⁰, —C(O)NR⁸S(O)₂R⁹, —OC(O)R⁸, —OC(O)NR⁹R¹⁰, —NR⁸C(O)R⁹, —NR⁸C(O)NR⁹R¹⁰, —S(O)R⁸, —S(O)₂R⁸, C₃-C₆ cycloalkyl and C₁-C₆ alkyl optionally substituted by halogen. In some embodiments, R⁶ is independently selected from the group consisting of C₁-C₆ alkyl, halogen, —CN, and —OR⁸.

In some embodiments of a compound of Formula (I), (II), or (III), A is selected from the group consisting of:

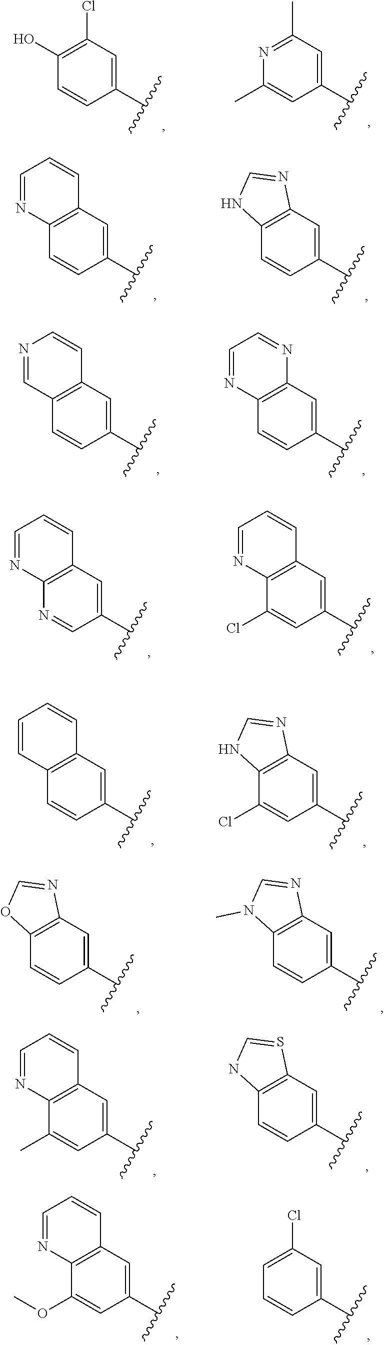

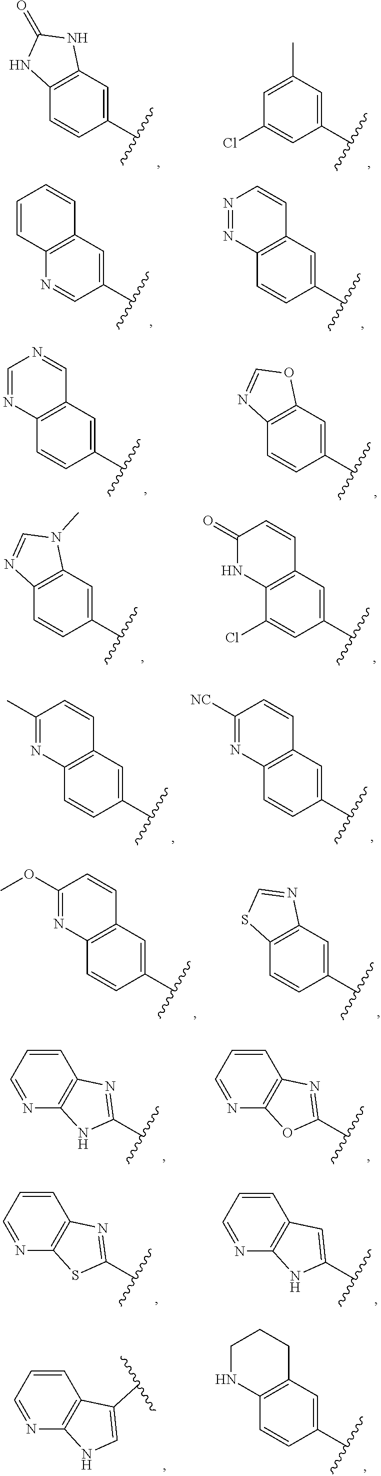

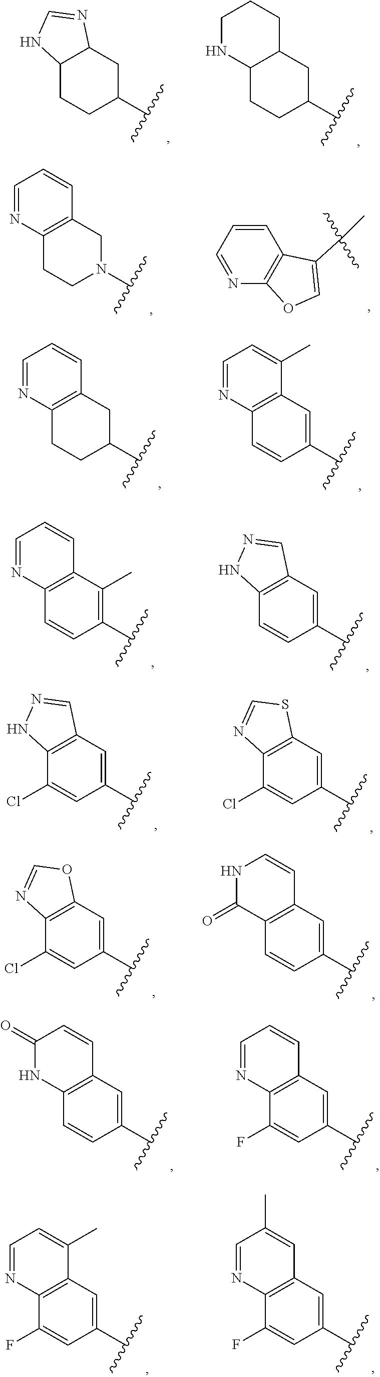

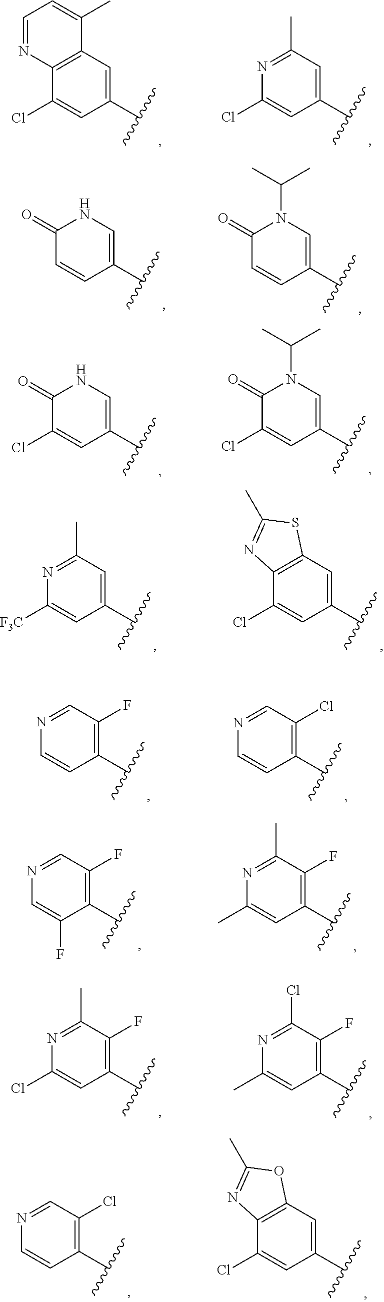

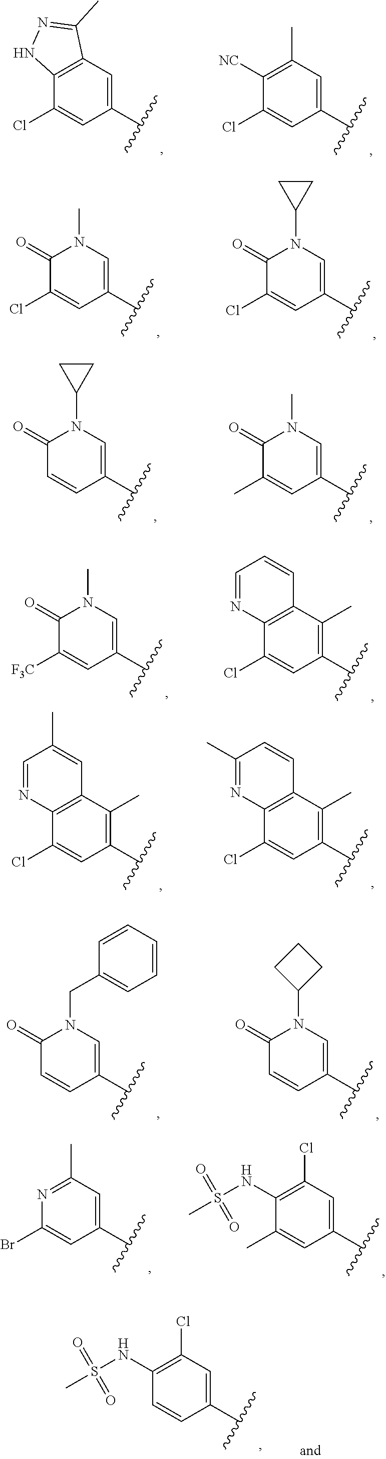

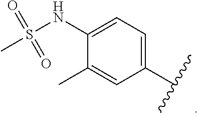 [SMILES: *c1ccc(NS(C)(=O)=O)c(C)c1]

In some embodiments of a compound of Formula (I), (II), or (III), A is selected from the group consisting of:

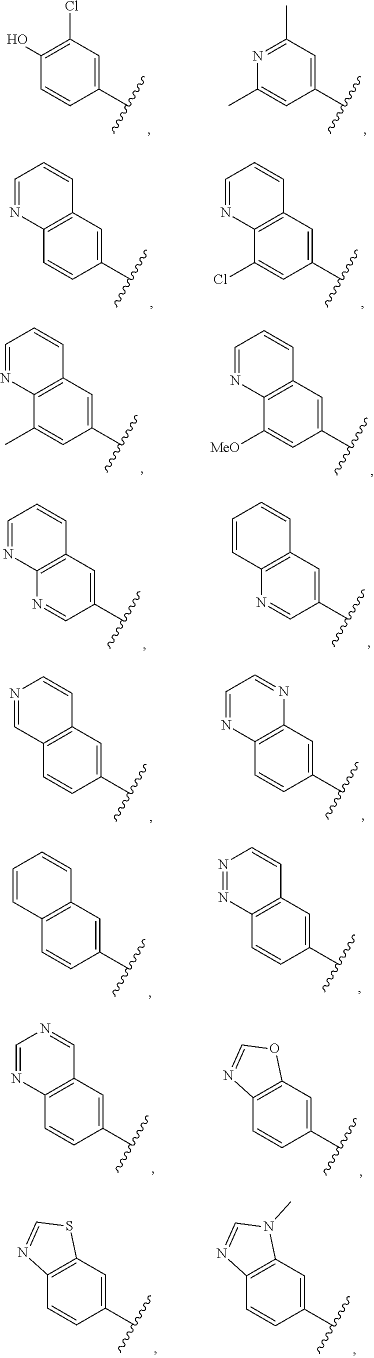

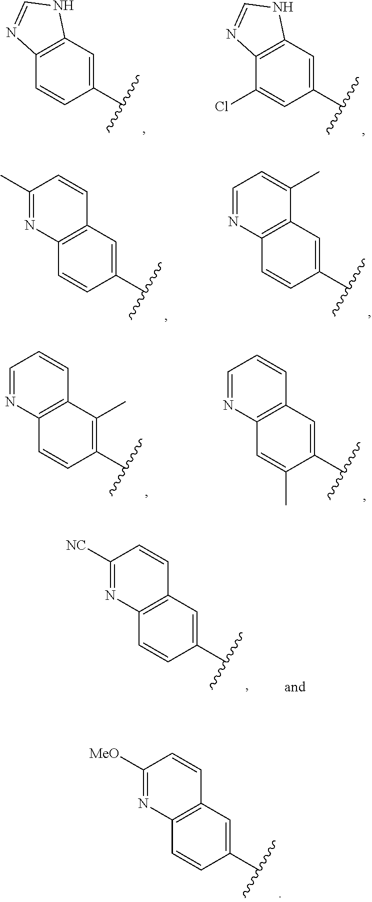

In some embodiments, A is

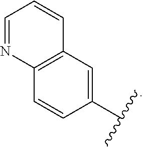 [SMILES: *c1ccc2ncccc2c1]

In some embodiments of a compound of Formula (I), (II), or (III), A is

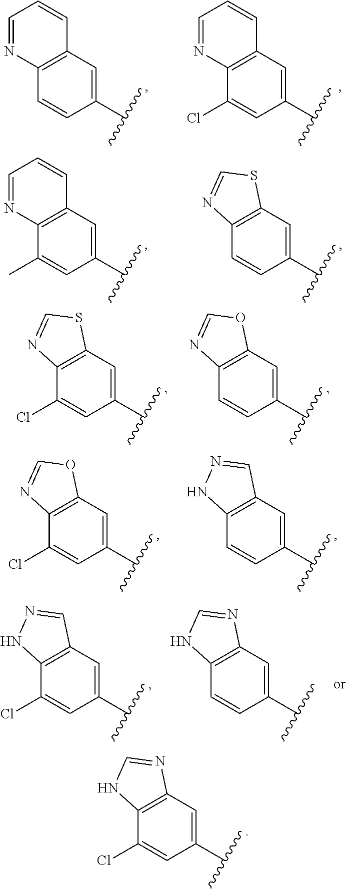

In some embodiments of a compound of Formula (I), (II), or (III), A is

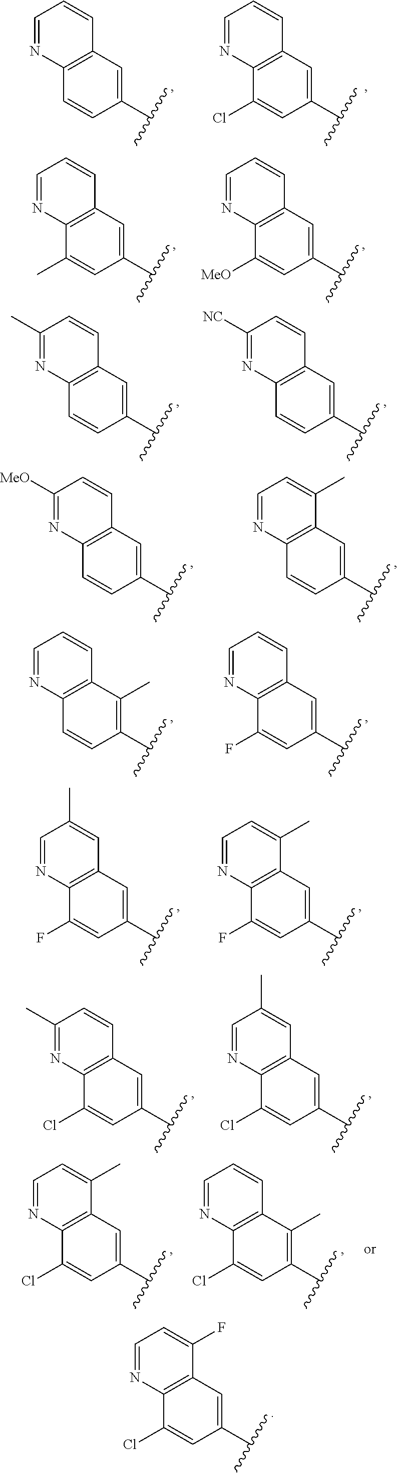

It is understood that each description of A may be combined with each description of R¹-R⁴ the same as if each and every combination were specifically and individually listed. It is similarly understood that each description of A may be combined with each description of B (and further with each description of R¹-R⁴) the same as if each and every combination were specifically and individually listed. For example, in one aspect, it is understood that each description of A may be combined in one aspect with a variation in which R¹, R³ are each hydrogen and one of R² and R⁴ is hydrogen and one of R² and R⁴ is oxo. In one such variation, each description of A is combined in one aspect with a variation in which R¹, R², R³ are each hydrogen and R⁴ is oxo. In another such variation, each description of A is combined in one aspect with a variation in which R¹, R³, R⁴ are each hydrogen and R² is oxo. Such embodiments may further be combined with each description of B.

In some embodiments of a compound of Formula (I), (II), or (III), B is phenyl, optionally further substituted with R⁷. In some embodiments, B is 5- to 6-membered heteroaryl optionally further substituted with R⁷. In some embodiments, B is pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridyl, pyridazinyl, pyrimidinyl, pyrazinyl, triazinyl, or tetrazinyl, each optionally substituted with R⁷. In some embodiments, B is furanyl, pyridinyl, oxazoyl, or oxadiazoyl, each optionally substituted with R⁷. In some of these embodiments, R⁷ is independently halogen, C₁-C₆ alkyl, or —OR⁸.

In some embodiments of a compound of Formula (I), (II), or (III), B is a 5- to 6-membered carbocycle optionally further substituted with R⁷. In some embodiments, B is a fully saturated 5- to 6-membered carbocycle optionally further substituted with R⁷. In some embodiments, B is cyclopentyl or cyclohexyl, optionally further substituted with R⁷. In some embodiments, B is a 5- to 6-membered carbocycle optionally substituted with one or more groups selected from halogen, —CN, —OR⁸, —SR⁸, —NR⁹R¹⁰, —NO₂, —C(O)R⁹, —C(O)OR⁸, —C(O)NR⁹R¹⁰, —C(O)NR⁹S(O)₂R⁹, —OC(O)R⁹, —OC(O)NR⁹R¹⁰, —NR⁸C(O)R⁹, —NR⁸C(O)NR⁹R¹⁰, —S(O)R⁸, —S(O)₂R⁸, C₃-C₆ cycloalkyl and C₁-C₆ alkyl optionally substituted by halogen. In some embodiments, B is a 5- to 6-membered carbocycle optionally substituted with halogen.

In some embodiments of a compound of Formula (I), (II), or (III), B is a 5- to 6-membered heterocycle optionally further substituted with R⁷. In some embodiments, B is a fully saturated 5- to 6-membered heterocycle optionally further substituted with R⁷. In some embodiments, B is pyrrolidinyl, pyrazolidinyl, imidazolidinyl, tetrahydrofuranyl, 1,3-dioxolanyl, tetrahydrothiophenyl, oxathiolanyl, sulfolanyl, piperidinyl, piperazinyl, tetrahydropyranyl, dioxanyl, thianyl, dithianyl, trithianyl, morpholinyl, thiomorpholinyl optionally further substituted with R⁷. In some embodiments, B is a 5- to 6-membered heterocycle optionally substituted with one or more groups selected from halogen, —CN, —OR⁸, —SR⁸, —NR⁹R¹⁰, —NO₂, —C(O)R⁹, —C(O)OR⁸, —C(O)NR⁹R¹⁰, —C(O)NR⁹S(O)₂R⁹, —OC(O)R⁹, —OC(O)NR⁹R¹⁰, —NR⁸C(O)R⁹, —NR⁹C(O)NR⁹R¹⁰, —S(O)R⁹, —S(O)₂R⁸, C₃-C₆ cycloalkyl and C₁-C₆ alkyl optionally substituted by halogen. In some embodiments, B is a 5- to 6-membered heterocycle optionally substituted with halogen.

In some embodiments of a compound of Formula (I), (II), or (III), B is a 9- to 10-membered heteroaryl optionally further substituted with R⁷. In some embodiments, B is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl and imidazothiazolyl, each optionally substituted with R⁷. In one aspect, such groups are unsubstituted. In another aspect, such groups are substituted with 1-3 R⁷, which may be the same or different. In some embodiments, B is a 9- to 10-membered heteroaryl optionally substituted with one or more groups selected from halogen, —CN, —OR⁸, —SR⁸, —NR⁹R¹⁰, —NO₂, —C(O)R⁹, —C(O)OR⁸, —C(O)NR⁹R¹⁰, —C(O)NR⁸S(O)₂R⁹, —OC(O)R⁸, —OC(O)NR⁹R¹⁰, —NR⁸C(O)R⁹, —NR⁸C(O)NR⁹R¹⁰, —S(O)R⁹, —S(O)₂R⁸, C₃-C₆ cycloalkyl and C₁-C₆ alkyl optionally substituted by halogen. In some embodiments, B is a 9- to 10-membered heteroaryl optionally substituted with halogen.

In some embodiments of a compound of Formula (I), (II), or (III), R⁷ is independently selected from the group consisting of halogen, —CN, —OR⁸, —SR⁸, —NR⁹R¹⁰, —NO₂, —C(O)R⁸, —C(O)OR⁸, —C(O)NR⁹R¹⁰, —C(O)NR⁸S(O)₂R⁹, —OC(O)R⁸, —OC(O)NR⁹R¹⁰, —NR⁸C(O)R⁹, —NR⁸C(O)NR⁹R¹⁰, —S(O)R⁸, —S(O)₂R⁸, C₃-C₆ cycloalkyl and C₁-C₆ alkyl optionally substituted by halogen. In some embodiments, R⁷ is halogen.

In some embodiments of a compound of Formula (I), (II), or (III), B is selected from the group consisting of:

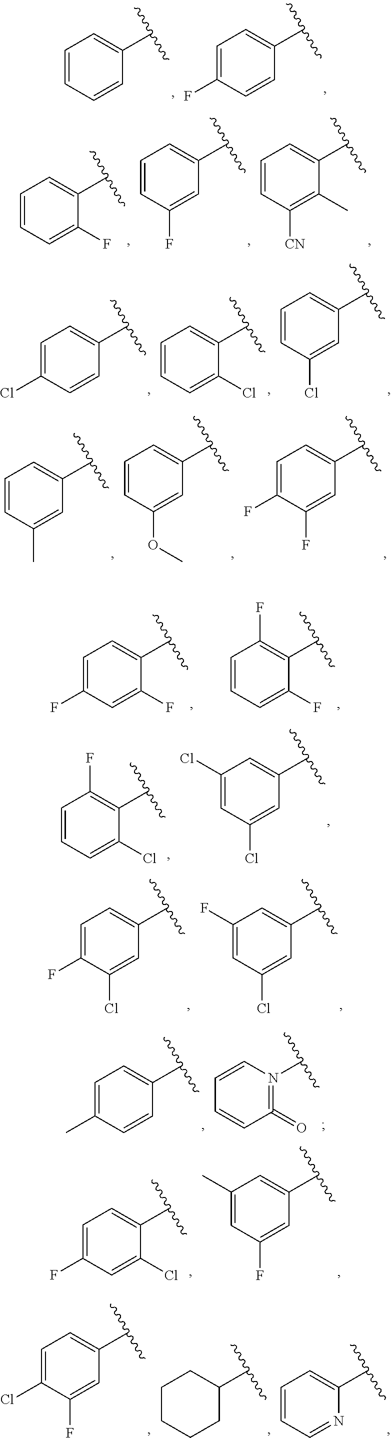

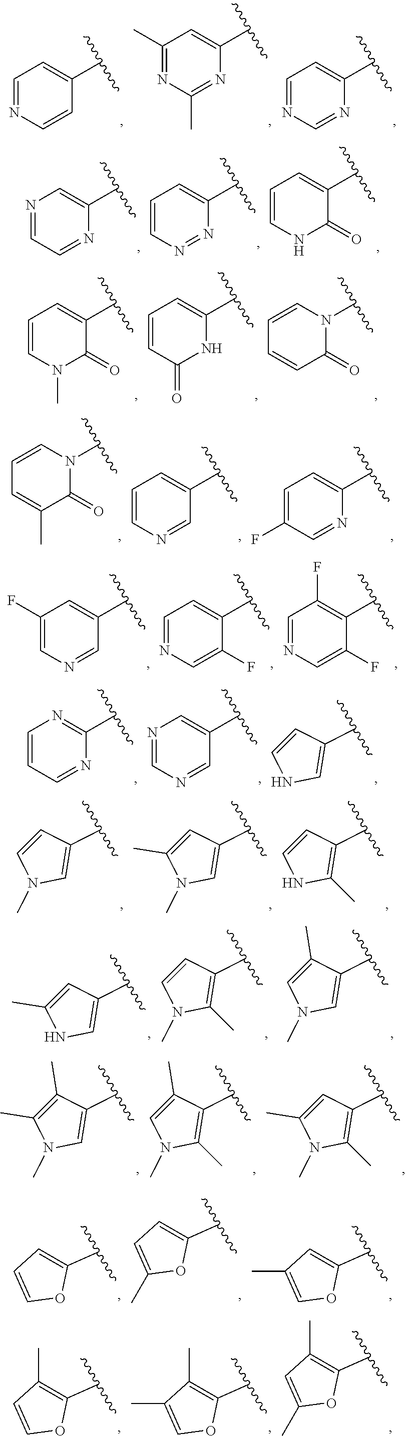

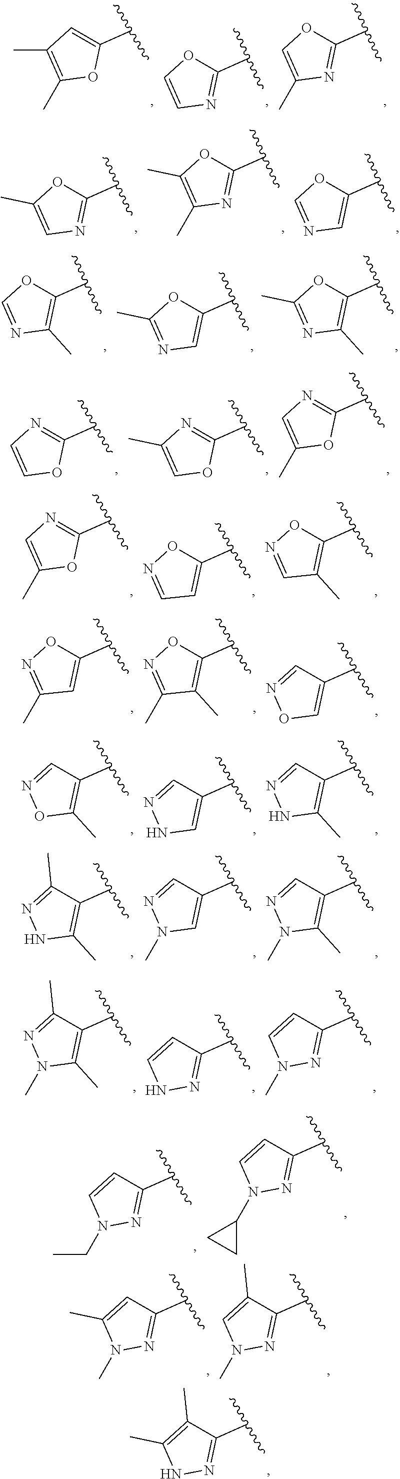

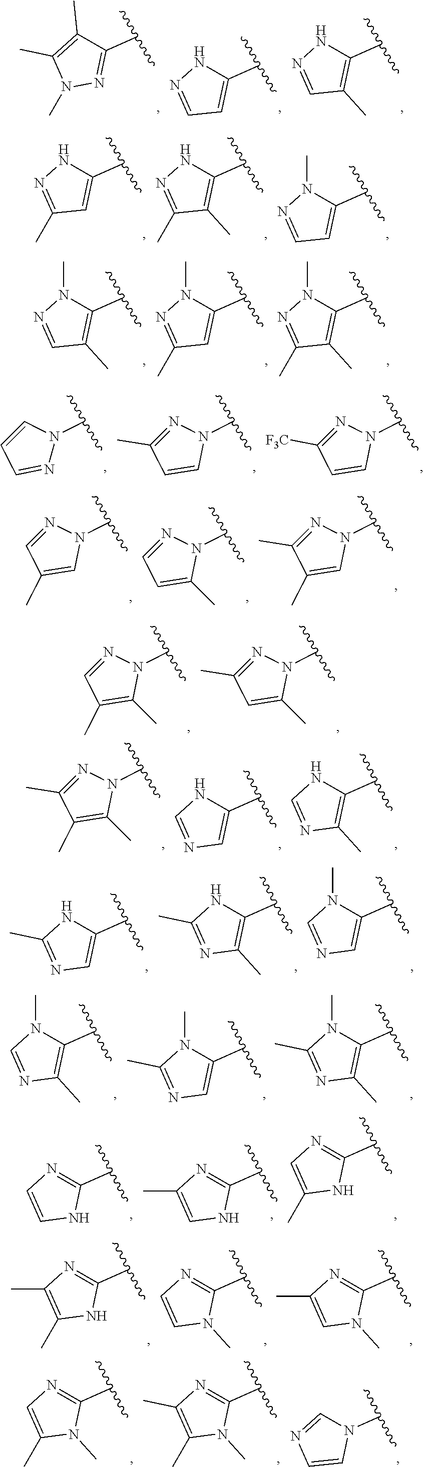

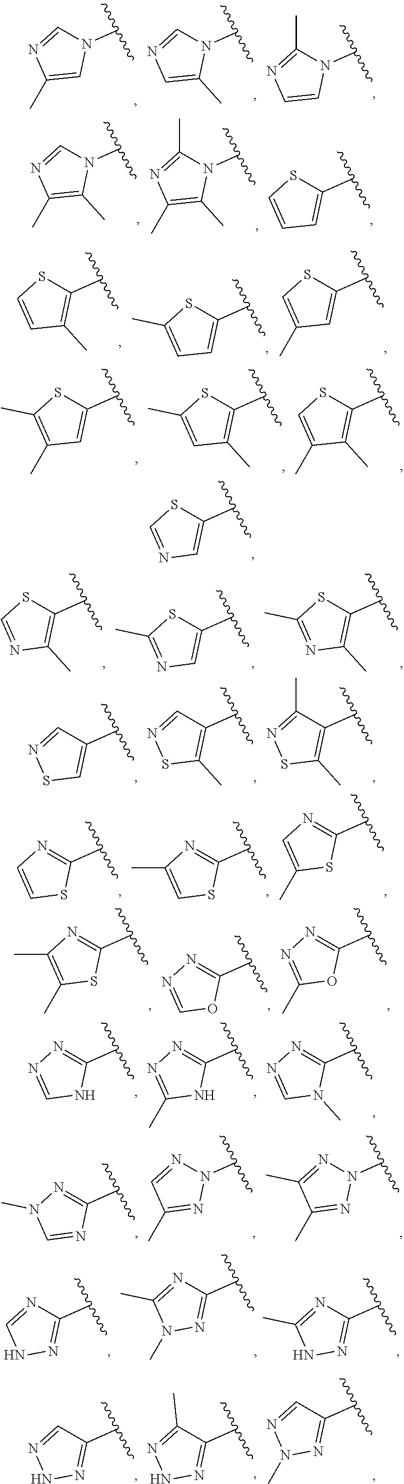

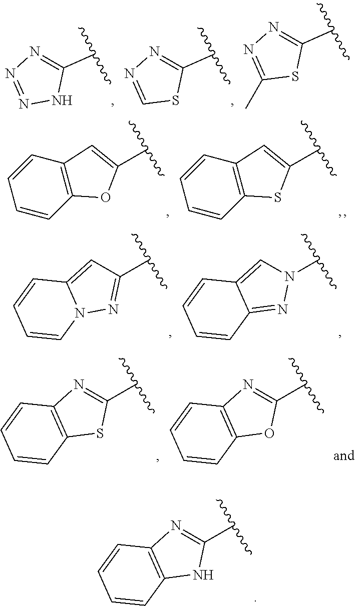

In some embodiments of a compound of Formula (I), (II), or (III), B is selected from the group consisting of:

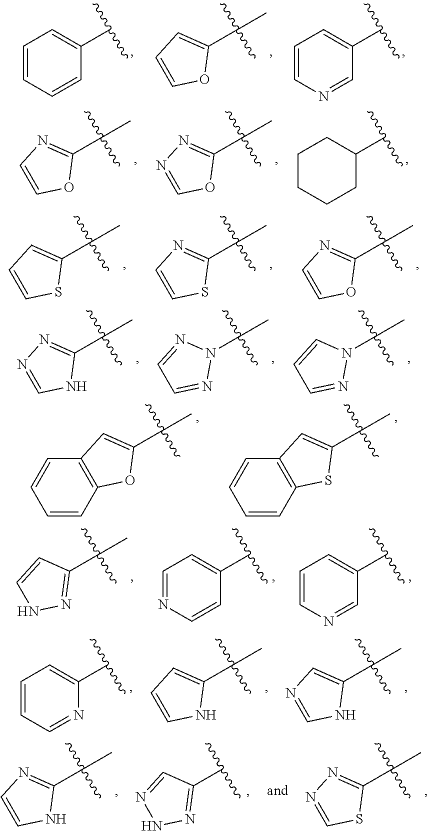

each of which is optionally substituted with R⁷. In one variation, such groups are unsubstituted. In another variation, such groups are substituted with 1, 2, 3, or 4 R⁷. In some of these embodiments, each R⁷ is independently C₁-C₆ alkyl, halogen, or —CN.

In some embodiments of a compound of Formula (I), (II), or (III) B is selected from the group consisting of:

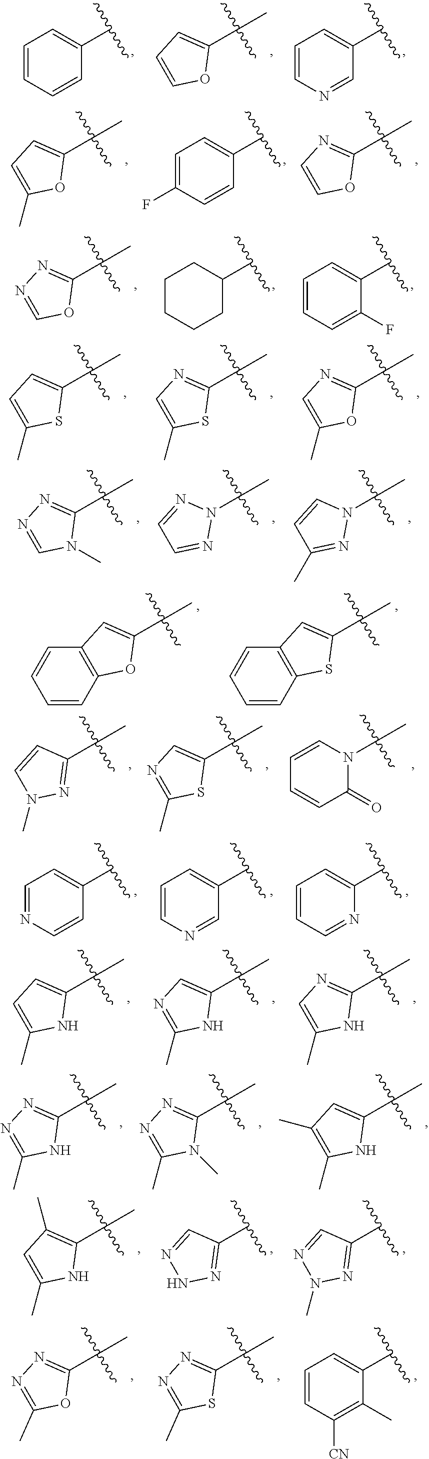

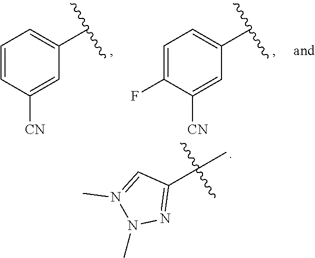

In some embodiments, B is

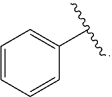 [SMILES: *c1ccccc1]

In some embodiments, B is

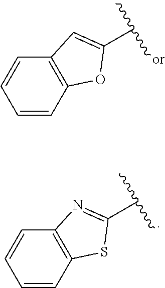 [SMILES: *c1cc2ccccc2o1 ; *c1nc2ccccc2s1]

In some embodiments of a compound of Formula (I), (II), or (III), A is C₆-C₁₂ aryl or 5- to 10-membered heteroaryl, each optionally further substituted with R⁶, and B is phenyl or 5- to 6-membered heteroaryl, each optionally further substituted with R⁷. In some embodiments, A is C₆-C₁₂ aryl, optionally further substituted with R⁶, and B is phenyl, optionally further substituted with R⁷. In some embodiments, A is C₆-C₁₂ aryl, optionally further substituted with R⁶, and B is 5- to 6-membered heteroaryl, optionally further substituted with R⁷. In some embodiments, A is 5- to 10-membered heteroaryl, optionally further substituted with R⁶, and B is phenyl, optionally further substituted with R⁷. In some embodiments, A is 5- to 10-membered heteroaryl, optionally further substituted with R⁶, and B is 5- to 6-membered heteroaryl, optionally further substituted with R⁷.

In some embodiments of a compound of Formula (I), (II), or (III), A is 9- to 10-membered carbocycle or 9- to 10-membered heterocycle, each optionally further substituted with R⁶, and B is phenyl, 5- to 6-membered heteroaryl, 5- to 6-membered carbocycle, or 5- to 6-membered heterocycle, each optionally further substituted with R⁷. In some embodiments, A is 9- to 10-membered carbocycle, optionally further substituted with R⁶, and B is phenyl, optionally further substituted with R⁷. In some embodiments, A is 9- to 10-membered carbocycle, optionally further substituted with R⁶, and B is 5- to 6-membered heteroaryl, optionally further substituted with R⁷. In some embodiments, A is 9- to 10-membered carbocycle, optionally further substituted with R⁶, and B is 5- to 6-membered carbocycle, optionally further substituted with R⁷. In some embodiments, A is 9- to 10-membered carbocycle, optionally further substituted with R⁶, and B is 5- to 6-membered heterocycle, optionally further substituted with R⁷.

In some embodiments of a compound of Formula (I), (II), or (III), A is C₆-C₁₂ aryl or 5- to 10-membered heteroaryl, each optionally further substituted with R⁶, and B is 5- to 6-membered carbocycle or 5- to 6-membered heterocycle, each optionally further substituted with R⁷. In some embodiments, A is C₆-C₁₂ aryl, optionally further substituted with R⁶, and B is 5- to 6-membered carbocycle, optionally further substituted with R⁷. In some embodiments, A is C₆-C₁₂ aryl, optionally further substituted with R⁶, and B is 5- to 6-membered heterocycle, optionally further substituted with R⁷. In some embodiments, A is 5- to 10-membered heteroaryl, optionally further substituted with R⁶, and B is 5- to 6-membered carbocycle, optionally further substituted with R⁷. In some embodiments, A is 5- to 10-membered heteroaryl, optionally further substituted with R⁶, and B is 5- to 6-membered heterocycle, optionally further substituted with R⁷. In some embodiments, when A is phenyl or pyridyl, either of which is optionally further substituted with R⁶, B is not a saturated heterocycle.

In some embodiments of a compound of Formula (I), (II), or (III), A is C₆-C₁₂ aryl or 5- to 10-membered heteroaryl, each optionally further substituted with R⁶, and B is 9- to 10-membered carbocycle, optionally further substituted with R⁷. In some embodiments, A is C₆-C₁₂ aryl, optionally further substituted with R⁶, and B is 9- to 10-membered carbocycle, optionally further substituted with R⁷. In some embodiments, A is 5- to 10-membered heteroaryl, optionally further substituted with R⁶, and B is 9- to 10-membered carbocycle, optionally further substituted with R⁷.

In some embodiments of a compound of Formula (I), (II), or (III), A is

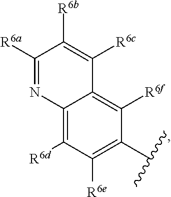

wherein R^{6a}, R^{6b}, R^{6c}, R^{6d}, R^{6e}, and R^{6f} are each independently H, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁹, —(C₁-C₃ alkylene)OC(O)R⁹, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{6a}, R^{6b}, R^{6c}, R^{6d}, R^{6e}, and R^{6f} is independently optionally substituted by halogen, oxo, —OR¹¹, —NR¹¹R¹², —C(O)R¹¹, —CN, —S(O)R¹¹, —S(O)₂R¹¹, —P(O)(OR¹¹)OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen.

In some embodiments of a compound of Formula (I), (II), or (III), A is

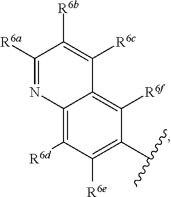

wherein R^{6a}, R^{6b}, R^{6c}, R^{6d}, R^{6e}, and R^{6f} are each independently H, C₁-C₆ alkyl, halogen, —CN, or —OC₁-C₆ alkyl. In some embodiments, R^{6a}, R^{6b}, R^{6c}, R^{6d}, R^{6e}, and R^{6f} are each H. In some embodiments, one of R^{6a}, R^{6b}, R^{6c}, R^{6d}, R^{6e}, and R^{6f} is Cl, F, Br, or I. In some embodiments, one of R^{6a}, R^{6b}, R^{6c}, R^{6d}, R^{6e}, and R^{6f} is Cl. In some embodiments, one of R^{6a}, R^{6b}, R^{6c}, R^{6d}, R^{6e}, and R^{6f} is halogen and the others are each H. In some embodiments, one of R^{6a}, R^{6b}, R^{6c}, R^{6d}, R^{6e}, and R^{6f} is halogen and one of R^{6a}, R^{6b}, R^{6c}, R^{6d}, R^{6e}, and R^{6f} is C₁-C₆ alkyl. In some embodiments, one of R^{6a}, R^{6b}, R^{6c}, R^{6d}, R^{6e}, and R^{6f} is Cl and one of R^{6a}, R^{6b}, R^{6c}, R^{6d}, R^{6e}, and R^{6f} is methyl. In some embodiments, R^{6a} is C₁-C₆ alkyl. In some embodiments, R^{6b} is C₁-C₆ alkyl. In some embodiments, R^{6c} is C₁-C₆ alkyl. In some embodiments, R^{6d} is C₁-C₆ alkyl. In some embodiments, R^{6e} is C₁-C₆ alkyl. In some embodiments, R^{6f} is C₁-C₆ alkyl. In some embodiments, R^{6a} is methyl, ethyl, n-propyl, isopropyl, n-butyl, isobutyl, secbutyl, or tertbutyl. In some embodiments, R^{6b} is methyl, ethyl, n-propyl, isopropyl, n-butyl, isobutyl, secbutyl, or tertbutyl. In some embodiments, R^{6c} is methyl, ethyl, n-propyl, isopropyl, n-butyl, isobutyl, secbutyl, or tertbutyl. In some embodiments, R^{6d} is methyl, ethyl, n-propyl, isopropyl, n-butyl, isobutyl, secbutyl, or tertbutyl. In some embodiments, R^{6e} is methyl, ethyl, n-propyl, isopropyl, n-butyl, isobutyl, secbutyl, or tertbutyl. In some embodiments, R^{6f} is methyl, ethyl, n-propyl, isopropyl, n-butyl, isobutyl, secbutyl, or tertbutyl. In some embodiments, R^{6a} is C₁-C₆ alkyl and R^{6b} is halogen. In some embodiments, R^{6a} is C₁-C₆ alkyl and R^{6c} is halogen. In some embodiments, R^{6a} is C₁-C₆ alkyl and R^{6d} is halogen. In some embodiments, R^{6a} is C₁-C₆ alkyl and R^{6e} is halogen. In some embodiments, R^{6a} is C₁-C₆ alkyl and R^{6f} is halogen. In some embodiments, R^{6b} is C₁-C₆ alkyl and R^{6a} is halogen. In some embodiments, R^{6b} is C₁-C₆ alkyl and R^{6c} is halogen. In some embodiments, R^{6b} is C₁-C₆ alkyl and R^{6a} is halogen. In some embodiments, R^{6b} is C₁-C₆ alkyl and R^{6e} is halogen. In some embodiments, R^{6b} is C₁-C₆ alkyl and R^{6f} is halogen. In some embodiments, R^{6c} is C₁-C₆ alkyl and R^{6a} is halogen. In some embodiments, R^{6c} is C₁-C₆ alkyl and R^{6b} is halogen. In some embodiments, R^{6c} is C₁-C₆ alkyl and R^{6a} is halogen. In some embodiments, R^{6c} is C₁-C₆ alkyl and R¹ is halogen. In some embodiments, R^{6c} is C₁-C₆ alkyl and R^{6f} is halogen. In some embodiments, R^{6d} is C₁-C₆ alkyl and R^{6a} is halogen. In some embodiments, R^{6d} is C₁-C₆ alkyl and R^{6b} is halogen. In some embodiments, R^{6d} is C₁-C₆ alkyl and R^{6c} is halogen. In some embodiments, R^{6d} is C₁-C₆ alkyl and R^{6e} is halogen. In some embodiments, R^{6d} is C₁-C₆ alkyl and R^{6f} is halogen. In some embodiments, R^{6e} is C₁-C₆ alkyl and R^{6a} is halogen. In some embodiments, R^{6e} is C₁-C₆ alkyl and R^{6b} is halogen. In some embodiments, R^{6e} is C₁-C₆ alkyl and R¹ is halogen. In some embodiments, R^{6e} is C₁-C₆ alkyl and R^{6a} is halogen. In some embodiments, R^{6e} is C₁-C₆ alkyl and R^{6f} is halogen. In some embodiments, R^{6f} is C₁-C₆ alkyl and R^{6a} is halogen. In some embodiments, R^{6f} is C₁-C₆ alkyl and R^{6b} is halogen. In some embodiments, R^{6f} is C₁-C₆ alkyl and R^{6c} is halogen. In some embodiments, R^{6f} is C₁-C₆ alkyl and R^{6a} is halogen. In some embodiments, R^{6f} is C₁-C₆ alkyl and R^{6e} is halogen. In some embodiments, two of R^{6a}, R^{6b}, R^{6c}, R^{6d}, R^{6e}, and R^{6f} are halogen. In some embodiments, two of R^{6a}, R^{6b}, R^{6c}, R^{6d}, R^{6e}, and R^{6f} are C₁-C₆ alkyl.

In some embodiments of a compound of Formula (I), (II), or (III), A is

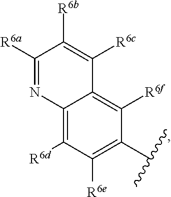

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is phenyl, optionally substituted with R⁷.

In some embodiments of a compound of Formula (I), (II), or (III), A is

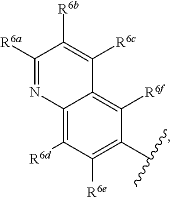

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is a 5- to 6-membered heteroaryl, optionally substituted with R⁷.

In some embodiments of a compound of Formula (I), (II), or (III), A is

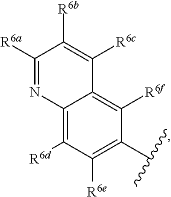

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is a 5- to 6-membered carbocycle, optionally substituted with R⁷.

In some embodiments of a compound of Formula (I), (II), or (III), A is

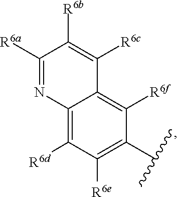

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is a 5- to 6-membered heterocycle, optionally substituted with R⁷.

In some embodiments of a compound of Formula (I), (II), or (III), A is

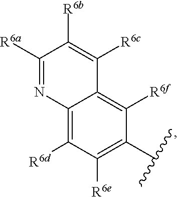

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is a 9- to 10-membered heteroaryl, optionally substituted with R⁷.

In some embodiments of a compound of Formula (I), (II), or (III), A is

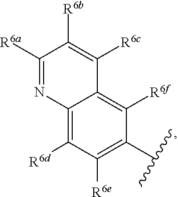

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is selected from the group consisting of:

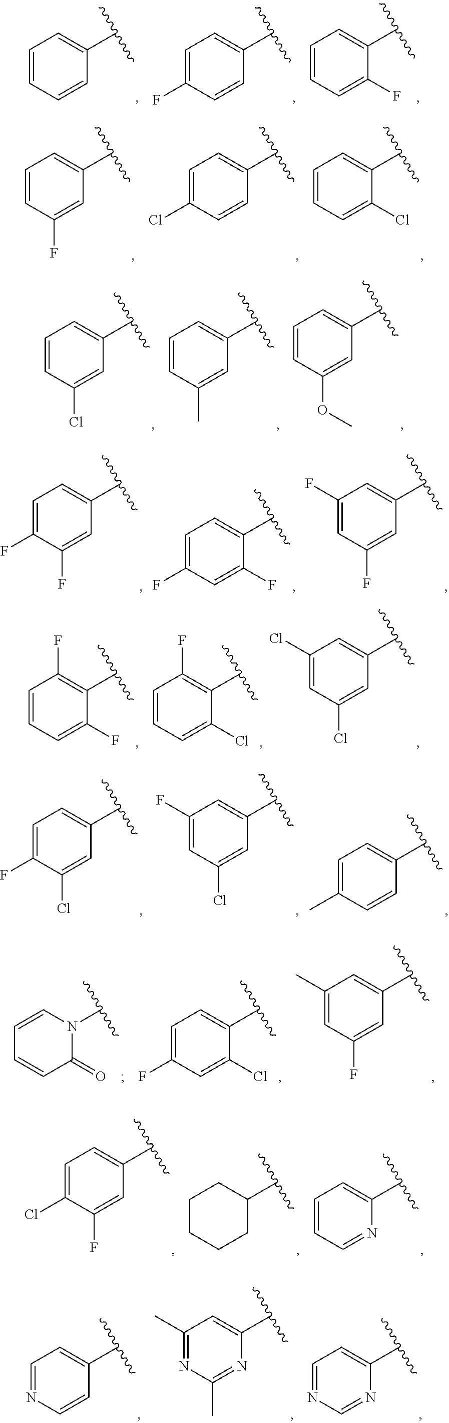

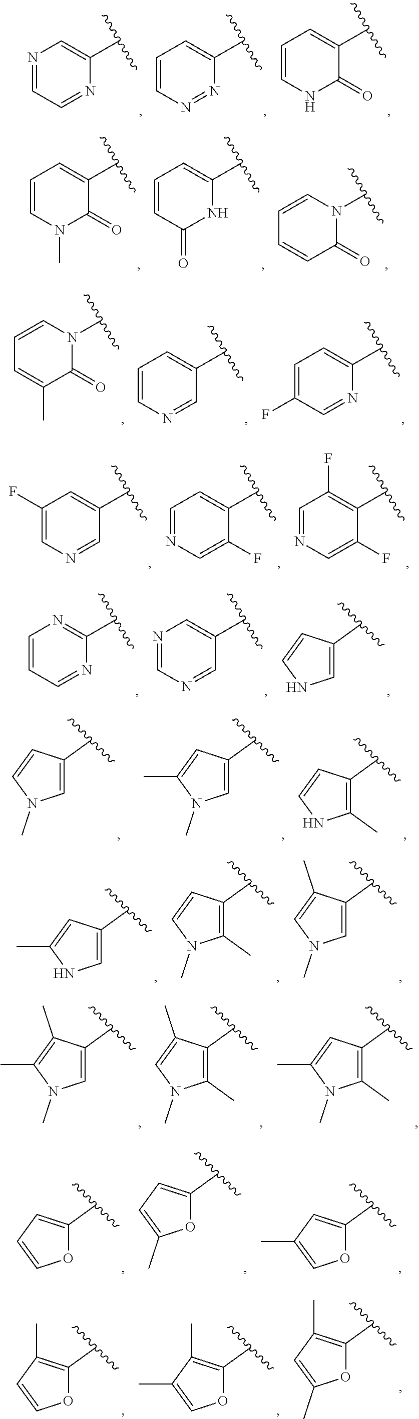

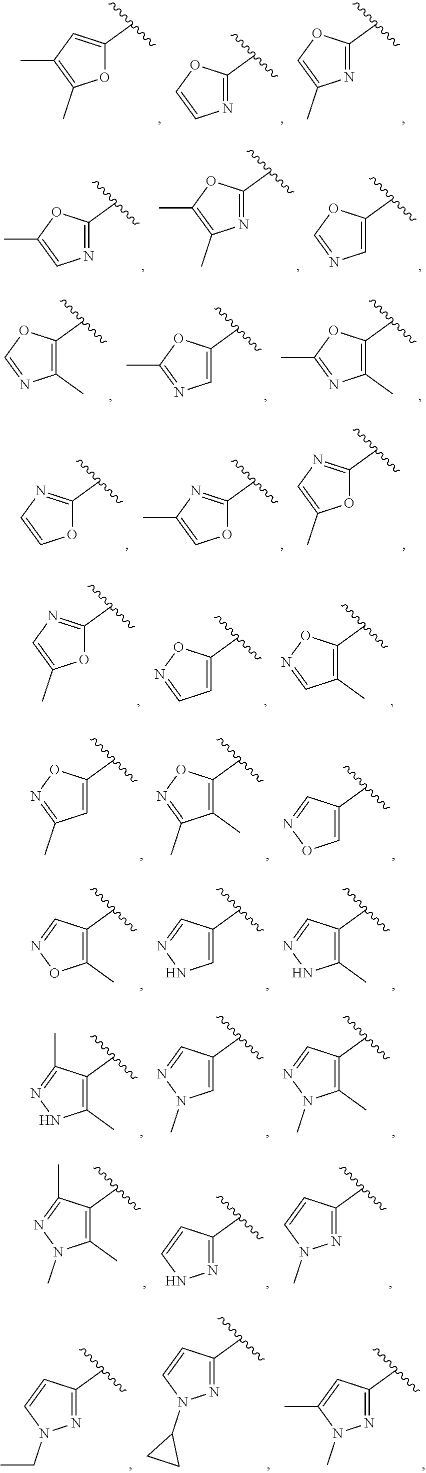

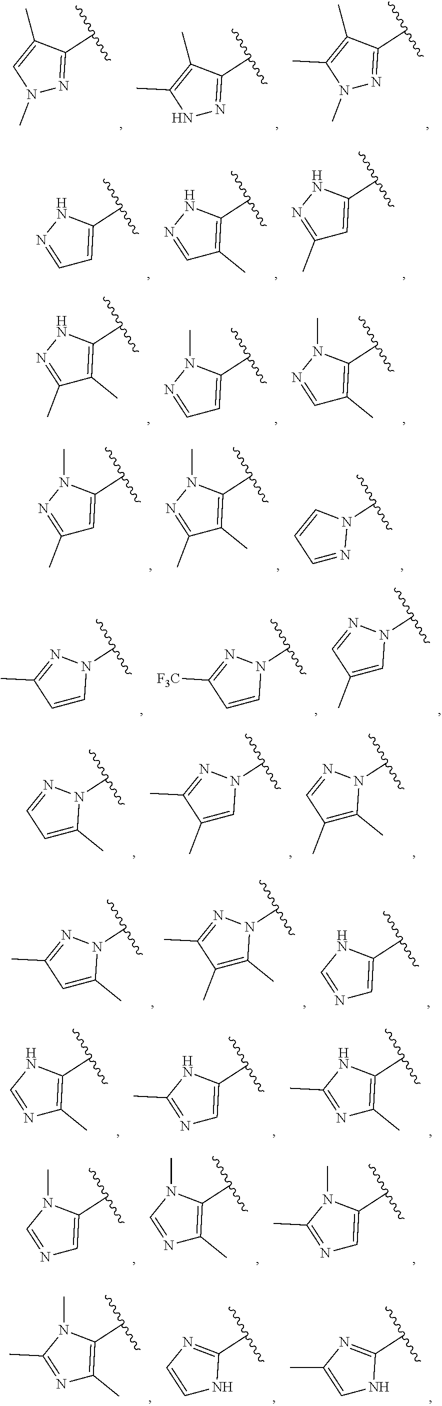

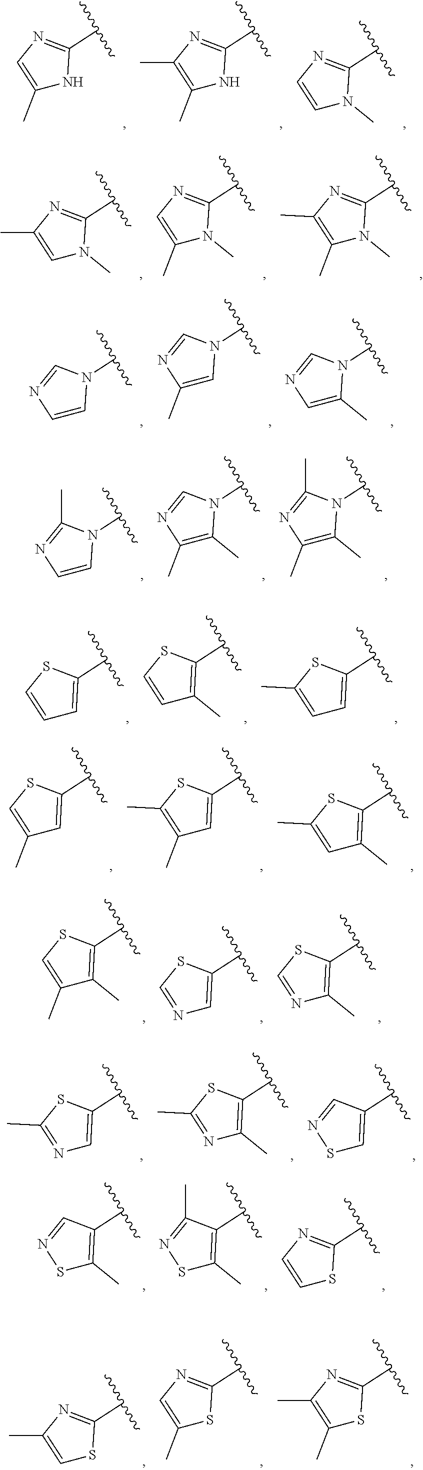

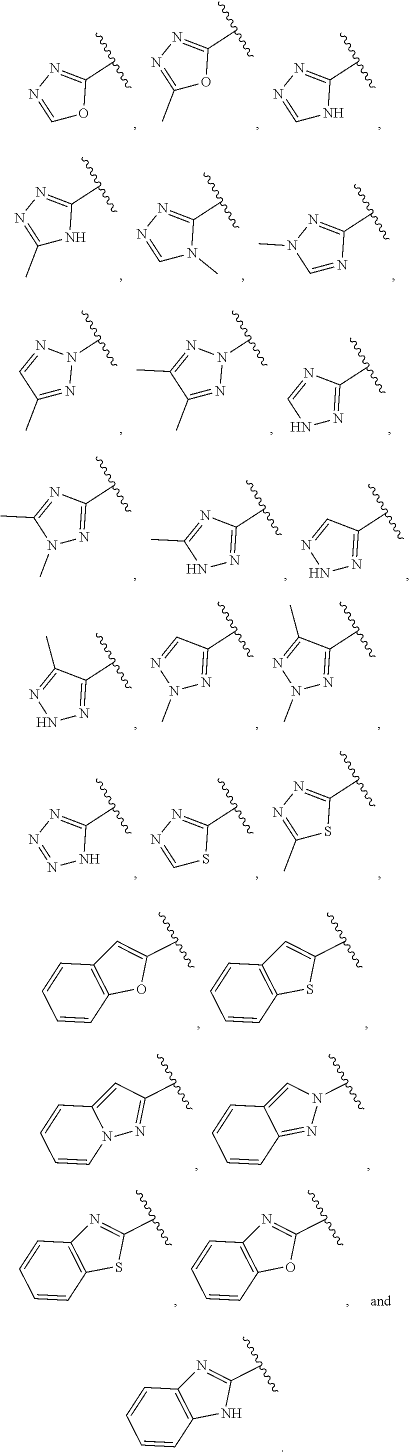

In some embodiments of a compound of Formula (I), (II), or (III), A is

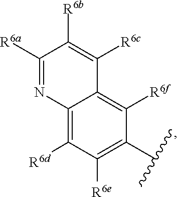

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is selected from the group consisting of:

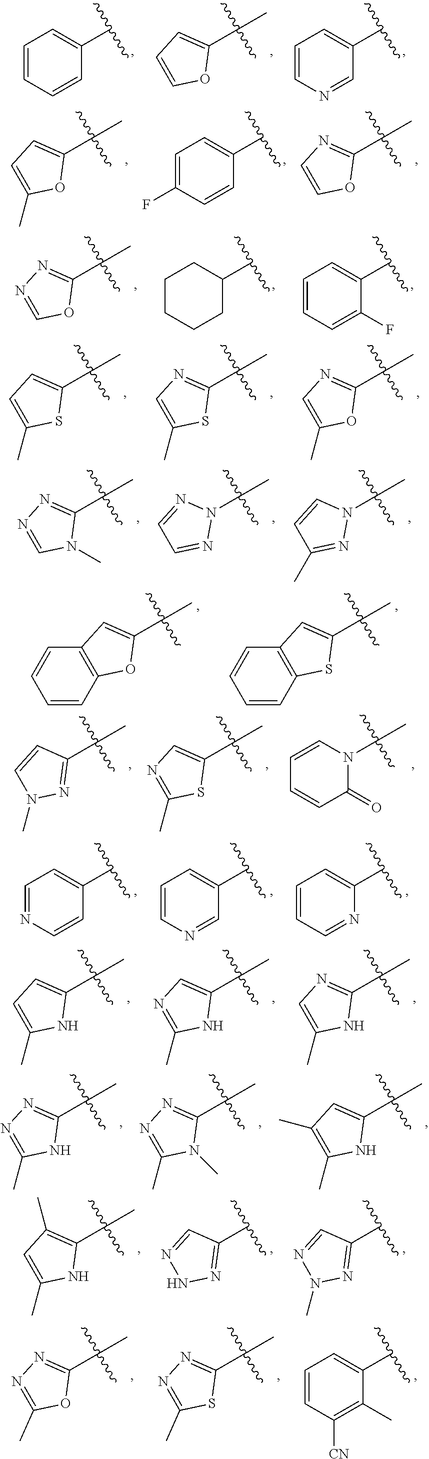

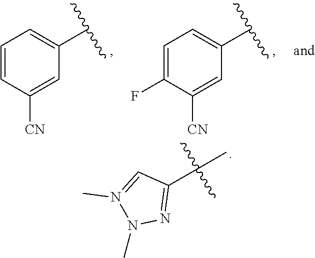

In some embodiments of a compound of Formula (I), (II), or (III), A is

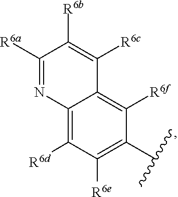

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is selected from the group consisting of

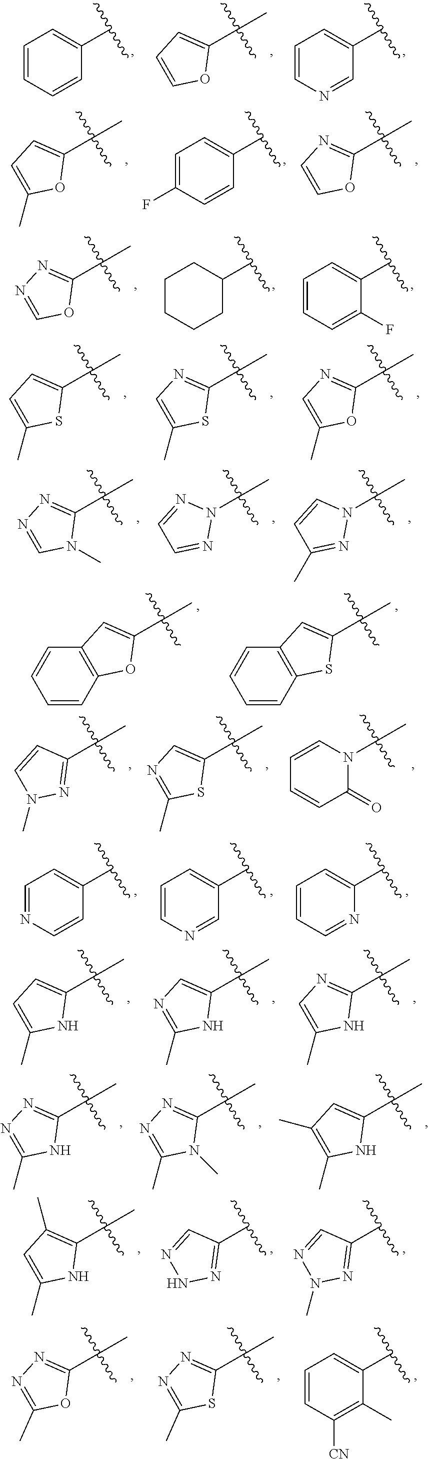

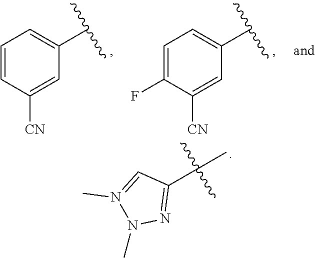

In some embodiments of a compound of Formula (I), (II), or (III), A is

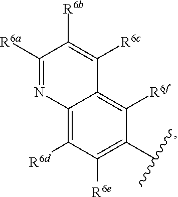

wherein one or two of R^{6a}, R^{6b}, R^{6c}, R^{6d}, R^{6e}, and R^{6f} are independently selected from the group consisting of C₁-C₆ alkyl, halogen, —CN, and —OC₁-C₆ alkyl, and the remainder of R^{6a}, R^{6b}, R^{6c}, R^{6d}, R^{6e}, and R^{6f} are each H; and B is selected from

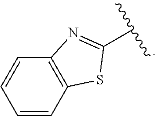 [SMILES: *c1nc2ccccc2s1]

In some embodiments of a compound of Formula (I), (II), or (III), A is

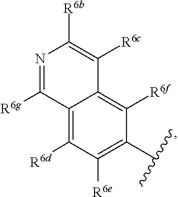

wherein R^{6b}, R^{6c}, R^{6d}, R^{6e}, R^{6f}, and R^{6g} are each independently H, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁹, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁹, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁹, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{6b}, R^{6c}, R^{6d}, R^{6e}, R^{6f}, and R^{6g} is independently optionally substituted by halogen, oxo, —OR¹¹, —NR¹¹R¹², —C(O)R¹¹, —CN, —S(O)R¹¹, —S(O)₂R¹¹, —P(O)(OR¹¹)OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen.

In some embodiments of a compound of Formula (I), (II), or (III), A is

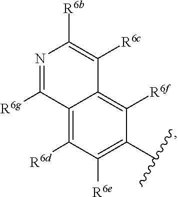

wherein R^{6b}, R^{6c}, R^{6d}, R^{6e}, R^{6f}, and R^{6g} are each independently H, C₁-C₆ alkyl, halogen, —CN, or —OC₁-C₆ alkyl. In some embodiments, R^{6b}, R^{6c}, R^{6d}, R^{6e}, R^{6f}, and R^{6g} are each H. In some embodiments, one of R^{6b}, R^{6c}, R^{6d}, R^{6e}, R^{6f}, and R^{6f} is Cl, F, Br, or I. In some embodiments, one of R^{6b}, R^{6c}, R^{6d}, R^{6e}, R^{6f}, and R^{6g} is Cl. In some embodiments, one of R^{6b}, R^{6c}, R^{6d}, R^{6e}, R^{6f}, and R^{6g} is halogen and the others are each H. In some embodiments, one of R^{6b}, R^{6c}, R^{6d}, R^{6e}, R^{6f}, and R^{6g} is halogen and one of R^{6b}, R^{6c}, R^{6d}, R^{6e}, R^{6f}, and R^{6g} is C₁-C₆ alkyl. In some embodiments, one of R^{6b}, R^{6c}, R^{6d}, R^{6e}, R^{6f}, and R^{6g} is Cl and one of R^{6b}, R^{6c}, R^{6d}, R^{6e}, R^{6f}, and R^{6g} is methyl. In some embodiments, R^{6g} is C₁-C₆ alkyl. In some embodiments, R^{6b} is C₁-C₆ alkyl. In some embodiments, R^{6c} is C₁-C₆ alkyl. In some embodiments, R^{6d} is C₁-C₆ alkyl. In some embodiments, R^{6e} is C₁-C₆ alkyl. In some embodiments, R^{6f} is C₁-C₆ alkyl. In some embodiments, R^{6g} is methyl, ethyl, n-propyl, isopropyl, n-butyl, isobutyl, secbutyl, or tertbutyl. In some embodiments, R^{6b} is methyl, ethyl, n-propyl, isopropyl, n-butyl, isobutyl, secbutyl, or tertbutyl. In some embodiments, R^{6c} is methyl, ethyl, n-propyl, isopropyl, n-butyl, isobutyl, secbutyl, or tertbutyl. In some embodiments, R^{6d} is methyl, ethyl, n-propyl, isopropyl, n-butyl, isobutyl, secbutyl, or tertbutyl. In some embodiments, R^{6e} is methyl, ethyl, n-propyl, isopropyl, n-butyl, isobutyl, secbutyl, or tertbutyl. In some embodiments, R^{6f} is methyl, ethyl, n-propyl, isopropyl, n-butyl, isobutyl, secbutyl, or tertbutyl. In some embodiments, R^{6g} is C₁-C₆ alkyl and R^{6b} is halogen. In some embodiments, R^{6g} is C₁-C₆ alkyl and R^{6c} is halogen. In some embodiments, R^{6g} is C₁-C₆ alkyl and R^{6d} is halogen. In some embodiments, R^{6g} is C₁-C₆ alkyl and R^{6e} is halogen. In some embodiments, R^{6g} is C₁-C₆ alkyl and R^{6f} is halogen. In some embodiments, R^{6b} is C₁-C₆ alkyl and R^{6g} is halogen. In some embodiments, R^{6b} is C₁-C₆ alkyl and R^{6c} is halogen. In some embodiments, R^{6b} is C₁-C₆ alkyl and R^{6d} is halogen. In some embodiments, R^{6b} is C₁-C₆ alkyl and R^{6e} is halogen. In some embodiments, R^{6b} is C₁-C₆ alkyl and R^{6f} is halogen. In some embodiments, R^{6c} is C₁-C₆ alkyl and R^{6g} is halogen. In some embodiments, R^{6c} is C₁-C₆ alkyl and R^{6b} is halogen. In some embodiments, R^{6c} is C₁-C₆ alkyl and R^{6d} is halogen. In some embodiments, R^{6c} is C₁-C₆ alkyl and R^{6e} is halogen. In some embodiments, R^{6c} is C₁-C₆ alkyl and R^{6f} is halogen. In some embodiments, R^{6d} is C₁-C₆ alkyl and R^{6g} is halogen. In some embodiments, R^{6d} is C₁-C₆ alkyl and R^{6b} is halogen. In some embodiments, R^{6d} is C₁-C₆ alkyl and R^{6c} is halogen. In some embodiments, R^{6d} is C₁-C₆ alkyl and R^{6e} is halogen. In some embodiments, R^{6d} is C₁-C₆ alkyl and R^{6f} is halogen. In some embodiments, R^{6e} is C₁-C₆ alkyl and R^{6g} is halogen. In some embodiments, R^{6e} is C₁-C₆ alkyl and R^{6b} is halogen. In some embodiments, R^{6e} is C₁-C₆ alkyl and R^{6c} is halogen. In some embodiments, R^{6e} is C₁-C₆ alkyl and R^{6d} is halogen. In some embodiments, R^{6e} is C₁-C₆ alkyl and R^{6f} is halogen. In some embodiments, R^{6f} is C₁-C₆ alkyl and R^{6g} is halogen. In some embodiments, R^{6f} is C₁-C₆ alkyl and R^{6b} is halogen. In some embodiments, R^{6f} is C₁-C₆ alkyl and R^{6c} is halogen. In some embodiments, R^{6f} is C₁-C₆ alkyl and R^{6d} is halogen. In some embodiments, R^{6f} is C₁-C₆ alkyl and R^{6e} is halogen. In some embodiments, two of R^{6b}, R^{6c}, R^{6d}, R^{6e}, R^{6f}, and R^{6f} are halogen. In some embodiments, two of R^{6b}, R^{6c}, R^{6d}, R^{6e}, R^{6f}, and R^{6g} are C₁-C₆ alkyl.

In some embodiments of a compound of Formula (I), (II), or (III), A is

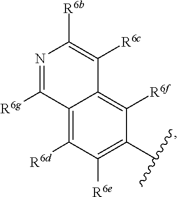

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is phenyl, optionally substituted with R⁷.

In some embodiments of a compound of Formula (I), (II), or (III), A is

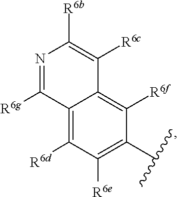

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is a 5- to 6-membered heteroaryl, optionally substituted with R⁷.

In some embodiments of a compound of Formula (I), (II), or (III), A is

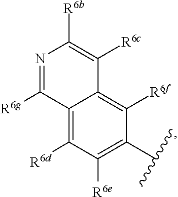

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is a 5- to 6-membered carbocycle, optionally substituted with R⁷.

In some embodiments of a compound of Formula (I), (II), or (III), A is

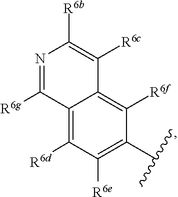

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is a 5- to 6-membered heterocycle, optionally substituted with R⁷.

In some embodiments of a compound of Formula (I), (II), or (III), A is

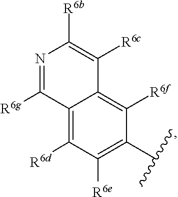

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is a 9- to 10-membered heteroaryl, optionally substituted with R⁷.

In some embodiments of a compound of Formula (I), (II), or (III), A is

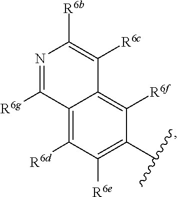

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is selected from the group consisting of:

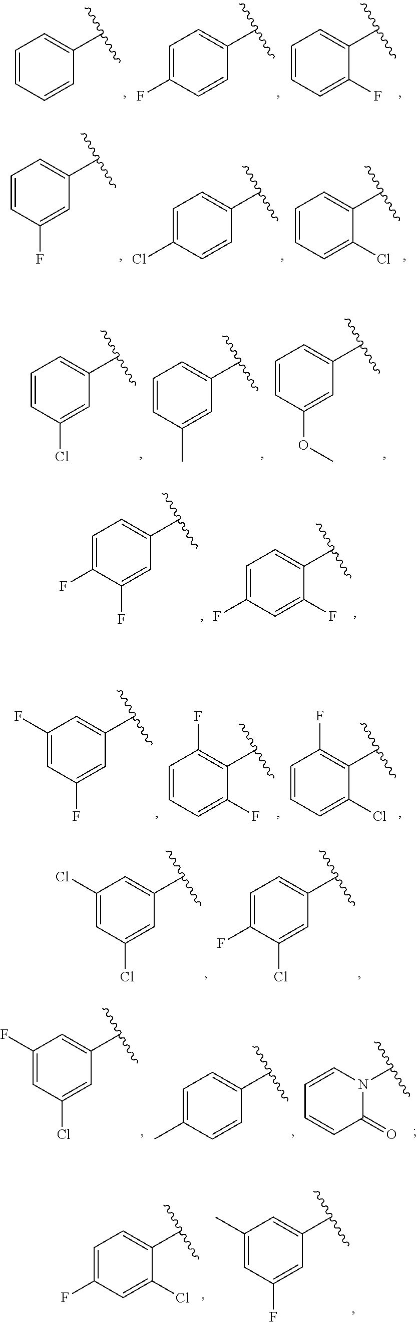

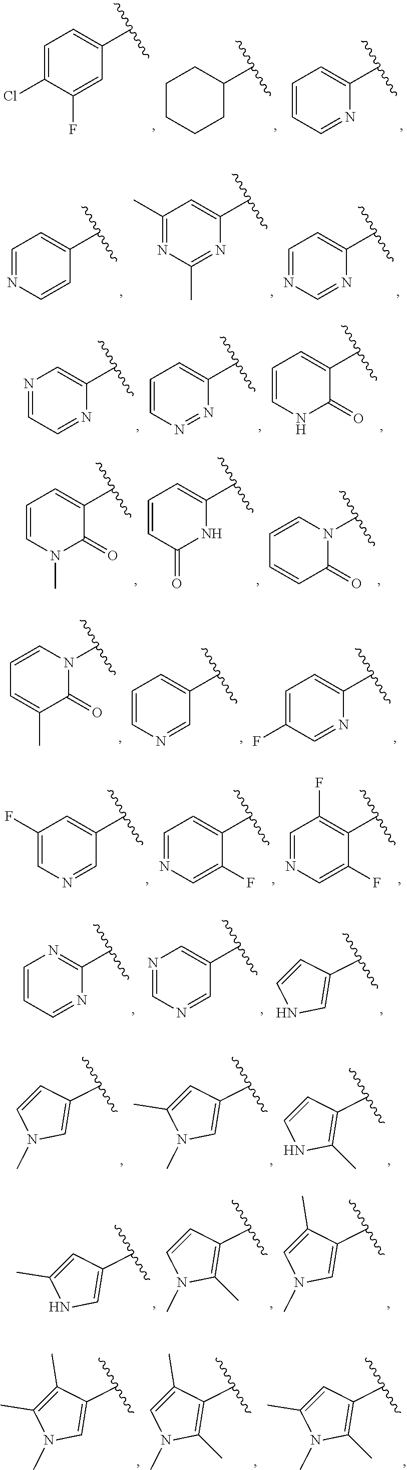

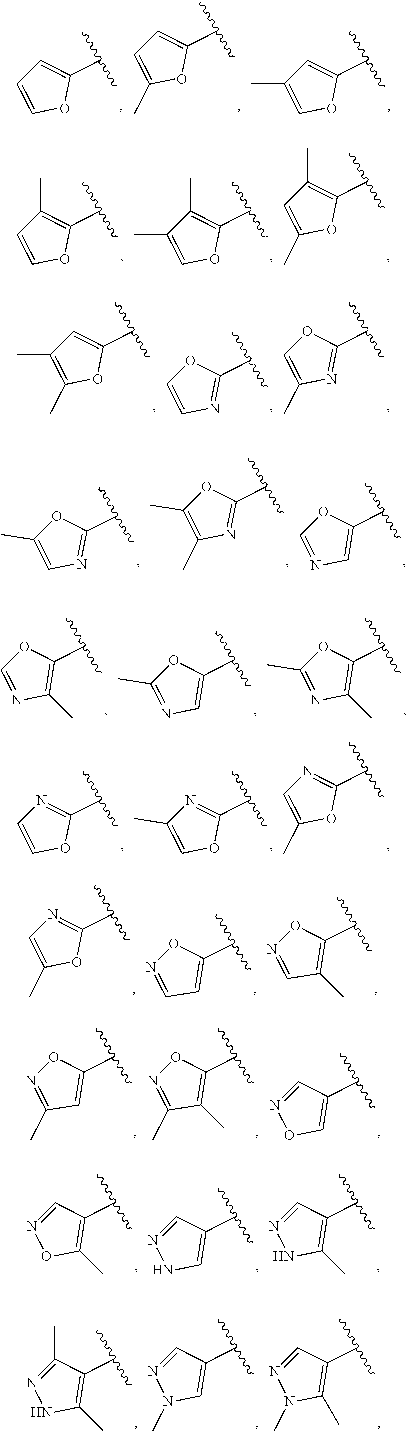

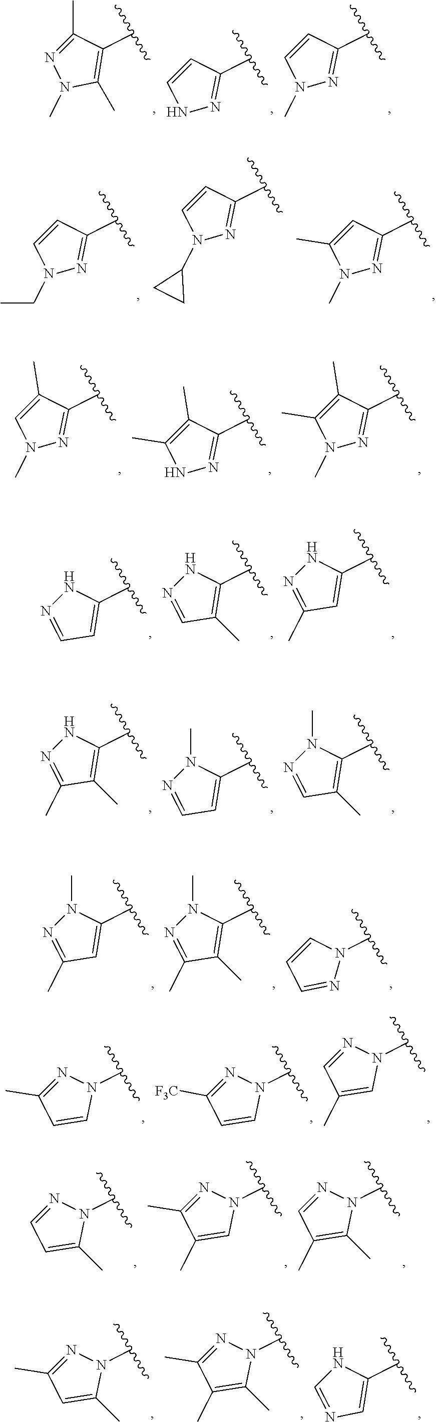

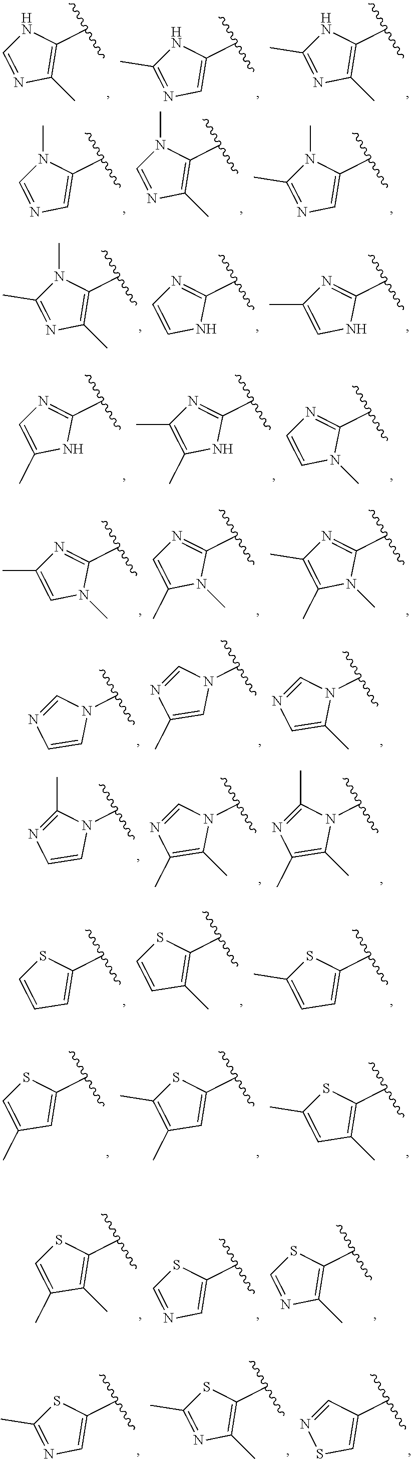

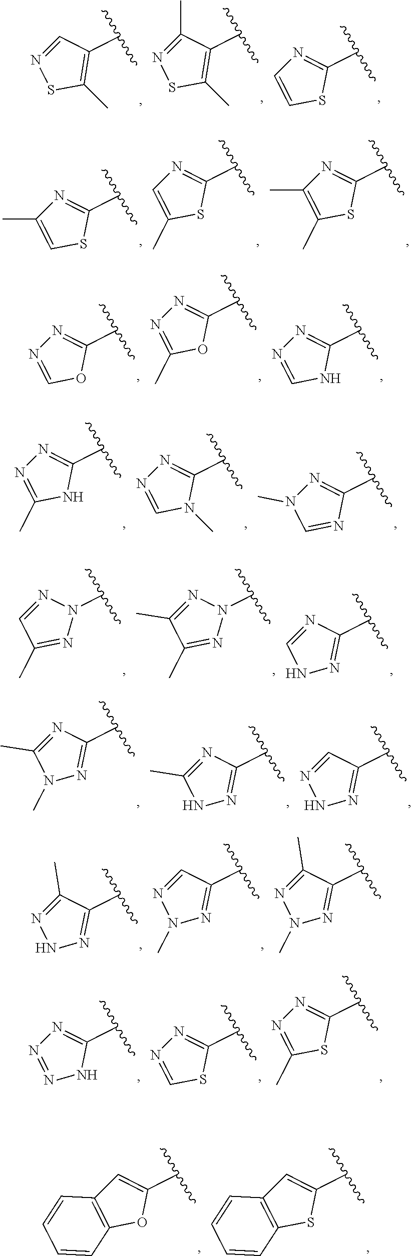

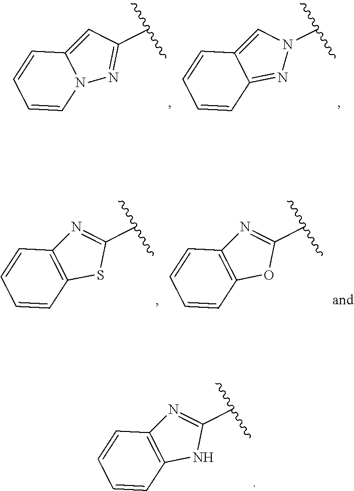 [SMILES: *c1cc2ccccn2n1 ; *n1cc2ccccc2n1 ; *c1nc2ccccc2s1 ; *c1nc2ccccc2o1 ; *c1nc2ccccc2[nH]1]

In some embodiments of a compound of Formula (I), (II), or (III), A is

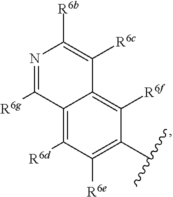

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is selected from the group consisting of:

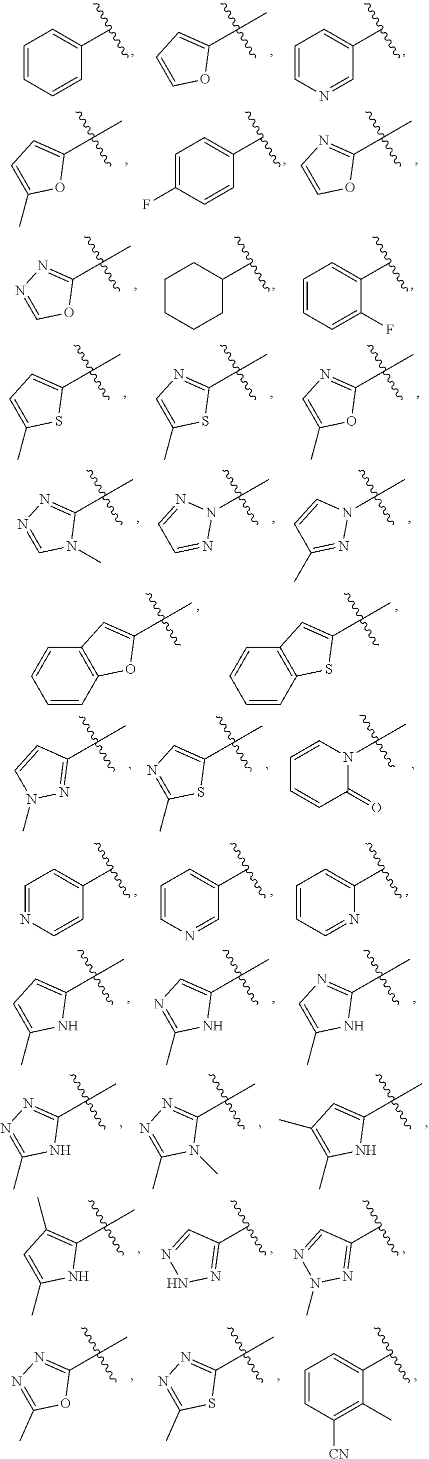

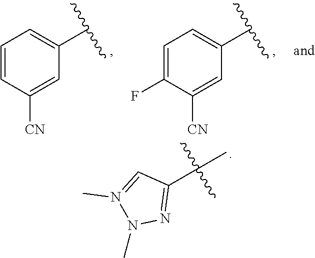

In some embodiments of a compound of Formula (I), (II), or (III), A is

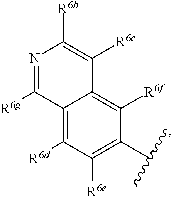

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is selected from the group consisting of

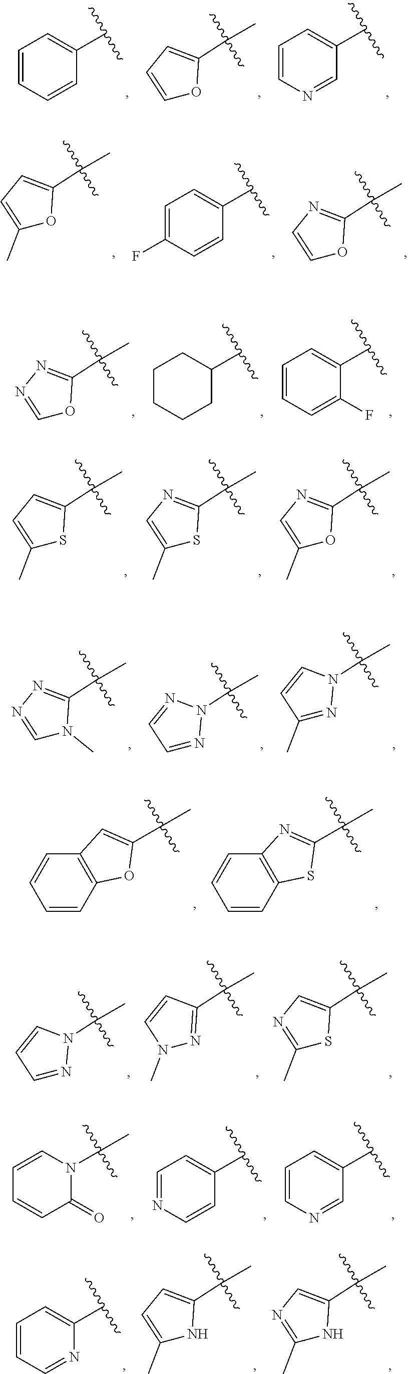

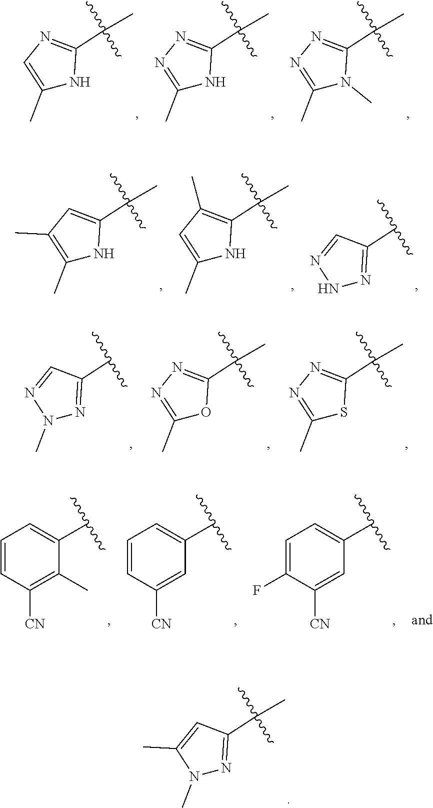

In some embodiments of a compound of Formula (I), (II), or (III), A is

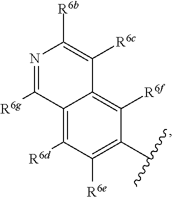

wherein one or two of R^{6b}, R^{6c}, R^{6d}, R^{6e}, R^{6f}, and R^{6g} are independently selected from the group consisting of C₁-C₆ alkyl, halogen, —CN, and —OC₁-C₆ alkyl, and the remainder of R^{6b}, R^{6c}, R^{6d}, R^{6e}, R^{6f}, and R^{6g} are each H; and B is selected from

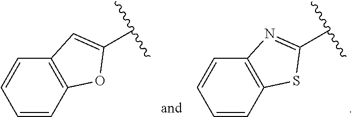 [SMILES: *c1cc2ccccc2o1 ; *c1nc2ccccc2s1]

In some embodiments of a compound of Formula (I), (II), or (III), A is

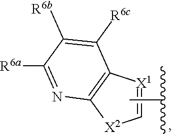

wherein X¹ is selected from the group consisting of N, C, and CH; X² is selected from the group consisting of NH, O, and S; and R^{6a}, R^{6b}, and R^{6c} are each independently H, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁹, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁹S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁹, —(C₁-C₃ alkylene)SR⁹, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁹, —(C₁-C₃ alkylene)OC(O)R⁹, —(C₁-C₃ alkylene)C(O)OR⁹, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁹S(O)R⁹, —C(O)(C₁-C₃ alkylene)NR⁹S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{6a}, R^{6b}, and R^{6c} is independently optionally substituted by halogen, oxo, —OR¹¹, —NR¹¹R¹², —C(O)R¹¹, —CN, —S(O)R¹¹, —S(O)₂R¹¹, —P(O)(OR¹))(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen.

In some embodiments of a compound of Formula (I), (II), or (III), A is

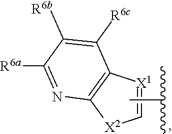

wherein X¹ is selected from the group consisting of N, C, and CH; X² is selected from the group consisting of NH, O, and S; and R^{6a}, R^{6b}, and R^{6c} are each independently H, C₁-C₆ alkyl, halogen, —CN, or —OC₁-C₆ alkyl. In some embodiments of a compound of Formula (I), (II), or (III), A is

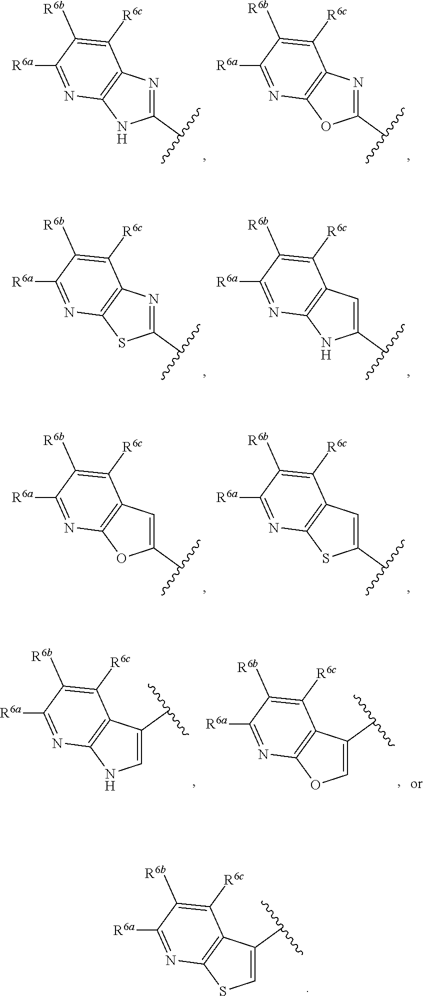

In some embodiments, R^{6a}, R^{6b}, and R^{6c} are each H. In some embodiments, one of R^{6a}, R^{6b}, and R^{6c} is Cl, F, Br, or I. In some embodiments, one of R^{6a}, R^{6b}, and R^{6c} is Cl. In some embodiments, one of R^{6a}, R^{6b}, and R^{6c} is halogen and the others are each H. In some embodiments, one of R^{6a}, R^{6b}, and R^{6c} is halogen and one of R^{6a}, R^{6b}, and R^{6c} is C₁-C₆ alkyl. In some embodiments, one of R^{6a}, R^{6b}, and R^{6c} is Cl and one of R^{6a}, R^{6b}, and R^{6c} is methyl. In some embodiments, R^{6a} is C₁-C₆ alkyl. In some embodiments, R^{6b} is C₁-C₆ alkyl. In some embodiments, R^{6c} is C₁-C₆ alkyl. In some embodiments, R^{6a} is methyl, ethyl, n-propyl, isopropyl, n-butyl, isobutyl, secbutyl, or tertbutyl. In some embodiments, R^{6b} is methyl, ethyl, n-propyl, isopropyl, n-butyl, isobutyl, secbutyl, or tertbutyl. In some embodiments, R^{6c} is methyl, ethyl, n-propyl, isopropyl, n-butyl, isobutyl, secbutyl, or tertbutyl. In some embodiments, R^{6a} is C₁-C₆ alkyl and R^{6b} is halogen. In some embodiments, R^{6c} is C₁-C₆ alkyl and R^{6a} is halogen. In some embodiments, R^{6b} is C₁-C₆ alkyl and R^{6c} is halogen. In some embodiments, R^{6a} is C₁-C₆ alkyl and R^{6b} is halogen. In some embodiments, R^{6c} is C₁-C₆ alkyl and R^{6a} is halogen. In some embodiments, R^{6b} is C₁-C₆ alkyl and R^{6c} is halogen. In some embodiments, two of R^{6a}, R^{6b}, and R^{6c} are halogen. In some embodiments, two of R^{6a}, R^{6b}, and R^{6c} are C₁-C₆ alkyl.

In some embodiments of a compound of Formula (I), (II), or (III), A is

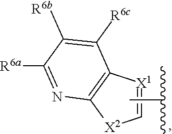

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is phenyl, optionally substituted with R⁷.

In some embodiments of a compound of Formula (I), (II), or (III), A is

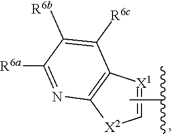

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is a 5- to 6-membered heteroaryl, optionally substituted with R⁷.

In some embodiments of a compound of Formula (I), (II), or (III), A is

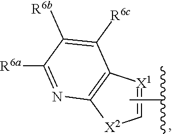

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is a 5- to 6-membered carbocycle, optionally substituted with R⁷.

In some embodiments of a compound of Formula (I), (II), or (III), A is

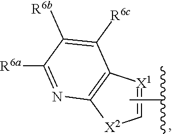

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is a 5- to 6-membered heterocycle, optionally substituted with R⁷.

In some embodiments of a compound of Formula (I), (II), or (III), A is

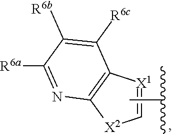

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is a 9- to 10-membered heteroaryl, optionally substituted with R⁷.

In some embodiments of a compound of Formula (I), (II), or (III), A is

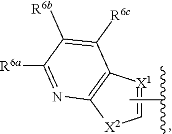

wherein one or two of R^{6a}, R^{6b}, and R^{6c} are independently selected from the group consisting of C₁-C₆ alkyl, halogen, —CN, and —OC₁-C₆ alkyl, d the remainder of R^{6a}, R^{6b}, and R^{6c} are each H; and B is selected from the group consisting of:

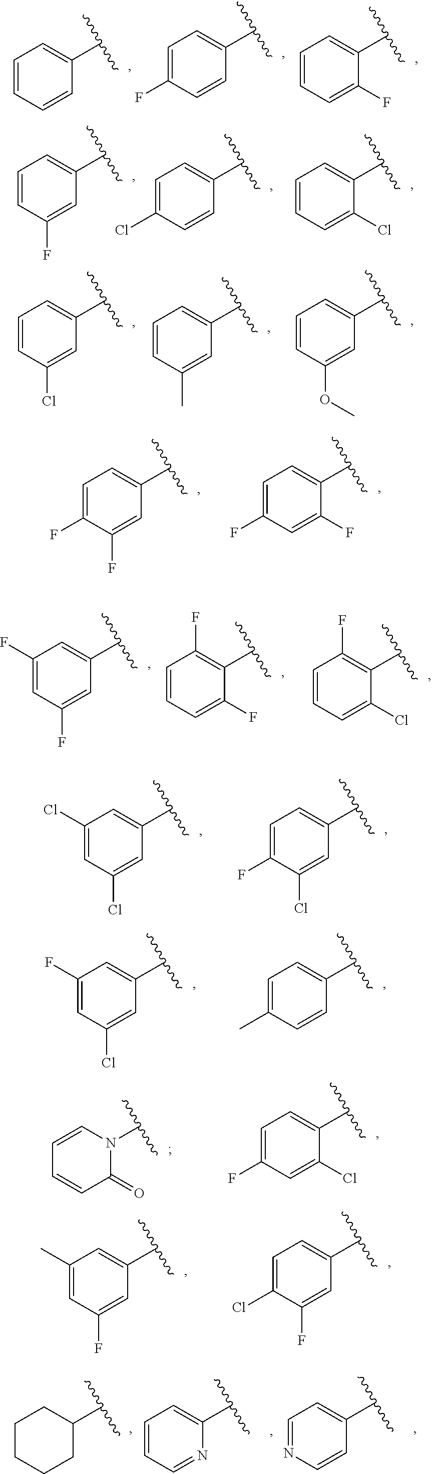

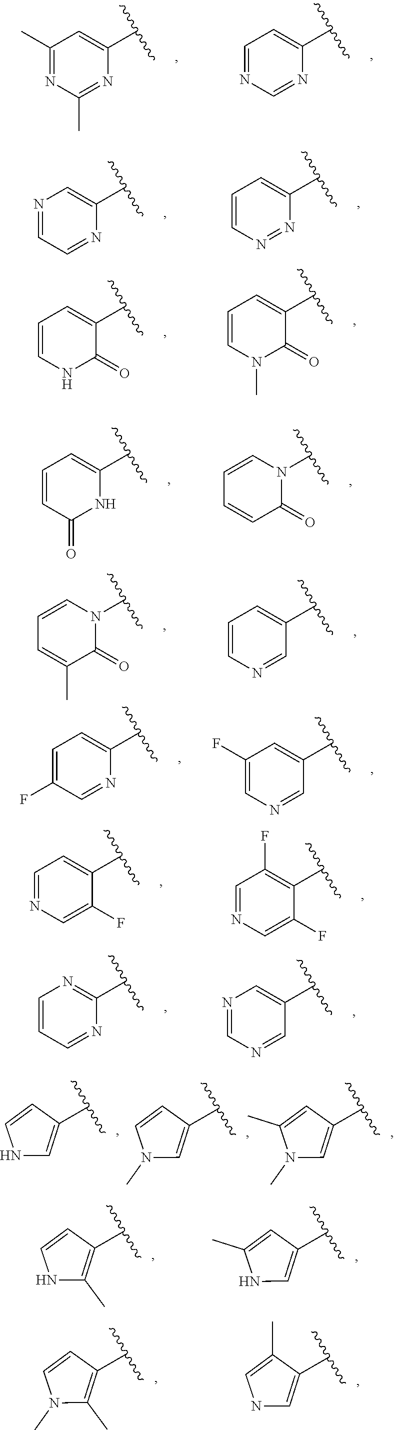

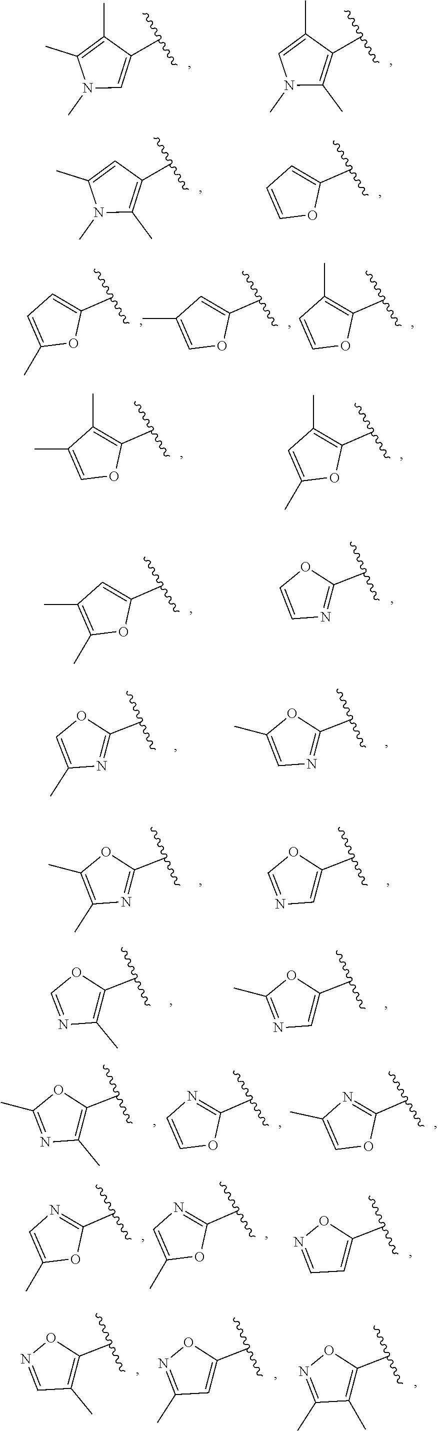

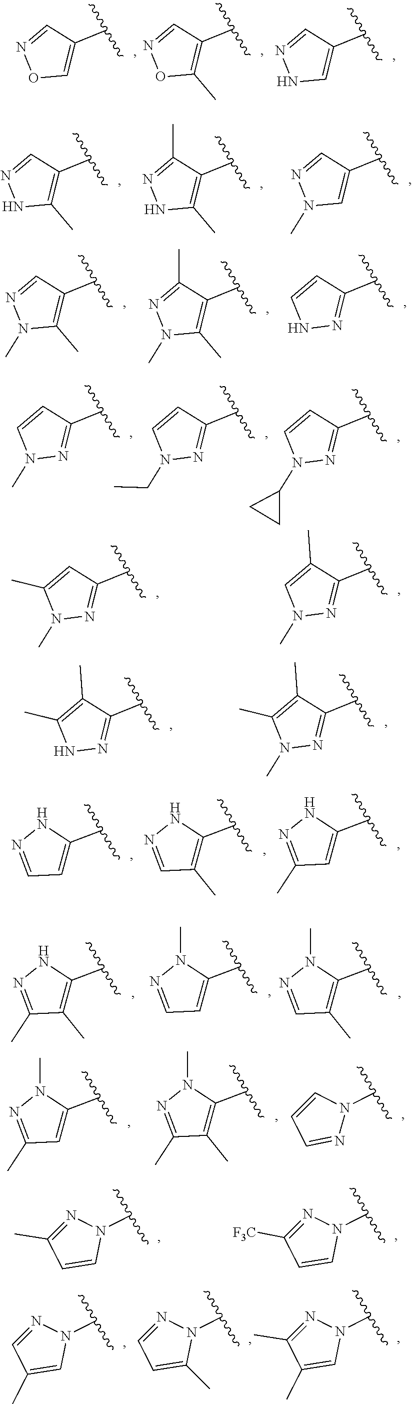

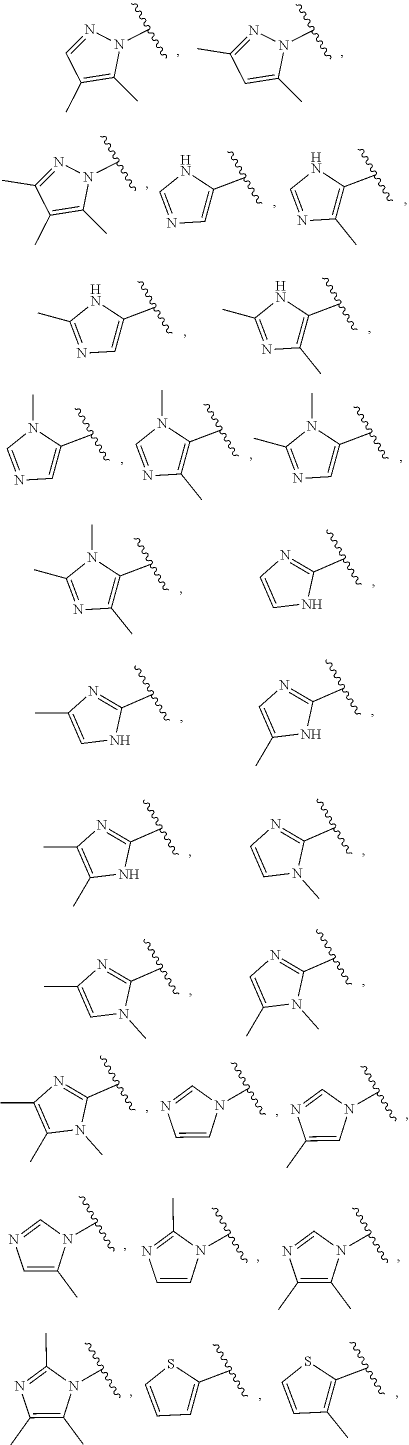

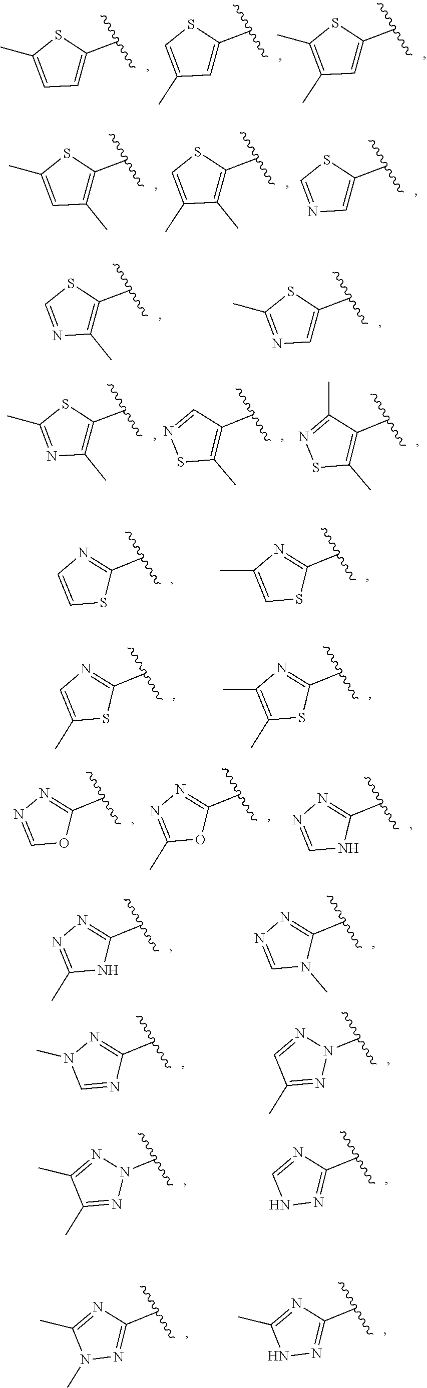

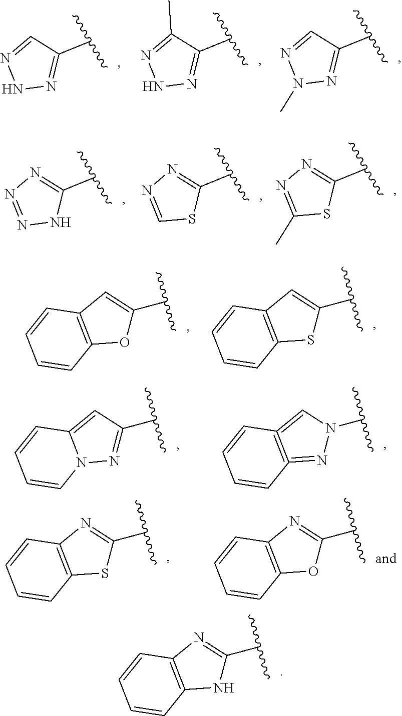

In some embodiments of a compound of Formula (I), (II), or (III), A is

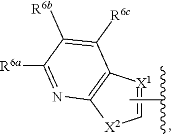

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is selected from the group consisting of:

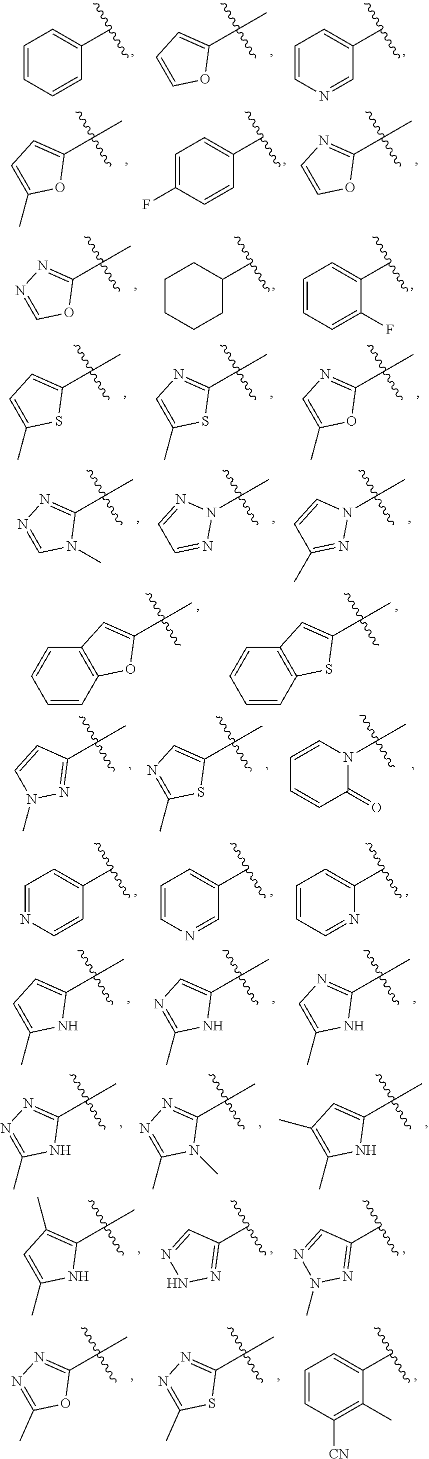

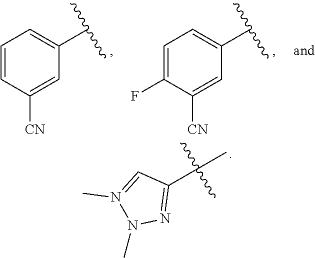

In some embodiments of a compound of Formula (I), (II), or (III), A is

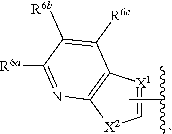

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is selected from the group consisting of

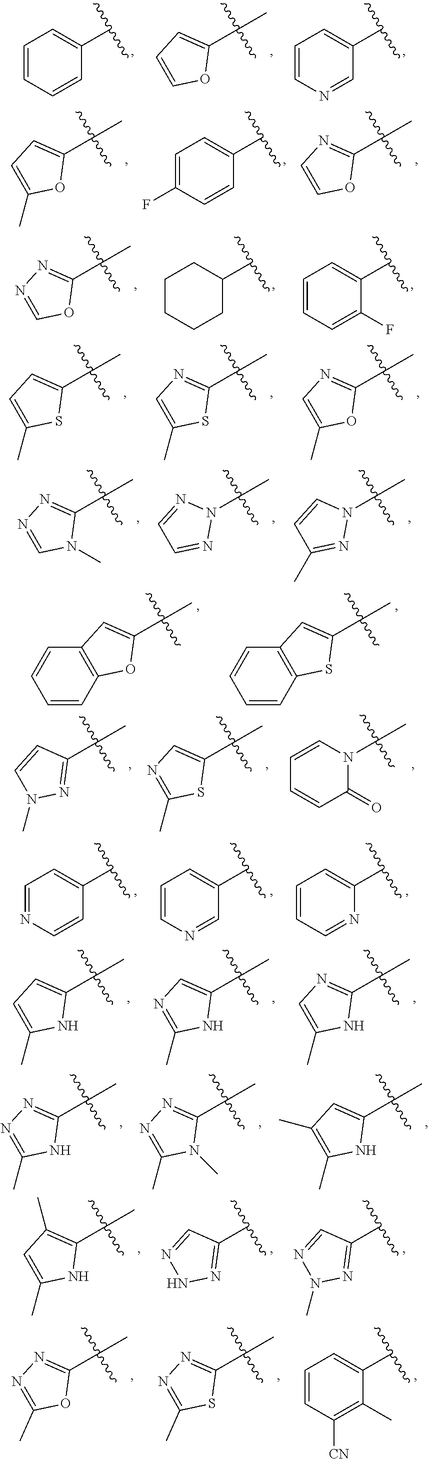

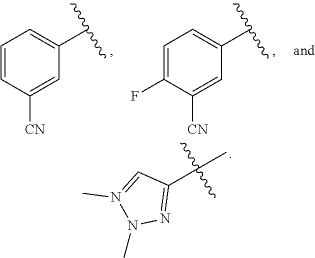

In some embodiments of a compound of Formula (I), (II), or (III), A is

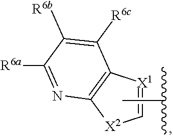

wherein one or two of R^{6a}, R^{6b}, and R^{6c} are independently selected from the group consisting of C₁-C₆ alkyl, halogen, —CN, and —OC₁-C₆ alkyl, and the remainder of R^{6a}, R^{6b}, and R^{6c} are each H; and B is selected from

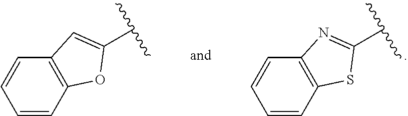 [SMILES: *c1cc2ccccc2o1 ; *c1nc2ccccc2s1]

In some embodiments of a compound of Formula (I), (II), or (III), A is

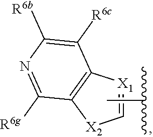

wherein X¹ is selected from the group consisting of N, C, and CH; X² is selected from the group consisting of NH, O, and S; and R^{6g}, R^{6b}, and R^{6c} are each independently H, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁹, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁹, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁹, —(C₁-C₃ alkylene)OC(O)R⁹, —(C₁-C₃ alkylene)C(O)OR⁹, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{6g}, R^{6b}, and R^{6c} is independently optionally substituted by halogen, oxo, —OR¹¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen.

In some embodiments of a compound of Formula (I), (II), or (III), A is

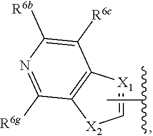

wherein X¹ is selected from the group consisting of N, C, and CH; X² is selected from the group consisting of NH, O, and S; and R^{6g}, R^{6b}, and R^{6c} are each independently H, C₁-C₆ alkyl, halogen, —CN, or —OC₁-C₆ alkyl. In some embodiments of a compound of Formula (I), (II), or (III), A is

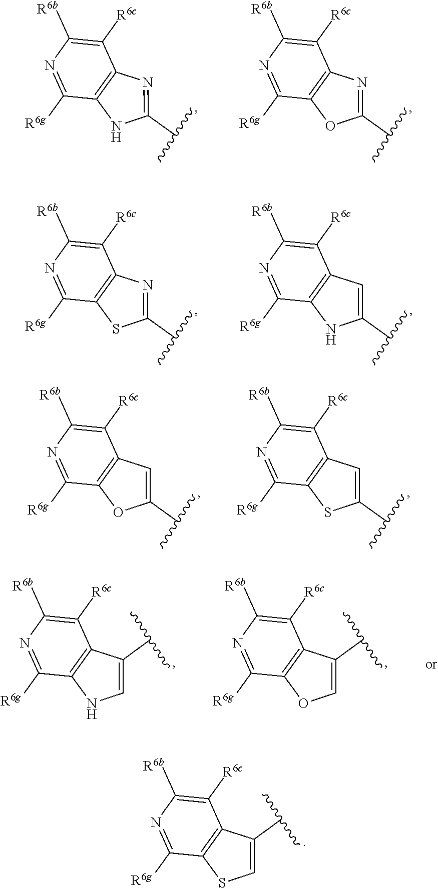

In some embodiments, R^{6g}, R^{6b}, and R^{6c} are each H. In some embodiments, one of R^{6g}, R^{6b}, and R^{6c} is Cl, F, Br, or I. In some embodiments, one of R^{6g}, R^{6b}, and R^{6c} is Cl. In some embodiments, one of R^{6g}, R^{6b}, and R^{6c} is halogen and the others are each H. In some embodiments, one of R^{6g}, R^{6b}, and R^{6c} is halogen and one of R^{6g}, R^{6b}, and R^{6c} is C₁-C₆ alkyl. In some embodiments, one of R^{6g}, R^{6b}, and R^{6c} is Cl and one of R^{6g}, R^{6b}, and R^{6c} is methyl. In some embodiments, R^{6g} is C₁-C₆ alkyl. In some embodiments, R^{6b} is C₁-C₆ alkyl. In some embodiments, R^{6c} is C₁-C₆ alkyl. In some embodiments, R^{6g} is methyl, ethyl, n-propyl, isopropyl, n-butyl, isobutyl, secbutyl, or tertbutyl. In some embodiments, R^{6b} is methyl, ethyl, n-propyl, isopropyl, n-butyl, isobutyl, secbutyl, or tertbutyl. In some embodiments, R^{6c} is methyl, ethyl, n-propyl, isopropyl, n-butyl, isobutyl, secbutyl, or tertbutyl. In some embodiments, R^{6g} is C₁-C₆ alkyl and R^{6b} is halogen. In some embodiments, R^{6g} is C₁-C₆ alkyl and R^{6c} is halogen. In some embodiments, R^{6b} is C₁-C₆ alkyl and R^{6g} is halogen. In some embodiments, R^{6b} is C₁-C₆ alkyl and R^{6c} is halogen. In some embodiments, R^{6c} is C₁-C₆ alkyl and R^{6g} is halogen. In some embodiments, R^{6c} is C₁-C₆ alkyl and R^{6b} is halogen. In some embodiments, two of R^{6g}, R^{6b}, and R^{6c} are halogen. In some embodiments, two of R^{6g}, R^{6b}, and R^{6c} are C₁-C₆ alkyl.

In some embodiments of a compound of Formula (I), (II), or (III), A is

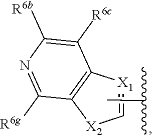

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is phenyl, optionally substituted with R⁷.

In some embodiments of a compound of Formula (I), (II), or (III), A is

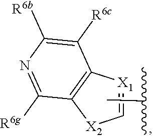

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is a 5- to 6-membered heteroaryl, optionally substituted with R⁷.

In some embodiments of a compound of Formula (I), (II), or (III), A is

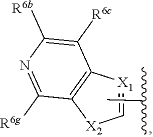

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is a 5- to 6-membered carbocycle, optionally substituted with R⁷.

In some embodiments of a compound of Formula (I), (II), or (III), A is

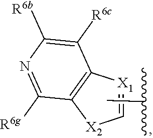

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R¹ are each H; and B is a 5- to 6-membered heterocycle, optionally substituted with R⁷.

In some embodiments of a compound of Formula (I), (II), or (III), A is

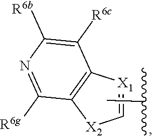

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is a 9- to 10-membered heteroaryl, optionally substituted with R⁷.

In some embodiments of a compound of Formula (I), (II), or (III), A is

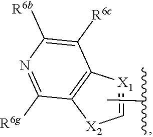

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is selected from the group consisting of:

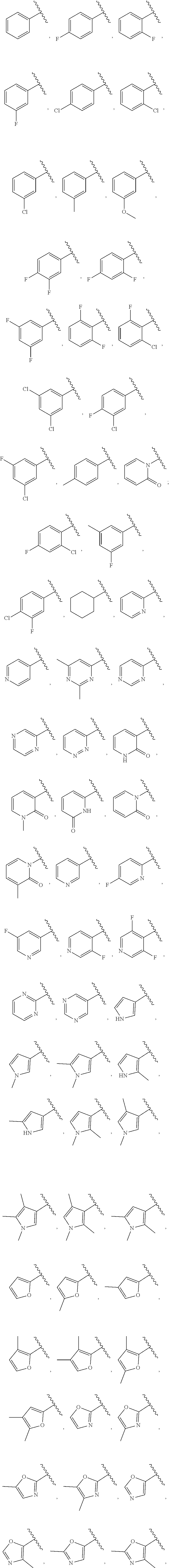

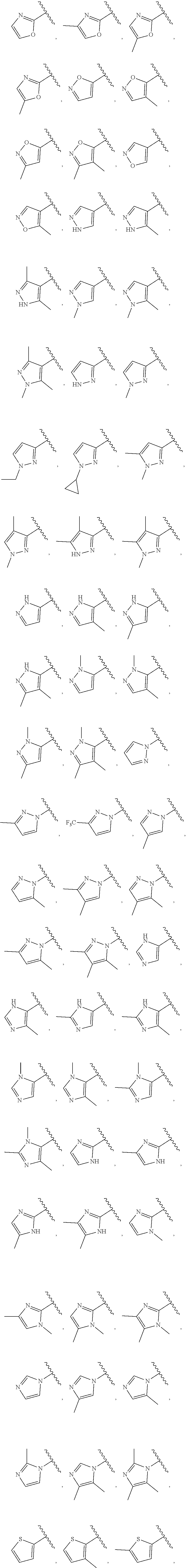

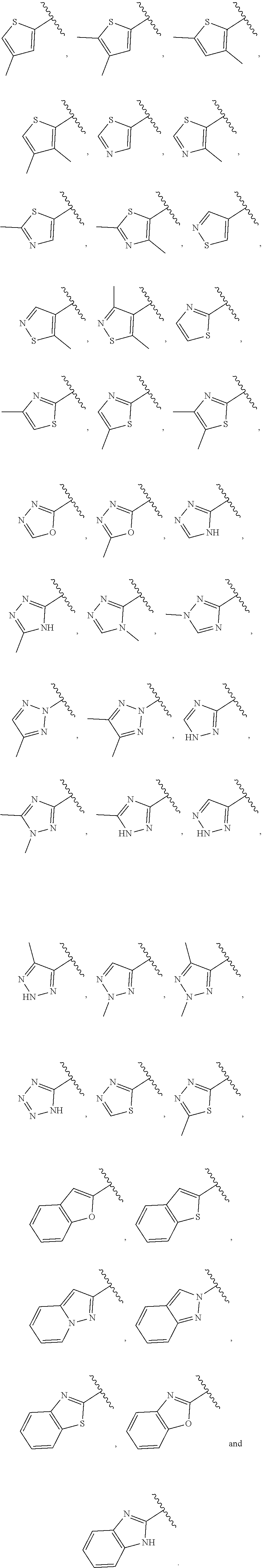

In some embodiments of a compound of Formula (I), (II), or (III), A is

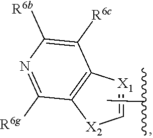

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is selected from the group consisting of:

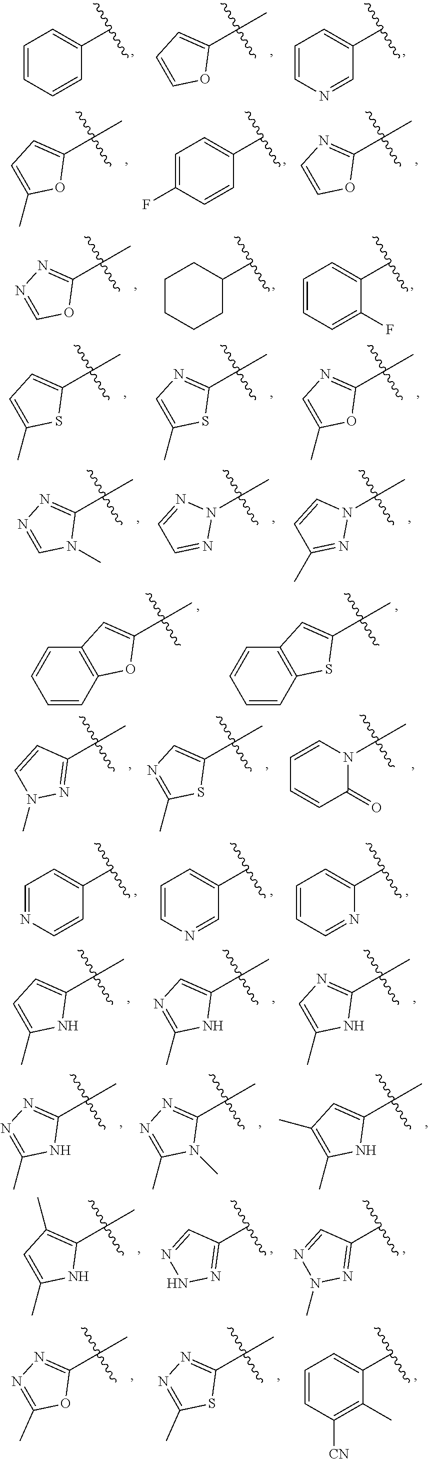

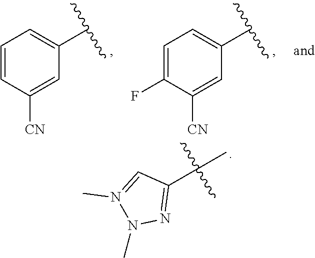

In some embodiments of a compound of Formula (I), (II), or (III), A is

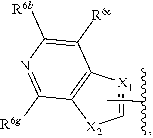

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is selected from the group consisting of

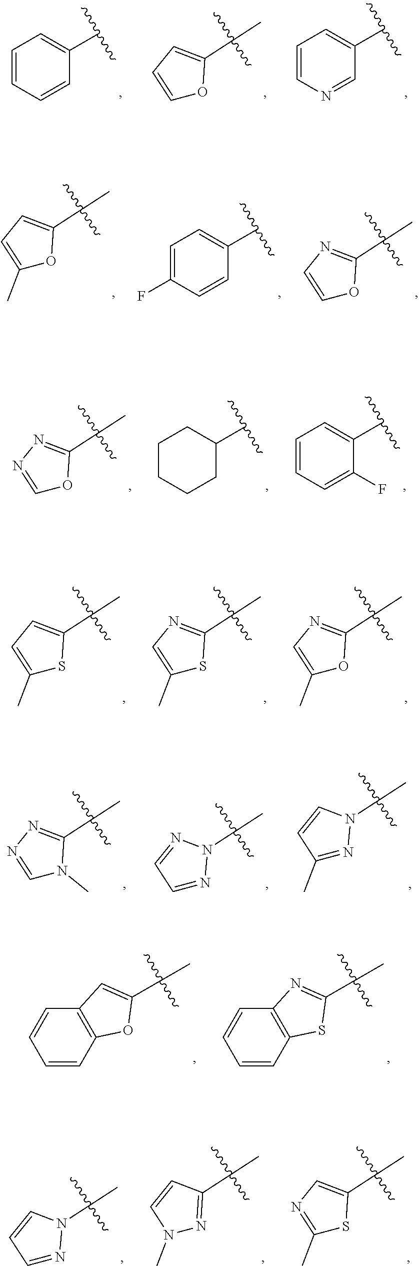

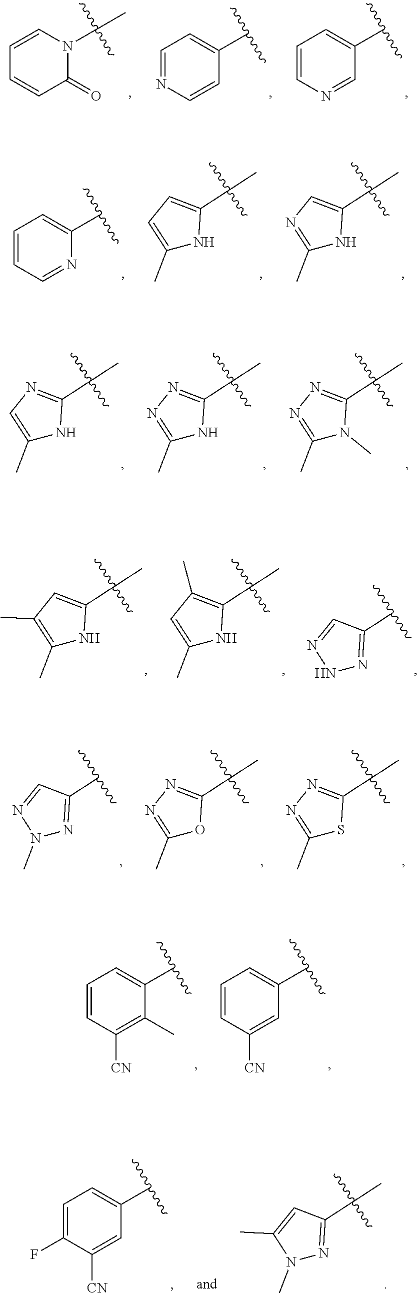

In some embodiments of a compound of Formula (I), (II), or (III), A is

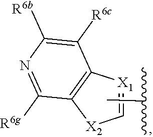

wherein one or two of R^{6g}, R^{6b}, and R^{6c} are independently selected from the group consisting of C₁-C₆ alkyl, halogen, —CN, and —OC₁-C₆ alkyl, and the remainder of R^{6g}, R^{6b}, and R^{6c} are each H; and B is selected from

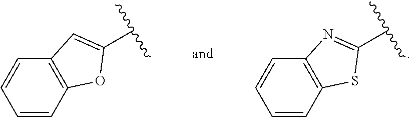 [SMILES: *c1cc2ccccc2o1 ; *c1nc2ccccc2s1]

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to A of formula (I) may be combined with every description, variation, embodiment or aspect of R¹, R², R³, R⁴, and B the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae (II) and (III), as detailed herein, and are equally described, the same as if each and every description, variation, embodiment or aspect were separately and individually listed for all formulae.

Also provided are salts of compounds referred to herein, such as pharmaceutically acceptable salts. The invention also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds are listed in Table 1. It is understood that individual enantiomers and diastereomers if not depicted are embraced herein and their corresponding structures can be readily determined therefrom.

TABLE 1

| Compound | |
|---|---|
| No. | Compound |
| 1.1 | 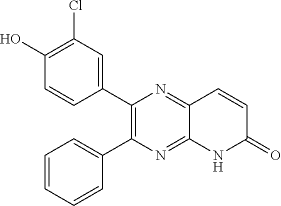 [SMILES: O=c1ccc2nc(-c3ccc(O)c(Cl)c3)c(-c3ccccc3)nc2[nH]1] |
| 1.2 | 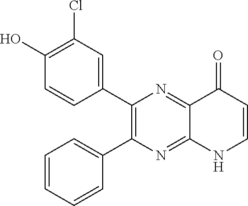 [SMILES: O=c1cc[nH]c2nc(-c3ccccc3)c(-c3ccc(O)c(Cl)c3)nc12] |
| 1.3 | 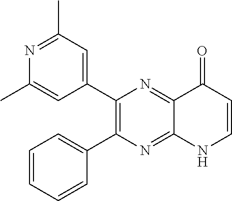 [SMILES: Cc1cc(-c2nc3c(=O)cc[nH]c3nc2-c2ccccc2)cc(C)n1] |
| 1.4 | 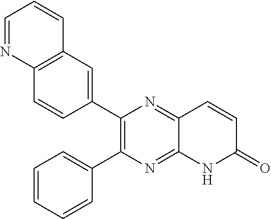 [SMILES: O=c1ccc2nc(-c3ccc4ncccc4c3)c(-c3ccccc3)nc2[nH]1] |
| 1.5 | 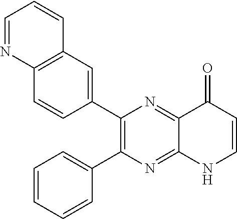 [SMILES: O=c1cc[nH]c2nc(-c3ccccc3)c(-c3ccc4ncccc4c3)nc12] |
| 1.6 | 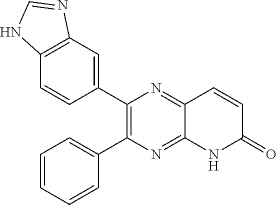 [SMILES: O=c1ccc2nc(-c3ccc4[nH]cnc4c3)c(-c3ccccc3)nc2[nH]1] |
| 1.7 | 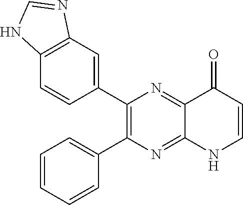 [SMILES: O=c1cc[nH]c2nc(-c3ccccc3)c(-c3ccc4[nH]cnc4c3)nc12] |
| 1.8 | 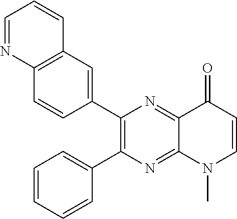 [SMILES: Cn1ccc(=O)c2nc(-c3ccc4ncccc4c3)c(-c3ccccc3)nc21] |
| 1.9 | 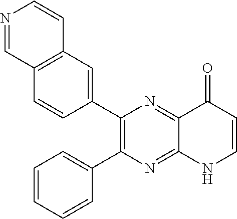 [SMILES: O=c1cc[nH]c2nc(-c3ccccc3)c(-c3ccc4cnccc4c3)nc12] |
| 1.10 | 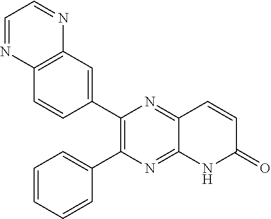 [SMILES: O=c1ccc2nc(-c3ccc4nccnc4c3)c(-c3ccccc3)nc2[nH]1] |
| 1.11 | 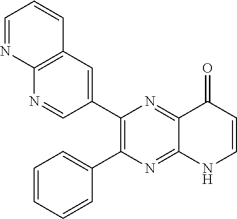 [SMILES: O=c1cc[nH]c2nc(-c3ccccc3)c(-c3cnc4ncccc4c3)nc12] |
| 1.12 | 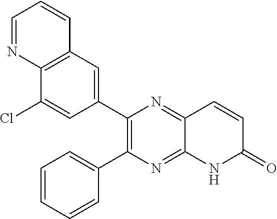 [SMILES: O=c1ccc2nc(-c3cc(Cl)c4ncccc4c3)c(-c3ccccc3)nc2[nH]1] |
| 1.13 | 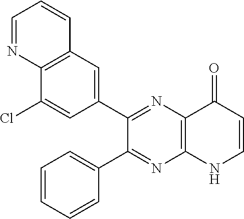 [SMILES: O=c1cc[nH]c2nc(-c3ccccc3)c(-c3cc(Cl)c4ncccc4c3)nc12] |
| 1.14 | 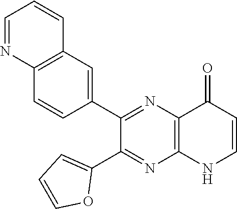 [SMILES: O=c1cc[nH]c2nc(-c3ccco3)c(-c3ccc4ncccc4c3)nc12] |
| 1.15 | 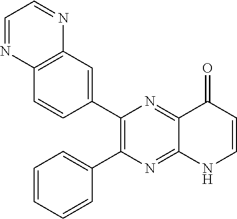 [SMILES: O=c1cc[nH]c2nc(-c3ccccc3)c(-c3ccc4nccnc4c3)nc12] |
| 1.16 | 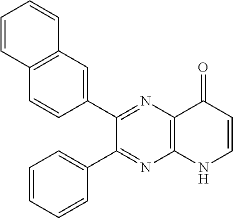 [SMILES: O=c1cc[nH]c2nc(-c3ccccc3)c(-c3ccc4ccccc4c3)nc12] |
| 1.17 | 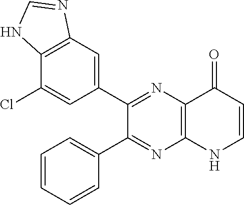 [SMILES: O=c1cc[nH]c2nc(-c3ccccc3)c(-c3cc(Cl)c4[nH]cnc4c3)nc12] |
| 1.18 | 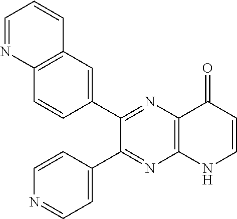 [SMILES: O=c1cc[nH]c2nc(-c3ccncc3)c(-c3ccc4ncccc4c3)nc12] |
| 1.19 | 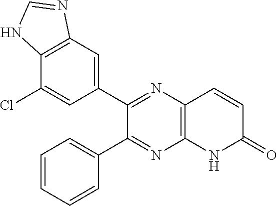 [SMILES: O=c1ccc2nc(-c3cc(Cl)c4[nH]cnc4c3)c(-c3ccccc3)nc2[nH]1] |
| 1.20 | 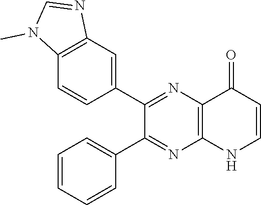 [SMILES: Cn1cnc2cc(-c3nc4c(=O)cc[nH]c4nc3-c3ccccc3)ccc21] |
| 1.21 | 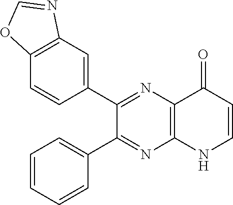 [SMILES: O=c1cc[nH]c2nc(-c3ccccc3)c(-c3ccc4ocnc4c3)nc12] |
| 1.22 | 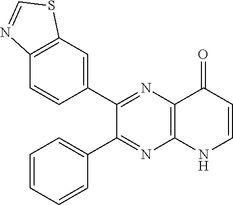 [SMILES: O=c1cc[nH]c2nc(-c3ccccc3)c(-c3ccc4ncsc4c3)nc12] |
| 1.23 | 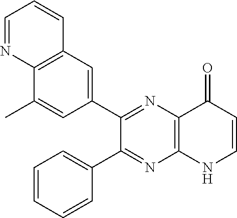 [SMILES: Cc1cc(-c2nc3c(=O)cc[nH]c3nc2-c2ccccc2)cc2cccnc12] |
| 1.24 | 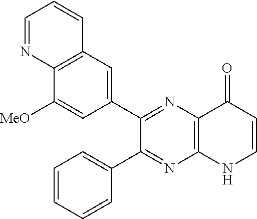 [SMILES: COc1cc(-c2nc3c(=O)cc[nH]c3nc2-c2ccccc2)cc2cccnc12] |
| 1.25 | 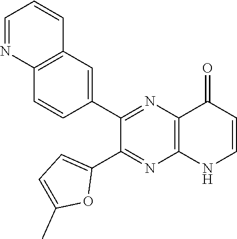 [SMILES: Cc1ccc(-c2nc3[nH]ccc(=O)c3nc2-c2ccc3ncccc3c2)o1] |
| 1.26 | 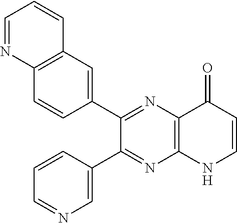 [SMILES: O=c1cc[nH]c2nc(-c3cccnc3)c(-c3ccc4ncccc4c3)nc12] |
| 1.27 | 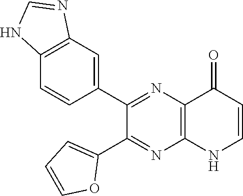 [SMILES: O=c1cc[nH]c2nc(-c3ccco3)c(-c3ccc4[nH]cnc4c3)nc12] |
| 1.28 | 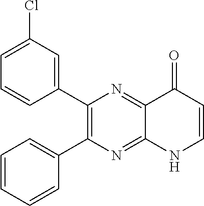 [SMILES: O=c1cc[nH]c2nc(-c3ccccc3)c(-c3cccc(Cl)c3)nc12] |
| 1.29 | 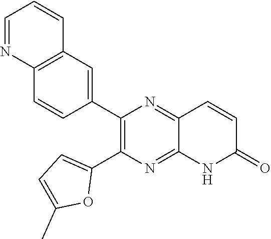 [SMILES: Cc1ccc(-c2nc3[nH]c(=O)ccc3nc2-c2ccc3ncccc3c2)o1] |
| 1.30 | 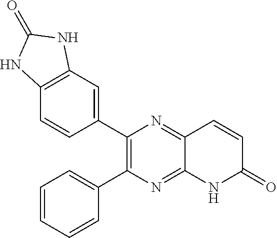 [SMILES: O=c1ccc2nc(-c3ccc4[nH]c(=O)[nH]c4c3)c(-c3ccccc3)nc2[nH]1] |
| 1.31 | 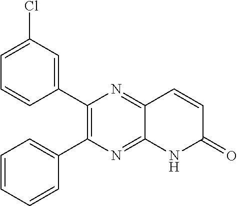 [SMILES: O=c1ccc2nc(-c3cccc(Cl)c3)c(-c3ccccc3)nc2[nH]1] |
| 1.32 | 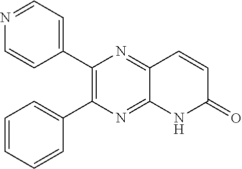 [SMILES: O=c1ccc2nc(-c3ccncc3)c(-c3ccccc3)nc2[nH]1] |
| 1.33 | 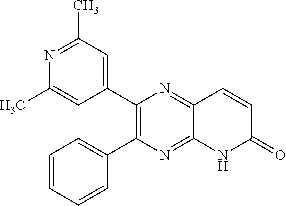 [SMILES: Cc1cc(-c2nc3ccc(=O)[nH]c3nc2-c2ccccc2)cc(C)n1] |
| 1.34 | 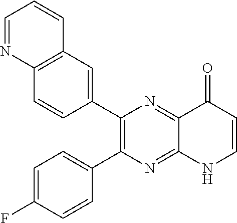 [SMILES: O=c1cc[nH]c2nc(-c3ccc(F)cc3)c(-c3ccc4ncccc4c3)nc12] |
| 1.35 | 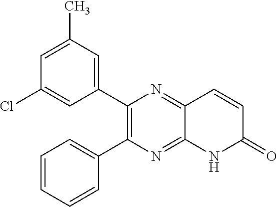 [SMILES: Cc1cc(Cl)cc(-c2nc3ccc(=O)[nH]c3nc2-c2ccccc2)c1] |
| 1.36 | 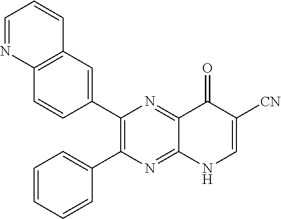 [SMILES: N#Cc1c[nH]c2nc(-c3ccccc3)c(-c3ccc4ncccc4c3)nc2c1=O] |
| 1.37 | 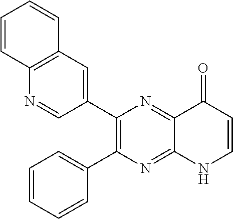 [SMILES: O=c1cc[nH]c2nc(-c3ccccc3)c(-c3cnc4ccccc4c3)nc12] |
| 1.38 | 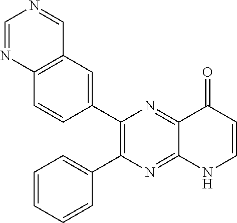 [SMILES: O=c1cc[nH]c2nc(-c3ccccc3)c(-c3ccc4ncncc4c3)nc12] |
| 1.39 | 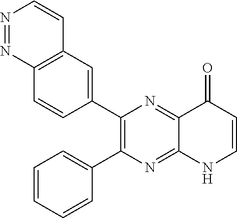 [SMILES: O=c1cc[nH]c2nc(-c3ccccc3)c(-c3ccc4nnccc4c3)nc12] |
| 1.40 | 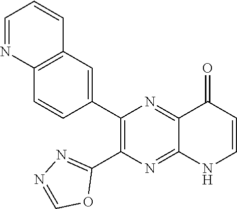 [SMILES: O=c1cc[nH]c2nc(-c3nnco3)c(-c3ccc4ncccc4c3)nc12] |
| 1.41 | 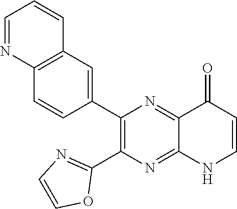 [SMILES: O=c1cc[nH]c2nc(-c3ncco3)c(-c3ccc4ncccc4c3)nc12] |
| 1.42 | 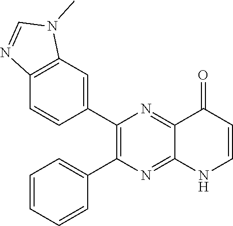 [SMILES: Cn1cnc2ccc(-c3nc4c(=O)cc[nH]c4nc3-c3ccccc3)cc21] |
| 1.43 | 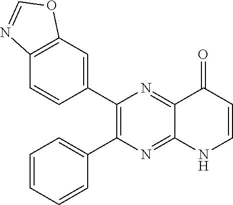 [SMILES: O=c1cc[nH]c2nc(-c3ccccc3)c(-c3ccc4ncoc4c3)nc12] |
| 1.44 | 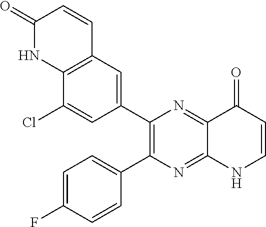 [SMILES: O=c1ccc2cc(-c3nc4c(=O)cc[nH]c4nc3-c3ccc(F)cc3)cc(Cl)c2[nH]1] |
| 1.45 | 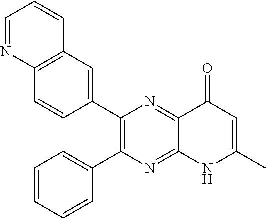 [SMILES: Cc1cc(=O)c2nc(-c3ccc4ncccc4c3)c(-c3ccccc3)nc2[nH]1] |
| 1.46 | 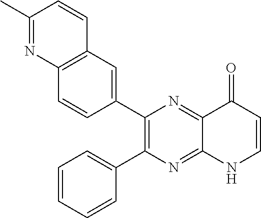 [SMILES: Cc1ccc2cc(-c3nc4c(=O)cc[nH]c4nc3-c3ccccc3)ccc2n1] |
| 1.47 | 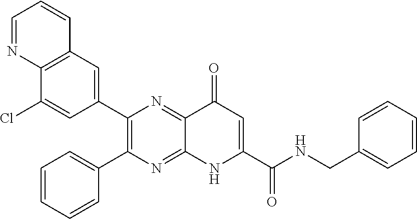 [SMILES: O=C(NCc1ccccc1)c1cc(=O)c2nc(-c3cc(Cl)c4ncccc4c3)c(-c3ccccc3)nc2[nH]1] |
| 1.48 | 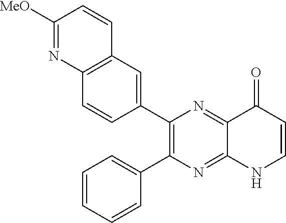 [SMILES: COc1ccc2cc(-c3nc4c(=O)cc[nH]c4nc3-c3ccccc3)ccc2n1] |
| 1.49 | 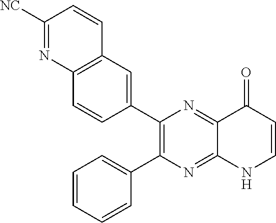 [SMILES: N#Cc1ccc2cc(-c3nc4c(=O)cc[nH]c4nc3-c3ccccc3)ccc2n1] |
| 1.50 | 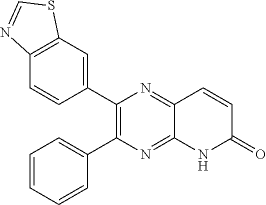 [SMILES: O=c1ccc2nc(-c3ccc4ncsc4c3)c(-c3ccccc3)nc2[nH]1] |
| 1.51 | 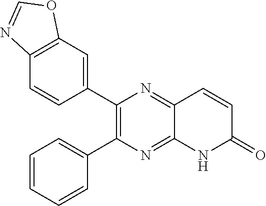 [SMILES: O=c1ccc2nc(-c3ccc4ncoc4c3)c(-c3ccccc3)nc2[nH]1] |
| 1.52 | 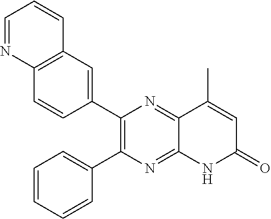 [SMILES: Cc1cc(=O)[nH]c2nc(-c3ccccc3)c(-c3ccc4ncccc4c3)nc12] |
| 1.53 | 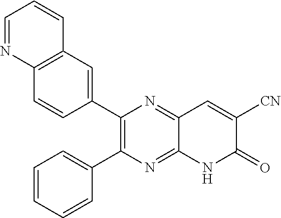 [SMILES: N#Cc1cc2nc(-c3ccc4ncccc4c3)c(-c3ccccc3)nc2[nH]c1=O] |
| 1.54 | 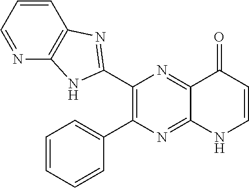 [SMILES: O=c1cc[nH]c2nc(-c3ccccc3)c(-c3nc4cccnc4[nH]3)nc12] |
| 1.55 | 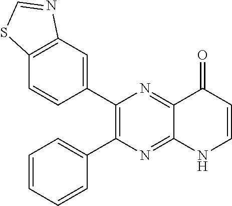 [SMILES: O=c1cc[nH]c2nc(-c3ccccc3)c(-c3ccc4scnc4c3)nc12] |
| 1.56 | 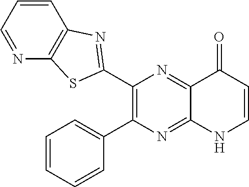 [SMILES: O=c1cc[nH]c2nc(-c3ccccc3)c(-c3nc4cccnc4s3)nc12] |
| 1.57 | 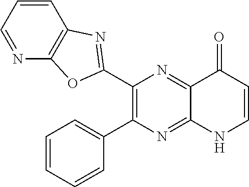 [SMILES: O=c1cc[nH]c2nc(-c3ccccc3)c(-c3nc4cccnc4o3)nc12] |
| 1.58 | 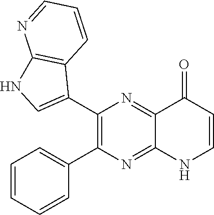 [SMILES: O=c1cc[nH]c2nc(-c3ccccc3)c(-c3c[nH]c4ncccc34)nc12] |
| 1.59 | 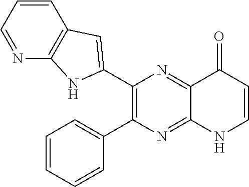 [SMILES: O=c1cc[nH]c2nc(-c3ccccc3)c(-c3cc4cccnc4[nH]3)nc12] |
| 1.60 | 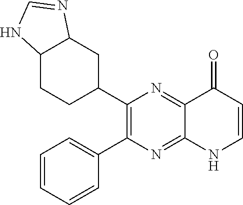 [SMILES: O=c1cc[nH]c2nc(-c3ccccc3)c(C3CCC4NC=NC4C3)nc12] |
| 1.61 | 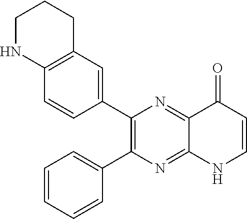 [SMILES: O=c1cc[nH]c2nc(-c3ccccc3)c(-c3ccc4c(c3)CCCN4)nc12] |
| 1.62 | 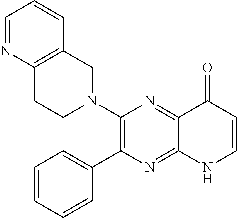 [SMILES: O=c1cc[nH]c2nc(-c3ccccc3)c(N3CCc4ncccc4C3)nc12] |
| 1.63 | 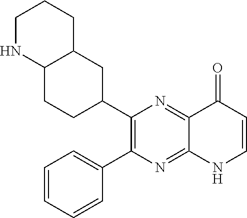 [SMILES: O=c1cc[nH]c2nc(-c3ccccc3)c(C3CCC4NCCCC4C3)nc12] |
| 1.64 | 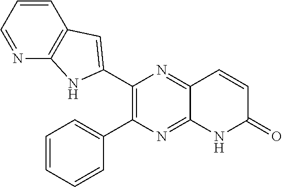 [SMILES: O=c1ccc2nc(-c3cc4cccnc4[nH]3)c(-c3ccccc3)nc2[nH]1] |
| 1.65 | 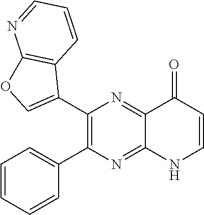 [SMILES: O=c1cc[nH]c2nc(-c3ccccc3)c(-c3coc4ncccc34)nc12] |
| 1.66 | 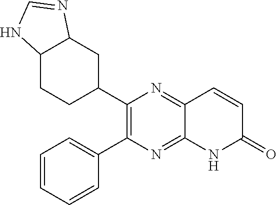 [SMILES: O=c1ccc2nc(C3CCC4NC=NC4C3)c(-c3ccccc3)nc2[nH]1] |
| 1.67 | 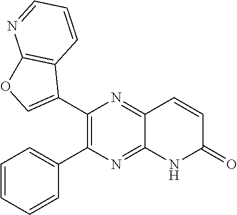 [SMILES: O=c1ccc2nc(-c3coc4ncccc34)c(-c3ccccc3)nc2[nH]1] |
| 1.68 | 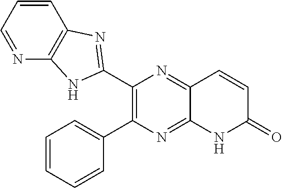 [SMILES: O=c1ccc2nc(-c3nc4cccnc4[nH]3)c(-c3ccccc3)nc2[nH]1] |
| 1.69 | 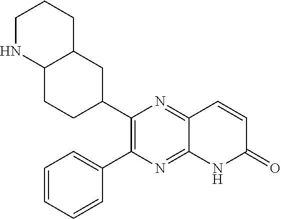 [SMILES: O=c1ccc2nc(C3CCC4NCCCC4C3)c(-c3ccccc3)nc2[nH]1] |
| 1.70 | 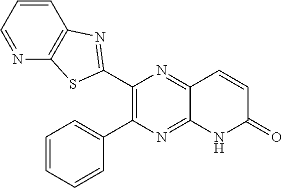 [SMILES: O=c1ccc2nc(-c3nc4cccnc4s3)c(-c3ccccc3)nc2[nH]1] |
| 1.71 | 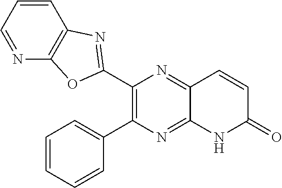 [SMILES: O=c1ccc2nc(-c3nc4cccnc4o3)c(-c3ccccc3)nc2[nH]1] |
| 1.72 | 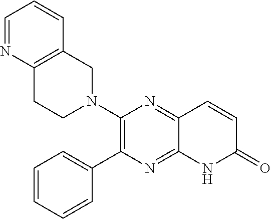 [SMILES: O=c1ccc2nc(N3CCc4ncccc4C3)c(-c3ccccc3)nc2[nH]1] |
| 1.73 | 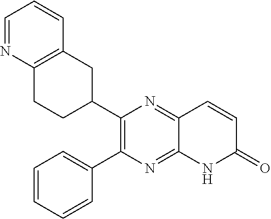 [SMILES: O=c1ccc2nc(C3CCc4ncccc4C3)c(-c3ccccc3)nc2[nH]1] |
| 1.74 | 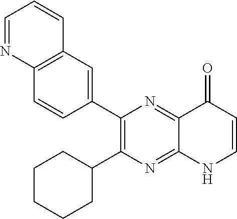 [SMILES: O=c1cc[nH]c2nc(C3CCCCC3)c(-c3ccc4ncccc4c3)nc12] |
| 1.75 | 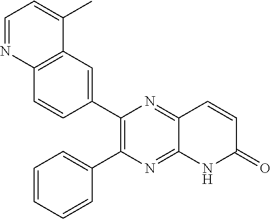 [SMILES: Cc1ccnc2ccc(-c3nc4ccc(=O)[nH]c4nc3-c3ccccc3)cc12] |
| 1.76 | 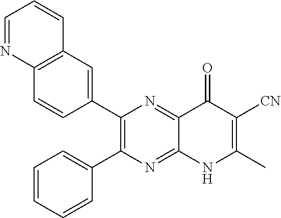 [SMILES: Cc1[nH]c2nc(-c3ccccc3)c(-c3ccc4ncccc4c3)nc2c(=O)c1C#N] |
| 1.77 | 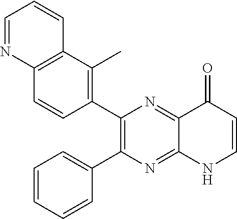 [SMILES: Cc1c(-c2nc3c(=O)cc[nH]c3nc2-c2ccccc2)ccc2ncccc12] |
| 1.78 | 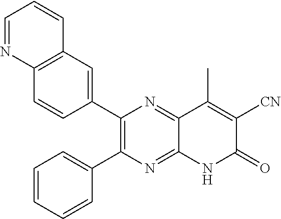 [SMILES: Cc1c(C#N)c(=O)[nH]c2nc(-c3ccccc3)c(-c3ccc4ncccc4c3)nc12] |
| 1.79 | 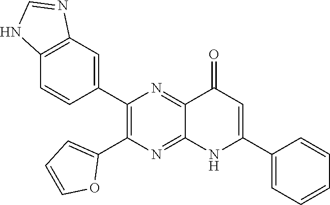 [SMILES: O=c1cc(-c2ccccc2)[nH]c2nc(-c3ccco3)c(-c3ccc4[nH]cnc4c3)nc12] |
| 1.80 | 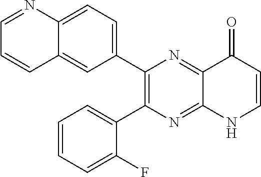 [SMILES: O=c1cc[nH]c2nc(-c3ccccc3F)c(-c3ccc4ncccc4c3)nc12] |
| 1.81 | 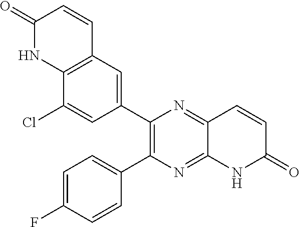 [SMILES: O=c1ccc2nc(-c3cc(Cl)c4[nH]c(=O)ccc4c3)c(-c3ccc(F)cc3)nc2[nH]1] |
| 1.82 | 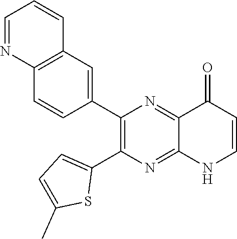 [SMILES: Cc1ccc(-c2nc3[nH]ccc(=O)c3nc2-c2ccc3ncccc3c2)s1] |
| 1.83 | 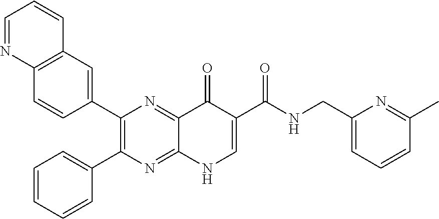 [SMILES: Cc1cccc(CNC(=O)c2c[nH]c3nc(-c4ccccc4)c(-c4ccc5ncccc5c4)nc3c2=O)n1] |
| 1.84 | 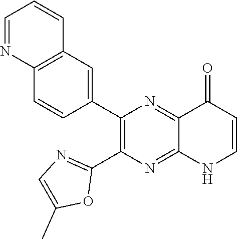 [SMILES: Cc1cnc(-c2nc3[nH]ccc(=O)c3nc2-c2ccc3ncccc3c2)o1] |
| 1.85 | 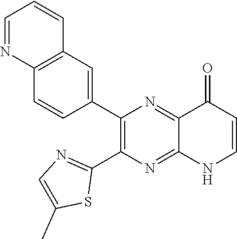 [SMILES: Cc1cnc(-c2nc3[nH]ccc(=O)c3nc2-c2ccc3ncccc3c2)s1] |
| 1.86 | 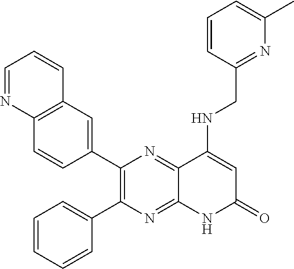 [SMILES: Cc1cccc(CNc2cc(=O)[nH]c3nc(-c4ccccc4)c(-c4ccc5ncccc5c4)nc23)n1] |
| 1.87 | 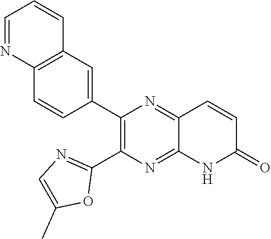 [SMILES: Cc1cnc(-c2nc3[nH]c(=O)ccc3nc2-c2ccc3ncccc3c2)o1] |
| 1.88 | 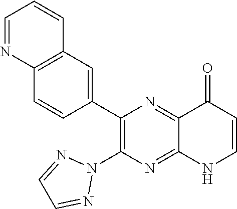 [SMILES: O=c1cc[nH]c2nc(-n3nccn3)c(-c3ccc4ncccc4c3)nc12] |
| 1.89 | 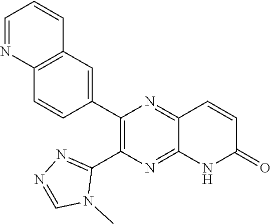 [SMILES: Cn1cnnc1-c1nc2[nH]c(=O)ccc2nc1-c1ccc2ncccc2c1] |
| 1.90 | 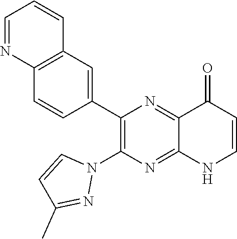 [SMILES: Cc1ccn(-c2nc3[nH]ccc(=O)c3nc2-c2ccc3ncccc3c2)n1] |
| 1.91 | 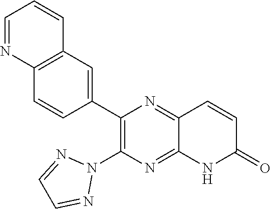 [SMILES: O=c1ccc2nc(-c3ccc4ncccc4c3)c(-n3nccn3)nc2[nH]1] |
| 1.92 | 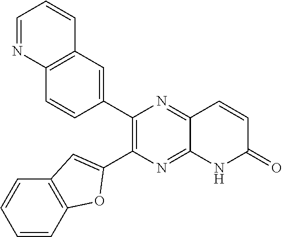 [SMILES: O=c1ccc2nc(-c3ccc4ncccc4c3)c(-c3cc4ccccc4o3)nc2[nH]1] |
| 1.93 | 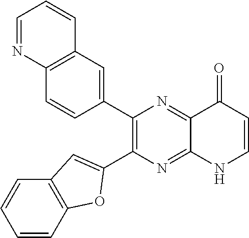 [SMILES: O=c1cc[nH]c2nc(-c3cc4ccccc4o3)c(-c3ccc4ncccc4c3)nc12] |
| 1.94 | 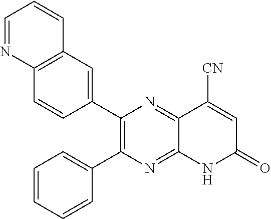 [SMILES: N#Cc1cc(=O)[nH]c2nc(-c3ccccc3)c(-c3ccc4ncccc4c3)nc12] |
| 1.95 | 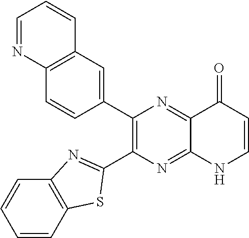 [SMILES: O=c1cc[nH]c2nc(-c3nc4ccccc4s3)c(-c3ccc4ncccc4c3)nc12] |
| 1.96 | 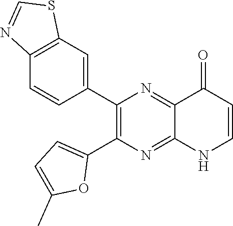 [SMILES: Cc1ccc(-c2nc3[nH]ccc(=O)c3nc2-c2ccc3ncsc3c2)o1] |
| 1.97 | 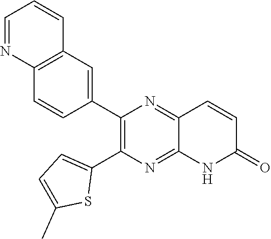 [SMILES: Cc1ccc(-c2nc3[nH]c(=O)ccc3nc2-c2ccc3ncccc3c2)s1] |
| 1.98 | 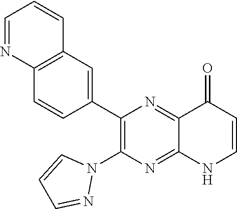 [SMILES: O=c1cc[nH]c2nc(-n3cccn3)c(-c3ccc4ncccc4c3)nc12] |
| 1.99 | 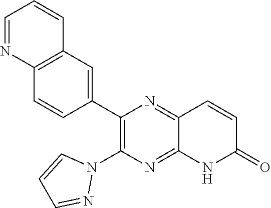 [SMILES: O=c1ccc2nc(-c3ccc4ncccc4c3)c(-n3cccn3)nc2[nH]1] |
| 1.100 | 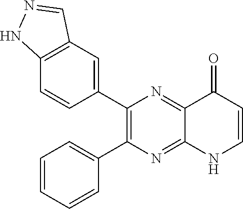 [SMILES: O=c1cc[nH]c2nc(-c3ccccc3)c(-c3ccc4[nH]ncc4c3)nc12] |
| 1.101 | 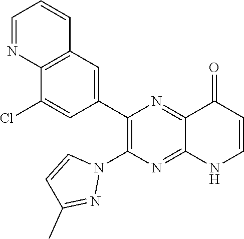 [SMILES: Cc1ccn(-c2nc3[nH]ccc(=O)c3nc2-c2cc(Cl)c3ncccc3c2)n1] |
| 1.102 | 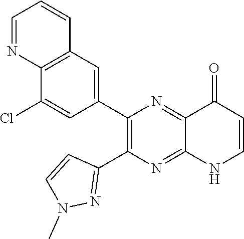 [SMILES: Cn1ccc(-c2nc3[nH]ccc(=O)c3nc2-c2cc(Cl)c3ncccc3c2)n1] |
| 1.103 | 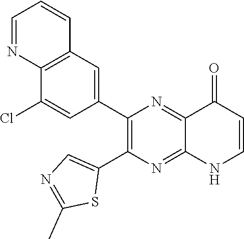 [SMILES: Cc1ncc(-c2nc3[nH]ccc(=O)c3nc2-c2cc(Cl)c3ncccc3c2)s1] |
| 1.104 | 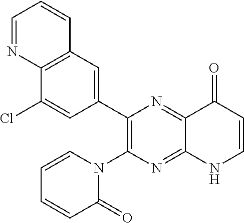 [SMILES: O=c1cc[nH]c2nc(-n3ccccc3=O)c(-c3cc(Cl)c4ncccc4c3)nc12] |
| 1.105 | 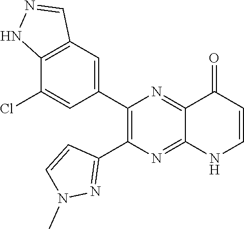 [SMILES: Cn1ccc(-c2nc3[nH]ccc(=O)c3nc2-c2cc(Cl)c3[nH]ncc3c2)n1] |
| 1.106 | 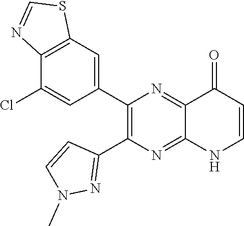 [SMILES: Cn1ccc(-c2nc3[nH]ccc(=O)c3nc2-c2cc(Cl)c3ncsc3c2)n1] |
| 1.107 | 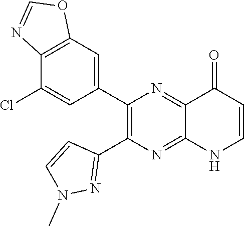 [SMILES: Cn1ccc(-c2nc3[nH]ccc(=O)c3nc2-c2cc(Cl)c3ncoc3c2)n1] |
| 1.108 | 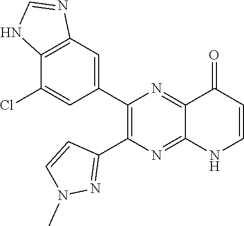 [SMILES: Cn1ccc(-c2nc3[nH]ccc(=O)c3nc2-c2cc(Cl)c3[nH]cnc3c2)n1] |
| 1.109 | 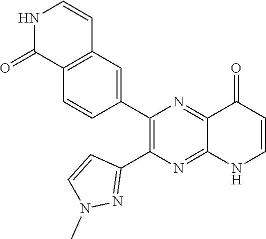 [SMILES: Cn1ccc(-c2nc3[nH]ccc(=O)c3nc2-c2ccc3c(=O)[nH]ccc3c2)n1] |
| 1.110 | 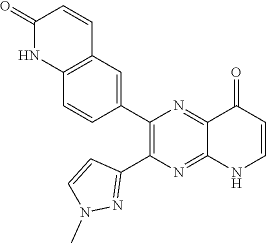 [SMILES: Cn1ccc(-c2nc3[nH]ccc(=O)c3nc2-c2ccc3[nH]c(=O)ccc3c2)n1] |
| 1.111 | 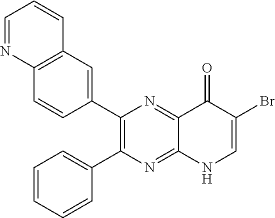 [SMILES: O=c1c(Br)c[nH]c2nc(-c3ccccc3)c(-c3ccc4ncccc4c3)nc12] |
| 1.112 | 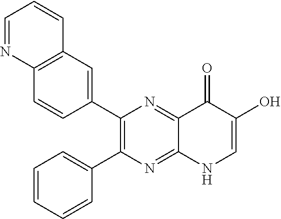 [SMILES: O=c1c(O)c[nH]c2nc(-c3ccccc3)c(-c3ccc4ncccc4c3)nc12] |
| 1.113 | 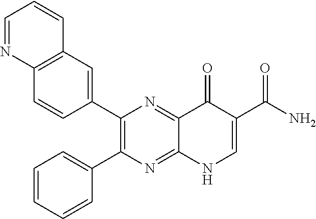 [SMILES: NC(=O)c1c[nH]c2nc(-c3ccccc3)c(-c3ccc4ncccc4c3)nc2c1=O] |
| 1.114 | 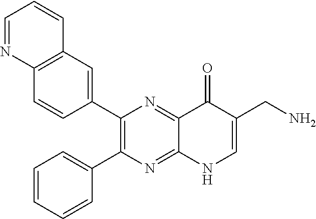 [SMILES: NCc1c[nH]c2nc(-c3ccccc3)c(-c3ccc4ncccc4c3)nc2c1=O] |
| 1.115 | 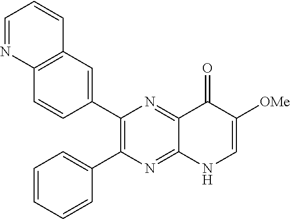 [SMILES: COc1c[nH]c2nc(-c3ccccc3)c(-c3ccc4ncccc4c3)nc2c1=O] |
| 1.116 | 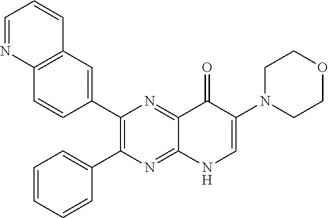 [SMILES: O=c1c(N2CCOCC2)c[nH]c2nc(-c3ccccc3)c(-c3ccc4ncccc4c3)nc12] |
| 1.117 | 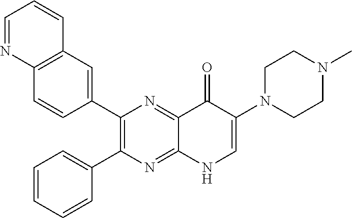 [SMILES: CN1CCN(c2c[nH]c3nc(-c4ccccc4)c(-c4ccc5ncccc5c4)nc3c2=O)CC1] |
| 1.118 | 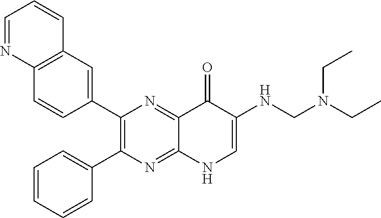 [SMILES: CCN(CC)CNc1c[nH]c2nc(-c3ccccc3)c(-c3ccc4ncccc4c3)nc2c1=O] |
| 1.119 | 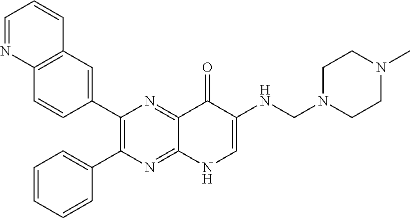 [SMILES: CN1CCN(CNc2c[nH]c3nc(-c4ccccc4)c(-c4ccc5ncccc5c4)nc3c2=O)CC1] |
| 1.120 | 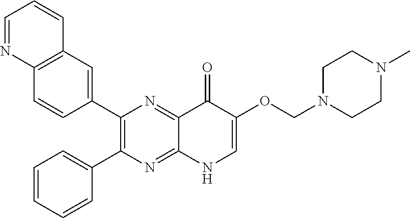 [SMILES: CN1CCN(COc2c[nH]c3nc(-c4ccccc4)c(-c4ccc5ncccc5c4)nc3c2=O)CC1] |
| 1.121 | 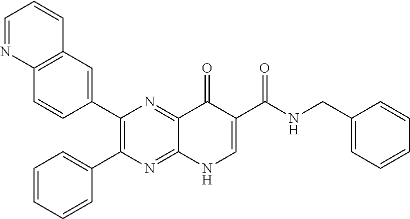 [SMILES: O=C(NCc1ccccc1)c1c[nH]c2nc(-c3ccccc3)c(-c3ccc4ncccc4c3)nc2c1=O] |
| 1.122 | 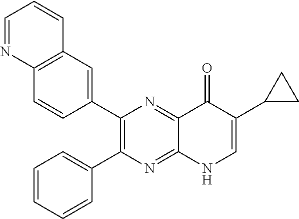 [SMILES: O=c1c(C2CC2)c[nH]c2nc(-c3ccccc3)c(-c3ccc4ncccc4c3)nc12] |
| 1.123 | 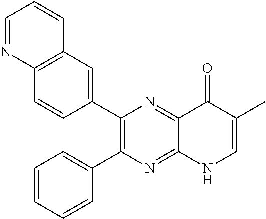 [SMILES: Cc1c[nH]c2nc(-c3ccccc3)c(-c3ccc4ncccc4c3)nc2c1=O] |
| 1.124 | 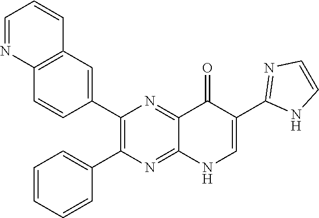 [SMILES: O=c1c(-c2ncc[nH]2)c[nH]c2nc(-c3ccccc3)c(-c3ccc4ncccc4c3)nc12] |
| 1.125 | 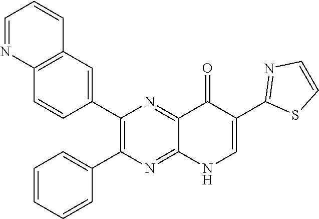 [SMILES: O=c1c(-c2nccs2)c[nH]c2nc(-c3ccccc3)c(-c3ccc4ncccc4c3)nc12] |
| 1.126 | 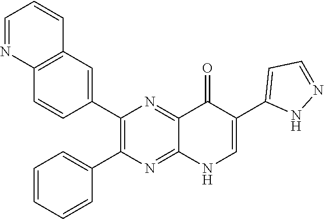 [SMILES: O=c1c(-c2ccn[nH]2)c[nH]c2nc(-c3ccccc3)c(-c3ccc4ncccc4c3)nc12] |
| 1.127 | 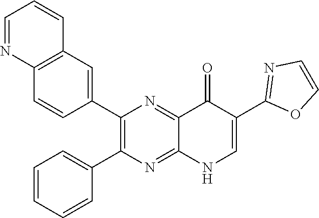 [SMILES: O=c1c(-c2ncco2)c[nH]c2nc(-c3ccccc3)c(-c3ccc4ncccc4c3)nc12] |
| 1.128 | 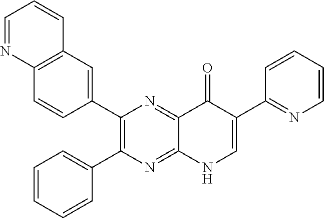 [SMILES: O=c1c(-c2ccccn2)c[nH]c2nc(-c3ccccc3)c(-c3ccc4ncccc4c3)nc12] |
| 1.129 | 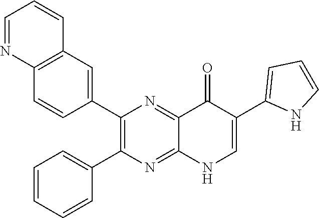 [SMILES: O=c1c(-c2ccc[nH]2)c[nH]c2nc(-c3ccccc3)c(-c3ccc4ncccc4c3)nc12] |
| 1.130 | 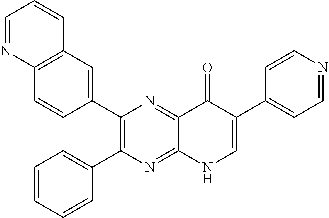 [SMILES: O=c1c(-c2ccncc2)c[nH]c2nc(-c3ccccc3)c(-c3ccc4ncccc4c3)nc12] |
| 1.131 | 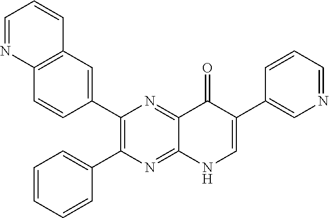 [SMILES: O=c1c(-c2cccnc2)c[nH]c2nc(-c3ccccc3)c(-c3ccc4ncccc4c3)nc12] |
| 1.132 | 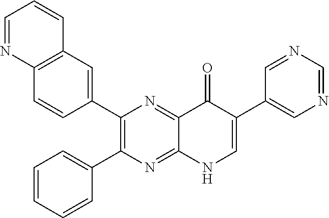 [SMILES: O=c1c(-c2cncnc2)c[nH]c2nc(-c3ccccc3)c(-c3ccc4ncccc4c3)nc12] |
| 1.133 | 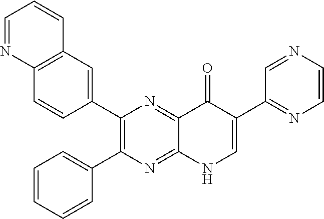 [SMILES: O=c1c(-c2cnccn2)c[nH]c2nc(-c3ccccc3)c(-c3ccc4ncccc4c3)nc12] |
| 1.134 | 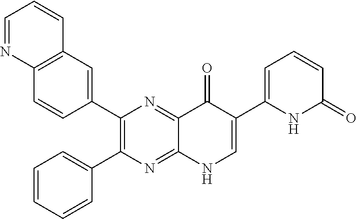 [SMILES: O=c1cccc(-c2c[nH]c3nc(-c4ccccc4)c(-c4ccc5ncccc5c4)nc3c2=O)[nH]1] |
| 1.135 | 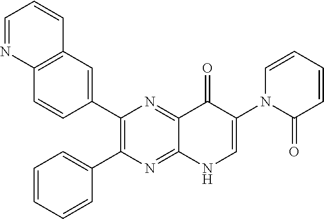 [SMILES: O=c1c(-n2ccccc2=O)c[nH]c2nc(-c3ccccc3)c(-c3ccc4ncccc4c3)nc12] |
| 1.136 | 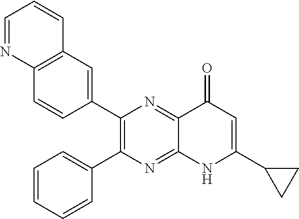 [SMILES: O=c1cc(C2CC2)[nH]c2nc(-c3ccccc3)c(-c3ccc4ncccc4c3)nc12] |
| 1.137 | 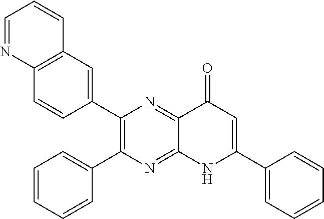 [SMILES: O=c1cc(-c2ccccc2)[nH]c2nc(-c3ccccc3)c(-c3ccc4ncccc4c3)nc12] |
| 1.138 | 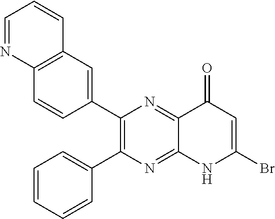 [SMILES: O=c1cc(Br)[nH]c2nc(-c3ccccc3)c(-c3ccc4ncccc4c3)nc12] |
| 1.139 | 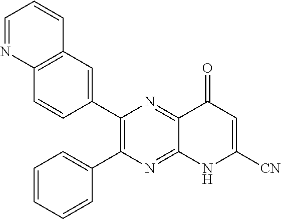 [SMILES: N#Cc1cc(=O)c2nc(-c3ccc4ncccc4c3)c(-c3ccccc3)nc2[nH]1] |
| 1.140 | 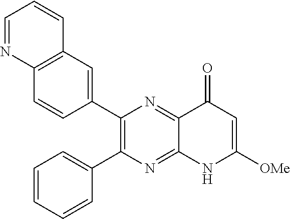 [SMILES: COc1cc(=O)c2nc(-c3ccc4ncccc4c3)c(-c3ccccc3)nc2[nH]1] |
| 1.141 | 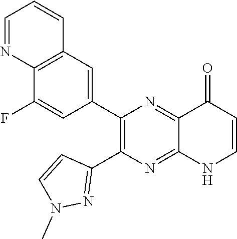 [SMILES: Cn1ccc(-c2nc3[nH]ccc(=O)c3nc2-c2cc(F)c3ncccc3c2)n1] |
| 1.142 | 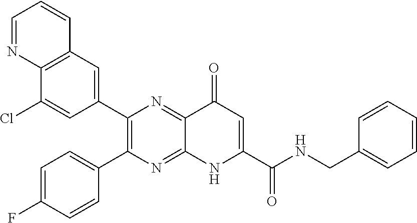 [SMILES: O=C(NCc1ccccc1)c1cc(=O)c2nc(-c3cc(Cl)c4ncccc4c3)c(-c3ccc(F)cc3)nc2[nH]1] |
| 1.143 | 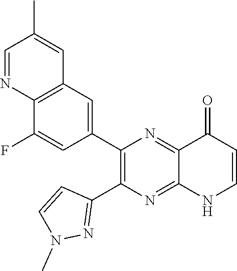 [SMILES: Cc1cnc2c(F)cc(-c3nc4c(=O)cc[nH]c4nc3-c3ccn(C)n3)cc2c1] |
| 1.144 | 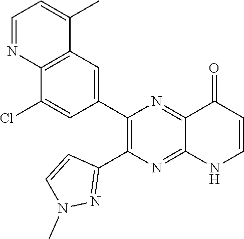 [SMILES: Cc1ccnc2c(Cl)cc(-c3nc4c(=O)cc[nH]c4nc3-c3ccn(C)n3)cc12] |
| 1.145 | 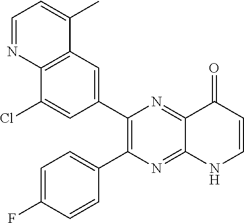 [SMILES: Cc1ccnc2c(Cl)cc(-c3nc4c(=O)cc[nH]c4nc3-c3ccc(F)cc3)cc12] |
| 1.146 | 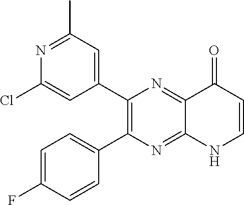 [SMILES: Cc1cc(-c2nc3c(=O)cc[nH]c3nc2-c2ccc(F)cc2)cc(Cl)n1] |
| 1.147 | 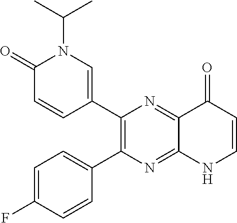 [SMILES: CC(C)n1cc(-c2nc3c(=O)cc[nH]c3nc2-c2ccc(F)cc2)ccc1=O] |
| 1.148 | 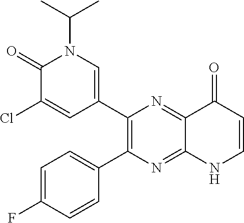 [SMILES: CC(C)n1cc(-c2nc3c(=O)cc[nH]c3nc2-c2ccc(F)cc2)cc(Cl)c1=O] |
| 1.149 | 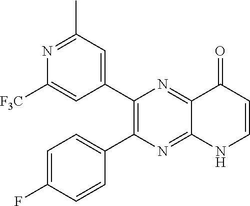 [SMILES: Cc1cc(-c2nc3c(=O)cc[nH]c3nc2-c2ccc(F)cc2)cc(C(F)(F)F)n1] |
| 1.150 | 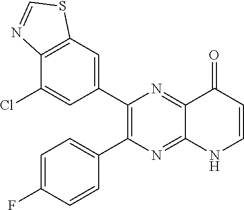 [SMILES: O=c1cc[nH]c2nc(-c3ccc(F)cc3)c(-c3cc(Cl)c4ncsc4c3)nc12] |
| 1.151 | 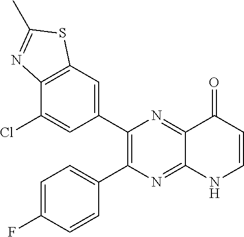 [SMILES: Cc1nc2c(Cl)cc(-c3nc4c(=O)cc[nH]c4nc3-c3ccc(F)cc3)cc2s1] |
| 1.152 | 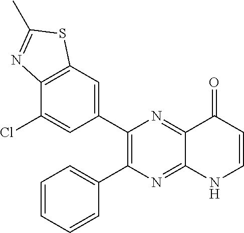 [SMILES: Cc1nc2c(Cl)cc(-c3nc4c(=O)cc[nH]c4nc3-c3ccccc3)cc2s1] |
| 1.153 | 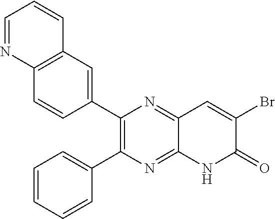 [SMILES: O=c1[nH]c2nc(-c3ccccc3)c(-c3ccc4ncccc4c3)nc2cc1Br] |
| 1.154 | 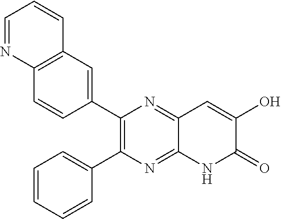 [SMILES: O=c1[nH]c2nc(-c3ccccc3)c(-c3ccc4ncccc4c3)nc2cc1O] |
| 1.155 | 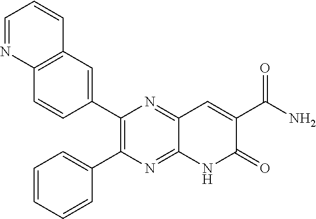 [SMILES: NC(=O)c1cc2nc(-c3ccc4ncccc4c3)c(-c3ccccc3)nc2[nH]c1=O] |
| 1.156 | 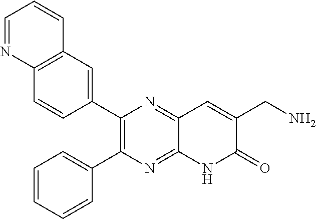 [SMILES: NCc1cc2nc(-c3ccc4ncccc4c3)c(-c3ccccc3)nc2[nH]c1=O] |
| 1.157 | 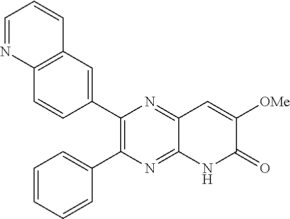 [SMILES: COc1cc2nc(-c3ccc4ncccc4c3)c(-c3ccccc3)nc2[nH]c1=O] |
| 1.158 | 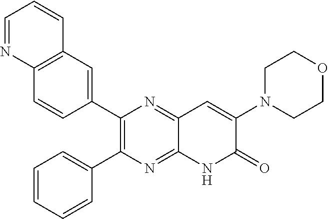 [SMILES: O=c1[nH]c2nc(-c3ccccc3)c(-c3ccc4ncccc4c3)nc2cc1N1CCOCC1] |
| 1.159 | 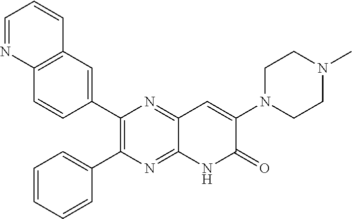 [SMILES: CN1CCN(c2cc3nc(-c4ccc5ncccc5c4)c(-c4ccccc4)nc3[nH]c2=O)CC1] |
| 1.160 | 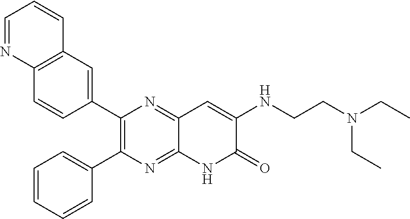 [SMILES: CCN(CC)CCNc1cc2nc(-c3ccc4ncccc4c3)c(-c3ccccc3)nc2[nH]c1=O] |
| 1.161 | 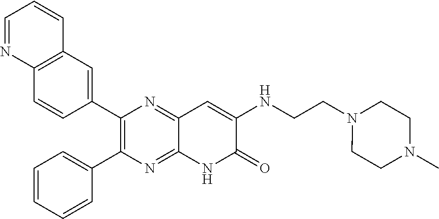 [SMILES: CN1CCN(CCNc2cc3nc(-c4ccc5ncccc5c4)c(-c4ccccc4)nc3[nH]c2=O)CC1] |
| 1.162 | 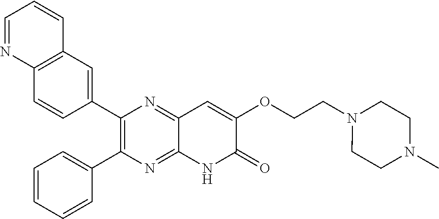 [SMILES: CN1CCN(CCOc2cc3nc(-c4ccc5ncccc5c4)c(-c4ccccc4)nc3[nH]c2=O)CC1] |
| 1.163 | 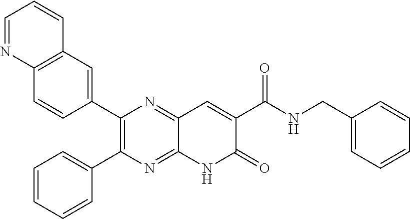 [SMILES: O=C(NCc1ccccc1)c1cc2nc(-c3ccc4ncccc4c3)c(-c3ccccc3)nc2[nH]c1=O] |
| 1.164 | 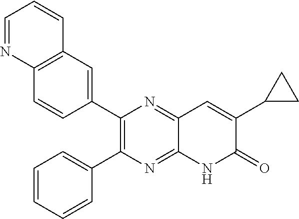 [SMILES: O=c1[nH]c2nc(-c3ccccc3)c(-c3ccc4ncccc4c3)nc2cc1C1CC1] |
| 1.165 | 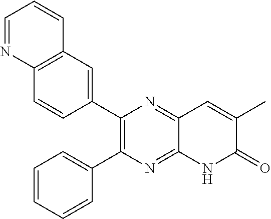 [SMILES: Cc1cc2nc(-c3ccc4ncccc4c3)c(-c3ccccc3)nc2[nH]c1=O] |
| 1.166 | 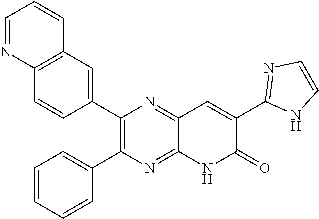 [SMILES: O=c1[nH]c2nc(-c3ccccc3)c(-c3ccc4ncccc4c3)nc2cc1-c1ncc[nH]1] |
| 1.167 | 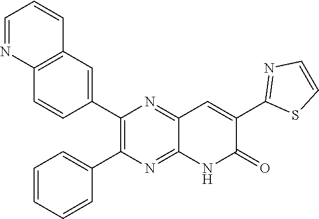 [SMILES: O=c1[nH]c2nc(-c3ccccc3)c(-c3ccc4ncccc4c3)nc2cc1-c1nccs1] |
| 1.168 | 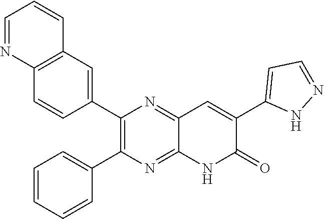 [SMILES: O=c1[nH]c2nc(-c3ccccc3)c(-c3ccc4ncccc4c3)nc2cc1-c1ccn[nH]1] |
| 1.169 | 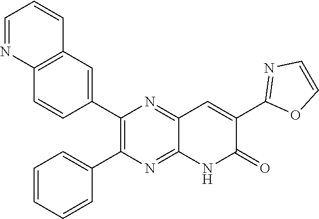 [SMILES: O=c1[nH]c2nc(-c3ccccc3)c(-c3ccc4ncccc4c3)nc2cc1-c1ncco1] |
| 1.170 | 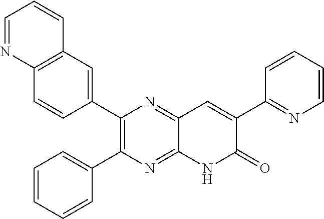 [SMILES: O=c1[nH]c2nc(-c3ccccc3)c(-c3ccc4ncccc4c3)nc2cc1-c1ccccn1] |
| 1.171 | 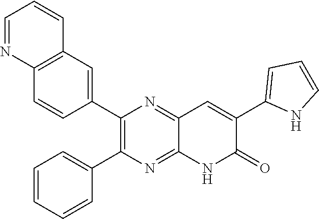 [SMILES: O=c1[nH]c2nc(-c3ccccc3)c(-c3ccc4ncccc4c3)nc2cc1-c1ccc[nH]1] |
| 1.172 | 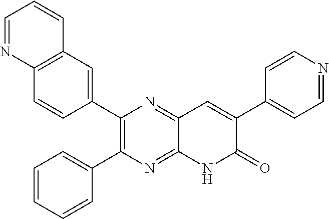 [SMILES: O=c1[nH]c2nc(-c3ccccc3)c(-c3ccc4ncccc4c3)nc2cc1-c1ccncc1] |
| 1.173 | 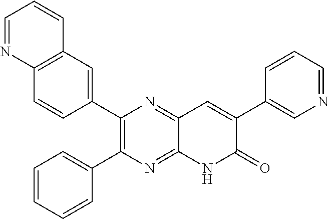 [SMILES: O=c1[nH]c2nc(-c3ccccc3)c(-c3ccc4ncccc4c3)nc2cc1-c1cccnc1] |
| 1.174 | 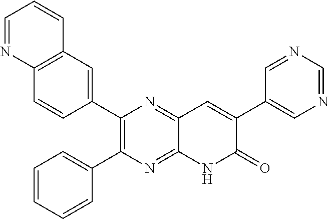 [SMILES: O=c1[nH]c2nc(-c3ccccc3)c(-c3ccc4ncccc4c3)nc2cc1-c1cncnc1] |
| 1.175 | 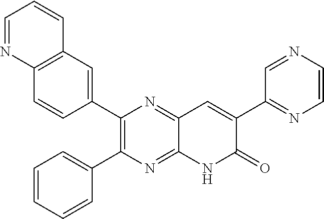 [SMILES: O=c1[nH]c2nc(-c3ccccc3)c(-c3ccc4ncccc4c3)nc2cc1-c1cnccn1] |
| 1.176 | 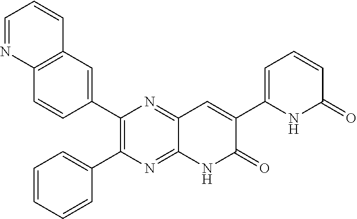 [SMILES: O=c1cccc(-c2cc3nc(-c4ccc5ncccc5c4)c(-c4ccccc4)nc3[nH]c2=O)[nH]1] |
| 1.177 | 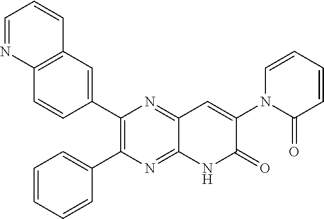 [SMILES: O=c1[nH]c2nc(-c3ccccc3)c(-c3ccc4ncccc4c3)nc2cc1-n1ccccc1=O] |
| 1.178 | 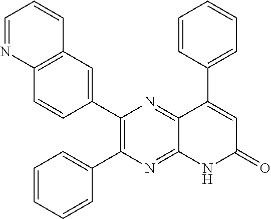 [SMILES: O=c1cc(-c2ccccc2)c2nc(-c3ccc4ncccc4c3)c(-c3ccccc3)nc2[nH]1] |
| 1.179 | 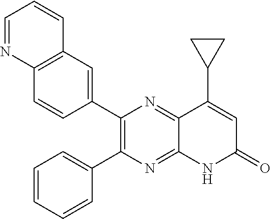 [SMILES: O=c1cc(C2CC2)c2nc(-c3ccc4ncccc4c3)c(-c3ccccc3)nc2[nH]1] |
| 1.180 | 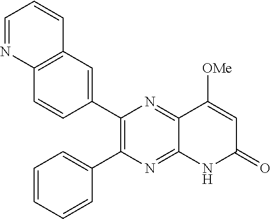 [SMILES: COc1cc(=O)[nH]c2nc(-c3ccccc3)c(-c3ccc4ncccc4c3)nc12] |
| 1.181 | 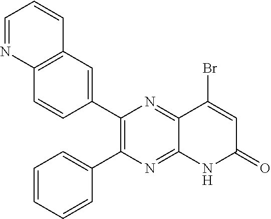 [SMILES: O=c1cc(Br)c2nc(-c3ccc4ncccc4c3)c(-c3ccccc3)nc2[nH]1] |
| 1.182 | 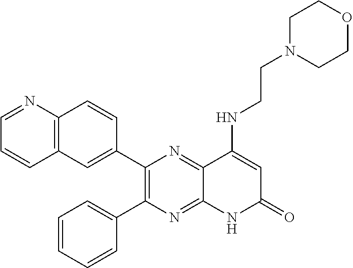 [SMILES: O=c1cc(NCCN2CCOCC2)c2nc(-c3ccc4ncccc4c3)c(-c3ccccc3)nc2[nH]1] |
| 1.183 | 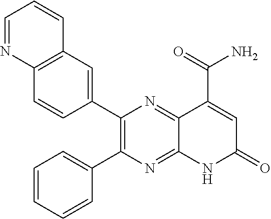 [SMILES: NC(=O)c1cc(=O)[nH]c2nc(-c3ccccc3)c(-c3ccc4ncccc4c3)nc12] |
| 1.184 | 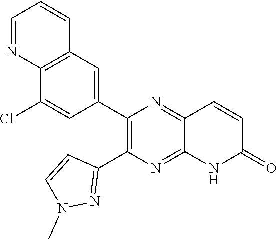 [SMILES: Cn1ccc(-c2nc3[nH]c(=O)ccc3nc2-c2cc(Cl)c3ncccc3c2)n1] |
| 1.185 | 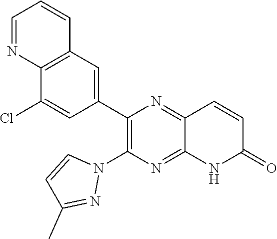 [SMILES: Cc1ccn(-c2nc3[nH]c(=O)ccc3nc2-c2cc(Cl)c3ncccc3c2)n1] |
| 1.186 | 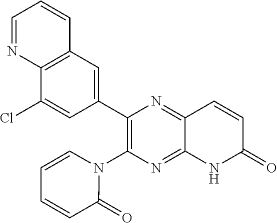 [SMILES: O=c1ccc2nc(-c3cc(Cl)c4ncccc4c3)c(-n3ccccc3=O)nc2[nH]1] |
| 1.187 | 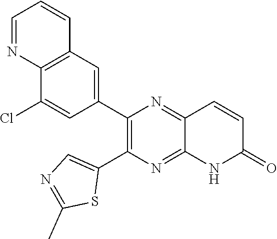 [SMILES: Cc1ncc(-c2nc3[nH]c(=O)ccc3nc2-c2cc(Cl)c3ncccc3c2)s1] |
| 1.188 | 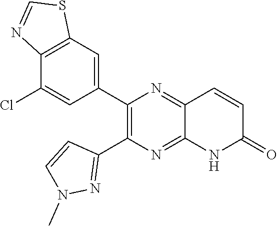 [SMILES: Cn1ccc(-c2nc3[nH]c(=O)ccc3nc2-c2cc(Cl)c3ncsc3c2)n1] |
| 1.189 | 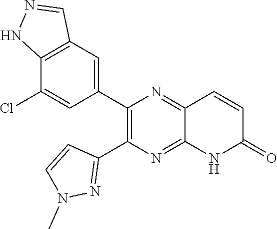 [SMILES: Cn1ccc(-c2nc3[nH]c(=O)ccc3nc2-c2cc(Cl)c3[nH]ncc3c2)n1] |
| 1.190 | 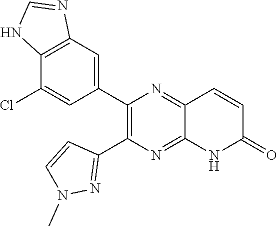 [SMILES: Cn1ccc(-c2nc3[nH]c(=O)ccc3nc2-c2cc(Cl)c3[nH]cnc3c2)n1] |
| 1.191 | 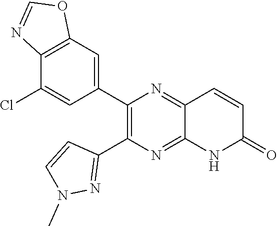 [SMILES: Cn1ccc(-c2nc3[nH]c(=O)ccc3nc2-c2cc(Cl)c3ncoc3c2)n1] |
| 1.192 | 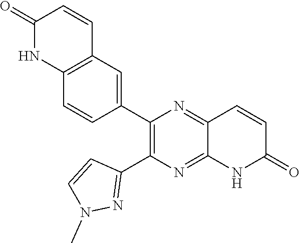 [SMILES: Cn1ccc(-c2nc3[nH]c(=O)ccc3nc2-c2ccc3[nH]c(=O)ccc3c2)n1] |
| 1.193 | 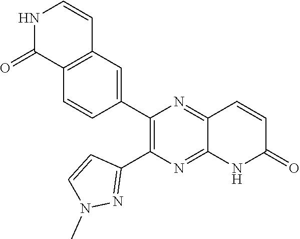 [SMILES: Cn1ccc(-c2nc3[nH]c(=O)ccc3nc2-c2ccc3c(=O)[nH]ccc3c2)n1] |
| 1.194 | 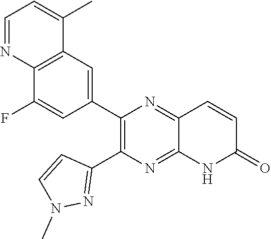 [SMILES: Cc1ccnc2c(F)cc(-c3nc4ccc(=O)[nH]c4nc3-c3ccn(C)n3)cc12] |
| 1.195 | 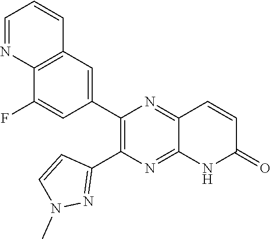 [SMILES: Cn1ccc(-c2nc3[nH]c(=O)ccc3nc2-c2cc(F)c3ncccc3c2)n1] |
| 1.196 | 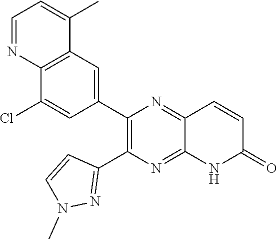 [SMILES: Cc1ccnc2c(Cl)cc(-c3nc4ccc(=O)[nH]c4nc3-c3ccn(C)n3)cc12] |
| 1.197 | 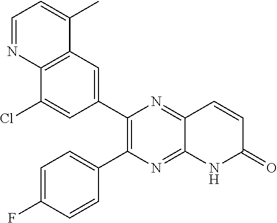 [SMILES: Cc1ccnc2c(Cl)cc(-c3nc4ccc(=O)[nH]c4nc3-c3ccc(F)cc3)cc12] |
| 1.198 | 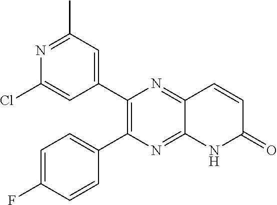 [SMILES: Cc1cc(-c2nc3ccc(=O)[nH]c3nc2-c2ccc(F)cc2)cc(Cl)n1] |
| 1.199 | 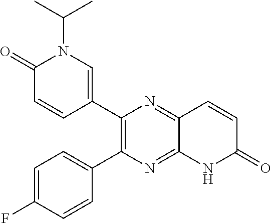 [SMILES: CC(C)n1cc(-c2nc3ccc(=O)[nH]c3nc2-c2ccc(F)cc2)ccc1=O] |
| 1.200 | 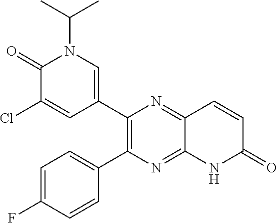 [SMILES: CC(C)n1cc(-c2nc3ccc(=O)[nH]c3nc2-c2ccc(F)cc2)cc(Cl)c1=O] |
| 1.201 | 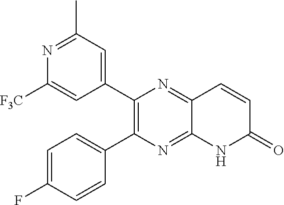 [SMILES: Cc1cc(-c2nc3ccc(=O)[nH]c3nc2-c2ccc(F)cc2)cc(C(F)(F)F)n1] |
| 1.202 | 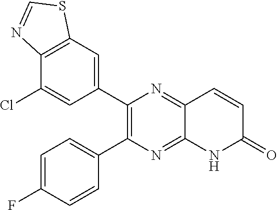 [SMILES: O=c1ccc2nc(-c3cc(Cl)c4ncsc4c3)c(-c3ccc(F)cc3)nc2[nH]1] |
| 1.203 | 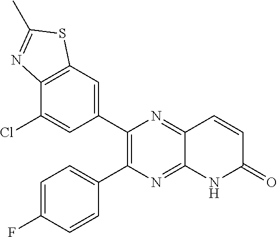 [SMILES: Cc1nc2c(Cl)cc(-c3nc4ccc(=O)[nH]c4nc3-c3ccc(F)cc3)cc2s1] |
| 1.204 | 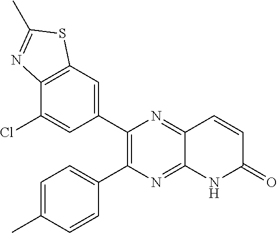 [SMILES: Cc1ccc(-c2nc3[nH]c(=O)ccc3nc2-c2cc(Cl)c3nc(C)sc3c2)cc1] |
| 1.205 | 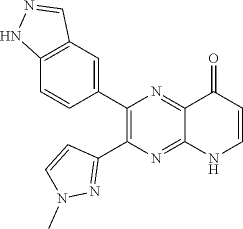 [SMILES: Cn1ccc(-c2nc3[nH]ccc(=O)c3nc2-c2ccc3[nH]ncc3c2)n1] |
| 1.206 | 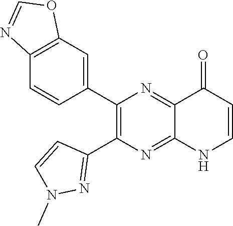 [SMILES: Cn1ccc(-c2nc3[nH]ccc(=O)c3nc2-c2ccc3ncoc3c2)n1] |
| 1.207 | 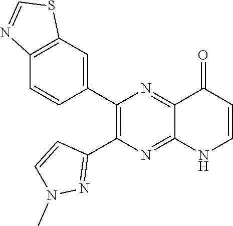 [SMILES: Cn1ccc(-c2nc3[nH]ccc(=O)c3nc2-c2ccc3ncsc3c2)n1] |
| 1.208 | 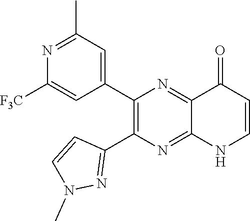 [SMILES: Cc1cc(-c2nc3c(=O)cc[nH]c3nc2-c2ccn(C)n2)cc(C(F)(F)F)n1] |
| 1.209 | 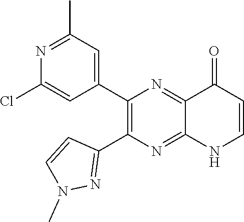 [SMILES: Cc1cc(-c2nc3c(=O)cc[nH]c3nc2-c2ccn(C)n2)cc(Cl)n1] |
| 1.210 | 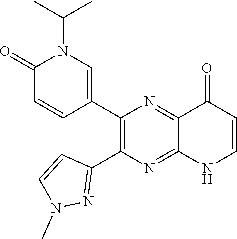 [SMILES: CC(C)n1cc(-c2nc3c(=O)cc[nH]c3nc2-c2ccn(C)n2)ccc1=O] |
| 1.211 | 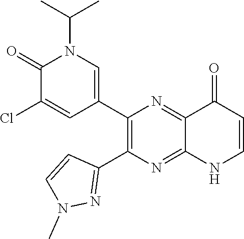 [SMILES: CC(C)n1cc(-c2nc3c(=O)cc[nH]c3nc2-c2ccn(C)n2)cc(Cl)c1=O] |
| 1.212 | 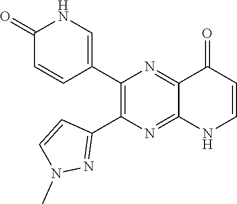 [SMILES: Cn1ccc(-c2nc3[nH]ccc(=O)c3nc2-c2ccc(=O)[nH]c2)n1] |
| 1.213 | 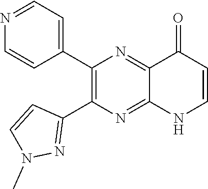 [SMILES: Cn1ccc(-c2nc3[nH]ccc(=O)c3nc2-c2ccncc2)n1] |
| 1.214 | 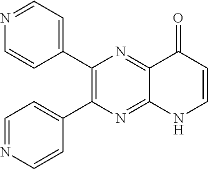 [SMILES: O=c1cc[nH]c2nc(-c3ccncc3)c(-c3ccncc3)nc12] |
| 1.215 | 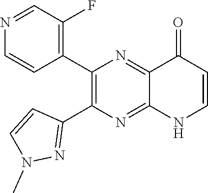 [SMILES: Cn1ccc(-c2nc3[nH]ccc(=O)c3nc2-c2ccncc2F)n1] |
| 1.216 | 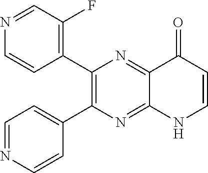 [SMILES: O=c1cc[nH]c2nc(-c3ccncc3)c(-c3ccncc3F)nc12] |
| 1.217 | 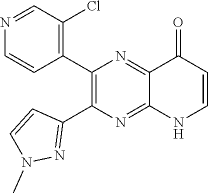 [SMILES: Cn1ccc(-c2nc3[nH]ccc(=O)c3nc2-c2ccncc2Cl)n1] |
| 1.218 | 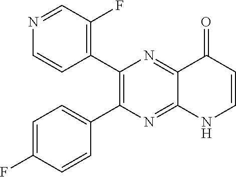 [SMILES: O=c1cc[nH]c2nc(-c3ccc(F)cc3)c(-c3ccncc3F)nc12] |
| 1.219 | 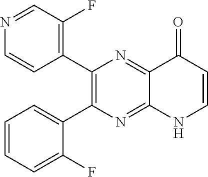 [SMILES: O=c1cc[nH]c2nc(-c3ccccc3F)c(-c3ccncc3F)nc12] |
| 1.220 | 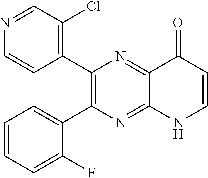 [SMILES: O=c1cc[nH]c2nc(-c3ccccc3F)c(-c3ccncc3Cl)nc12] |
| 1.221 | 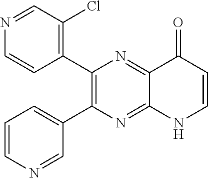 [SMILES: O=c1cc[nH]c2nc(-c3cccnc3)c(-c3ccncc3Cl)nc12] |
| 1.222 | 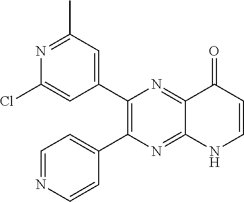 [SMILES: Cc1cc(-c2nc3c(=O)cc[nH]c3nc2-c2ccncc2)cc(Cl)n1] |
| 1.223 | 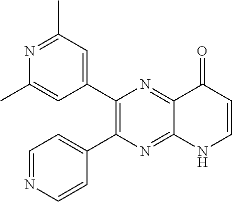 [SMILES: Cc1cc(-c2nc3c(=O)cc[nH]c3nc2-c2ccncc2)cc(C)n1] |
| 1.224 | 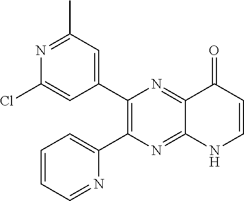 [SMILES: Cc1cc(-c2nc3c(=O)cc[nH]c3nc2-c2ccccn2)cc(Cl)n1] |
| 1.225 | 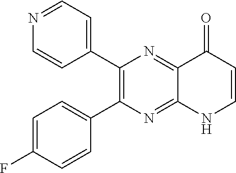 [SMILES: O=c1cc[nH]c2nc(-c3ccc(F)cc3)c(-c3ccncc3)nc12] |
| 1.226 | 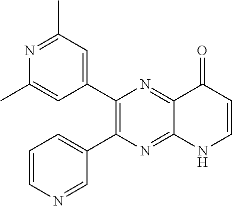 [SMILES: Cc1cc(-c2nc3c(=O)cc[nH]c3nc2-c2cccnc2)cc(C)n1] |
| 1.227 | 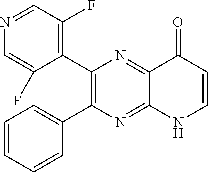 [SMILES: O=c1cc[nH]c2nc(-c3ccccc3)c(-c3c(F)cncc3F)nc12] |
| 1.228 | 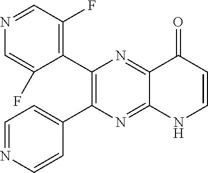 [SMILES: O=c1cc[nH]c2nc(-c3ccncc3)c(-c3c(F)cncc3F)nc12] |
| 1.229 | 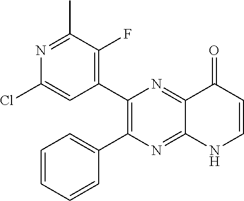 [SMILES: Cc1nc(Cl)cc(-c2nc3c(=O)cc[nH]c3nc2-c2ccccc2)c1F] |
| 1.230 | 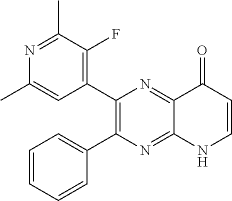 [SMILES: Cc1cc(-c2nc3c(=O)cc[nH]c3nc2-c2ccccc2)c(F)c(C)n1] |
| 1.231 | 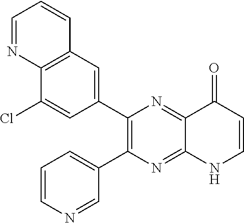 [SMILES: O=c1cc[nH]c2nc(-c3cccnc3)c(-c3cc(Cl)c4ncccc4c3)nc12] |
| 1.232 | 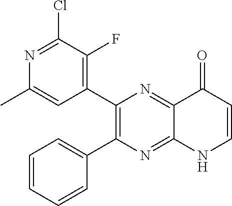 [SMILES: Cc1cc(-c2nc3c(=O)cc[nH]c3nc2-c2ccccc2)c(F)c(Cl)n1] |
| 1.233 | 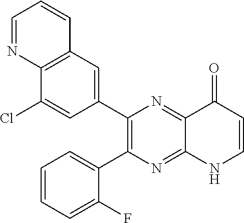 [SMILES: O=c1cc[nH]c2nc(-c3ccccc3F)c(-c3cc(Cl)c4ncccc4c3)nc12] |
| 1.234 | 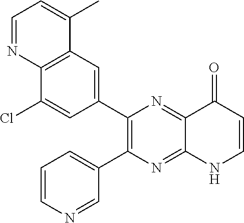 [SMILES: Cc1ccnc2c(Cl)cc(-c3nc4c(=O)cc[nH]c4nc3-c3cccnc3)cc12] |
| 1.235 | 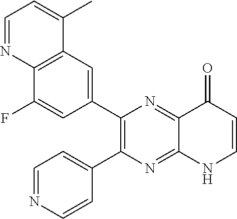 [SMILES: Cc1ccnc2c(F)cc(-c3nc4c(=O)cc[nH]c4nc3-c3ccncc3)cc12] |
| 1.236 | 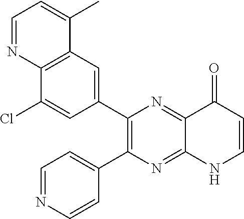 [SMILES: Cc1ccnc2c(Cl)cc(-c3nc4c(=O)cc[nH]c4nc3-c3ccncc3)cc12] |
| 1.237 | 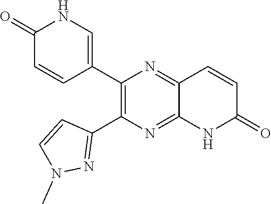 [SMILES: Cn1ccc(-c2nc3[nH]c(=O)ccc3nc2-c2ccc(=O)[nH]c2)n1] |
| 1.238 | 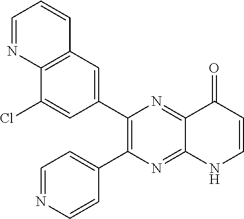 [SMILES: O=c1cc[nH]c2nc(-c3ccncc3)c(-c3cc(Cl)c4ncccc4c3)nc12] |
| 1.239 | 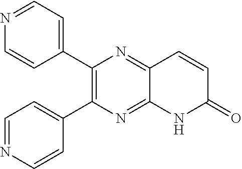 [SMILES: O=c1ccc2nc(-c3ccncc3)c(-c3ccncc3)nc2[nH]1] |
| 1.240 | 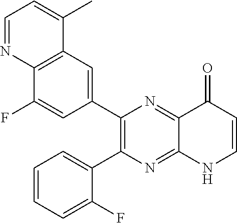 [SMILES: Cc1ccnc2c(F)cc(-c3nc4c(=O)cc[nH]c4nc3-c3ccccc3F)cc12] |
| 1.241 | 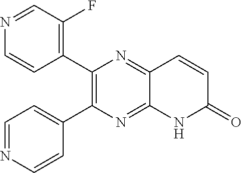 [SMILES: O=c1ccc2nc(-c3ccncc3F)c(-c3ccncc3)nc2[nH]1] |
| 1.242 | 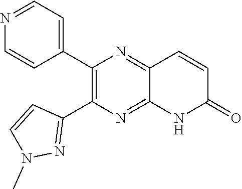 [SMILES: Cn1ccc(-c2nc3[nH]c(=O)ccc3nc2-c2ccncc2)n1] |
| 1.243 | 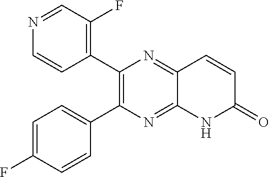 [SMILES: O=c1ccc2nc(-c3ccncc3F)c(-c3ccc(F)cc3)nc2[nH]1] |
| 1.244 | 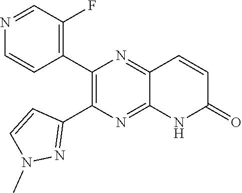 [SMILES: Cn1ccc(-c2nc3[nH]c(=O)ccc3nc2-c2ccncc2F)n1] |
| 1.245 | 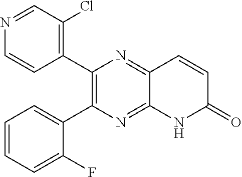 [SMILES: O=c1ccc2nc(-c3ccncc3Cl)c(-c3ccccc3F)nc2[nH]1] |
| 1.246 | 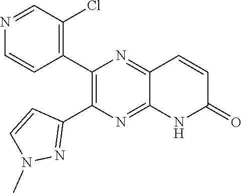 [SMILES: Cn1ccc(-c2nc3[nH]c(=O)ccc3nc2-c2ccncc2Cl)n1] |
| 1.247 | 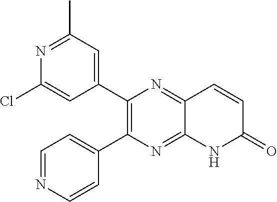 [SMILES: Cc1cc(-c2nc3ccc(=O)[nH]c3nc2-c2ccncc2)cc(Cl)n1] |
| 1.248 | 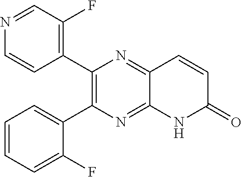 [SMILES: O=c1ccc2nc(-c3ccncc3F)c(-c3ccccc3F)nc2[nH]1] |
| 1.249 | 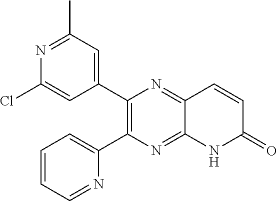 [SMILES: Cc1cc(-c2nc3ccc(=O)[nH]c3nc2-c2ccccn2)cc(Cl)n1] |
| 1.250 | 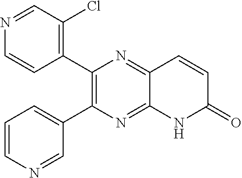 [SMILES: O=c1ccc2nc(-c3ccncc3Cl)c(-c3cccnc3)nc2[nH]1] |
| 1.251 | 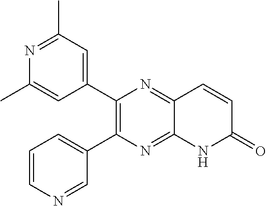 [SMILES: Cc1cc(-c2nc3ccc(=O)[nH]c3nc2-c2cccnc2)cc(C)n1] |
| 1.252 | 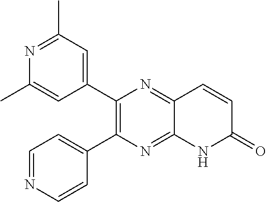 [SMILES: Cc1cc(-c2nc3ccc(=O)[nH]c3nc2-c2ccncc2)cc(C)n1] |
| 1.253 | 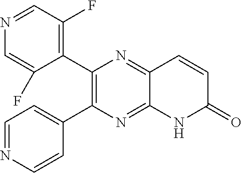 [SMILES: O=c1ccc2nc(-c3c(F)cncc3F)c(-c3ccncc3)nc2[nH]1] |
| 1.254 | 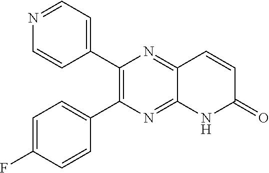 [SMILES: O=c1ccc2nc(-c3ccncc3)c(-c3ccc(F)cc3)nc2[nH]1] |
| 1.255 | 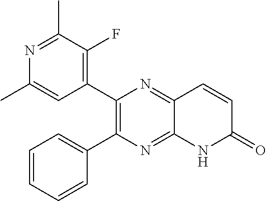 [SMILES: Cc1cc(-c2nc3ccc(=O)[nH]c3nc2-c2ccccc2)c(F)c(C)n1] |
| 1.256 | 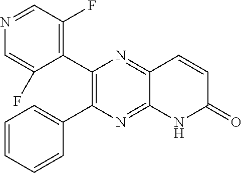 [SMILES: O=c1ccc2nc(-c3c(F)cncc3F)c(-c3ccccc3)nc2[nH]1] |
| 1.257 | 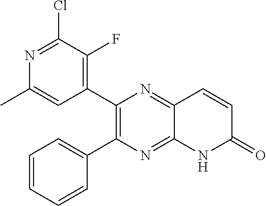 [SMILES: Cc1cc(-c2nc3ccc(=O)[nH]c3nc2-c2ccccc2)c(F)c(Cl)n1] |
| 1.258 | 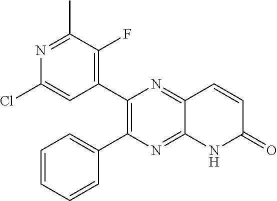 [SMILES: Cc1nc(Cl)cc(-c2nc3ccc(=O)[nH]c3nc2-c2ccccc2)c1F] |
| 1.259 | 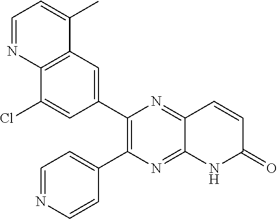 [SMILES: Cc1ccnc2c(Cl)cc(-c3nc4ccc(=O)[nH]c4nc3-c3ccncc3)cc12] |
| 1.260 | 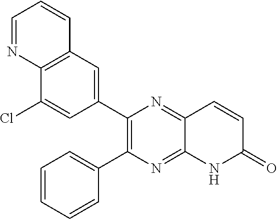 [SMILES: O=c1ccc2nc(-c3cc(Cl)c4ncccc4c3)c(-c3ccccc3)nc2[nH]1] |
| 1.261 | 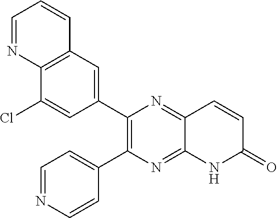 [SMILES: O=c1ccc2nc(-c3cc(Cl)c4ncccc4c3)c(-c3ccncc3)nc2[nH]1] |
| 1.262 | 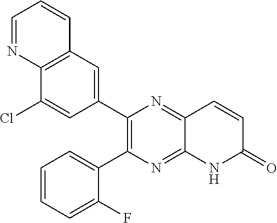 [SMILES: O=c1ccc2nc(-c3cc(Cl)c4ncccc4c3)c(-c3ccccc3F)nc2[nH]1] |
| 1.263 | 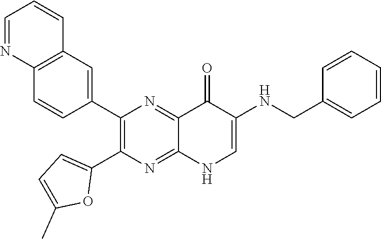 [SMILES: Cc1ccc(-c2nc3[nH]cc(NCc4ccccc4)c(=O)c3nc2-c2ccc3ncccc3c2)o1] |
| 1.264 | 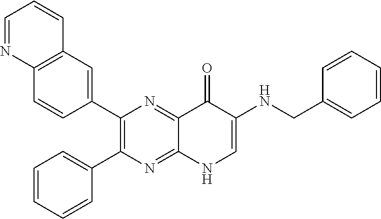 [SMILES: O=c1c(NCc2ccccc2)c[nH]c2nc(-c3ccccc3)c(-c3ccc4ncccc4c3)nc12] |
| 1.265 | 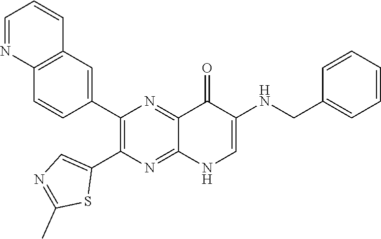 [SMILES: Cc1ncc(-c2nc3[nH]cc(NCc4ccccc4)c(=O)c3nc2-c2ccc3ncccc3c2)s1] |
| 1.266 | 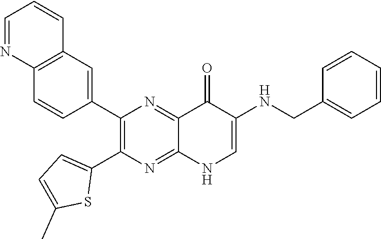 [SMILES: Cc1ccc(-c2nc3[nH]cc(NCc4ccccc4)c(=O)c3nc2-c2ccc3ncccc3c2)s1] |
| 1.267 | 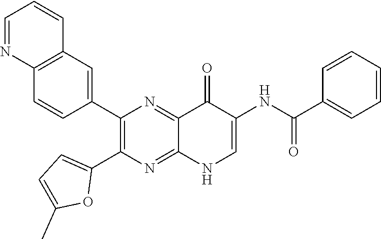 [SMILES: Cc1ccc(-c2nc3[nH]cc(NC(=O)c4ccccc4)c(=O)c3nc2-c2ccc3ncccc3c2)o1] |
| 1.268 | 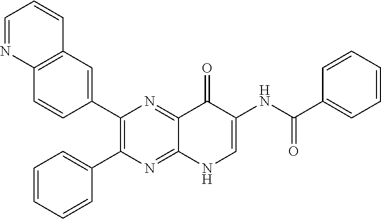 [SMILES: O=C(Nc1c[nH]c2nc(-c3ccccc3)c(-c3ccc4ncccc4c3)nc2c1=O)c1ccccc1] |
| 1.269 | 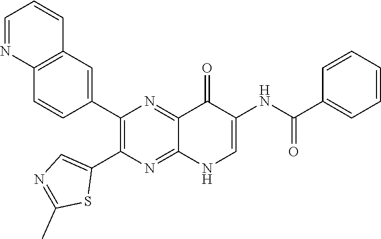 [SMILES: Cc1ncc(-c2nc3[nH]cc(NC(=O)c4ccccc4)c(=O)c3nc2-c2ccc3ncccc3c2)s1] |
| 1.270 | 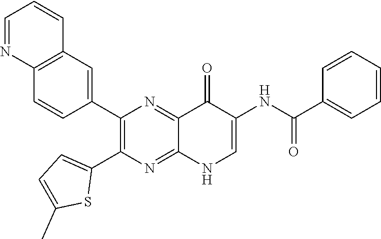 [SMILES: Cc1ccc(-c2nc3[nH]cc(NC(=O)c4ccccc4)c(=O)c3nc2-c2ccc3ncccc3c2)s1] |
| 1.271 | 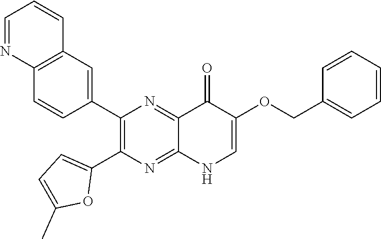 [SMILES: Cc1ccc(-c2nc3[nH]cc(OCc4ccccc4)c(=O)c3nc2-c2ccc3ncccc3c2)o1] |
| 1.272 | 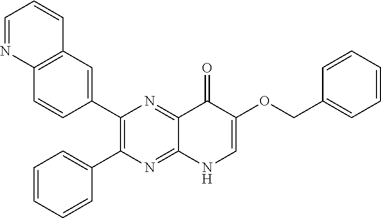 [SMILES: O=c1c(OCc2ccccc2)c[nH]c2nc(-c3ccccc3)c(-c3ccc4ncccc4c3)nc12] |
| 1.273 | 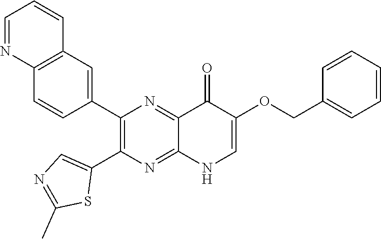 [SMILES: Cc1ncc(-c2nc3[nH]cc(OCc4ccccc4)c(=O)c3nc2-c2ccc3ncccc3c2)s1] |
| 1.274 | 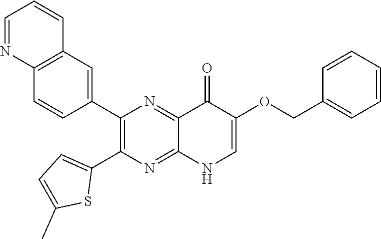 [SMILES: Cc1ccc(-c2nc3[nH]cc(OCc4ccccc4)c(=O)c3nc2-c2ccc3ncccc3c2)s1] |
| 1.275 | 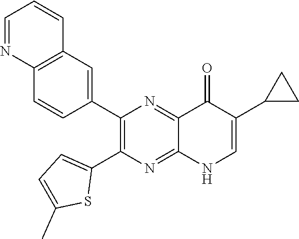 [SMILES: Cc1ccc(-c2nc3[nH]cc(C4CC4)c(=O)c3nc2-c2ccc3ncccc3c2)s1] |
| 1.276 | 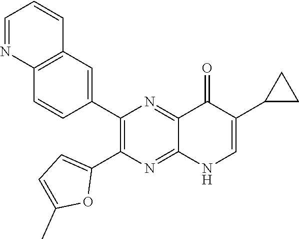 [SMILES: Cc1ccc(-c2nc3[nH]cc(C4CC4)c(=O)c3nc2-c2ccc3ncccc3c2)o1] |
| 1.277 | 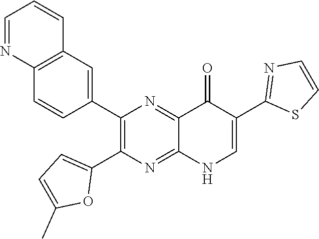 [SMILES: Cc1ccc(-c2nc3[nH]cc(-c4nccs4)c(=O)c3nc2-c2ccc3ncccc3c2)o1] |
| 1.278 | 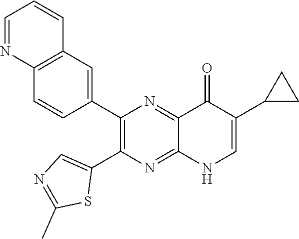 [SMILES: Cc1ncc(-c2nc3[nH]cc(C4CC4)c(=O)c3nc2-c2ccc3ncccc3c2)s1] |
| 1.279 | 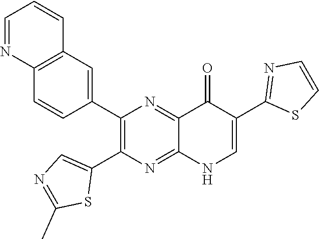 [SMILES: Cc1ncc(-c2nc3[nH]cc(-c4nccs4)c(=O)c3nc2-c2ccc3ncccc3c2)s1] |
| 1.280 | 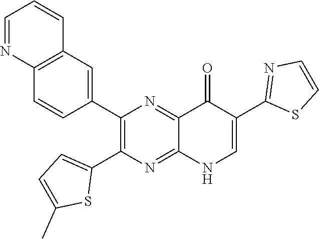 [SMILES: Cc1ccc(-c2nc3[nH]cc(-c4nccs4)c(=O)c3nc2-c2ccc3ncccc3c2)s1] |
| 1.281 | 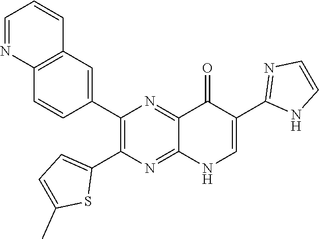 [SMILES: Cc1ccc(-c2nc3[nH]cc(-c4ncc[nH]4)c(=O)c3nc2-c2ccc3ncccc3c2)s1] |
| 1.282 | 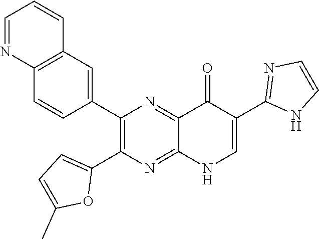 [SMILES: Cc1ccc(-c2nc3[nH]cc(-c4ncc[nH]4)c(=O)c3nc2-c2ccc3ncccc3c2)o1] |
| 1.283 | 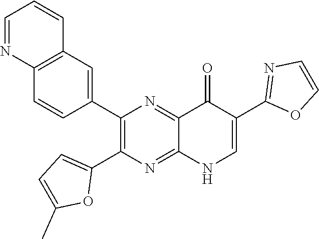 [SMILES: Cc1ccc(-c2nc3[nH]cc(-c4ncco4)c(=O)c3nc2-c2ccc3ncccc3c2)o1] |
| 1.284 | 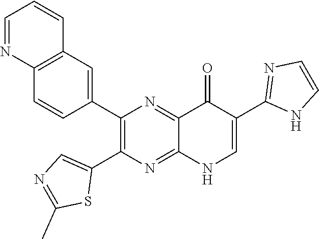 [SMILES: Cc1ncc(-c2nc3[nH]cc(-c4ncc[nH]4)c(=O)c3nc2-c2ccc3ncccc3c2)s1] |
| 1.285 | 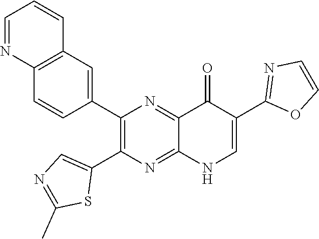 [SMILES: Cc1ncc(-c2nc3[nH]cc(-c4ncco4)c(=O)c3nc2-c2ccc3ncccc3c2)s1] |
| 1.286 | 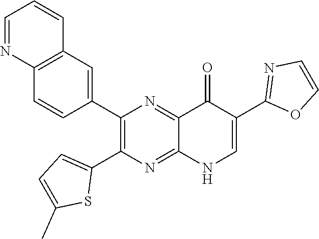 [SMILES: Cc1ccc(-c2nc3[nH]cc(-c4ncco4)c(=O)c3nc2-c2ccc3ncccc3c2)s1] |
| 1.287 | 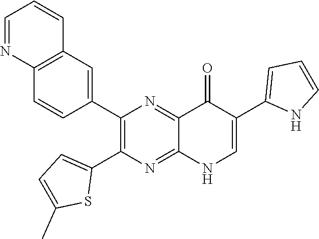 [SMILES: Cc1ccc(-c2nc3[nH]cc(-c4ccc[nH]4)c(=O)c3nc2-c2ccc3ncccc3c2)s1] |
| 1.288 | 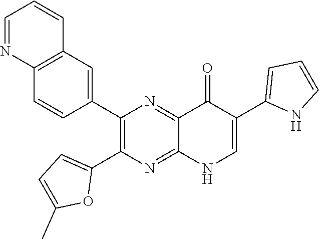 [SMILES: Cc1ccc(-c2nc3[nH]cc(-c4ccc[nH]4)c(=O)c3nc2-c2ccc3ncccc3c2)o1] |
| 1.289 | 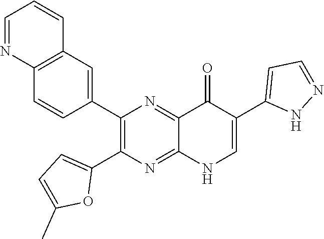 [SMILES: Cc1ccc(-c2nc3[nH]cc(-c4ccn[nH]4)c(=O)c3nc2-c2ccc3ncccc3c2)o1] |
| 1.290 | 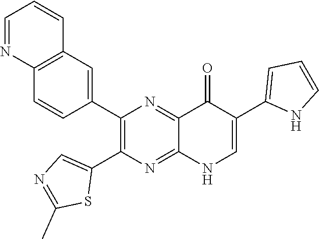 [SMILES: Cc1ncc(-c2nc3[nH]cc(-c4ccc[nH]4)c(=O)c3nc2-c2ccc3ncccc3c2)s1] |
| 1.291 | 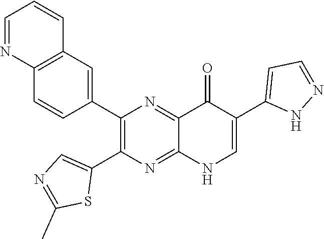 [SMILES: Cc1ncc(-c2nc3[nH]cc(-c4ccn[nH]4)c(=O)c3nc2-c2ccc3ncccc3c2)s1] |
| 1.292 | 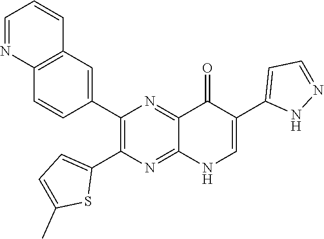 [SMILES: Cc1ccc(-c2nc3[nH]cc(-c4ccn[nH]4)c(=O)c3nc2-c2ccc3ncccc3c2)s1] |
| 1.293 | 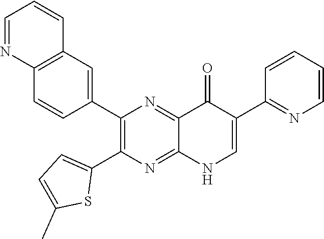 [SMILES: Cc1ccc(-c2nc3[nH]cc(-c4ccccn4)c(=O)c3nc2-c2ccc3ncccc3c2)s1] |
| 1.294 | 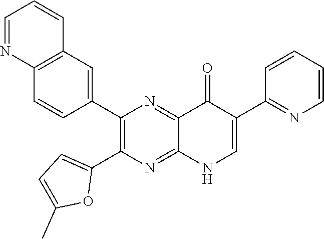 [SMILES: Cc1ccc(-c2nc3[nH]cc(-c4ccccn4)c(=O)c3nc2-c2ccc3ncccc3c2)o1] |
| 1.295 | 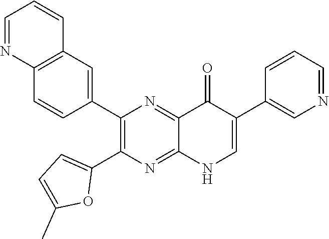 [SMILES: Cc1ccc(-c2nc3[nH]cc(-c4cccnc4)c(=O)c3nc2-c2ccc3ncccc3c2)o1] |
| 1.296 | 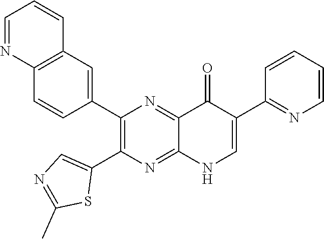 [SMILES: Cc1ncc(-c2nc3[nH]cc(-c4ccccn4)c(=O)c3nc2-c2ccc3ncccc3c2)s1] |
| 1.297 | 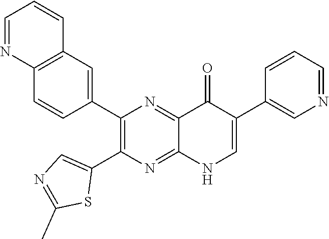 [SMILES: Cc1ncc(-c2nc3[nH]cc(-c4cccnc4)c(=O)c3nc2-c2ccc3ncccc3c2)s1] |
| 1.298 | 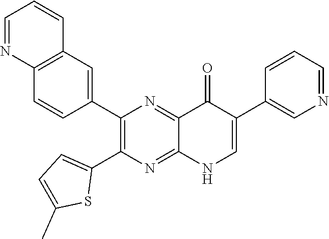 [SMILES: Cc1ccc(-c2nc3[nH]cc(-c4cccnc4)c(=O)c3nc2-c2ccc3ncccc3c2)s1] |
| 1.299 | 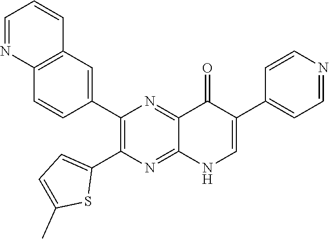 [SMILES: Cc1ccc(-c2nc3[nH]cc(-c4ccncc4)c(=O)c3nc2-c2ccc3ncccc3c2)s1] |
| 1.300 | 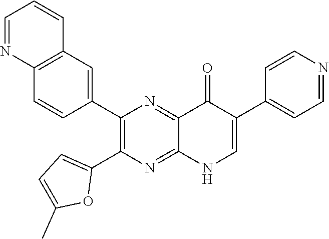 [SMILES: Cc1ccc(-c2nc3[nH]cc(-c4ccncc4)c(=O)c3nc2-c2ccc3ncccc3c2)o1] |
| 1.301 | 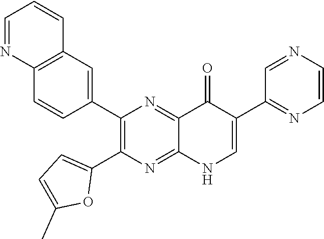 [SMILES: Cc1ccc(-c2nc3[nH]cc(-c4cnccn4)c(=O)c3nc2-c2ccc3ncccc3c2)o1] |
| 1.302 | 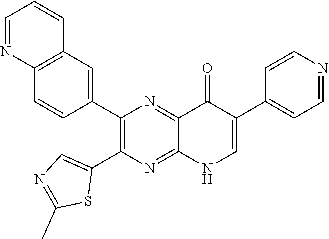 [SMILES: Cc1ncc(-c2nc3[nH]cc(-c4ccncc4)c(=O)c3nc2-c2ccc3ncccc3c2)s1] |
| 1.303 | 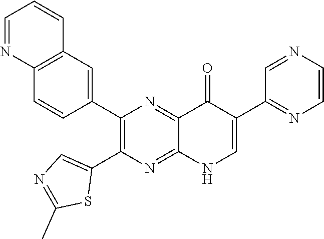 [SMILES: Cc1ncc(-c2nc3[nH]cc(-c4cnccn4)c(=O)c3nc2-c2ccc3ncccc3c2)s1] |
| 1.304 | 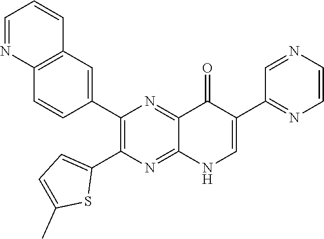 [SMILES: Cc1ccc(-c2nc3[nH]cc(-c4cnccn4)c(=O)c3nc2-c2ccc3ncccc3c2)s1] |
| 1.305 | 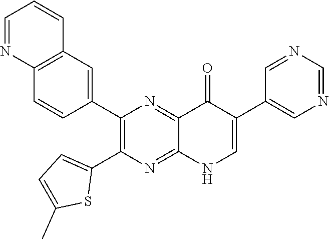 [SMILES: Cc1ccc(-c2nc3[nH]cc(-c4cncnc4)c(=O)c3nc2-c2ccc3ncccc3c2)s1] |
| 1.306 | 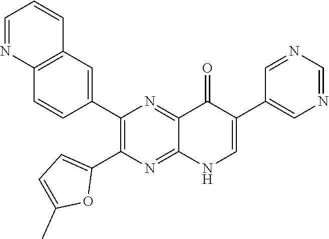 [SMILES: Cc1ccc(-c2nc3[nH]cc(-c4cncnc4)c(=O)c3nc2-c2ccc3ncccc3c2)o1] |
| 1.307 | 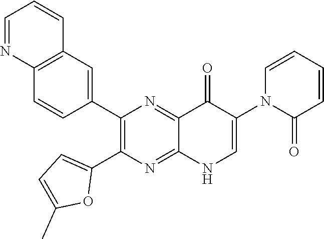 [SMILES: Cc1ccc(-c2nc3[nH]cc(-n4ccccc4=O)c(=O)c3nc2-c2ccc3ncccc3c2)o1] |
| 1.308 | 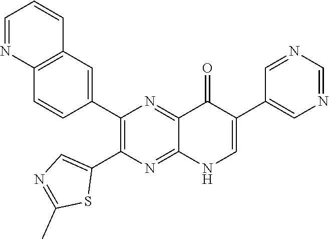 [SMILES: Cc1ncc(-c2nc3[nH]cc(-c4cncnc4)c(=O)c3nc2-c2ccc3ncccc3c2)s1] |
| 1.309 | 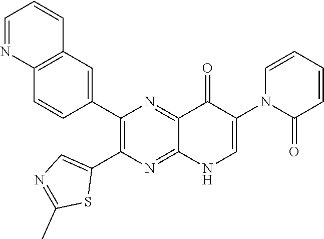 [SMILES: Cc1ncc(-c2nc3[nH]cc(-n4ccccc4=O)c(=O)c3nc2-c2ccc3ncccc3c2)s1] |
| 1.310 | 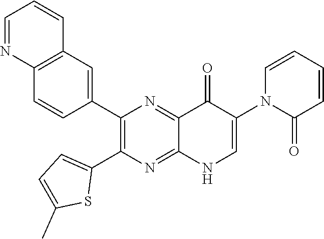 [SMILES: Cc1ccc(-c2nc3[nH]cc(-n4ccccc4=O)c(=O)c3nc2-c2ccc3ncccc3c2)s1] |
| 1.311 | 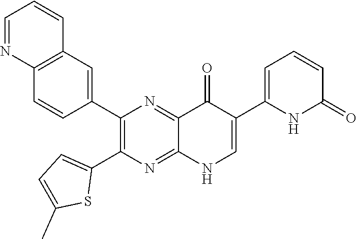 [SMILES: Cc1ccc(-c2nc3[nH]cc(-c4cccc(=O)[nH]4)c(=O)c3nc2-c2ccc3ncccc3c2)s1] |
| 1.312 | 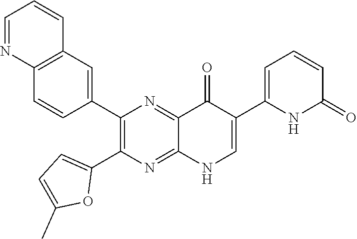 [SMILES: Cc1ccc(-c2nc3[nH]cc(-c4cccc(=O)[nH]4)c(=O)c3nc2-c2ccc3ncccc3c2)o1] |
| 1.313 | 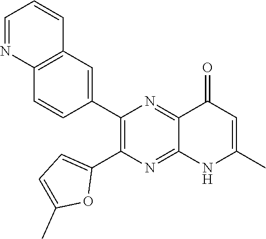 [SMILES: Cc1cc(=O)c2nc(-c3ccc4ncccc4c3)c(-c3ccc(C)o3)nc2[nH]1] |
| 1.314 | 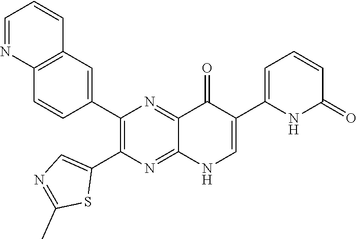 [SMILES: Cc1ncc(-c2nc3[nH]cc(-c4cccc(=O)[nH]4)c(=O)c3nc2-c2ccc3ncccc3c2)s1] |
| 1.315 | 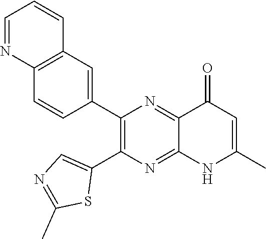 [SMILES: Cc1cc(=O)c2nc(-c3ccc4ncccc4c3)c(-c3cnc(C)s3)nc2[nH]1] |
| 1.316 | 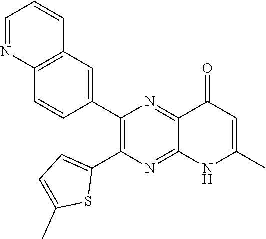 [SMILES: Cc1cc(=O)c2nc(-c3ccc4ncccc4c3)c(-c3ccc(C)s3)nc2[nH]1] |
| 1.317 | 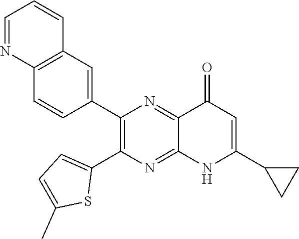 [SMILES: Cc1ccc(-c2nc3[nH]c(C4CC4)cc(=O)c3nc2-c2ccc3ncccc3c2)s1] |
| 1.318 | 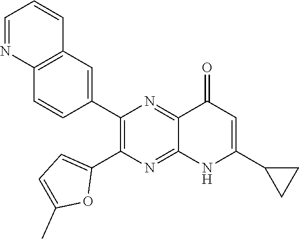 [SMILES: Cc1ccc(-c2nc3[nH]c(C4CC4)cc(=O)c3nc2-c2ccc3ncccc3c2)o1] |
| 1.319 | 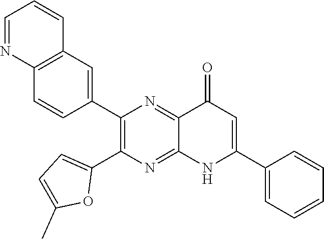 [SMILES: Cc1ccc(-c2nc3[nH]c(-c4ccccc4)cc(=O)c3nc2-c2ccc3ncccc3c2)o1] |
| 1.320 | 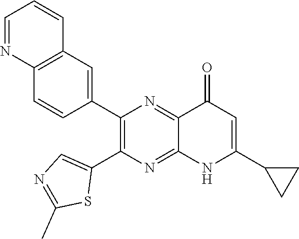 [SMILES: Cc1ncc(-c2nc3[nH]c(C4CC4)cc(=O)c3nc2-c2ccc3ncccc3c2)s1] |
| 1.321 | 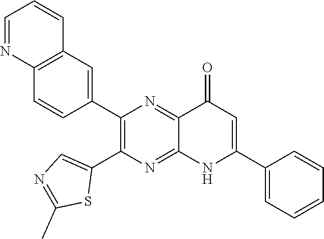 [SMILES: Cc1ncc(-c2nc3[nH]c(-c4ccccc4)cc(=O)c3nc2-c2ccc3ncccc3c2)s1] |
| 1.322 | 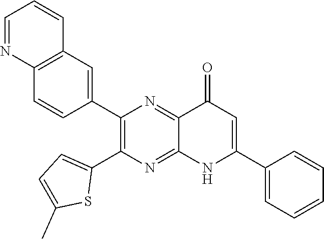 [SMILES: Cc1ccc(-c2nc3[nH]c(-c4ccccc4)cc(=O)c3nc2-c2ccc3ncccc3c2)s1] |
| 1.323 | 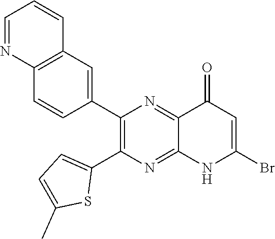 [SMILES: Cc1ccc(-c2nc3[nH]c(Br)cc(=O)c3nc2-c2ccc3ncccc3c2)s1] |
| 1.324 | 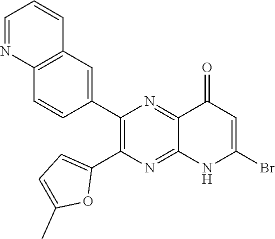 [SMILES: Cc1ccc(-c2nc3[nH]c(Br)cc(=O)c3nc2-c2ccc3ncccc3c2)o1] |
| 1.325 | 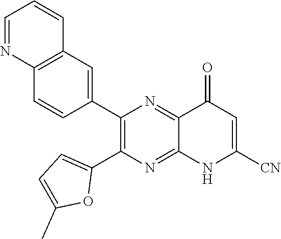 [SMILES: Cc1ccc(-c2nc3[nH]c(C#N)cc(=O)c3nc2-c2ccc3ncccc3c2)o1] |
| 1.326 | 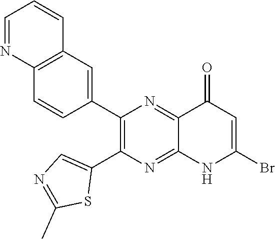 [SMILES: Cc1ncc(-c2nc3[nH]c(Br)cc(=O)c3nc2-c2ccc3ncccc3c2)s1] |
| 1.327 | 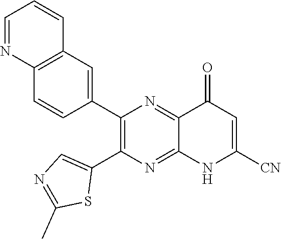 [SMILES: Cc1ncc(-c2nc3[nH]c(C#N)cc(=O)c3nc2-c2ccc3ncccc3c2)s1] |
| 1.328 | 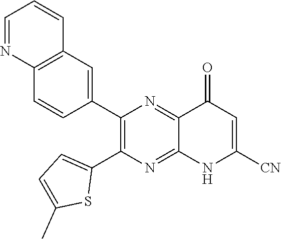 [SMILES: Cc1ccc(-c2nc3[nH]c(C#N)cc(=O)c3nc2-c2ccc3ncccc3c2)s1] |
| 1.329 | 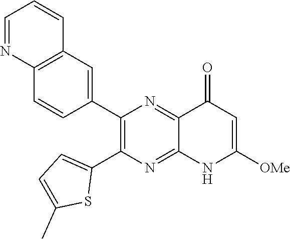 [SMILES: COc1cc(=O)c2nc(-c3ccc4ncccc4c3)c(-c3ccc(C)s3)nc2[nH]1] |
| 1.330 | 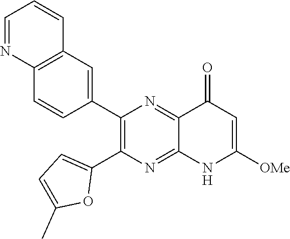 [SMILES: COc1cc(=O)c2nc(-c3ccc4ncccc4c3)c(-c3ccc(C)o3)nc2[nH]1] |
| 1.331 | 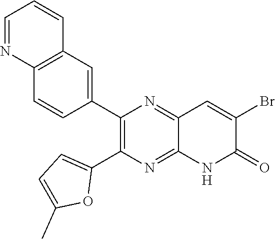 [SMILES: Cc1ccc(-c2nc3[nH]c(=O)c(Br)cc3nc2-c2ccc3ncccc3c2)o1] |
| 1.332 | 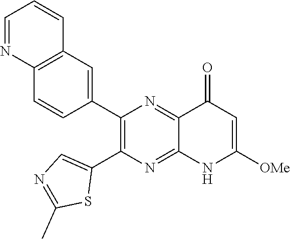 [SMILES: COc1cc(=O)c2nc(-c3ccc4ncccc4c3)c(-c3cnc(C)s3)nc2[nH]1] |
| 1.333 | 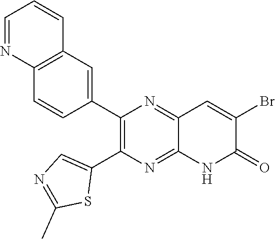 [SMILES: Cc1ncc(-c2nc3[nH]c(=O)c(Br)cc3nc2-c2ccc3ncccc3c2)s1] |
| 1.334 | 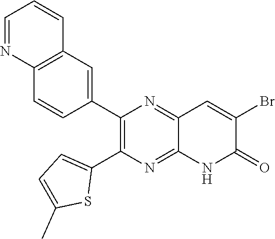 [SMILES: Cc1ccc(-c2nc3[nH]c(=O)c(Br)cc3nc2-c2ccc3ncccc3c2)s1] |
| 1.335 | 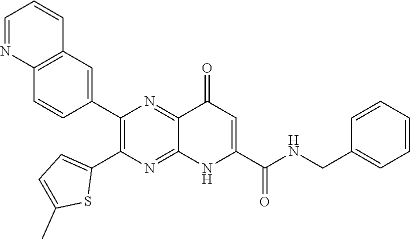 [SMILES: Cc1ccc(-c2nc3[nH]c(C(=O)NCc4ccccc4)cc(=O)c3nc2-c2ccc3ncccc3c2)s1] |
| 1.336 | 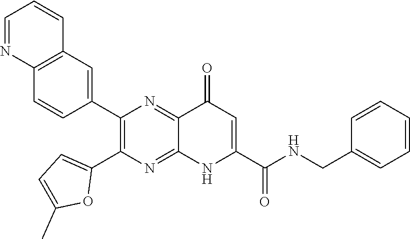 [SMILES: Cc1ccc(-c2nc3[nH]c(C(=O)NCc4ccccc4)cc(=O)c3nc2-c2ccc3ncccc3c2)o1] |
| 1.337 | 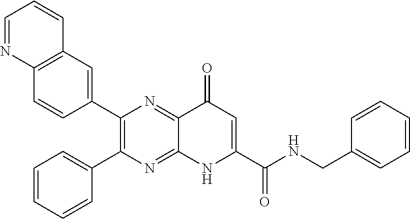 [SMILES: O=C(NCc1ccccc1)c1cc(=O)c2nc(-c3ccc4ncccc4c3)c(-c3ccccc3)nc2[nH]1] |
| 1.338 | 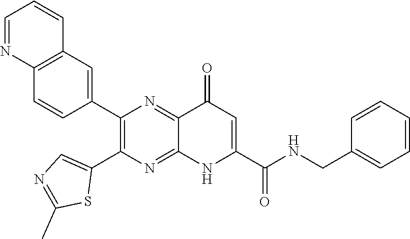 [SMILES: Cc1ncc(-c2nc3[nH]c(C(=O)NCc4ccccc4)cc(=O)c3nc2-c2ccc3ncccc3c2)s1] |
| 1.339 | 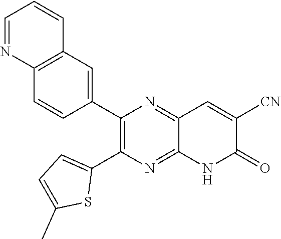 [SMILES: Cc1ccc(-c2nc3[nH]c(=O)c(C#N)cc3nc2-c2ccc3ncccc3c2)s1] |
| 1.340 | 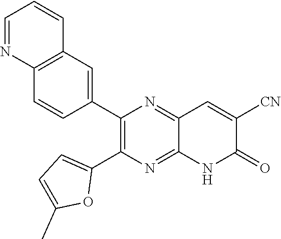 [SMILES: Cc1ccc(-c2nc3[nH]c(=O)c(C#N)cc3nc2-c2ccc3ncccc3c2)o1] |
| 1.341 | 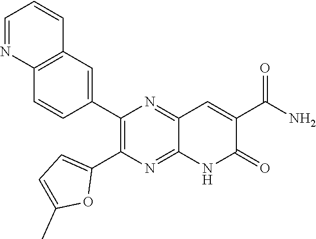 [SMILES: Cc1ccc(-c2nc3[nH]c(=O)c(C(N)=O)cc3nc2-c2ccc3ncccc3c2)o1] |
| 1.342 | 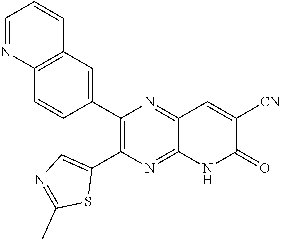 [SMILES: Cc1ncc(-c2nc3[nH]c(=O)c(C#N)cc3nc2-c2ccc3ncccc3c2)s1] |
| 1.343 | 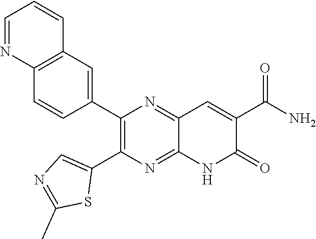 [SMILES: Cc1ncc(-c2nc3[nH]c(=O)c(C(N)=O)cc3nc2-c2ccc3ncccc3c2)s1] |
| 1.344 | 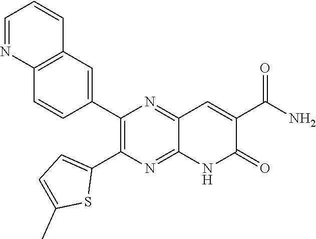 [SMILES: Cc1ccc(-c2nc3[nH]c(=O)c(C(N)=O)cc3nc2-c2ccc3ncccc3c2)s1] |
| 1.345 | 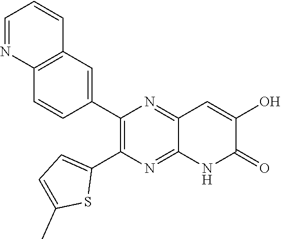 [SMILES: Cc1ccc(-c2nc3[nH]c(=O)c(O)cc3nc2-c2ccc3ncccc3c2)s1] |
| 1.346 | 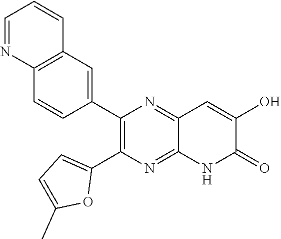 [SMILES: Cc1ccc(-c2nc3[nH]c(=O)c(O)cc3nc2-c2ccc3ncccc3c2)o1] |
| 1.347 | 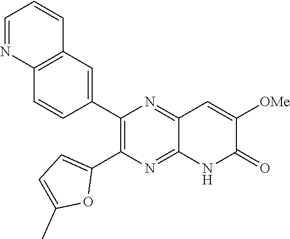 [SMILES: COc1cc2nc(-c3ccc4ncccc4c3)c(-c3ccc(C)o3)nc2[nH]c1=O] |
| 1.348 | 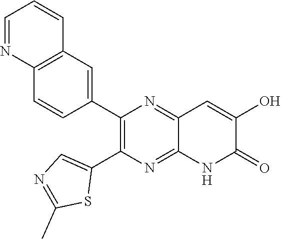 [SMILES: Cc1ncc(-c2nc3[nH]c(=O)c(O)cc3nc2-c2ccc3ncccc3c2)s1] |
| 1.349 | 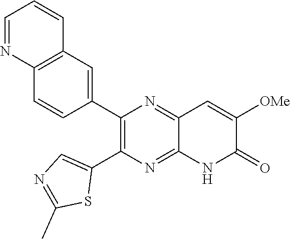 [SMILES: COc1cc2nc(-c3ccc4ncccc4c3)c(-c3cnc(C)s3)nc2[nH]c1=O] |
| 1.350 | 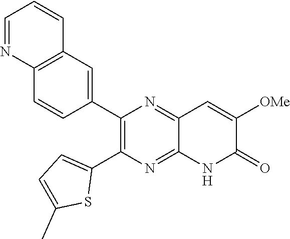 [SMILES: COc1cc2nc(-c3ccc4ncccc4c3)c(-c3ccc(C)s3)nc2[nH]c1=O] |
| 1.351 | 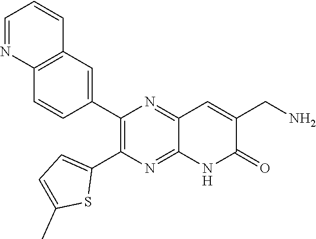 [SMILES: Cc1ccc(-c2nc3[nH]c(=O)c(CN)cc3nc2-c2ccc3ncccc3c2)s1] |
| 1.352 | 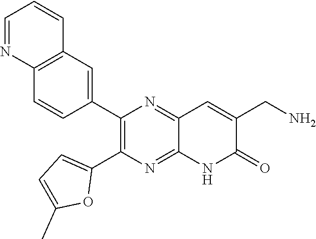 [SMILES: Cc1ccc(-c2nc3[nH]c(=O)c(CN)cc3nc2-c2ccc3ncccc3c2)o1] |
| 1.353 | 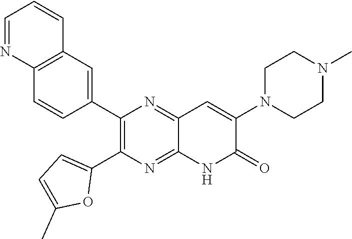 [SMILES: Cc1ccc(-c2nc3[nH]c(=O)c(N4CCN(C)CC4)cc3nc2-c2ccc3ncccc3c2)o1] |
| 1.354 | 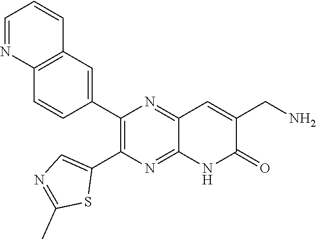 [SMILES: Cc1ncc(-c2nc3[nH]c(=O)c(CN)cc3nc2-c2ccc3ncccc3c2)s1] |
| 1.355 | 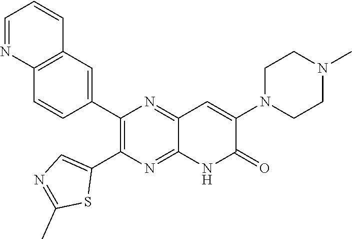 [SMILES: Cc1ncc(-c2nc3[nH]c(=O)c(N4CCN(C)CC4)cc3nc2-c2ccc3ncccc3c2)s1] |
| 1.356 | 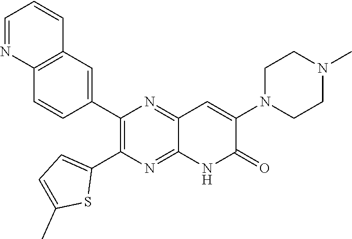 [SMILES: Cc1ccc(-c2nc3[nH]c(=O)c(N4CCN(C)CC4)cc3nc2-c2ccc3ncccc3c2)s1] |
| 1.357 | 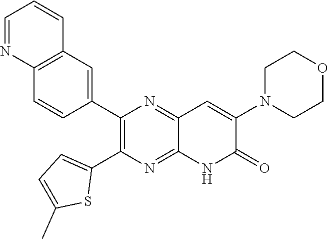 [SMILES: Cc1ccc(-c2nc3[nH]c(=O)c(N4CCOCC4)cc3nc2-c2ccc3ncccc3c2)s1] |
| 1.358 | 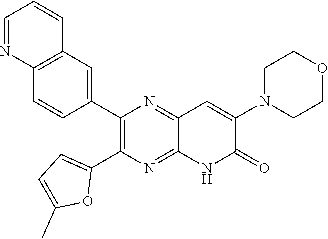 [SMILES: Cc1ccc(-c2nc3[nH]c(=O)c(N4CCOCC4)cc3nc2-c2ccc3ncccc3c2)o1] |
| 1.359 | 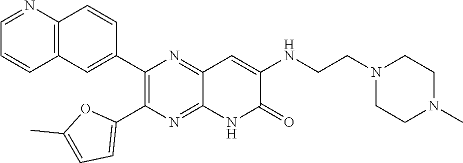 [SMILES: Cc1ccc(-c2nc3[nH]c(=O)c(NCCN4CCN(C)CC4)cc3nc2-c2ccc3ncccc3c2)o1] |
| 1.360 | 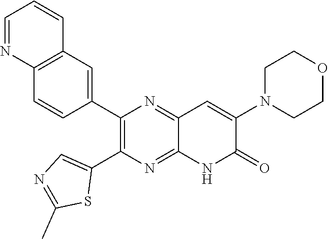 [SMILES: Cc1ncc(-c2nc3[nH]c(=O)c(N4CCOCC4)cc3nc2-c2ccc3ncccc3c2)s1] |
| 1.361 | 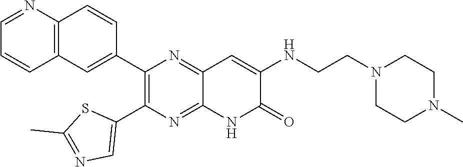 [SMILES: Cc1ncc(-c2nc3[nH]c(=O)c(NCCN4CCN(C)CC4)cc3nc2-c2ccc3ncccc3c2)s1] |
| 1.362 | 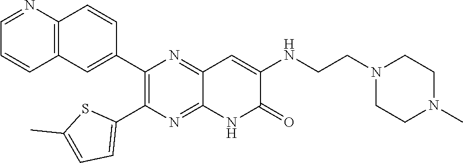 [SMILES: Cc1ccc(-c2nc3[nH]c(=O)c(NCCN4CCN(C)CC4)cc3nc2-c2ccc3ncccc3c2)s1] |
| 1.363 | 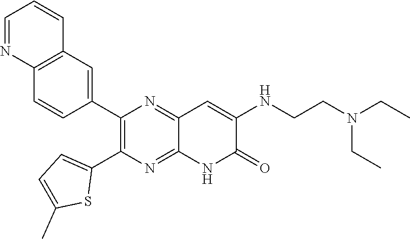 [SMILES: CCN(CC)CCNc1cc2nc(-c3ccc4ncccc4c3)c(-c3ccc(C)s3)nc2[nH]c1=O] |
| 1.364 | 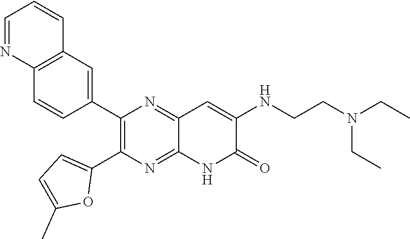 [SMILES: CCN(CC)CCNc1cc2nc(-c3ccc4ncccc4c3)c(-c3ccc(C)o3)nc2[nH]c1=O] |
| 1.365 | 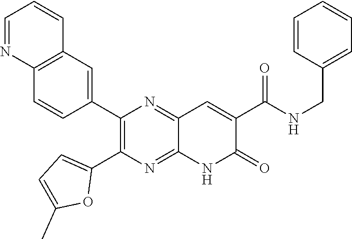 [SMILES: Cc1ccc(-c2nc3[nH]c(=O)c(C(=O)NCc4ccccc4)cc3nc2-c2ccc3ncccc3c2)o1] |
| 1.366 | 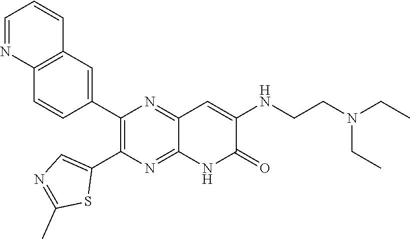 [SMILES: CCN(CC)CCNc1cc2nc(-c3ccc4ncccc4c3)c(-c3cnc(C)s3)nc2[nH]c1=O] |
| 1.367 | 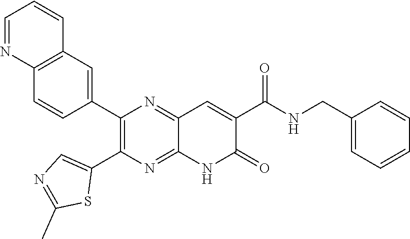 [SMILES: Cc1ncc(-c2nc3[nH]c(=O)c(C(=O)NCc4ccccc4)cc3nc2-c2ccc3ncccc3c2)s1] |
| 1.368 | 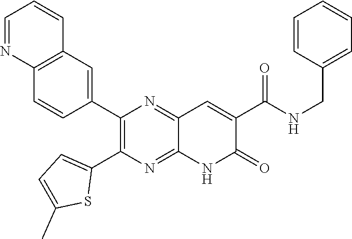 [SMILES: Cc1ccc(-c2nc3[nH]c(=O)c(C(=O)NCc4ccccc4)cc3nc2-c2ccc3ncccc3c2)s1] |
| 1.369 | 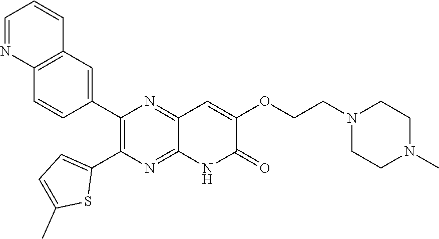 [SMILES: Cc1ccc(-c2nc3[nH]c(=O)c(OCCN4CCN(C)CC4)cc3nc2-c2ccc3ncccc3c2)s1] |
| 1.370 | 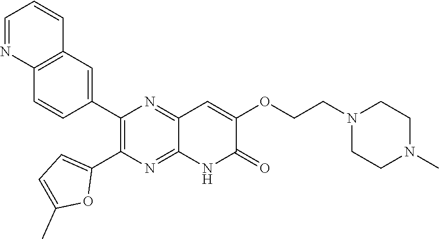 [SMILES: Cc1ccc(-c2nc3[nH]c(=O)c(OCCN4CCN(C)CC4)cc3nc2-c2ccc3ncccc3c2)o1] |
| 1.371 | 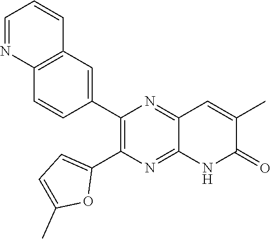 [SMILES: Cc1ccc(-c2nc3[nH]c(=O)c(C)cc3nc2-c2ccc3ncccc3c2)o1] |
| 1.372 | 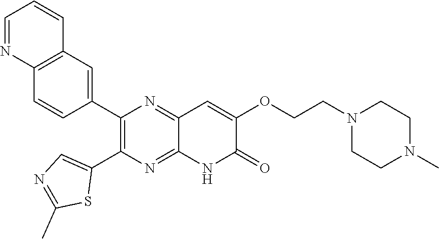 [SMILES: Cc1ncc(-c2nc3[nH]c(=O)c(OCCN4CCN(C)CC4)cc3nc2-c2ccc3ncccc3c2)s1] |
| 1.373 | 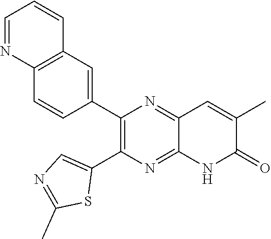 [SMILES: Cc1ncc(-c2nc3[nH]c(=O)c(C)cc3nc2-c2ccc3ncccc3c2)s1] |
| 1.374 | 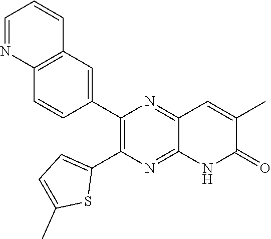 [SMILES: Cc1ccc(-c2nc3[nH]c(=O)c(C)cc3nc2-c2ccc3ncccc3c2)s1] |
| 1.375 | 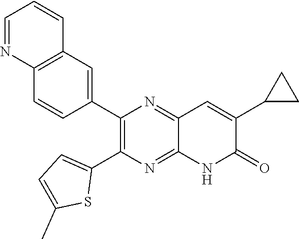 [SMILES: Cc1ccc(-c2nc3[nH]c(=O)c(C4CC4)cc3nc2-c2ccc3ncccc3c2)s1] |
| 1.376 | 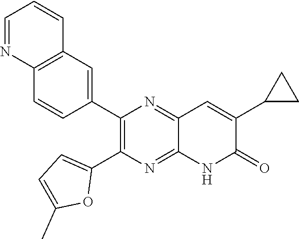 [SMILES: Cc1ccc(-c2nc3[nH]c(=O)c(C4CC4)cc3nc2-c2ccc3ncccc3c2)o1] |
| 1.377 | 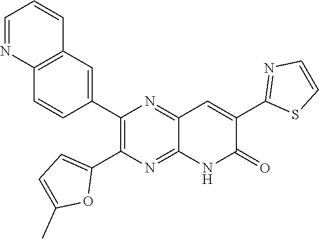 [SMILES: Cc1ccc(-c2nc3[nH]c(=O)c(-c4nccs4)cc3nc2-c2ccc3ncccc3c2)o1] |
| 1.378 | 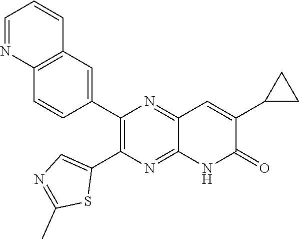 [SMILES: Cc1ncc(-c2nc3[nH]c(=O)c(C4CC4)cc3nc2-c2ccc3ncccc3c2)s1] |
| 1.379 | 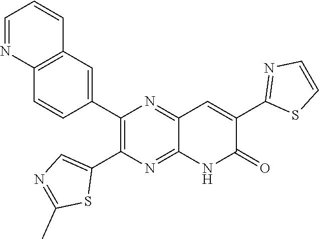 [SMILES: Cc1ncc(-c2nc3[nH]c(=O)c(-c4nccs4)cc3nc2-c2ccc3ncccc3c2)s1] |
| 1.380 | 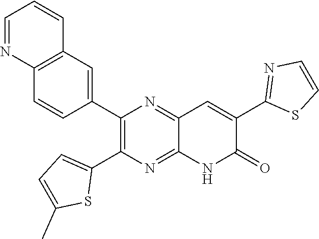 [SMILES: Cc1ccc(-c2nc3[nH]c(=O)c(-c4nccs4)cc3nc2-c2ccc3ncccc3c2)s1] |
| 1.381 | 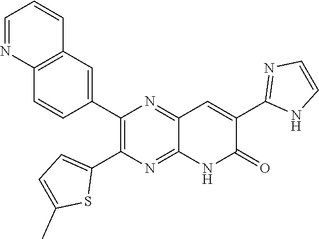 [SMILES: Cc1ccc(-c2nc3[nH]c(=O)c(-c4ncc[nH]4)cc3nc2-c2ccc3ncccc3c2)s1] |
| 1.382 | 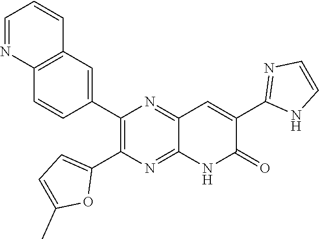 [SMILES: Cc1ccc(-c2nc3[nH]c(=O)c(-c4ncc[nH]4)cc3nc2-c2ccc3ncccc3c2)o1] |
| 1.383 | 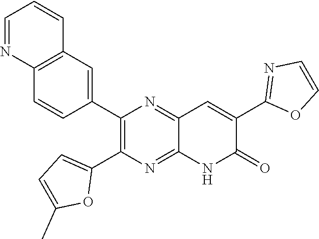 [SMILES: Cc1ccc(-c2nc3[nH]c(=O)c(-c4ncco4)cc3nc2-c2ccc3ncccc3c2)o1] |
| 1.384 | 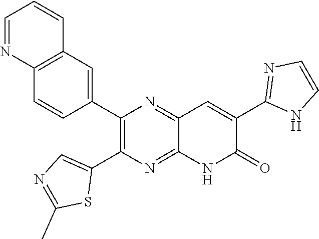 [SMILES: Cc1ncc(-c2nc3[nH]c(=O)c(-c4ncc[nH]4)cc3nc2-c2ccc3ncccc3c2)s1] |
| 1.385 | 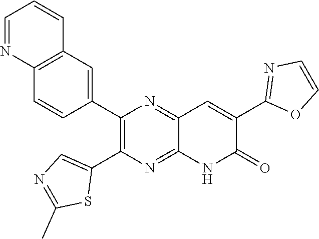 [SMILES: Cc1ncc(-c2nc3[nH]c(=O)c(-c4ncco4)cc3nc2-c2ccc3ncccc3c2)s1] |
| 1.386 | 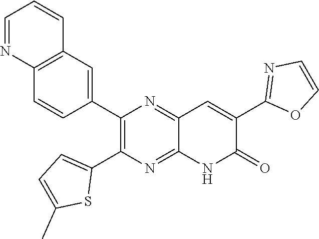 [SMILES: Cc1ccc(-c2nc3[nH]c(=O)c(-c4ncco4)cc3nc2-c2ccc3ncccc3c2)s1] |
| 1.387 | 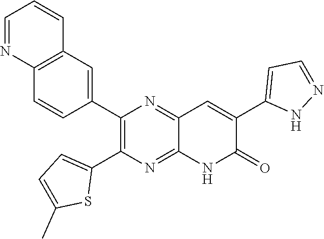 [SMILES: Cc1ccc(-c2nc3[nH]c(=O)c(-c4ccn[nH]4)cc3nc2-c2ccc3ncccc3c2)s1] |
| 1.388 | 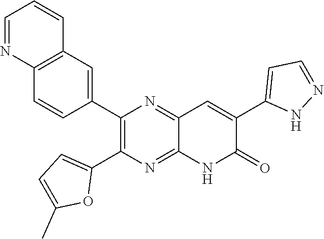 [SMILES: Cc1ccc(-c2nc3[nH]c(=O)c(-c4ccn[nH]4)cc3nc2-c2ccc3ncccc3c2)o1] |
| 1.389 | 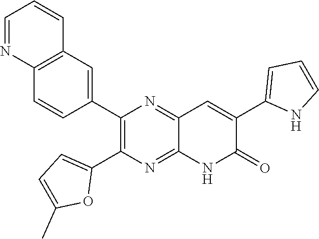 [SMILES: Cc1ccc(-c2nc3[nH]c(=O)c(-c4ccc[nH]4)cc3nc2-c2ccc3ncccc3c2)o1] |
| 1.390 | 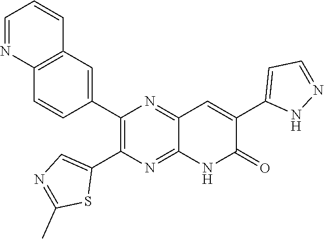 [SMILES: Cc1ncc(-c2nc3[nH]c(=O)c(-c4ccn[nH]4)cc3nc2-c2ccc3ncccc3c2)s1] |
| 1.391 | 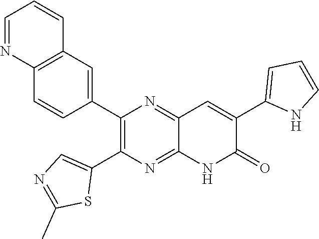 [SMILES: Cc1ncc(-c2nc3[nH]c(=O)c(-c4ccc[nH]4)cc3nc2-c2ccc3ncccc3c2)s1] |
| 1.392 | 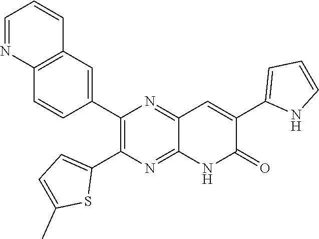 [SMILES: Cc1ccc(-c2nc3[nH]c(=O)c(-c4ccc[nH]4)cc3nc2-c2ccc3ncccc3c2)s1] |
| 1.393 | 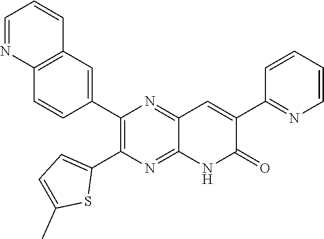 [SMILES: Cc1ccc(-c2nc3[nH]c(=O)c(-c4ccccn4)cc3nc2-c2ccc3ncccc3c2)s1] |
| 1.394 | 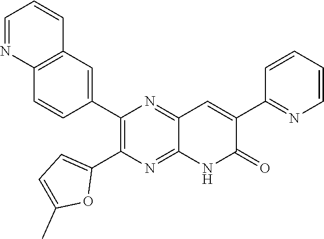 [SMILES: Cc1ccc(-c2nc3[nH]c(=O)c(-c4ccccn4)cc3nc2-c2ccc3ncccc3c2)o1] |
| 1.395 | 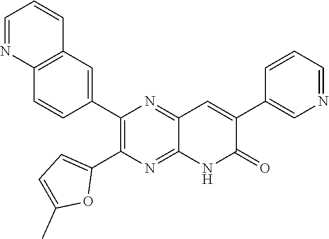 [SMILES: Cc1ccc(-c2nc3[nH]c(=O)c(-c4cccnc4)cc3nc2-c2ccc3ncccc3c2)o1] |
| 1.396 | 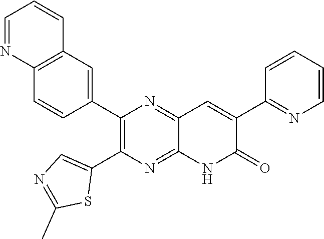 [SMILES: Cc1ncc(-c2nc3[nH]c(=O)c(-c4ccccn4)cc3nc2-c2ccc3ncccc3c2)s1] |
| 1.397 | 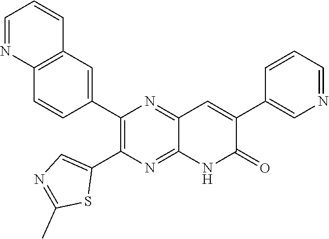 [SMILES: Cc1ncc(-c2nc3[nH]c(=O)c(-c4cccnc4)cc3nc2-c2ccc3ncccc3c2)s1] |
| 1.398 | 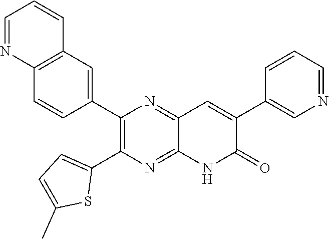 [SMILES: Cc1ccc(-c2nc3[nH]c(=O)c(-c4cccnc4)cc3nc2-c2ccc3ncccc3c2)s1] |
| 1.399 | 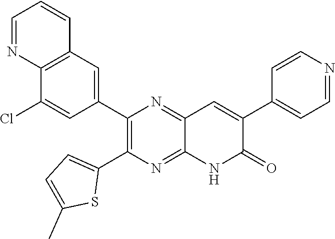 [SMILES: Cc1ccc(-c2nc3[nH]c(=O)c(-c4ccncc4)cc3nc2-c2cc(Cl)c3ncccc3c2)s1] |
| 1.400 | 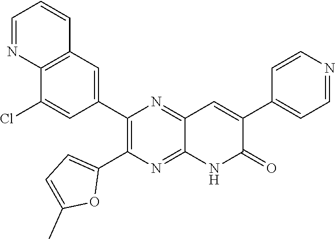 [SMILES: Cc1ccc(-c2nc3[nH]c(=O)c(-c4ccncc4)cc3nc2-c2cc(Cl)c3ncccc3c2)o1] |
| 1.401 | 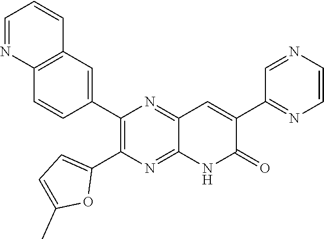 [SMILES: Cc1ccc(-c2nc3[nH]c(=O)c(-c4cnccn4)cc3nc2-c2ccc3ncccc3c2)o1] |
| 1.402 | 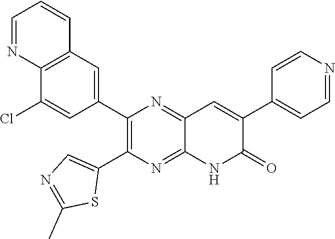 [SMILES: Cc1ncc(-c2nc3[nH]c(=O)c(-c4ccncc4)cc3nc2-c2cc(Cl)c3ncccc3c2)s1] |
| 1.403 | 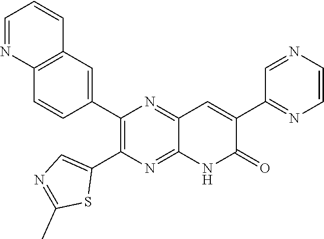 [SMILES: Cc1ncc(-c2nc3[nH]c(=O)c(-c4cnccn4)cc3nc2-c2ccc3ncccc3c2)s1] |
| 1.404 | 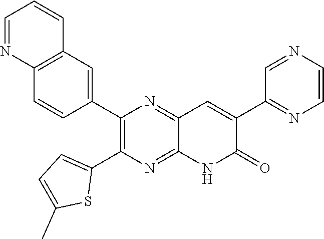 [SMILES: Cc1ccc(-c2nc3[nH]c(=O)c(-c4cnccn4)cc3nc2-c2ccc3ncccc3c2)s1] |
| 1.405 | 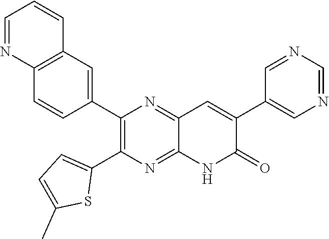 [SMILES: Cc1ccc(-c2nc3[nH]c(=O)c(-c4cncnc4)cc3nc2-c2ccc3ncccc3c2)s1] |
| 1.406 | 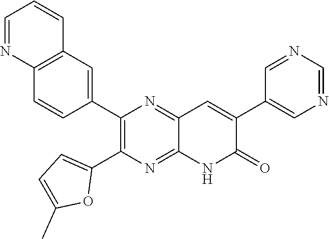 [SMILES: Cc1ccc(-c2nc3[nH]c(=O)c(-c4cncnc4)cc3nc2-c2ccc3ncccc3c2)o1] |
| 1.407 | 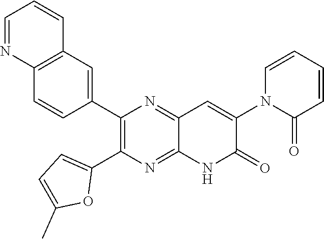 [SMILES: Cc1ccc(-c2nc3[nH]c(=O)c(-n4ccccc4=O)cc3nc2-c2ccc3ncccc3c2)o1] |
| 1.408 | 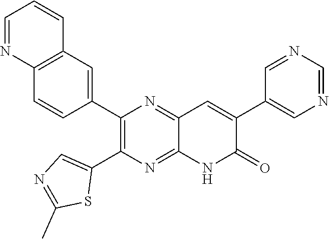 [SMILES: Cc1ncc(-c2nc3[nH]c(=O)c(-c4cncnc4)cc3nc2-c2ccc3ncccc3c2)s1] |
| 1.409 | 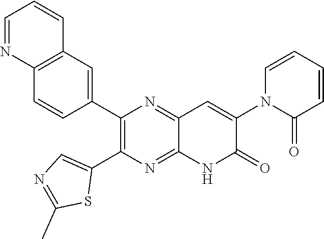 [SMILES: Cc1ncc(-c2nc3[nH]c(=O)c(-n4ccccc4=O)cc3nc2-c2ccc3ncccc3c2)s1] |
| 1.410 | 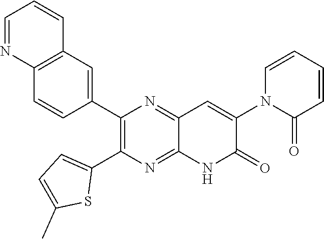 [SMILES: Cc1ccc(-c2nc3[nH]c(=O)c(-n4ccccc4=O)cc3nc2-c2ccc3ncccc3c2)s1] |
| 1.411 | 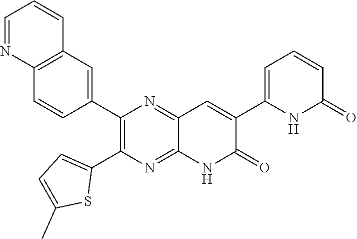 [SMILES: Cc1ccc(-c2nc3[nH]c(=O)c(-c4cccc(=O)[nH]4)cc3nc2-c2ccc3ncccc3c2)s1] |
| 1.412 | 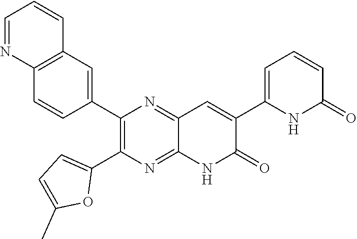 [SMILES: Cc1ccc(-c2nc3[nH]c(=O)c(-c4cccc(=O)[nH]4)cc3nc2-c2ccc3ncccc3c2)o1] |
| 1.413 | 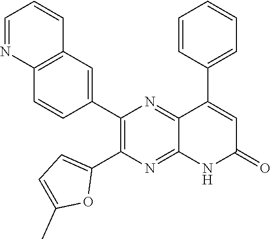 [SMILES: Cc1ccc(-c2nc3[nH]c(=O)cc(-c4ccccc4)c3nc2-c2ccc3ncccc3c2)o1] |
| 1.414 | 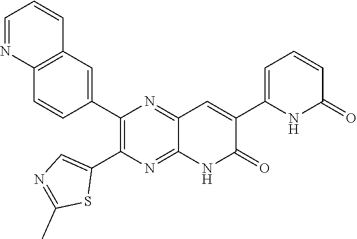 [SMILES: Cc1ncc(-c2nc3[nH]c(=O)c(-c4cccc(=O)[nH]4)cc3nc2-c2ccc3ncccc3c2)s1] |
| 1.415 | 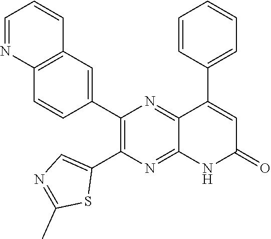 [SMILES: Cc1ncc(-c2nc3[nH]c(=O)cc(-c4ccccc4)c3nc2-c2ccc3ncccc3c2)s1] |
| 1.416 | 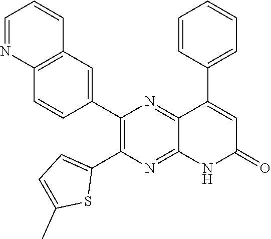 [SMILES: Cc1ccc(-c2nc3[nH]c(=O)cc(-c4ccccc4)c3nc2-c2ccc3ncccc3c2)s1] |
| 1.417 | 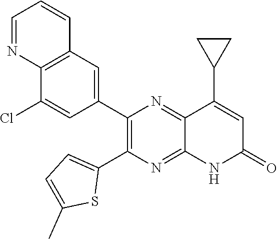 [SMILES: Cc1ccc(-c2nc3[nH]c(=O)cc(C4CC4)c3nc2-c2cc(Cl)c3ncccc3c2)s1] |
| 1.418 | 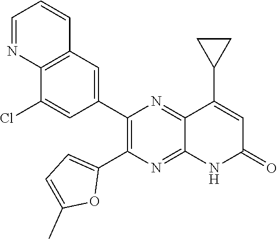 [SMILES: Cc1ccc(-c2nc3[nH]c(=O)cc(C4CC4)c3nc2-c2cc(Cl)c3ncccc3c2)o1] |
| 1.419 | 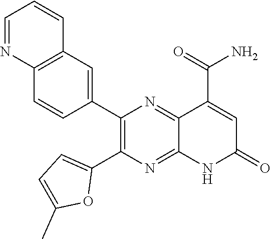 [SMILES: Cc1ccc(-c2nc3[nH]c(=O)cc(C(N)=O)c3nc2-c2ccc3ncccc3c2)o1] |
| 1.420 | 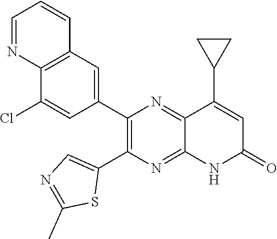 [SMILES: Cc1ncc(-c2nc3[nH]c(=O)cc(C4CC4)c3nc2-c2cc(Cl)c3ncccc3c2)s1] |
| 1.421 | 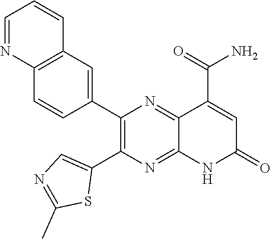 [SMILES: Cc1ncc(-c2nc3[nH]c(=O)cc(C(N)=O)c3nc2-c2ccc3ncccc3c2)s1] |
| 1.422 | 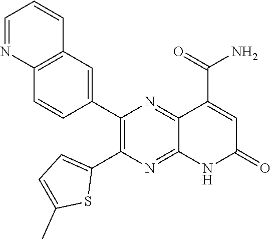 [SMILES: Cc1ccc(-c2nc3[nH]c(=O)cc(C(N)=O)c3nc2-c2ccc3ncccc3c2)s1] |
| 1.423 | 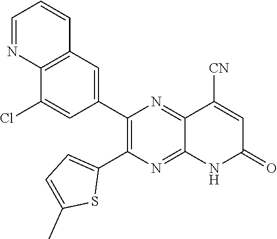 [SMILES: Cc1ccc(-c2nc3[nH]c(=O)cc(C#N)c3nc2-c2cc(Cl)c3ncccc3c2)s1] |
| 1.424 | 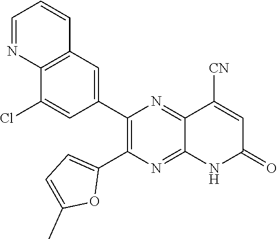 [SMILES: Cc1ccc(-c2nc3[nH]c(=O)cc(C#N)c3nc2-c2cc(Cl)c3ncccc3c2)o1] |
| 1.425 | 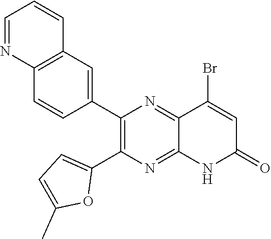 [SMILES: Cc1ccc(-c2nc3[nH]c(=O)cc(Br)c3nc2-c2ccc3ncccc3c2)o1] |
| 1.426 | 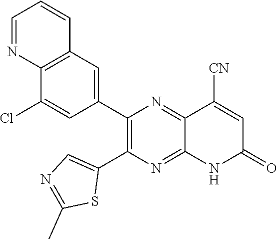 [SMILES: Cc1ncc(-c2nc3[nH]c(=O)cc(C#N)c3nc2-c2cc(Cl)c3ncccc3c2)s1] |
| 1.427 | 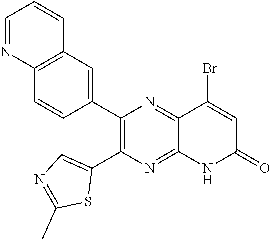 [SMILES: Cc1ncc(-c2nc3[nH]c(=O)cc(Br)c3nc2-c2ccc3ncccc3c2)s1] |
| 1.428 | 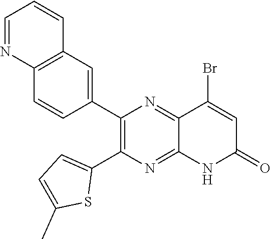 [SMILES: Cc1ccc(-c2nc3[nH]c(=O)cc(Br)c3nc2-c2ccc3ncccc3c2)s1] |
| 1.429 | 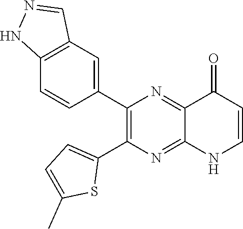 [SMILES: Cc1ccc(-c2nc3[nH]ccc(=O)c3nc2-c2ccc3[nH]ncc3c2)s1] |
| 1.430 | 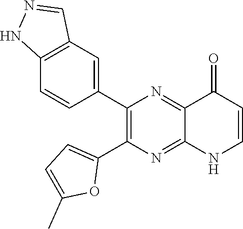 [SMILES: Cc1ccc(-c2nc3[nH]ccc(=O)c3nc2-c2ccc3[nH]ncc3c2)o1] |
| 1.431 | 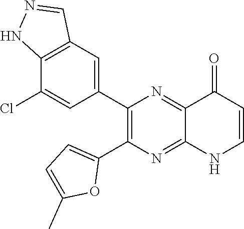 [SMILES: Cc1ccc(-c2nc3[nH]ccc(=O)c3nc2-c2cc(Cl)c3[nH]ncc3c2)o1] |
| 1.432 | 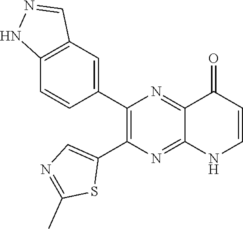 [SMILES: Cc1ncc(-c2nc3[nH]ccc(=O)c3nc2-c2ccc3[nH]ncc3c2)s1] |
| 1.433 | 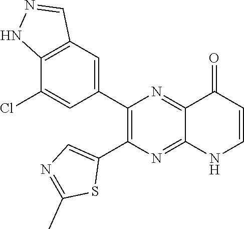 [SMILES: Cc1ncc(-c2nc3[nH]ccc(=O)c3nc2-c2cc(Cl)c3[nH]ncc3c2)s1] |
| 1.434 | 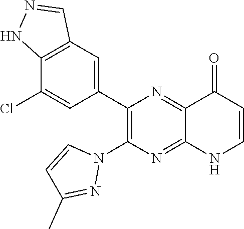 [SMILES: Cc1ccn(-c2nc3[nH]ccc(=O)c3nc2-c2cc(Cl)c3[nH]ncc3c2)n1] |
| 1.435 | 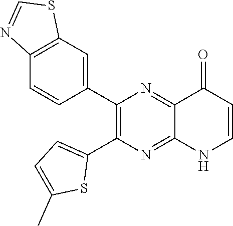 [SMILES: Cc1ccc(-c2nc3[nH]ccc(=O)c3nc2-c2ccc3ncsc3c2)s1] |
| 1.436 | 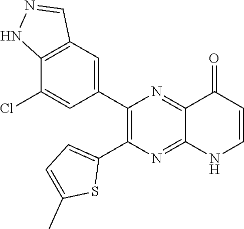 [SMILES: Cc1ccc(-c2nc3[nH]ccc(=O)c3nc2-c2cc(Cl)c3[nH]ncc3c2)s1] |
| 1.437 | 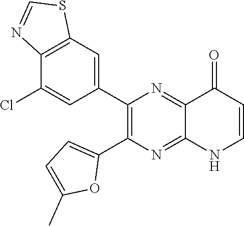 [SMILES: Cc1ccc(-c2nc3[nH]ccc(=O)c3nc2-c2cc(Cl)c3ncsc3c2)o1] |
| 1.438 | 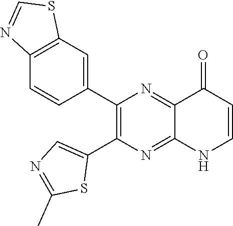 [SMILES: Cc1ncc(-c2nc3[nH]ccc(=O)c3nc2-c2ccc3ncsc3c2)s1] |
| 1.439 | 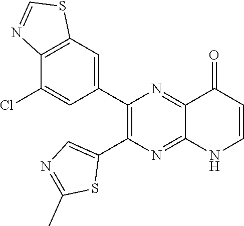 [SMILES: Cc1ncc(-c2nc3[nH]ccc(=O)c3nc2-c2cc(Cl)c3ncsc3c2)s1] |
| 1.440 | 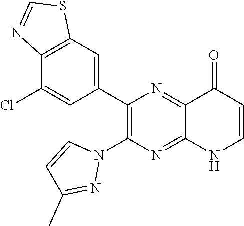 [SMILES: Cc1ccn(-c2nc3[nH]ccc(=O)c3nc2-c2cc(Cl)c3ncsc3c2)n1] |
| 1.441 | 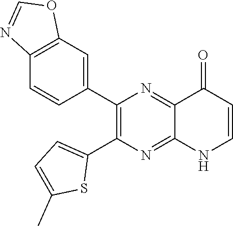 [SMILES: Cc1ccc(-c2nc3[nH]ccc(=O)c3nc2-c2ccc3ncoc3c2)s1] |
| 1.442 | 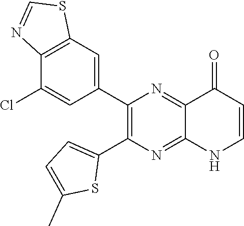 [SMILES: Cc1ccc(-c2nc3[nH]ccc(=O)c3nc2-c2cc(Cl)c3ncsc3c2)s1] |
| 1.443 | 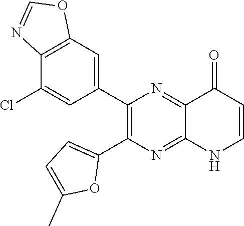 [SMILES: Cc1ccc(-c2nc3[nH]ccc(=O)c3nc2-c2cc(Cl)c3ncoc3c2)o1] |
| 1.444 | 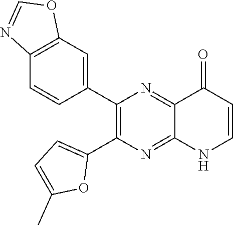 [SMILES: Cc1ccc(-c2nc3[nH]ccc(=O)c3nc2-c2ccc3ncoc3c2)o1] |
| 1.445 | 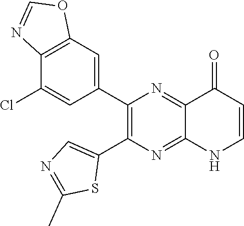 [SMILES: Cc1ncc(-c2nc3[nH]ccc(=O)c3nc2-c2cc(Cl)c3ncoc3c2)s1] |
| 1.446 | 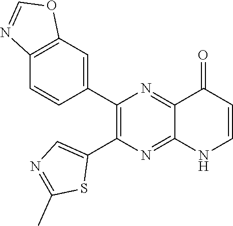 [SMILES: Cc1ncc(-c2nc3[nH]ccc(=O)c3nc2-c2ccc3ncoc3c2)s1] |
| 1.447 | 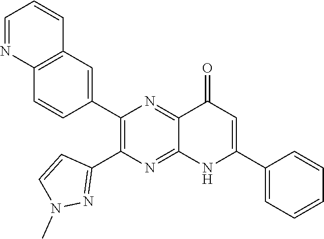 [SMILES: Cn1ccc(-c2nc3[nH]c(-c4ccccc4)cc(=O)c3nc2-c2ccc3ncccc3c2)n1] |
| 1.448 | 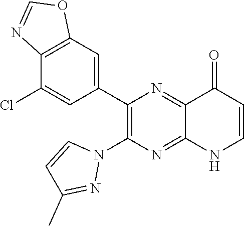 [SMILES: Cc1ccn(-c2nc3[nH]ccc(=O)c3nc2-c2cc(Cl)c3ncoc3c2)n1] |
| 1.449 | 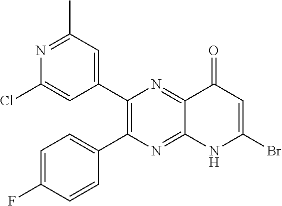 [SMILES: Cc1cc(-c2nc3c(=O)cc(Br)[nH]c3nc2-c2ccc(F)cc2)cc(Cl)n1] |
| 1.450 | 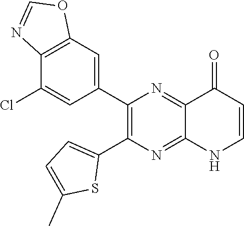 [SMILES: Cc1ccc(-c2nc3[nH]ccc(=O)c3nc2-c2cc(Cl)c3ncoc3c2)s1] |
| 1.451 | 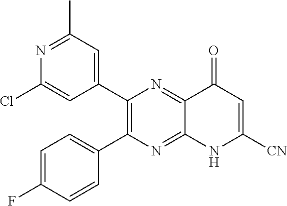 [SMILES: Cc1cc(-c2nc3c(=O)cc(C#N)[nH]c3nc2-c2ccc(F)cc2)cc(Cl)n1] |
| 1.452 | 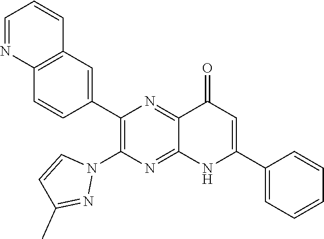 [SMILES: Cc1ccn(-c2nc3[nH]c(-c4ccccc4)cc(=O)c3nc2-c2ccc3ncccc3c2)n1] |
| 1.453 | 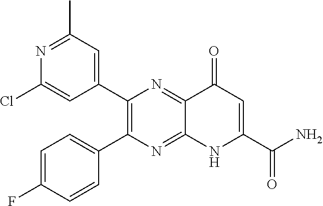 [SMILES: Cc1cc(-c2nc3c(=O)cc(C(N)=O)[nH]c3nc2-c2ccc(F)cc2)cc(Cl)n1] |
| 1.454 | 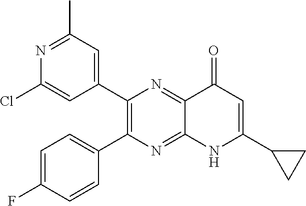 [SMILES: Cc1cc(-c2nc3c(=O)cc(C4CC4)[nH]c3nc2-c2ccc(F)cc2)cc(Cl)n1] |
| 1.455 | 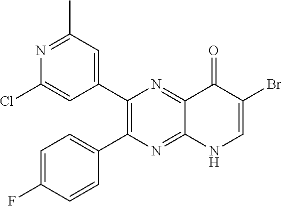 [SMILES: Cc1cc(-c2nc3c(=O)c(Br)c[nH]c3nc2-c2ccc(F)cc2)cc(Cl)n1] |
| 1.456 | 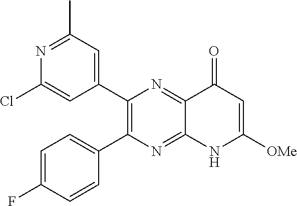 [SMILES: COc1cc(=O)c2nc(-c3cc(C)nc(Cl)c3)c(-c3ccc(F)cc3)nc2[nH]1] |
| 1.457 | 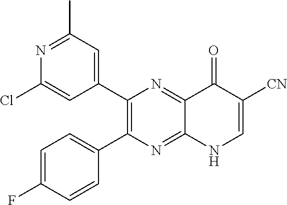 [SMILES: Cc1cc(-c2nc3c(=O)c(C#N)c[nH]c3nc2-c2ccc(F)cc2)cc(Cl)n1] |
| 1.458 | 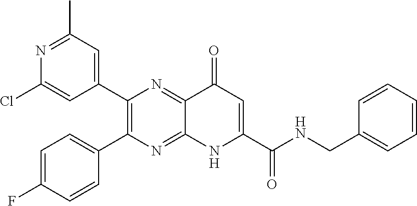 [SMILES: Cc1cc(-c2nc3c(=O)cc(C(=O)NCc4ccccc4)[nH]c3nc2-c2ccc(F)cc2)cc(Cl)n1] |
| 1.459 | 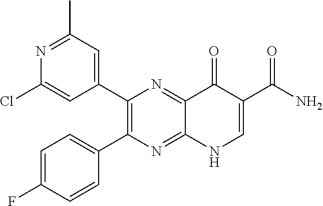 [SMILES: Cc1cc(-c2nc3c(=O)c(C(N)=O)c[nH]c3nc2-c2ccc(F)cc2)cc(Cl)n1] |
| 1.460 | 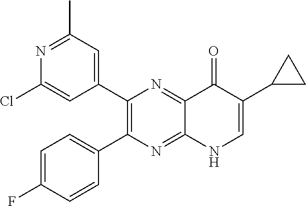 [SMILES: Cc1cc(-c2nc3c(=O)c(C4CC4)c[nH]c3nc2-c2ccc(F)cc2)cc(Cl)n1] |
| 1.461 | 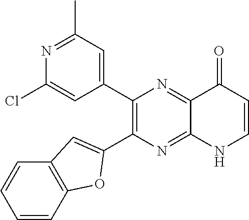 [SMILES: Cc1cc(-c2nc3c(=O)cc[nH]c3nc2-c2cc3ccccc3o2)cc(Cl)n1] |
| 1.462 | 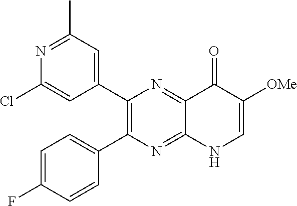 [SMILES: COc1c[nH]c2nc(-c3ccc(F)cc3)c(-c3cc(C)nc(Cl)c3)nc2c1=O] |
| 1.463 | 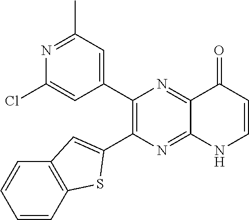 [SMILES: Cc1cc(-c2nc3c(=O)cc[nH]c3nc2-c2cc3ccccc3s2)cc(Cl)n1] |
| 1.464 | 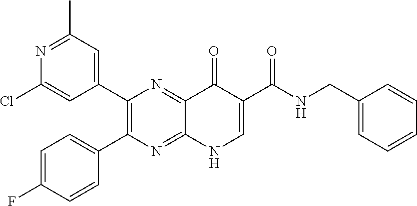 [SMILES: Cc1cc(-c2nc3c(=O)c(C(=O)NCc4ccccc4)c[nH]c3nc2-c2ccc(F)cc2)cc(Cl)n1] |
| 1.465 | 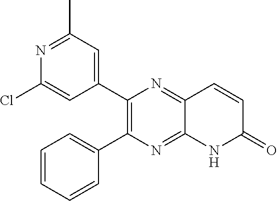 [SMILES: Cc1cc(-c2nc3ccc(=O)[nH]c3nc2-c2ccccc2)cc(Cl)n1] |
| 1.466 | 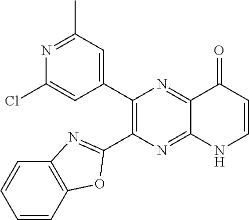 [SMILES: Cc1cc(-c2nc3c(=O)cc[nH]c3nc2-c2nc3ccccc3o2)cc(Cl)n1] |
| 1.467 | 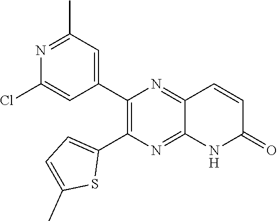 [SMILES: Cc1cc(-c2nc3ccc(=O)[nH]c3nc2-c2ccc(C)s2)cc(Cl)n1] |
| 1.468 | 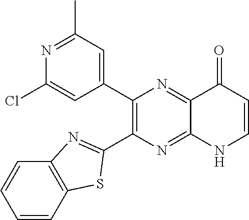 [SMILES: Cc1cc(-c2nc3c(=O)cc[nH]c3nc2-c2nc3ccccc3s2)cc(Cl)n1] |
| 1.469 | 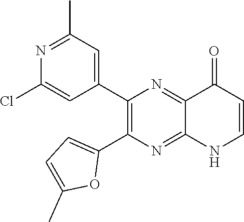 [SMILES: Cc1cc(-c2nc3c(=O)cc[nH]c3nc2-c2ccc(C)o2)cc(Cl)n1] |
| 1.470 | 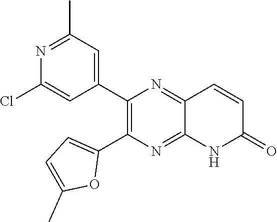 [SMILES: Cc1cc(-c2nc3ccc(=O)[nH]c3nc2-c2ccc(C)o2)cc(Cl)n1] |
| 1.471 | 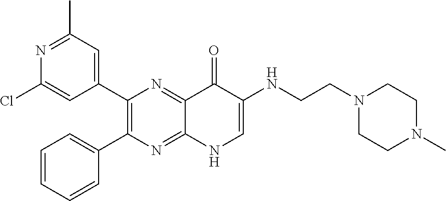 [SMILES: Cc1cc(-c2nc3c(=O)c(NCCN4CCN(C)CC4)c[nH]c3nc2-c2ccccc2)cc(Cl)n1] |
| 1.472 | 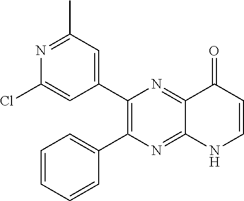 [SMILES: Cc1cc(-c2nc3c(=O)cc[nH]c3nc2-c2ccccc2)cc(Cl)n1] |
| 1.473 | 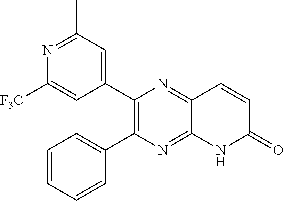 [SMILES: Cc1cc(-c2nc3ccc(=O)[nH]c3nc2-c2ccccc2)cc(C(F)(F)F)n1] |
| 1.474 | 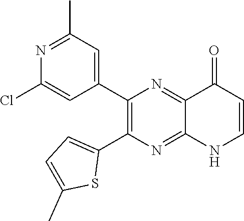 [SMILES: Cc1cc(-c2nc3c(=O)cc[nH]c3nc2-c2ccc(C)s2)cc(Cl)n1] |
| 1.475 | 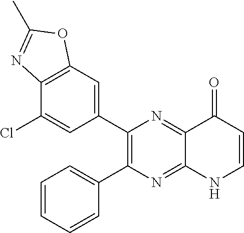 [SMILES: Cc1nc2c(Cl)cc(-c3nc4c(=O)cc[nH]c4nc3-c3ccccc3)cc2o1] |
| 1.476 | 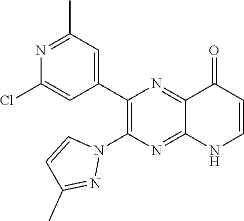 [SMILES: Cc1cc(-c2nc3c(=O)cc[nH]c3nc2-n2ccc(C)n2)cc(Cl)n1] |
| 1.477 | 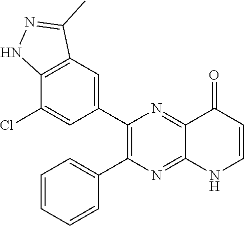 [SMILES: Cc1n[nH]c2c(Cl)cc(-c3nc4c(=O)cc[nH]c4nc3-c3ccccc3)cc12] |
| 1.478 | 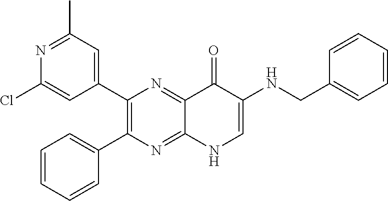 [SMILES: Cc1cc(-c2nc3c(=O)c(NCc4ccccc4)c[nH]c3nc2-c2ccccc2)cc(Cl)n1] |
| 1.479 | 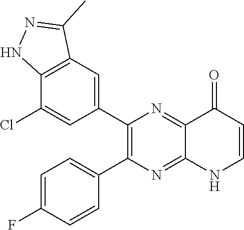 [SMILES: Cc1n[nH]c2c(Cl)cc(-c3nc4c(=O)cc[nH]c4nc3-c3ccc(F)cc3)cc12] |
| 1.480 | 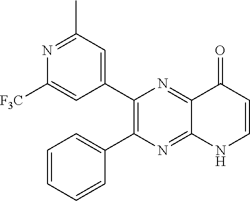 [SMILES: Cc1cc(-c2nc3c(=O)cc[nH]c3nc2-c2ccccc2)cc(C(F)(F)F)n1] |
| 1.481 | 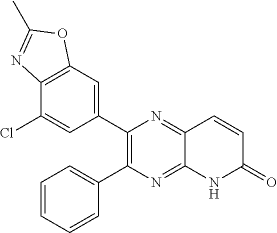 [SMILES: Cc1nc2c(Cl)cc(-c3nc4ccc(=O)[nH]c4nc3-c3ccccc3)cc2o1] |
| 1.482 | 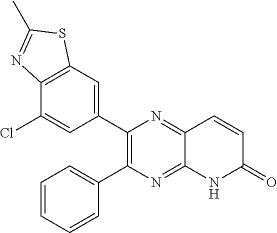 [SMILES: Cc1nc2c(Cl)cc(-c3nc4ccc(=O)[nH]c4nc3-c3ccccc3)cc2s1] |
| 1.483 | 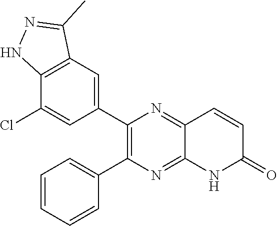 [SMILES: Cc1n[nH]c2c(Cl)cc(-c3nc4ccc(=O)[nH]c4nc3-c3ccccc3)cc12] |
| 1.484 | 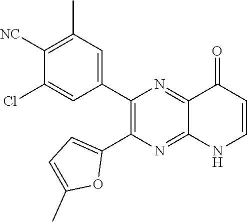 [SMILES: Cc1ccc(-c2nc3[nH]ccc(=O)c3nc2-c2cc(C)c(C#N)c(Cl)c2)o1] |
| 1.485 | 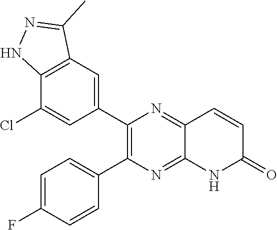 [SMILES: Cc1n[nH]c2c(Cl)cc(-c3nc4ccc(=O)[nH]c4nc3-c3ccc(F)cc3)cc12] |
| 1.486 | 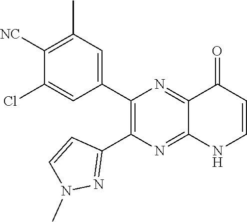 [SMILES: Cc1cc(-c2nc3c(=O)cc[nH]c3nc2-c2ccn(C)n2)cc(Cl)c1C#N] |
| 1.487 | 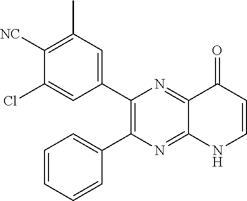 [SMILES: Cc1cc(-c2nc3c(=O)cc[nH]c3nc2-c2ccccc2)cc(Cl)c1C#N] |
| 1.488 | 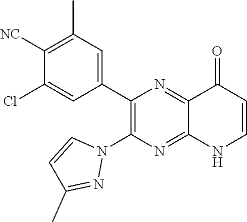 [SMILES: Cc1ccn(-c2nc3[nH]ccc(=O)c3nc2-c2cc(C)c(C#N)c(Cl)c2)n1] |
| 1.489 | 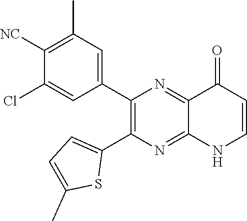 [SMILES: Cc1ccc(-c2nc3[nH]ccc(=O)c3nc2-c2cc(C)c(C#N)c(Cl)c2)s1] |
| 1.490 | 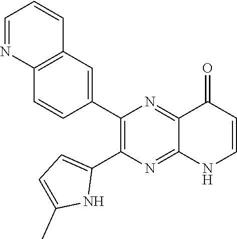 [SMILES: Cc1ccc(-c2nc3[nH]ccc(=O)c3nc2-c2ccc3ncccc3c2)[nH]1] |
| 1.491 | 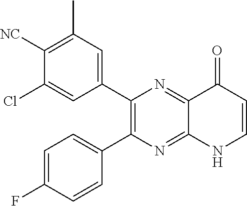 [SMILES: Cc1cc(-c2nc3c(=O)cc[nH]c3nc2-c2ccc(F)cc2)cc(Cl)c1C#N] |
| 1.492 | 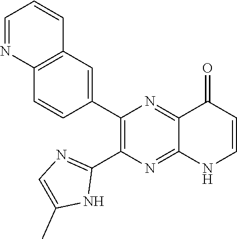 [SMILES: Cc1cnc(-c2nc3[nH]ccc(=O)c3nc2-c2ccc3ncccc3c2)[nH]1] |
| 1.493 | 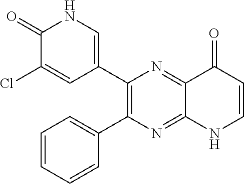 [SMILES: O=c1[nH]cc(-c2nc3c(=O)cc[nH]c3nc2-c2ccccc2)cc1Cl] |
| 1.494 | 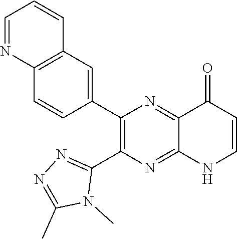 [SMILES: Cc1nnc(-c2nc3[nH]ccc(=O)c3nc2-c2ccc3ncccc3c2)n1C] |
| 1.495 | 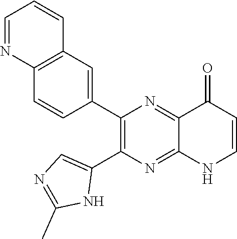 [SMILES: Cc1ncc(-c2nc3[nH]ccc(=O)c3nc2-c2ccc3ncccc3c2)[nH]1] |
| 1.496 | 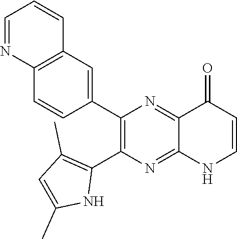 [SMILES: Cc1cc(C)c(-c2nc3[nH]ccc(=O)c3nc2-c2ccc3ncccc3c2)[nH]1] |
| 1.497 | 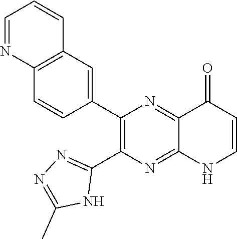 [SMILES: Cc1nnc(-c2nc3[nH]ccc(=O)c3nc2-c2ccc3ncccc3c2)[nH]1] |
| 1.498 | 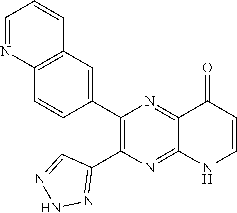 [SMILES: O=c1cc[nH]c2nc(-c3cn[nH]n3)c(-c3ccc4ncccc4c3)nc12] |
| 1.499 | 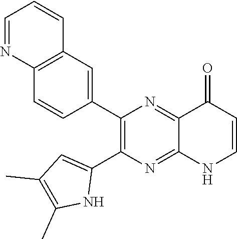 [SMILES: Cc1cc(-c2nc3[nH]ccc(=O)c3nc2-c2ccc3ncccc3c2)[nH]c1C] |
| 1.500 | 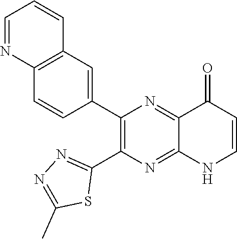 [SMILES: Cc1nnc(-c2nc3[nH]ccc(=O)c3nc2-c2ccc3ncccc3c2)s1] |
| 1.501 | 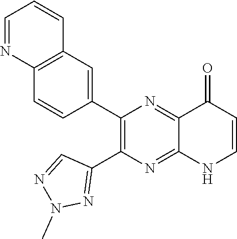 [SMILES: Cn1ncc(-c2nc3[nH]ccc(=O)c3nc2-c2ccc3ncccc3c2)n1] |
| 1.502 | 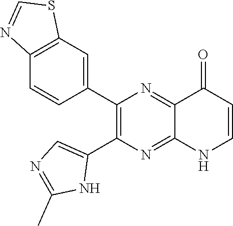 [SMILES: Cc1ncc(-c2nc3[nH]ccc(=O)c3nc2-c2ccc3ncsc3c2)[nH]1] |
| 1.503 | 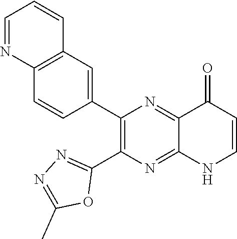 [SMILES: Cc1nnc(-c2nc3[nH]ccc(=O)c3nc2-c2ccc3ncccc3c2)o1] |
| 1.504 | 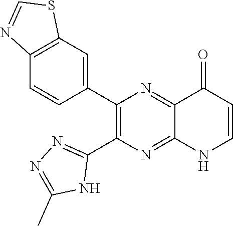 [SMILES: Cc1nnc(-c2nc3[nH]ccc(=O)c3nc2-c2ccc3ncsc3c2)[nH]1] |
| 1.505 | 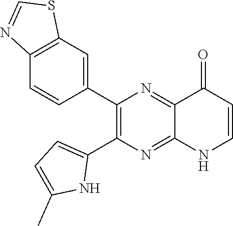 [SMILES: Cc1ccc(-c2nc3[nH]ccc(=O)c3nc2-c2ccc3ncsc3c2)[nH]1] |
| 1.506 | 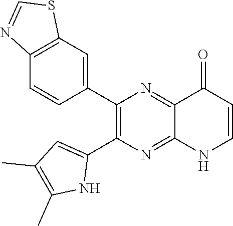 [SMILES: Cc1cc(-c2nc3[nH]ccc(=O)c3nc2-c2ccc3ncsc3c2)[nH]c1C] |
| 1.507 | 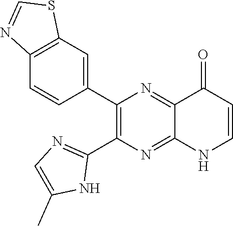 [SMILES: Cc1cnc(-c2nc3[nH]ccc(=O)c3nc2-c2ccc3ncsc3c2)[nH]1] |
| 1.508 | 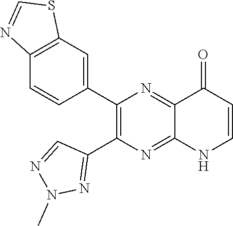 [SMILES: Cn1ncc(-c2nc3[nH]ccc(=O)c3nc2-c2ccc3ncsc3c2)n1] |
| 1.509 | 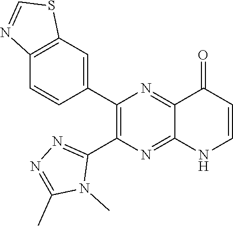 [SMILES: Cc1nnc(-c2nc3[nH]ccc(=O)c3nc2-c2ccc3ncsc3c2)n1C] |
| 1.510 | 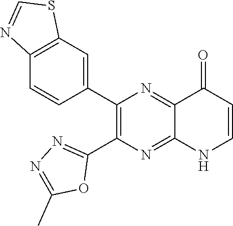 [SMILES: Cc1nnc(-c2nc3[nH]ccc(=O)c3nc2-c2ccc3ncsc3c2)o1] |
| 1.511 | 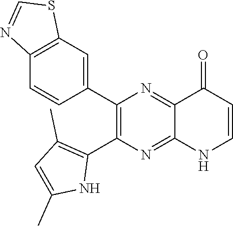 [SMILES: Cc1cc(C)c(-c2nc3[nH]ccc(=O)c3nc2-c2ccc3ncsc3c2)[nH]1] |
| 1.512 | 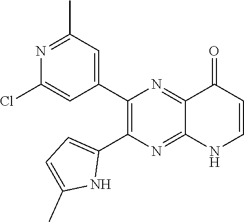 [SMILES: Cc1cc(-c2nc3c(=O)cc[nH]c3nc2-c2ccc(C)[nH]2)cc(Cl)n1] |
| 1.513 | 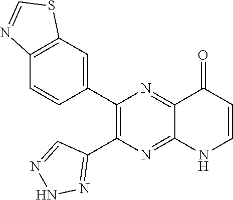 [SMILES: O=c1cc[nH]c2nc(-c3cn[nH]n3)c(-c3ccc4ncsc4c3)nc12] |
| 1.514 | 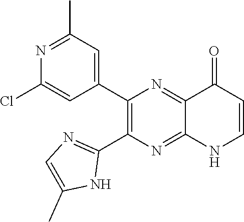 [SMILES: Cc1cc(-c2nc3c(=O)cc[nH]c3nc2-c2ncc(C)[nH]2)cc(Cl)n1] |
| 1.515 | 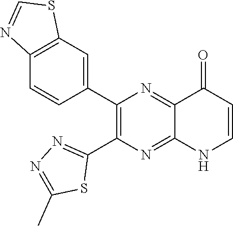 [SMILES: Cc1nnc(-c2nc3[nH]ccc(=O)c3nc2-c2ccc3ncsc3c2)s1] |
| 1.516 | 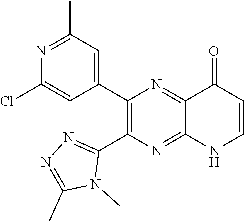 [SMILES: Cc1cc(-c2nc3c(=O)cc[nH]c3nc2-c2nnc(C)n2C)cc(Cl)n1] |
| 1.517 | 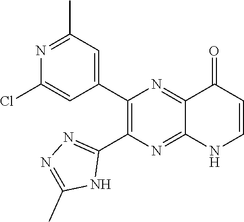 [SMILES: Cc1cc(-c2nc3c(=O)cc[nH]c3nc2-c2nnc(C)[nH]2)cc(Cl)n1] |
| 1.518 | 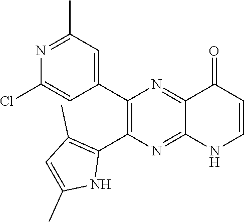 [SMILES: Cc1cc(-c2nc3c(=O)cc[nH]c3nc2-c2[nH]c(C)cc2C)cc(Cl)n1] |
| 1.519 | 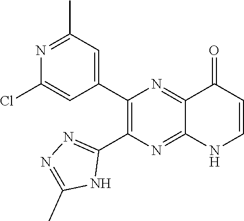 [SMILES: Cc1cc(-c2nc3c(=O)cc[nH]c3nc2-c2nnc(C)[nH]2)cc(Cl)n1] |
| 1.520 | 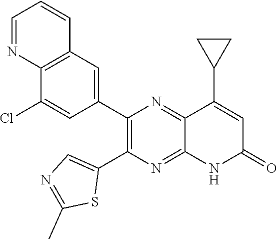 [SMILES: Cc1ncc(-c2nc3[nH]c(=O)cc(C4CC4)c3nc2-c2cc(Cl)c3ncccc3c2)s1] |
| 1.521 | 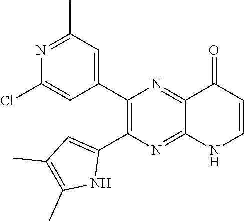 [SMILES: Cc1cc(-c2nc3c(=O)cc[nH]c3nc2-c2cc(C)c(C)[nH]2)cc(Cl)n1] |
| 1.522 | 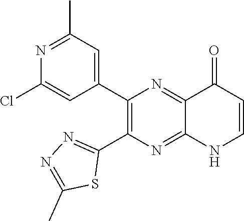 [SMILES: Cc1cc(-c2nc3c(=O)cc[nH]c3nc2-c2nnc(C)s2)cc(Cl)n1] |
| 1.523 | 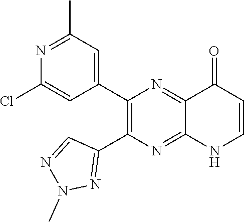 [SMILES: Cc1cc(-c2nc3c(=O)cc[nH]c3nc2-c2cnn(C)n2)cc(Cl)n1] |
| 1.524 | 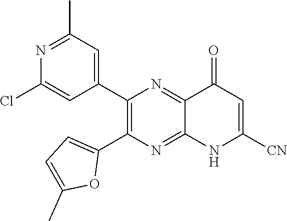 [SMILES: Cc1cc(-c2nc3c(=O)cc(C#N)[nH]c3nc2-c2ccc(C)o2)cc(Cl)n1] |
| 1.525 | 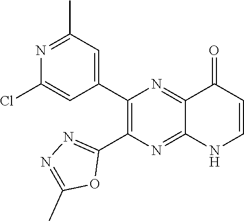 [SMILES: Cc1cc(-c2nc3c(=O)cc[nH]c3nc2-c2nnc(C)o2)cc(Cl)n1] |
| 1.526 | 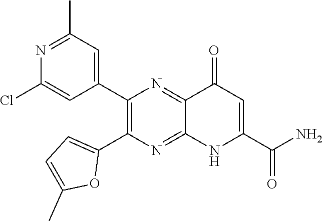 [SMILES: Cc1cc(-c2nc3c(=O)cc(C(N)=O)[nH]c3nc2-c2ccc(C)o2)cc(Cl)n1] |
| 1.527 | 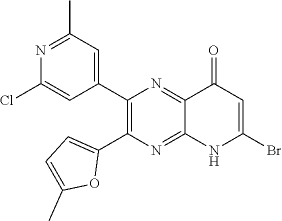 [SMILES: Cc1cc(-c2nc3c(=O)cc(Br)[nH]c3nc2-c2ccc(C)o2)cc(Cl)n1] |
| 1.528 | 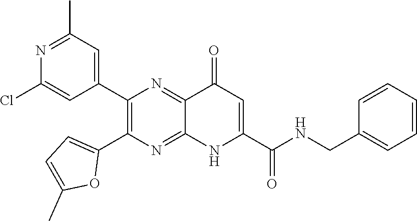 [SMILES: Cc1cc(-c2nc3c(=O)cc(C(=O)NCc4ccccc4)[nH]c3nc2-c2ccc(C)o2)cc(Cl)n1] |
| 1.529 | 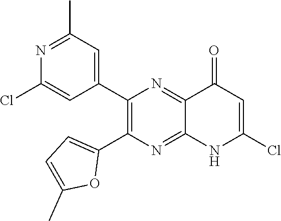 [SMILES: Cc1cc(-c2nc3c(=O)cc(Cl)[nH]c3nc2-c2ccc(C)o2)cc(Cl)n1] |
| 1.530 | 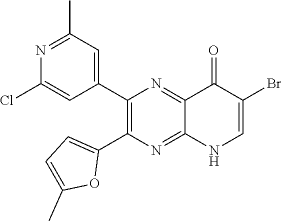 [SMILES: Cc1cc(-c2nc3c(=O)c(Br)c[nH]c3nc2-c2ccc(C)o2)cc(Cl)n1] |
| 1.531 | 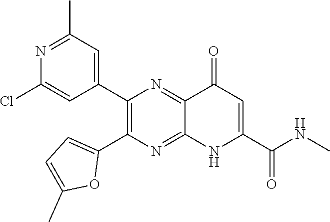 [SMILES: CNC(=O)c1cc(=O)c2nc(-c3cc(C)nc(Cl)c3)c(-c3ccc(C)o3)nc2[nH]1] |
| 1.532 | 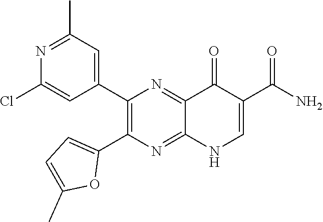 [SMILES: Cc1cc(-c2nc3c(=O)c(C(N)=O)c[nH]c3nc2-c2ccc(C)o2)cc(Cl)n1] |
| 1.533 | 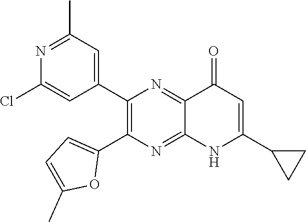 [SMILES: Cc1cc(-c2nc3c(=O)cc(C4CC4)[nH]c3nc2-c2ccc(C)o2)cc(Cl)n1] |
| 1.534 | 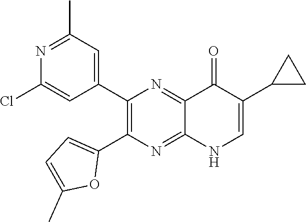 [SMILES: Cc1cc(-c2nc3c(=O)c(C4CC4)c[nH]c3nc2-c2ccc(C)o2)cc(Cl)n1] |
| 1.535 | 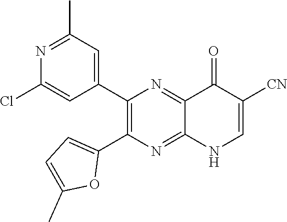 [SMILES: Cc1cc(-c2nc3c(=O)c(C#N)c[nH]c3nc2-c2ccc(C)o2)cc(Cl)n1] |
| 1.536 | 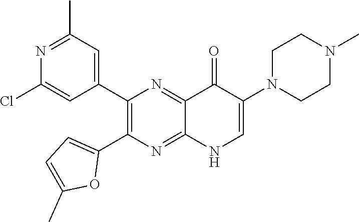 [SMILES: Cc1cc(-c2nc3c(=O)c(N4CCN(C)CC4)c[nH]c3nc2-c2ccc(C)o2)cc(Cl)n1] |
| 1.537 | 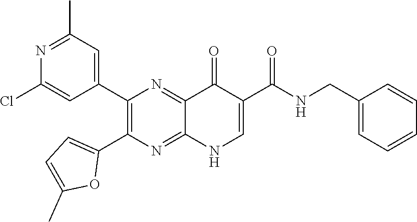 [SMILES: Cc1cc(-c2nc3c(=O)c(C(=O)NCc4ccccc4)c[nH]c3nc2-c2ccc(C)o2)cc(Cl)n1] |
| 1.538 | 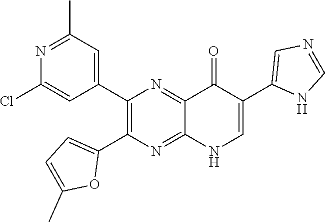 [SMILES: Cc1cc(-c2nc3c(=O)c(-c4cnc[nH]4)c[nH]c3nc2-c2ccc(C)o2)cc(Cl)n1] |
| 1.539 | 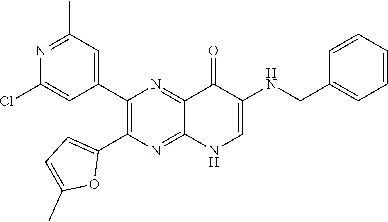 [SMILES: Cc1cc(-c2nc3c(=O)c(NCc4ccccc4)c[nH]c3nc2-c2ccc(C)o2)cc(Cl)n1] |
| 1.540 | 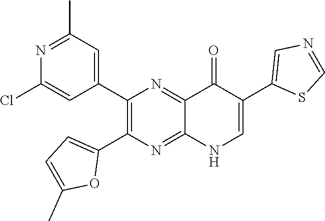 [SMILES: Cc1cc(-c2nc3c(=O)c(-c4cncs4)c[nH]c3nc2-c2ccc(C)o2)cc(Cl)n1] |
| 1.541 | 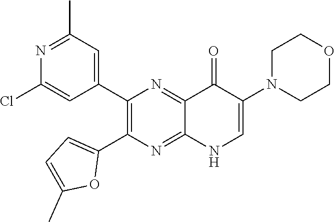 [SMILES: Cc1cc(-c2nc3c(=O)c(N4CCOCC4)c[nH]c3nc2-c2ccc(C)o2)cc(Cl)n1] |
| 1.542 | 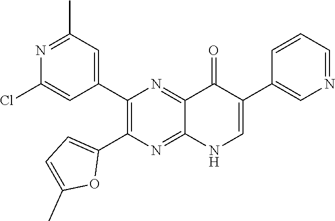 [SMILES: Cc1cc(-c2nc3c(=O)c(-c4cccnc4)c[nH]c3nc2-c2ccc(C)o2)cc(Cl)n1] |
| 1.543 | 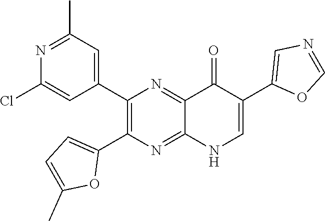 [SMILES: Cc1cc(-c2nc3c(=O)c(-c4cnco4)c[nH]c3nc2-c2ccc(C)o2)cc(Cl)n1] |
| 1.544 | 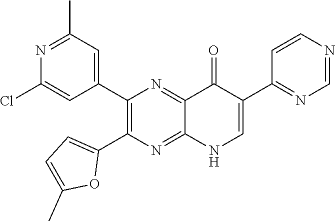 [SMILES: Cc1cc(-c2nc3c(=O)c(-c4ccncn4)c[nH]c3nc2-c2ccc(C)o2)cc(Cl)n1] |
| 1.545 | 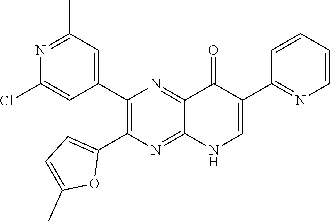 [SMILES: Cc1cc(-c2nc3c(=O)c(-c4ccccn4)c[nH]c3nc2-c2ccc(C)o2)cc(Cl)n1] |
| 1.546 | 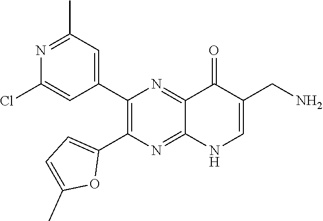 [SMILES: Cc1cc(-c2nc3c(=O)c(CN)c[nH]c3nc2-c2ccc(C)o2)cc(Cl)n1] |
| 1.547 | 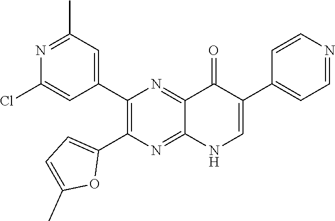 [SMILES: Cc1cc(-c2nc3c(=O)c(-c4ccncc4)c[nH]c3nc2-c2ccc(C)o2)cc(Cl)n1] |
| 1.548 | 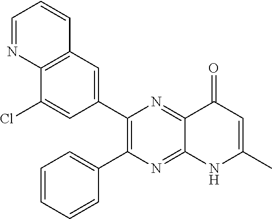 [SMILES: Cc1cc(=O)c2nc(-c3cc(Cl)c4ncccc4c3)c(-c3ccccc3)nc2[nH]1] |
| 1.549 | 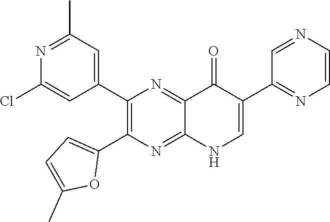 [SMILES: Cc1cc(-c2nc3c(=O)c(-c4cnccn4)c[nH]c3nc2-c2ccc(C)o2)cc(Cl)n1] |
| 1.550 | 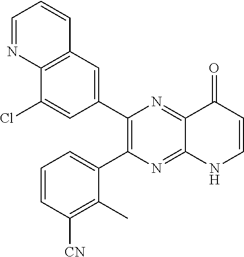 [SMILES: Cc1c(C#N)cccc1-c1nc2[nH]ccc(=O)c2nc1-c1cc(Cl)c2ncccc2c1] |
| 1.551 | 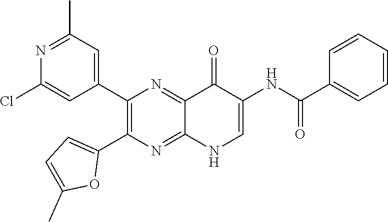 [SMILES: Cc1cc(-c2nc3c(=O)c(NC(=O)c4ccccc4)c[nH]c3nc2-c2ccc(C)o2)cc(Cl)n1] |
| 1.552 | 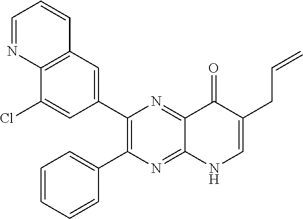 [SMILES: C=CCc1c[nH]c2nc(-c3ccccc3)c(-c3cc(Cl)c4ncccc4c3)nc2c1=O] |
| 1.553 | 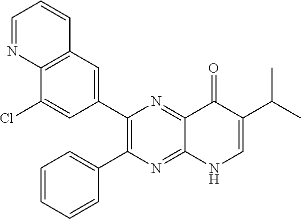 [SMILES: CC(C)c1c[nH]c2nc(-c3ccccc3)c(-c3cc(Cl)c4ncccc4c3)nc2c1=O] |
| 1.554 | 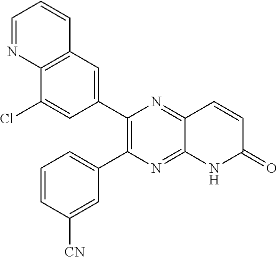 [SMILES: N#Cc1cccc(-c2nc3[nH]c(=O)ccc3nc2-c2cc(Cl)c3ncccc3c2)c1] |
| 1.555 | 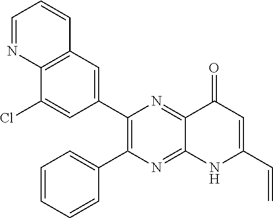 [SMILES: C=Cc1cc(=O)c2nc(-c3cc(Cl)c4ncccc4c3)c(-c3ccccc3)nc2[nH]1] |
| 1.556 | 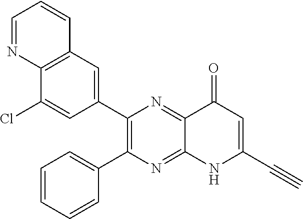 [SMILES: C#Cc1cc(=O)c2nc(-c3cc(Cl)c4ncccc4c3)c(-c3ccccc3)nc2[nH]1] |
| 1.557 | 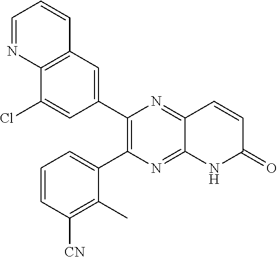 [SMILES: Cc1c(C#N)cccc1-c1nc2[nH]c(=O)ccc2nc1-c1cc(Cl)c2ncccc2c1] |
| 1.558 | 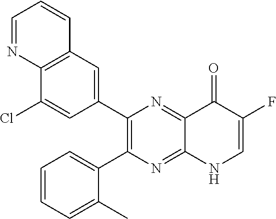 [SMILES: Cc1ccccc1-c1nc2[nH]cc(F)c(=O)c2nc1-c1cc(Cl)c2ncccc2c1] |
| 1.559 | 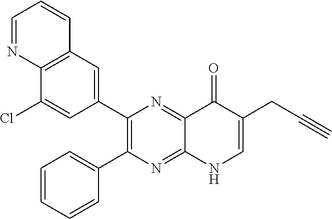 [SMILES: C#CCc1c[nH]c2nc(-c3ccccc3)c(-c3cc(Cl)c4ncccc4c3)nc2c1=O] |
| 1.560 | 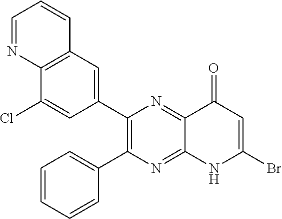 [SMILES: O=c1cc(Br)[nH]c2nc(-c3ccccc3)c(-c3cc(Cl)c4ncccc4c3)nc12] |
| 1.561 | 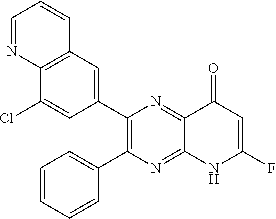 [SMILES: O=c1cc(F)[nH]c2nc(-c3ccccc3)c(-c3cc(Cl)c4ncccc4c3)nc12] |
| 1.562 | 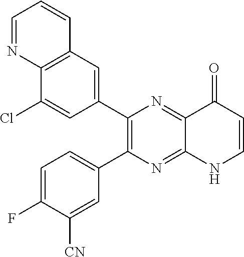 [SMILES: N#Cc1cc(-c2nc3[nH]ccc(=O)c3nc2-c2cc(Cl)c3ncccc3c2)ccc1F] |
| 1.563 | 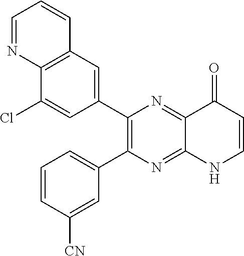 [SMILES: N#Cc1cccc(-c2nc3[nH]ccc(=O)c3nc2-c2cc(Cl)c3ncccc3c2)c1] |
| 1.564 | 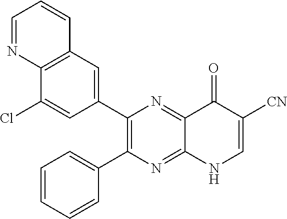 [SMILES: N#Cc1c[nH]c2nc(-c3ccccc3)c(-c3cc(Cl)c4ncccc4c3)nc2c1=O] |
| 1.565 | 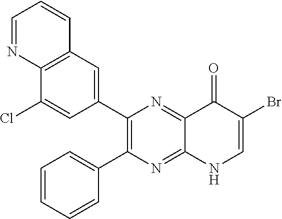 [SMILES: O=c1c(Br)c[nH]c2nc(-c3ccccc3)c(-c3cc(Cl)c4ncccc4c3)nc12] |
| 1.566 | 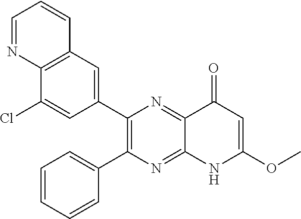 [SMILES: COc1cc(=O)c2nc(-c3cc(Cl)c4ncccc4c3)c(-c3ccccc3)nc2[nH]1] |
| 1.567 | 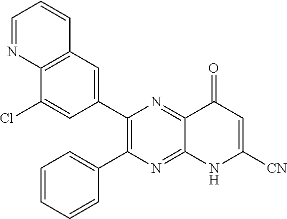 [SMILES: N#Cc1cc(=O)c2nc(-c3cc(Cl)c4ncccc4c3)c(-c3ccccc3)nc2[nH]1] |
| 1.568 | 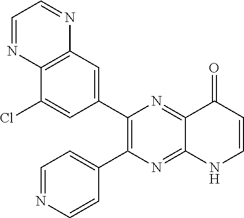 [SMILES: O=c1cc[nH]c2nc(-c3ccncc3)c(-c3cc(Cl)c4nccnc4c3)nc12] |
| 1.569 | 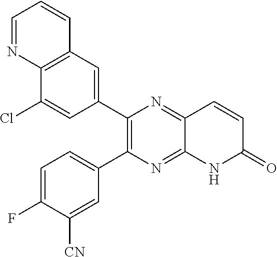 [SMILES: N#Cc1cc(-c2nc3[nH]c(=O)ccc3nc2-c2cc(Cl)c3ncccc3c2)ccc1F] |
| 1.570 | 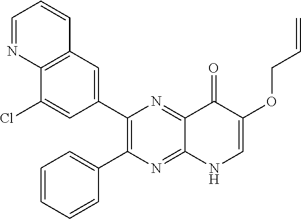 [SMILES: C=CCOc1c[nH]c2nc(-c3ccccc3)c(-c3cc(Cl)c4ncccc4c3)nc2c1=O] |
| 1.571 | 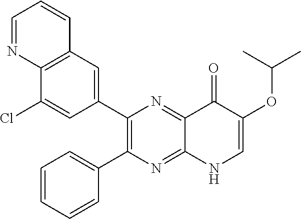 [SMILES: CC(C)Oc1c[nH]c2nc(-c3ccccc3)c(-c3cc(Cl)c4ncccc4c3)nc2c1=O] |
| 1.572 | 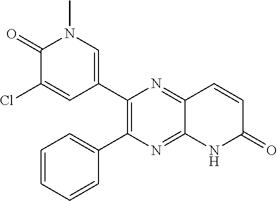 [SMILES: Cn1cc(-c2nc3ccc(=O)[nH]c3nc2-c2ccccc2)cc(Cl)c1=O] |
| 1.573 | 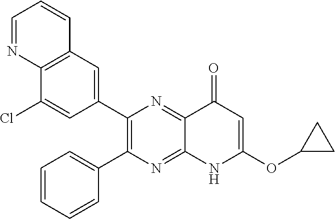 [SMILES: O=c1cc(OC2CC2)[nH]c2nc(-c3ccccc3)c(-c3cc(Cl)c4ncccc4c3)nc12] |
| 1.574 | 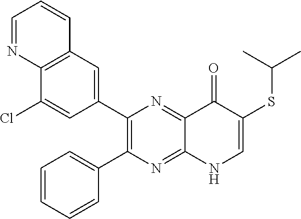 [SMILES: CC(C)Sc1c[nH]c2nc(-c3ccccc3)c(-c3cc(Cl)c4ncccc4c3)nc2c1=O] |
| 1.575 | 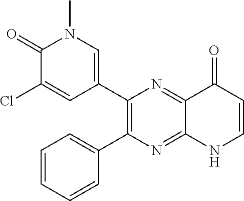 [SMILES: Cn1cc(-c2nc3c(=O)cc[nH]c3nc2-c2ccccc2)cc(Cl)c1=O] |
| 1.576 | 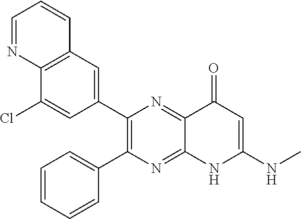 [SMILES: CNc1cc(=O)c2nc(-c3cc(Cl)c4ncccc4c3)c(-c3ccccc3)nc2[nH]1] |
| 1.577 | 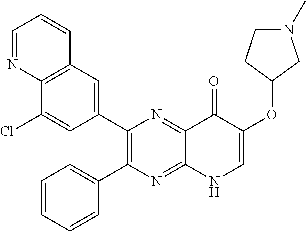 [SMILES: CN1CCC(Oc2c[nH]c3nc(-c4ccccc4)c(-c4cc(Cl)c5ncccc5c4)nc3c2=O)C1] |
| 1.578 | 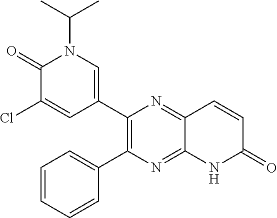 [SMILES: CC(C)n1cc(-c2nc3ccc(=O)[nH]c3nc2-c2ccccc2)cc(Cl)c1=O] |
| 1.579 | 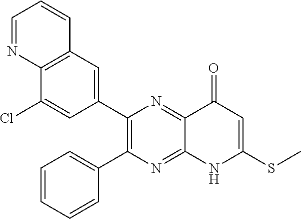 [SMILES: CSc1cc(=O)c2nc(-c3cc(Cl)c4ncccc4c3)c(-c3ccccc3)nc2[nH]1] |
| 1.580 | 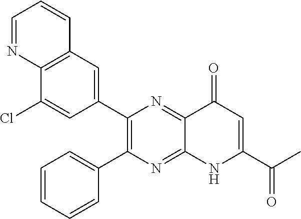 [SMILES: CC(=O)c1cc(=O)c2nc(-c3cc(Cl)c4ncccc4c3)c(-c3ccccc3)nc2[nH]1] |
| 1.581 | 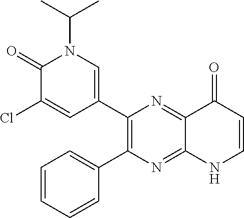 [SMILES: CC(C)n1cc(-c2nc3c(=O)cc[nH]c3nc2-c2ccccc2)cc(Cl)c1=O] |
| 1.582 | 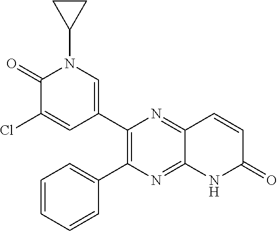 [SMILES: O=c1ccc2nc(-c3cc(Cl)c(=O)n(C4CC4)c3)c(-c3ccccc3)nc2[nH]1] |
| 1.583 | 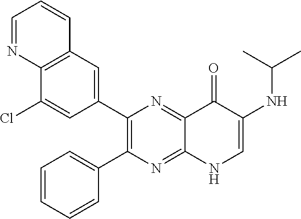 [SMILES: CC(C)Nc1c[nH]c2nc(-c3ccccc3)c(-c3cc(Cl)c4ncccc4c3)nc2c1=O] |
| 1.584 | 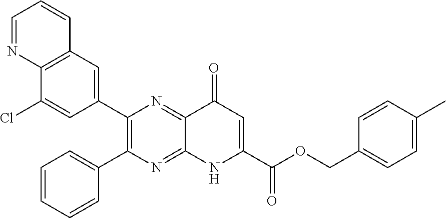 [SMILES: Cc1ccc(COC(=O)c2cc(=O)c3nc(-c4cc(Cl)c5ncccc5c4)c(-c4ccccc4)nc3[nH]2)cc1] |
| 1.585 | 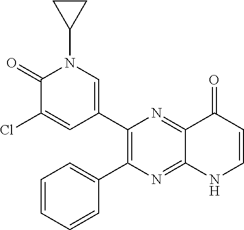 [SMILES: O=c1cc[nH]c2nc(-c3ccccc3)c(-c3cc(Cl)c(=O)n(C4CC4)c3)nc12] |
| 1.586 | 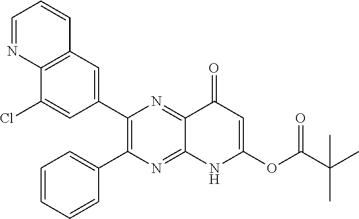 [SMILES: CC(C)(C)C(=O)Oc1cc(=O)c2nc(-c3cc(Cl)c4ncccc4c3)c(-c3ccccc3)nc2[nH]1] |
| 1.587 | 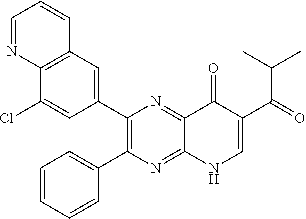 [SMILES: CC(C)C(=O)c1c[nH]c2nc(-c3ccccc3)c(-c3cc(Cl)c4ncccc4c3)nc2c1=O] |
| 1.588 | 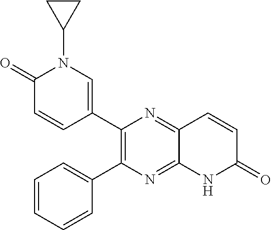 [SMILES: O=c1ccc2nc(-c3ccc(=O)n(C4CC4)c3)c(-c3ccccc3)nc2[nH]1] |
| 1.589 | 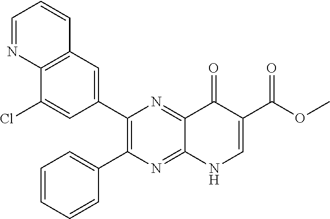 [SMILES: COC(=O)c1c[nH]c2nc(-c3ccccc3)c(-c3cc(Cl)c4ncccc4c3)nc2c1=O] |
| 1.590 | 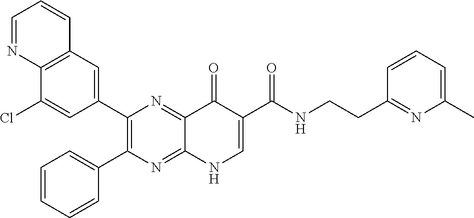 [SMILES: Cc1cccc(CCNC(=O)c2c[nH]c3nc(-c4ccccc4)c(-c4cc(Cl)c5ncccc5c4)nc3c2=O)n1] |
| 1.591 | 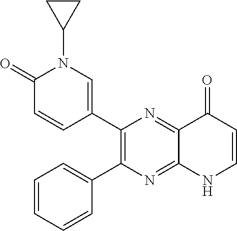 [SMILES: O=c1cc[nH]c2nc(-c3ccccc3)c(-c3ccc(=O)n(C4CC4)c3)nc12] |
| 1.592 | 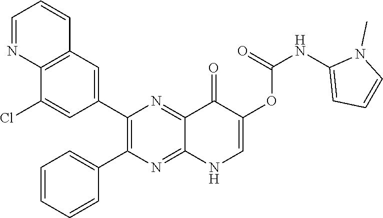 [SMILES: Cn1cccc1NC(=O)Oc1c[nH]c2nc(-c3ccccc3)c(-c3cc(Cl)c4ncccc4c3)nc2c1=O] |
| 1.593 | 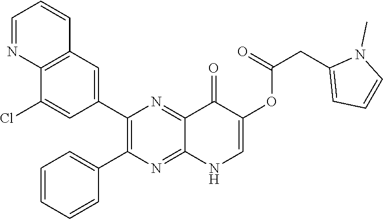 [SMILES: Cn1cccc1CC(=O)Oc1c[nH]c2nc(-c3ccccc3)c(-c3cc(Cl)c4ncccc4c3)nc2c1=O] |
| 1.594 | 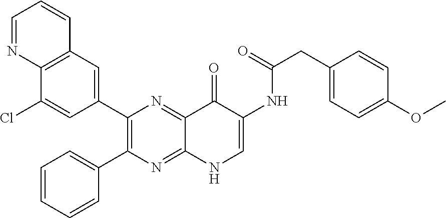 [SMILES: COc1ccc(CC(=O)Nc2c[nH]c3nc(-c4ccccc4)c(-c4cc(Cl)c5ncccc5c4)nc3c2=O)cc1] |
| 1.595 | 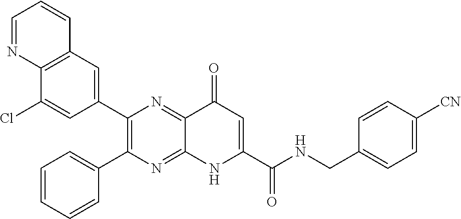 [SMILES: N#Cc1ccc(CNC(=O)c2cc(=O)c3nc(-c4cc(Cl)c5ncccc5c4)c(-c4ccccc4)nc3[nH]2)cc1] |
| 1.596 | 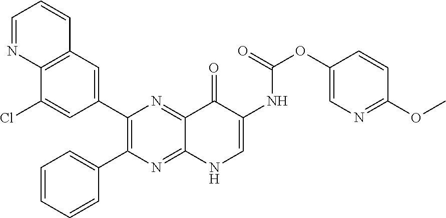 [SMILES: COc1ccc(OC(=O)Nc2c[nH]c3nc(-c4ccccc4)c(-c4cc(Cl)c5ncccc5c4)nc3c2=O)cn1] |
| 1.597 | 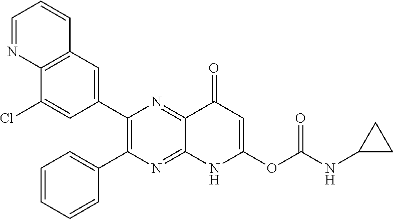 [SMILES: O=C(NC1CC1)Oc1cc(=O)c2nc(-c3cc(Cl)c4ncccc4c3)c(-c3ccccc3)nc2[nH]1] |
| 1.598 | 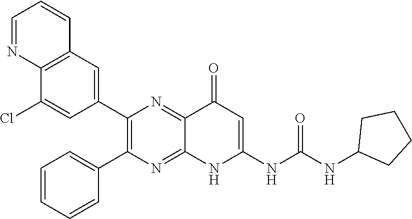 [SMILES: O=C(Nc1cc(=O)c2nc(-c3cc(Cl)c4ncccc4c3)c(-c3ccccc3)nc2[nH]1)NC1CCCC1] |
| 1.599 | 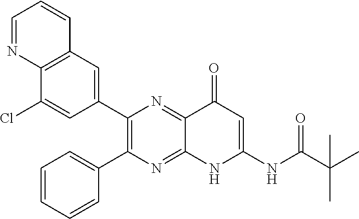 [SMILES: CC(C)(C)C(=O)Nc1cc(=O)c2nc(-c3cc(Cl)c4ncccc4c3)c(-c3ccccc3)nc2[nH]1] |
| 1.600 | 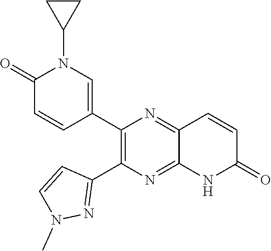 [SMILES: Cn1ccc(-c2nc3[nH]c(=O)ccc3nc2-c2ccc(=O)n(C3CC3)c2)n1] |
| 1.601 | 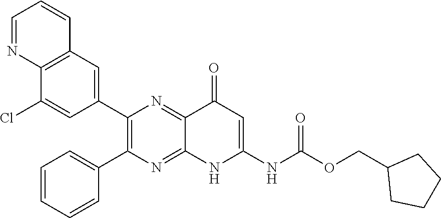 [SMILES: O=C(Nc1cc(=O)c2nc(-c3cc(Cl)c4ncccc4c3)c(-c3ccccc3)nc2[nH]1)OCC1CCCC1] |
| 1.602 | 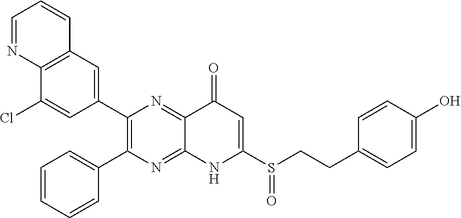 [SMILES: O=c1cc(S(=O)CCc2ccc(O)cc2)[nH]c2nc(-c3ccccc3)c(-c3cc(Cl)c4ncccc4c3)nc12] |
| 1.603 | 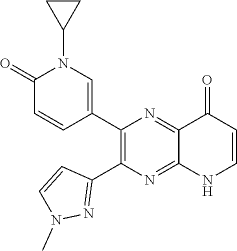 [SMILES: Cn1ccc(-c2nc3[nH]ccc(=O)c3nc2-c2ccc(=O)n(C3CC3)c2)n1] |
| 1.604 | 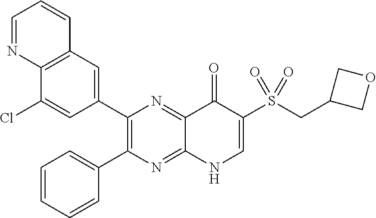 [SMILES: O=c1c(S(=O)(=O)CC2COC2)c[nH]c2nc(-c3ccccc3)c(-c3cc(Cl)c4ncccc4c3)nc12] |
| 1.605 | 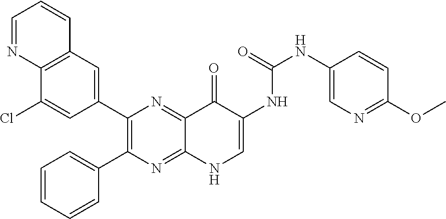 [SMILES: COc1ccc(NC(=O)Nc2c[nH]c3nc(-c4ccccc4)c(-c4cc(Cl)c5ncccc5c4)nc3c2=O)cn1] |
| 1.606 | 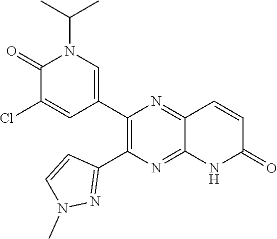 [SMILES: CC(C)n1cc(-c2nc3ccc(=O)[nH]c3nc2-c2ccn(C)n2)cc(Cl)c1=O] |
| 1.607 | 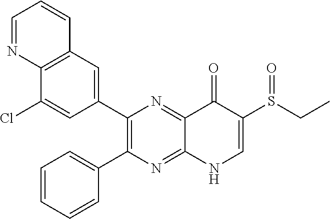 [SMILES: CCS(=O)c1c[nH]c2nc(-c3ccccc3)c(-c3cc(Cl)c4ncccc4c3)nc2c1=O] |
| 1.608 | 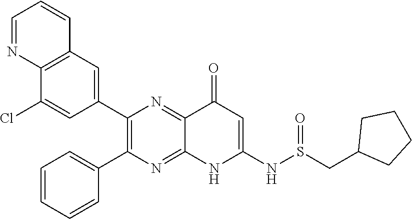 [SMILES: O=c1cc(NS(=O)CC2CCCC2)[nH]c2nc(-c3ccccc3)c(-c3cc(Cl)c4ncccc4c3)nc12] |
| 1.609 | 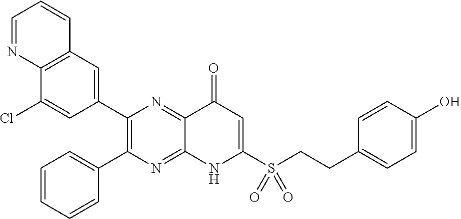 [SMILES: O=c1cc(S(=O)(=O)CCc2ccc(O)cc2)[nH]c2nc(-c3ccccc3)c(-c3cc(Cl)c4ncccc4c3)nc12] |
| 1.610 | 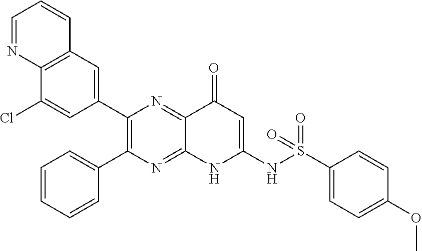 [SMILES: COc1ccc(S(=O)(=O)Nc2cc(=O)c3nc(-c4cc(Cl)c5ncccc5c4)c(-c4ccccc4)nc3[nH]2)cc1] |
| 1.611 | 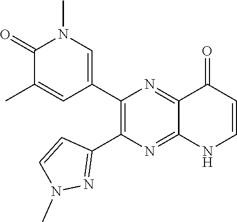 [SMILES: Cc1cc(-c2nc3c(=O)cc[nH]c3nc2-c2ccn(C)n2)cn(C)c1=O] |
| 1.612 | 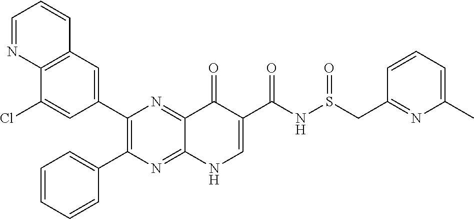 [SMILES: Cc1cccc(CS(=O)NC(=O)c2c[nH]c3nc(-c4ccccc4)c(-c4cc(Cl)c5ncccc5c4)nc3c2=O)n1] |
| 1.613 | 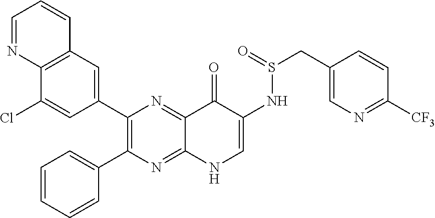 [SMILES: O=c1c(NS(=O)Cc2ccc(C(F)(F)F)nc2)c[nH]c2nc(-c3ccccc3)c(-c3cc(Cl)c4ncccc4c3)nc12] |
| 1.614 | 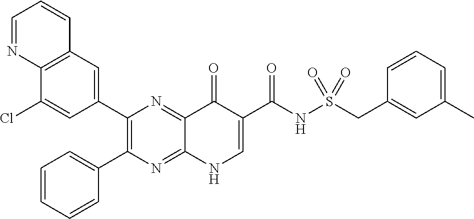 [SMILES: Cc1cccc(CS(=O)(=O)NC(=O)c2c[nH]c3nc(-c4ccccc4)c(-c4cc(Cl)c5ncccc5c4)nc3c2=O)c1] |
| 1.615 | 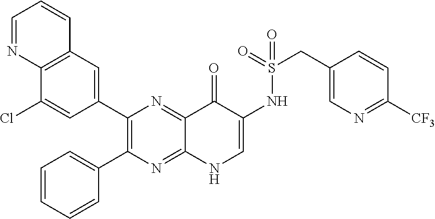 [SMILES: O=c1c(NS(=O)(=O)Cc2ccc(C(F)(F)F)nc2)c[nH]c2nc(-c3ccccc3)c(-c3cc(Cl)c4ncccc4c3)nc12] |
| 1.616 | 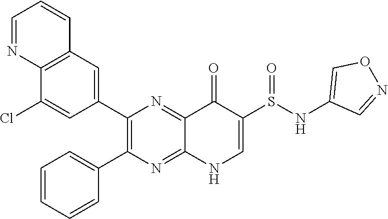 [SMILES: O=c1c(S(=O)Nc2cnoc2)c[nH]c2nc(-c3ccccc3)c(-c3cc(Cl)c4ncccc4c3)nc12] |
| 1.617 | 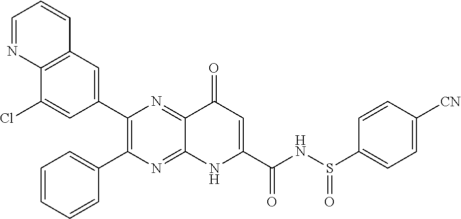 [SMILES: N#Cc1ccc(S(=O)NC(=O)c2cc(=O)c3nc(-c4cc(Cl)c5ncccc5c4)c(-c4ccccc4)nc3[nH]2)cc1] |
| 1.618 | 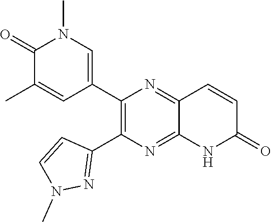 [SMILES: Cc1cc(-c2nc3ccc(=O)[nH]c3nc2-c2ccn(C)n2)cn(C)c1=O] |
| 1.619 | 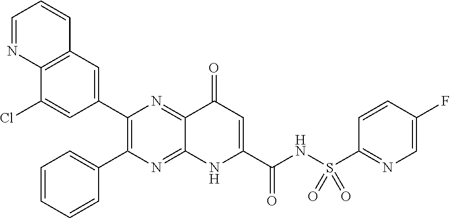 [SMILES: O=C(NS(=O)(=O)c1ccc(F)cn1)c1cc(=O)c2nc(-c3cc(Cl)c4ncccc4c3)c(-c3ccccc3)nc2[nH]1] |
| 1.620 | 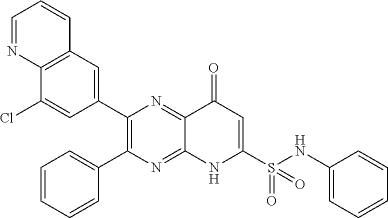 [SMILES: O=c1cc(S(=O)(=O)Nc2ccccc2)[nH]c2nc(-c3ccccc3)c(-c3cc(Cl)c4ncccc4c3)nc12] |
| 1.621 | 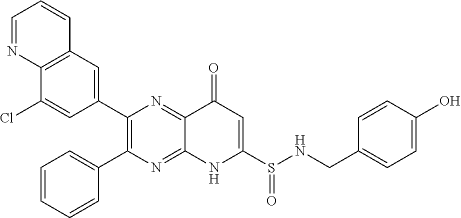 [SMILES: O=c1cc(S(=O)NCc2ccc(O)cc2)[nH]c2nc(-c3ccccc3)c(-c3cc(Cl)c4ncccc4c3)nc12] |
| 1.622 | 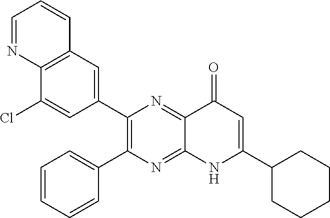 [SMILES: O=c1cc(C2CCCCC2)[nH]c2nc(-c3ccccc3)c(-c3cc(Cl)c4ncccc4c3)nc12] |
| 1.623 | 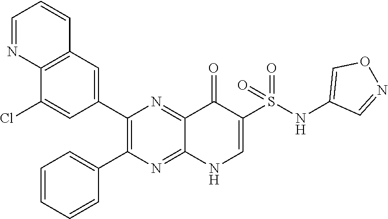 [SMILES: O=c1c(S(=O)(=O)Nc2cnoc2)c[nH]c2nc(-c3ccccc3)c(-c3cc(Cl)c4ncccc4c3)nc12] |
| 1.624 | 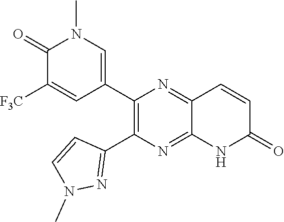 [SMILES: Cn1ccc(-c2nc3[nH]c(=O)ccc3nc2-c2cc(C(F)(F)F)c(=O)n(C)c2)n1] |
| 1.625 | 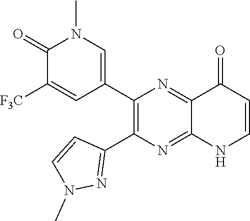 [SMILES: Cn1ccc(-c2nc3[nH]ccc(=O)c3nc2-c2cc(C(F)(F)F)c(=O)n(C)c2)n1] |
| 1.626 | 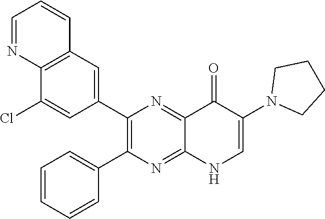 [SMILES: O=c1c(N2CCCC2)c[nH]c2nc(-c3ccccc3)c(-c3cc(Cl)c4ncccc4c3)nc12] |
| 1.627 | 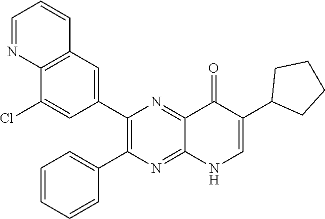 [SMILES: O=c1c(C2CCCC2)c[nH]c2nc(-c3ccccc3)c(-c3cc(Cl)c4ncccc4c3)nc12] |
| 1.628 | 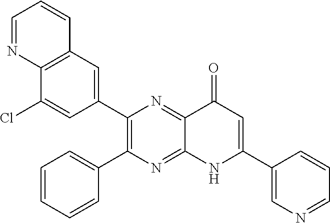 [SMILES: O=c1cc(-c2cccnc2)[nH]c2nc(-c3ccccc3)c(-c3cc(Cl)c4ncccc4c3)nc12] |
| 1.629 | 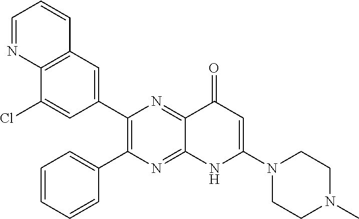 [SMILES: CN1CCN(c2cc(=O)c3nc(-c4cc(Cl)c5ncccc5c4)c(-c4ccccc4)nc3[nH]2)CC1] |
| 1.630 | 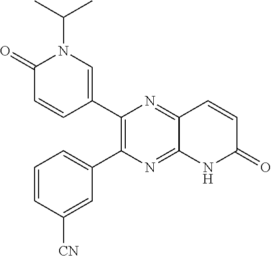 [SMILES: CC(C)n1cc(-c2nc3ccc(=O)[nH]c3nc2-c2cccc(C#N)c2)ccc1=O] |
| 1.631 | 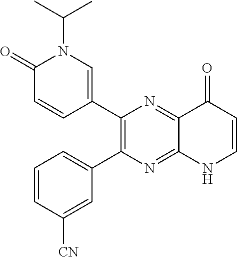 [SMILES: CC(C)n1cc(-c2nc3c(=O)cc[nH]c3nc2-c2cccc(C#N)c2)ccc1=O] |
| 1.632 | 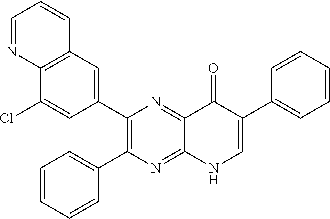 [SMILES: O=c1c(-c2ccccc2)c[nH]c2nc(-c3ccccc3)c(-c3cc(Cl)c4ncccc4c3)nc12] |
| 1.633 | 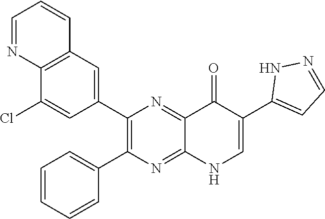 [SMILES: O=c1c(-c2ccn[nH]2)c[nH]c2nc(-c3ccccc3)c(-c3cc(Cl)c4ncccc4c3)nc12] |
| 1.634 | 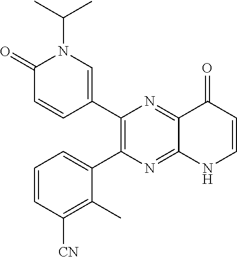 [SMILES: Cc1c(C#N)cccc1-c1nc2[nH]ccc(=O)c2nc1-c1ccc(=O)n(C(C)C)c1] |
| 1.635 | 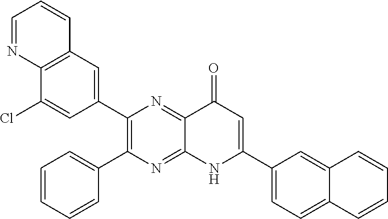 [SMILES: O=c1cc(-c2ccc3ccccc3c2)[nH]c2nc(-c3ccccc3)c(-c3cc(Cl)c4ncccc4c3)nc12] |
| 1.636 | 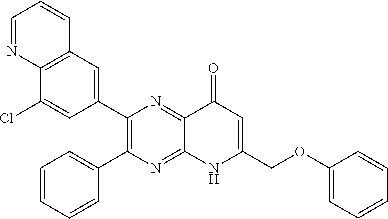 [SMILES: O=c1cc(COc2ccccc2)[nH]c2nc(-c3ccccc3)c(-c3cc(Cl)c4ncccc4c3)nc12] |
| 1.637 | 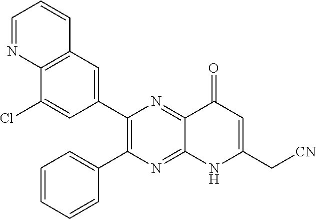 [SMILES: N#CCc1cc(=O)c2nc(-c3cc(Cl)c4ncccc4c3)c(-c3ccccc3)nc2[nH]1] |
| 1.638 | 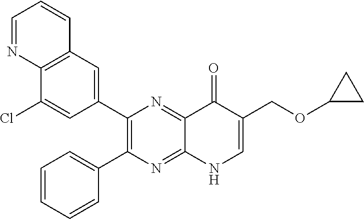 [SMILES: O=c1c(COC2CC2)c[nH]c2nc(-c3ccccc3)c(-c3cc(Cl)c4ncccc4c3)nc12] |
| 1.639 | 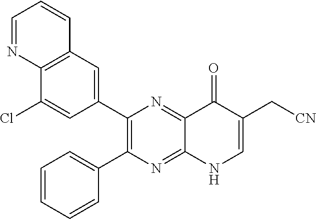 [SMILES: N#CCc1c[nH]c2nc(-c3ccccc3)c(-c3cc(Cl)c4ncccc4c3)nc2c1=O] |
| 1.640 | 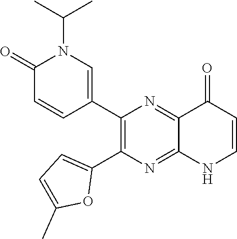 [SMILES: Cc1ccc(-c2nc3[nH]ccc(=O)c3nc2-c2ccc(=O)n(C(C)C)c2)o1] |
| 1.641 | 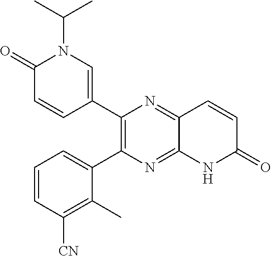 [SMILES: Cc1c(C#N)cccc1-c1nc2[nH]c(=O)ccc2nc1-c1ccc(=O)n(C(C)C)c1] |
| 1.642 | 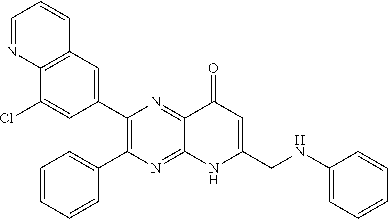 [SMILES: O=c1cc(CNc2ccccc2)[nH]c2nc(-c3ccccc3)c(-c3cc(Cl)c4ncccc4c3)nc12] |
| 1.643 | 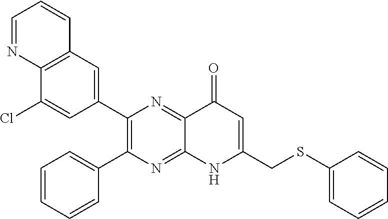 [SMILES: O=c1cc(CSc2ccccc2)[nH]c2nc(-c3ccccc3)c(-c3cc(Cl)c4ncccc4c3)nc12] |
| 1.644 | 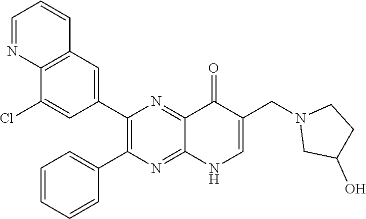 [SMILES: O=c1c(CN2CCC(O)C2)c[nH]c2nc(-c3ccccc3)c(-c3cc(Cl)c4ncccc4c3)nc12] |
| 1.645 | 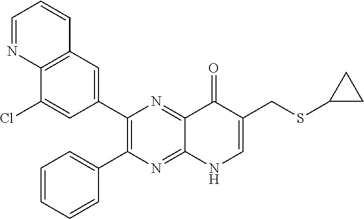 [SMILES: O=c1c(CSC2CC2)c[nH]c2nc(-c3ccccc3)c(-c3cc(Cl)c4ncccc4c3)nc12] |
| 1.646 | 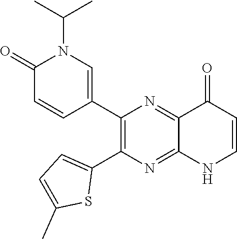 [SMILES: Cc1ccc(-c2nc3[nH]ccc(=O)c3nc2-c2ccc(=O)n(C(C)C)c2)s1] |
| 1.647 | 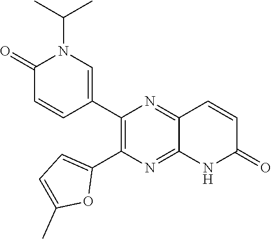 [SMILES: Cc1ccc(-c2nc3[nH]c(=O)ccc3nc2-c2ccc(=O)n(C(C)C)c2)o1] |
| 1.648 | 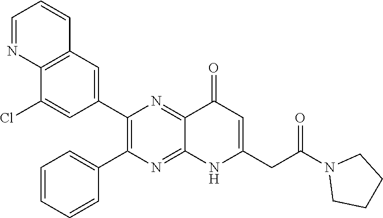 [SMILES: O=C(Cc1cc(=O)c2nc(-c3cc(Cl)c4ncccc4c3)c(-c3ccccc3)nc2[nH]1)N1CCCC1] |
| 1.649 | 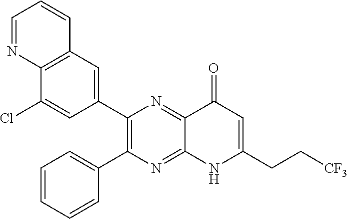 [SMILES: O=c1cc(CCC(F)(F)F)[nH]c2nc(-c3ccccc3)c(-c3cc(Cl)c4ncccc4c3)nc12] |
| 1.650 | 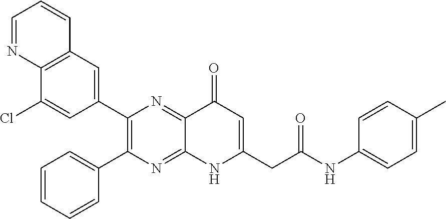 [SMILES: Cc1ccc(NC(=O)Cc2cc(=O)c3nc(-c4cc(Cl)c5ncccc5c4)c(-c4ccccc4)nc3[nH]2)cc1] |
| 1.651 | 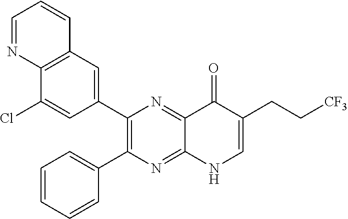 [SMILES: O=c1c(CCC(F)(F)F)c[nH]c2nc(-c3ccccc3)c(-c3cc(Cl)c4ncccc4c3)nc12] |
| 1.652 | 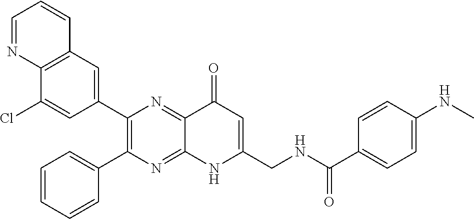 [SMILES: CNc1ccc(C(=O)NCc2cc(=O)c3nc(-c4cc(Cl)c5ncccc5c4)c(-c4ccccc4)nc3[nH]2)cc1] |
| 1.653 | 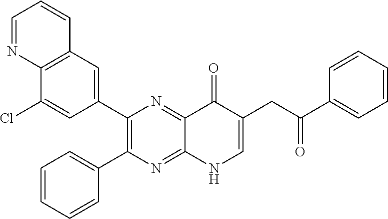 [SMILES: O=C(Cc1c[nH]c2nc(-c3ccccc3)c(-c3cc(Cl)c4ncccc4c3)nc2c1=O)c1ccccc1] |
| 1.654 | 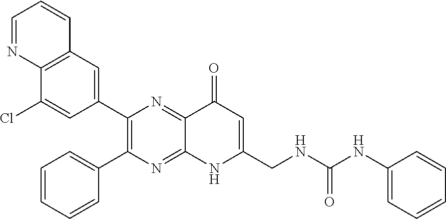 [SMILES: O=C(NCc1cc(=O)c2nc(-c3cc(Cl)c4ncccc4c3)c(-c3ccccc3)nc2[nH]1)Nc1ccccc1] |
| 1.655 | 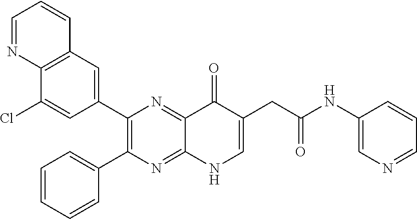 [SMILES: O=C(Cc1c[nH]c2nc(-c3ccccc3)c(-c3cc(Cl)c4ncccc4c3)nc2c1=O)Nc1cccnc1] |
| 1.656 | 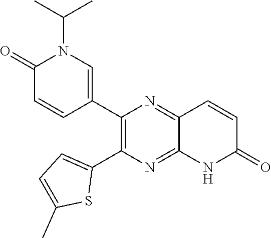 [SMILES: Cc1ccc(-c2nc3[nH]c(=O)ccc3nc2-c2ccc(=O)n(C(C)C)c2)s1] |
| 1.657 | 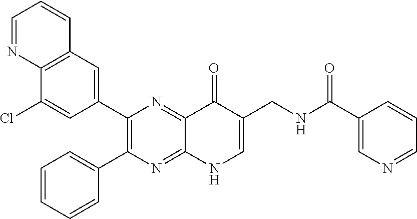 [SMILES: O=C(NCc1c[nH]c2nc(-c3ccccc3)c(-c3cc(Cl)c4ncccc4c3)nc2c1=O)c1cccnc1] |
| 1.658 | 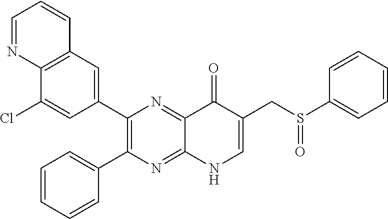 [SMILES: O=c1c(CS(=O)c2ccccc2)c[nH]c2nc(-c3ccccc3)c(-c3cc(Cl)c4ncccc4c3)nc12] |
| 1.659 | 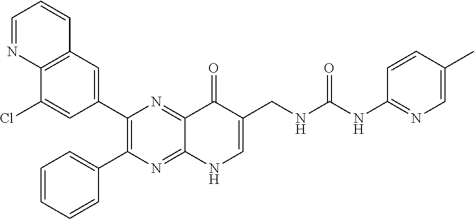 [SMILES: Cc1ccc(NC(=O)NCc2c[nH]c3nc(-c4ccccc4)c(-c4cc(Cl)c5ncccc5c4)nc3c2=O)nc1] |
| 1.660 | 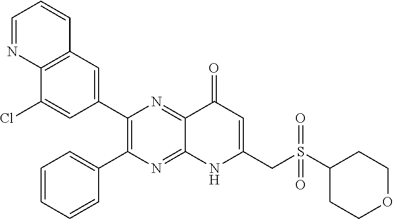 [SMILES: O=c1cc(CS(=O)(=O)C2CCOCC2)[nH]c2nc(-c3ccccc3)c(-c3cc(Cl)c4ncccc4c3)nc12] |
| 1.661 | 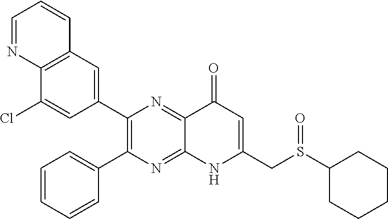 [SMILES: O=c1cc(CS(=O)C2CCCCC2)[nH]c2nc(-c3ccccc3)c(-c3cc(Cl)c4ncccc4c3)nc12] |
| 1.662 | 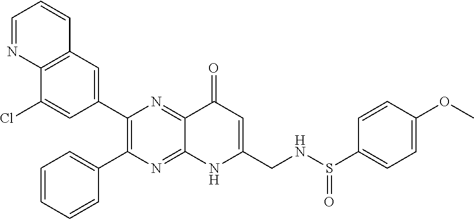 [SMILES: COc1ccc(S(=O)NCc2cc(=O)c3nc(-c4cc(Cl)c5ncccc5c4)c(-c4ccccc4)nc3[nH]2)cc1] |
| 1.663 | 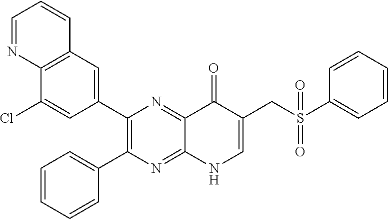 [SMILES: O=c1c(CS(=O)(=O)c2ccccc2)c[nH]c2nc(-c3ccccc3)c(-c3cc(Cl)c4ncccc4c3)nc12] |
| 1.664 | 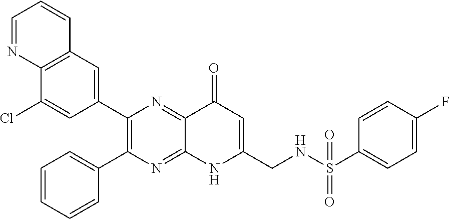 [SMILES: O=c1cc(CNS(=O)(=O)c2ccc(F)cc2)[nH]c2nc(-c3ccccc3)c(-c3cc(Cl)c4ncccc4c3)nc12] |
| 1.665 | 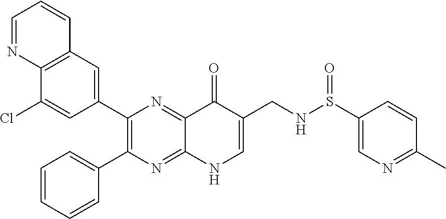 [SMILES: Cc1ccc(S(=O)NCc2c[nH]c3nc(-c4ccccc4)c(-c4cc(Cl)c5ncccc5c4)nc3c2=O)cn1] |
| 1.666 | 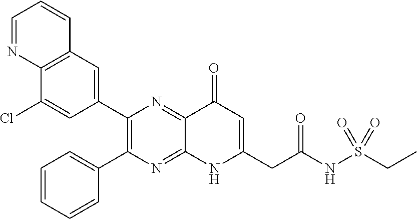 [SMILES: CCS(=O)(=O)NC(=O)Cc1cc(=O)c2nc(-c3cc(Cl)c4ncccc4c3)c(-c3ccccc3)nc2[nH]1] |
| 1.667 | 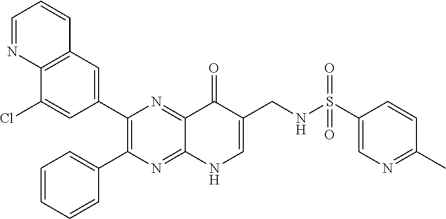 [SMILES: Cc1ccc(S(=O)(=O)NCc2c[nH]c3nc(-c4ccccc4)c(-c4cc(Cl)c5ncccc5c4)nc3c2=O)cn1] |
| 1.668 | 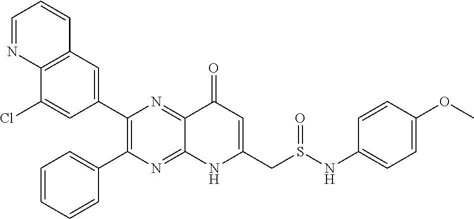 [SMILES: COc1ccc(NS(=O)Cc2cc(=O)c3nc(-c4cc(Cl)c5ncccc5c4)c(-c4ccccc4)nc3[nH]2)cc1] |
| 1.669 | 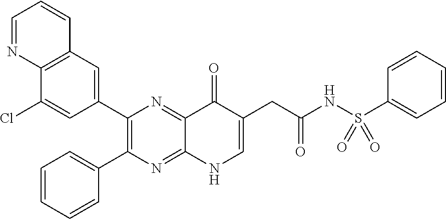 [SMILES: O=C(Cc1c[nH]c2nc(-c3ccccc3)c(-c3cc(Cl)c4ncccc4c3)nc2c1=O)NS(=O)(=O)c1ccccc1] |
| 1.670 | 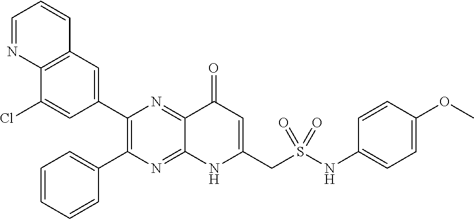 [SMILES: COc1ccc(NS(=O)(=O)Cc2cc(=O)c3nc(-c4cc(Cl)c5ncccc5c4)c(-c4ccccc4)nc3[nH]2)cc1] |
| 1.671 | 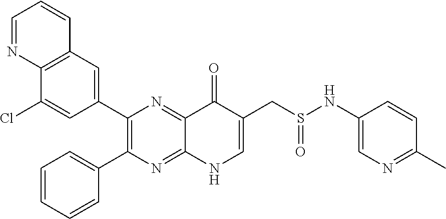 [SMILES: Cc1ccc(NS(=O)Cc2c[nH]c3nc(-c4ccccc4)c(-c4cc(Cl)c5ncccc5c4)nc3c2=O)cn1] |
| 1.672 | 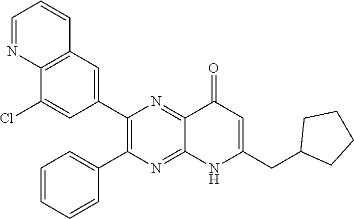 [SMILES: O=c1cc(CC2CCCC2)[nH]c2nc(-c3ccccc3)c(-c3cc(Cl)c4ncccc4c3)nc12] |
| 1.673 | 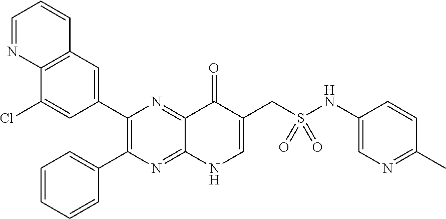 [SMILES: Cc1ccc(NS(=O)(=O)Cc2c[nH]c3nc(-c4ccccc4)c(-c4cc(Cl)c5ncccc5c4)nc3c2=O)cn1] |
| 1.674 | 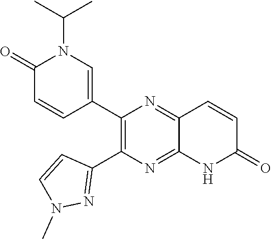 [SMILES: CC(C)n1cc(-c2nc3ccc(=O)[nH]c3nc2-c2ccn(C)n2)ccc1=O] |
| 1.675 | 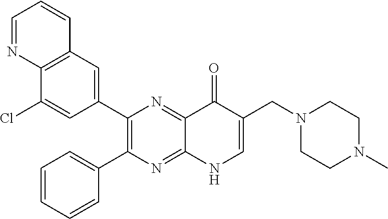 [SMILES: CN1CCN(Cc2c[nH]c3nc(-c4ccccc4)c(-c4cc(Cl)c5ncccc5c4)nc3c2=O)CC1] |
| 1.676 | 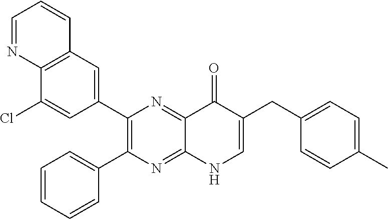 [SMILES: Cc1ccc(Cc2c[nH]c3nc(-c4ccccc4)c(-c4cc(Cl)c5ncccc5c4)nc3c2=O)cc1] |
| 1.677 | 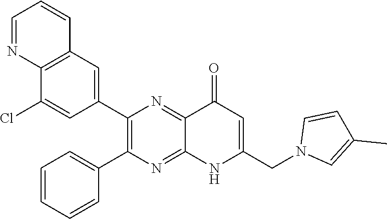 [SMILES: Cc1ccn(Cc2cc(=O)c3nc(-c4cc(Cl)c5ncccc5c4)c(-c4ccccc4)nc3[nH]2)c1] |
| 1.678 | 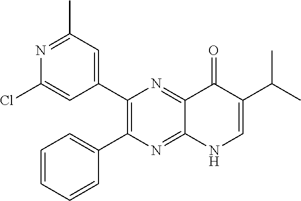 [SMILES: Cc1cc(-c2nc3c(=O)c(C(C)C)c[nH]c3nc2-c2ccccc2)cc(Cl)n1] |
| 1.679 | 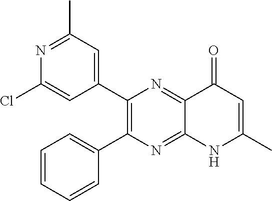 [SMILES: Cc1cc(-c2nc3c(=O)cc(C)[nH]c3nc2-c2ccccc2)cc(Cl)n1] |
| 1.680 | 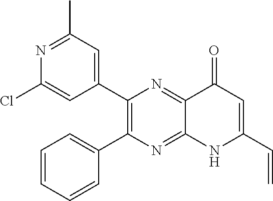 [SMILES: C=Cc1cc(=O)c2nc(-c3cc(C)nc(Cl)c3)c(-c3ccccc3)nc2[nH]1] |
| 1.681 | 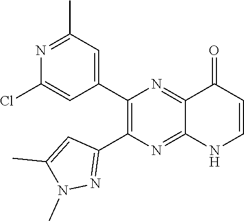 [SMILES: Cc1cc(-c2nc3c(=O)cc[nH]c3nc2-c2cc(C)n(C)n2)cc(Cl)n1] |
| 1.682 | 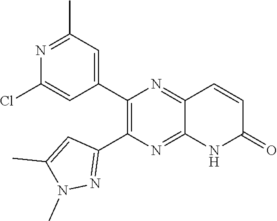 [SMILES: Cc1cc(-c2nc3ccc(=O)[nH]c3nc2-c2cc(C)n(C)n2)cc(Cl)n1] |
| 1.683 | 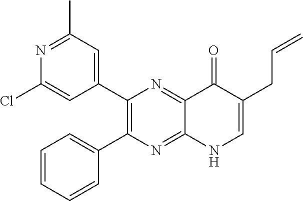 [SMILES: C=CCc1c[nH]c2nc(-c3ccccc3)c(-c3cc(C)nc(Cl)c3)nc2c1=O] |
| 1.684 | 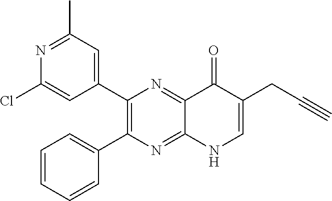 [SMILES: C#CCc1c[nH]c2nc(-c3ccccc3)c(-c3cc(C)nc(Cl)c3)nc2c1=O] |
| 1.685 | 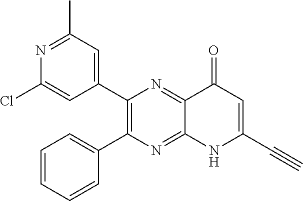 [SMILES: C#Cc1cc(=O)c2nc(-c3cc(C)nc(Cl)c3)c(-c3ccccc3)nc2[nH]1] |
| 1.686 | 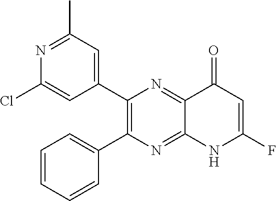 [SMILES: Cc1cc(-c2nc3c(=O)cc(F)[nH]c3nc2-c2ccccc2)cc(Cl)n1] |
| 1.687 | 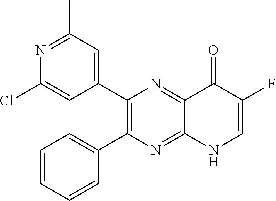 [SMILES: Cc1cc(-c2nc3c(=O)c(F)c[nH]c3nc2-c2ccccc2)cc(Cl)n1] |
| 1.688 | 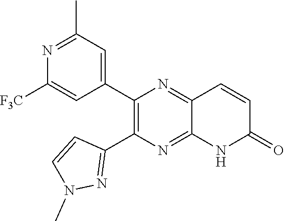 [SMILES: Cc1cc(-c2nc3ccc(=O)[nH]c3nc2-c2ccn(C)n2)cc(C(F)(F)F)n1] |
| 1.689 | 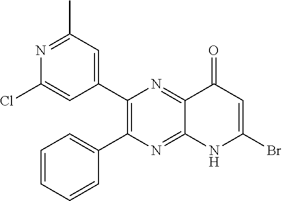 [SMILES: Cc1cc(-c2nc3c(=O)cc(Br)[nH]c3nc2-c2ccccc2)cc(Cl)n1] |
| 1.690 | 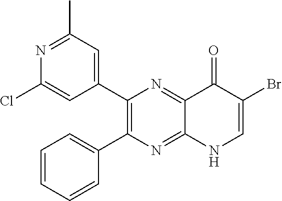 [SMILES: Cc1cc(-c2nc3c(=O)c(Br)c[nH]c3nc2-c2ccccc2)cc(Cl)n1] |
| 1.691 | 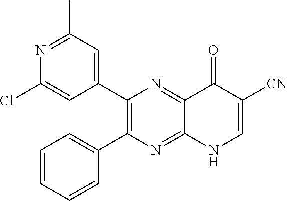 [SMILES: Cc1cc(-c2nc3c(=O)c(C#N)c[nH]c3nc2-c2ccccc2)cc(Cl)n1] |
| 1.692 | 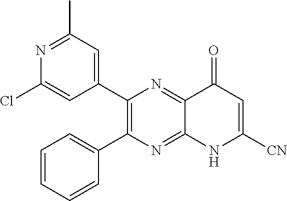 [SMILES: Cc1cc(-c2nc3c(=O)cc(C#N)[nH]c3nc2-c2ccccc2)cc(Cl)n1] |
| 1.693 | 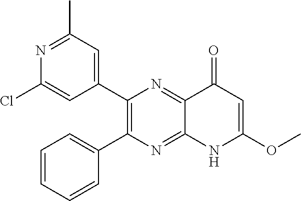 [SMILES: COc1cc(=O)c2nc(-c3cc(C)nc(Cl)c3)c(-c3ccccc3)nc2[nH]1] |
| 1.694 | 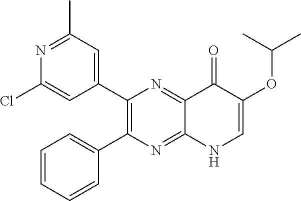 [SMILES: Cc1cc(-c2nc3c(=O)c(OC(C)C)c[nH]c3nc2-c2ccccc2)cc(Cl)n1] |
| 1.695 | 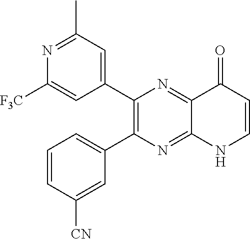 [SMILES: Cc1cc(-c2nc3c(=O)cc[nH]c3nc2-c2cccc(C#N)c2)cc(C(F)(F)F)n1] |
| 1.696 | 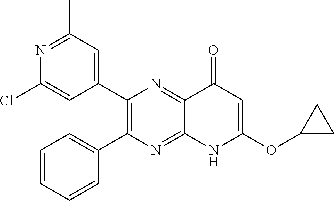 [SMILES: Cc1cc(-c2nc3c(=O)cc(OC4CC4)[nH]c3nc2-c2ccccc2)cc(Cl)n1] |
| 1.697 | 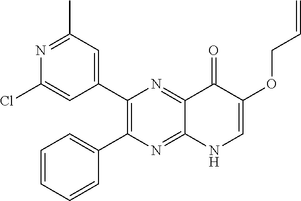 [SMILES: C=CCOc1c[nH]c2nc(-c3ccccc3)c(-c3cc(C)nc(Cl)c3)nc2c1=O] |
| 1.698 | 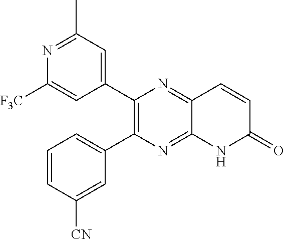 [SMILES: Cc1cc(-c2nc3ccc(=O)[nH]c3nc2-c2cccc(C#N)c2)cc(C(F)(F)F)n1] |
| 1.699 | 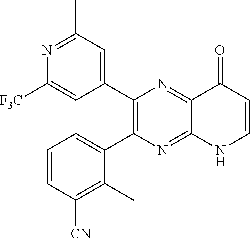 [SMILES: Cc1cc(-c2nc3c(=O)cc[nH]c3nc2-c2cccc(C#N)c2C)cc(C(F)(F)F)n1] |
| 1.700 | 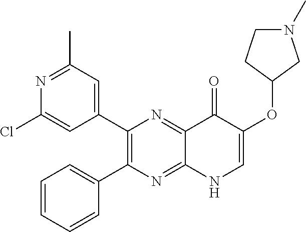 [SMILES: Cc1cc(-c2nc3c(=O)c(OC4CCN(C)C4)c[nH]c3nc2-c2ccccc2)cc(Cl)n1] |
| 1.701 | 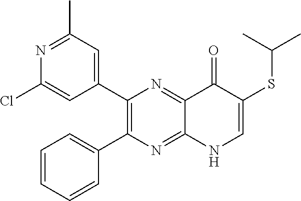 [SMILES: Cc1cc(-c2nc3c(=O)c(SC(C)C)c[nH]c3nc2-c2ccccc2)cc(Cl)n1] |
| 1.702 | 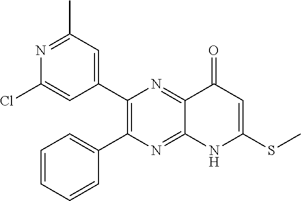 [SMILES: CSc1cc(=O)c2nc(-c3cc(C)nc(Cl)c3)c(-c3ccccc3)nc2[nH]1] |
| 1.703 | 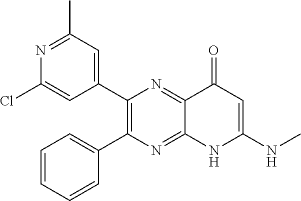 [SMILES: CNc1cc(=O)c2nc(-c3cc(C)nc(Cl)c3)c(-c3ccccc3)nc2[nH]1] |
| 1.704 | 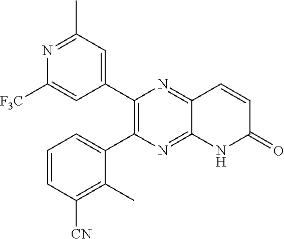 [SMILES: Cc1cc(-c2nc3ccc(=O)[nH]c3nc2-c2cccc(C#N)c2C)cc(C(F)(F)F)n1] |
| 1.705 | 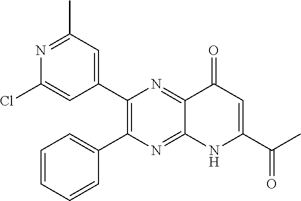 [SMILES: CC(=O)c1cc(=O)c2nc(-c3cc(C)nc(Cl)c3)c(-c3ccccc3)nc2[nH]1] |
| 1.706 | 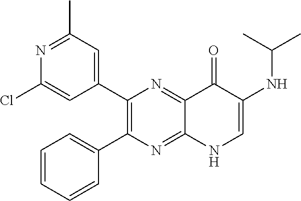 [SMILES: Cc1cc(-c2nc3c(=O)c(NC(C)C)c[nH]c3nc2-c2ccccc2)cc(Cl)n1] |
| 1.707 | 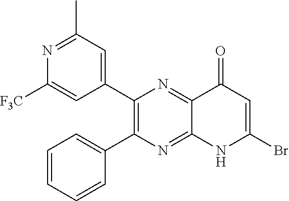 [SMILES: Cc1cc(-c2nc3c(=O)cc(Br)[nH]c3nc2-c2ccccc2)cc(C(F)(F)F)n1] |
| 1.708 | 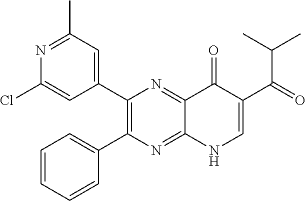 [SMILES: Cc1cc(-c2nc3c(=O)c(C(=O)C(C)C)c[nH]c3nc2-c2ccccc2)cc(Cl)n1] |
| 1.709 | 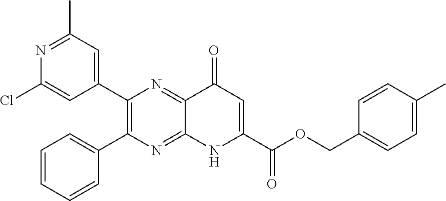 [SMILES: Cc1ccc(COC(=O)c2cc(=O)c3nc(-c4cc(C)nc(Cl)c4)c(-c4ccccc4)nc3[nH]2)cc1] |
| 1.710 | 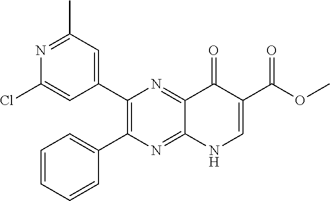 [SMILES: COC(=O)c1c[nH]c2nc(-c3ccccc3)c(-c3cc(C)nc(Cl)c3)nc2c1=O] |
| 1.711 | 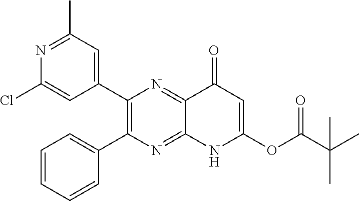 [SMILES: Cc1cc(-c2nc3c(=O)cc(OC(=O)C(C)(C)C)[nH]c3nc2-c2ccccc2)cc(Cl)n1] |
| 1.712 | 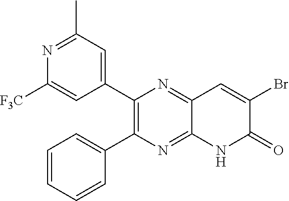 [SMILES: Cc1cc(-c2nc3cc(Br)c(=O)[nH]c3nc2-c2ccccc2)cc(C(F)(F)F)n1] |
| 1.713 | 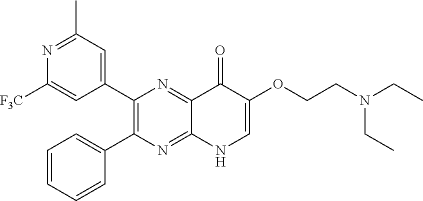 [SMILES: CCN(CC)CCOc1c[nH]c2nc(-c3ccccc3)c(-c3cc(C)nc(C(F)(F)F)c3)nc2c1=O] |
| 1.714 | 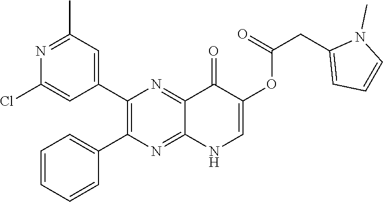 [SMILES: Cc1cc(-c2nc3c(=O)c(OC(=O)Cc4cccn4C)c[nH]c3nc2-c2ccccc2)cc(Cl)n1] |
| 1.715 | 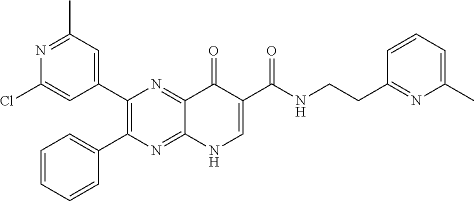 [SMILES: Cc1cc(-c2nc3c(=O)c(C(=O)NCCc4cccc(C)n4)c[nH]c3nc2-c2ccccc2)cc(Cl)n1] |
| 1.716 | 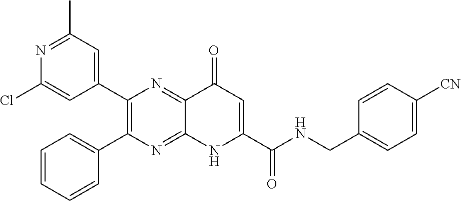 [SMILES: Cc1cc(-c2nc3c(=O)cc(C(=O)NCc4ccc(C#N)cc4)[nH]c3nc2-c2ccccc2)cc(Cl)n1] |
| 1.717 | 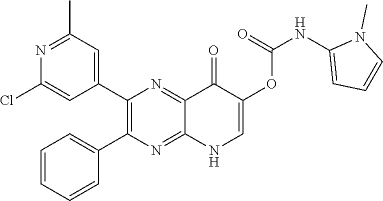 [SMILES: Cc1cc(-c2nc3c(=O)c(OC(=O)Nc4cccn4C)c[nH]c3nc2-c2ccccc2)cc(Cl)n1] |
| 1.718 | 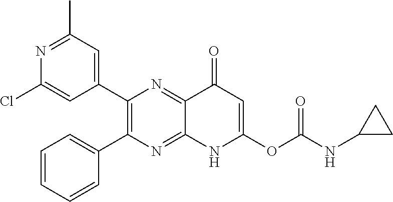 [SMILES: Cc1cc(-c2nc3c(=O)cc(OC(=O)NC4CC4)[nH]c3nc2-c2ccccc2)cc(Cl)n1] |
| 1.719 | 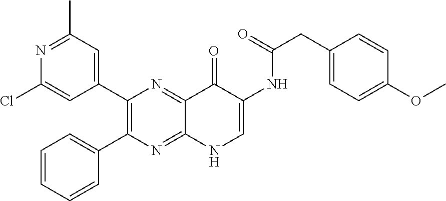 [SMILES: COc1ccc(CC(=O)Nc2c[nH]c3nc(-c4ccccc4)c(-c4cc(C)nc(Cl)c4)nc3c2=O)cc1] |
| 1.720 | 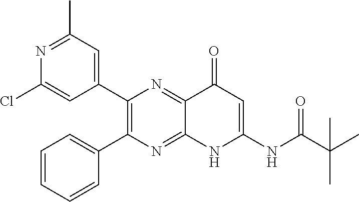 [SMILES: Cc1cc(-c2nc3c(=O)cc(NC(=O)C(C)(C)C)[nH]c3nc2-c2ccccc2)cc(Cl)n1] |
| 1.721 | 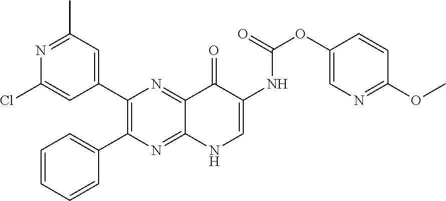 [SMILES: COc1ccc(OC(=O)Nc2c[nH]c3nc(-c4ccccc4)c(-c4cc(C)nc(Cl)c4)nc3c2=O)cn1] |
| 1.722 | 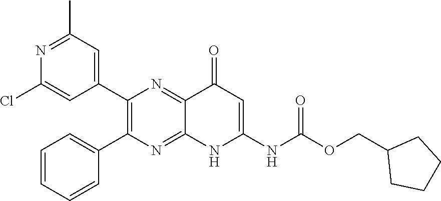 [SMILES: Cc1cc(-c2nc3c(=O)cc(NC(=O)OCC4CCCC4)[nH]c3nc2-c2ccccc2)cc(Cl)n1] |
| 1.723 | 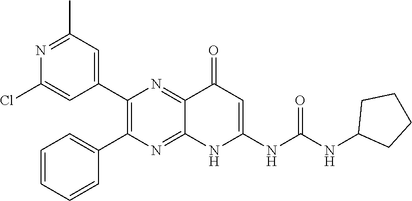 [SMILES: Cc1cc(-c2nc3c(=O)cc(NC(=O)NC4CCCC4)[nH]c3nc2-c2ccccc2)cc(Cl)n1] |
| 1.724 | 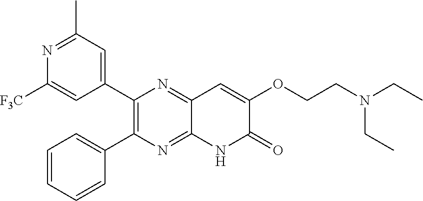 [SMILES: CCN(CC)CCOc1cc2nc(-c3cc(C)nc(C(F)(F)F)c3)c(-c3ccccc3)nc2[nH]c1=O] |
| 1.725 | 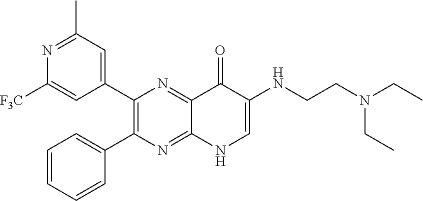 [SMILES: CCN(CC)CCNc1c[nH]c2nc(-c3ccccc3)c(-c3cc(C)nc(C(F)(F)F)c3)nc2c1=O] |
| 1.726 | 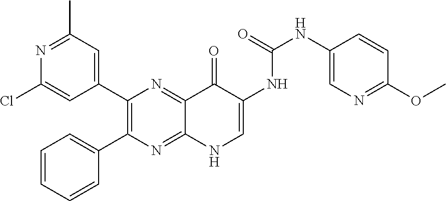 [SMILES: COc1ccc(NC(=O)Nc2c[nH]c3nc(-c4ccccc4)c(-c4cc(C)nc(Cl)c4)nc3c2=O)cn1] |
| 1.727 | 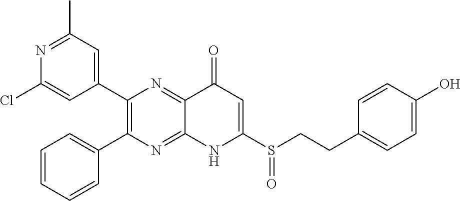 [SMILES: Cc1cc(-c2nc3c(=O)cc(S(=O)CCc4ccc(O)cc4)[nH]c3nc2-c2ccccc2)cc(Cl)n1] |
| 1.728 | 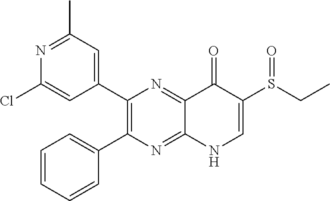 [SMILES: CCS(=O)c1c[nH]c2nc(-c3ccccc3)c(-c3cc(C)nc(Cl)c3)nc2c1=O] |
| 1.729 | 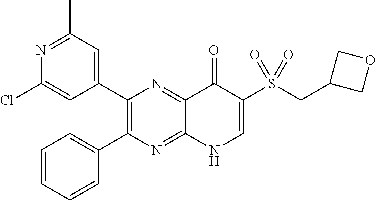 [SMILES: Cc1cc(-c2nc3c(=O)c(S(=O)(=O)CC4COC4)c[nH]c3nc2-c2ccccc2)cc(Cl)n1] |
| 1.730 | 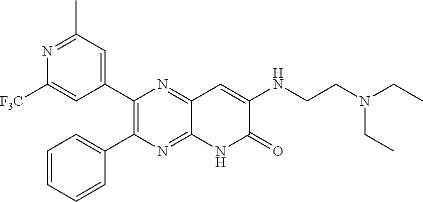 [SMILES: CCN(CC)CCNc1cc2nc(-c3cc(C)nc(C(F)(F)F)c3)c(-c3ccccc3)nc2[nH]c1=O] |
| 1.731 | 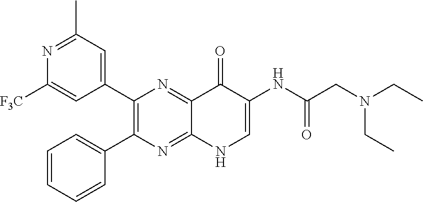 [SMILES: CCN(CC)CC(=O)Nc1c[nH]c2nc(-c3ccccc3)c(-c3cc(C)nc(C(F)(F)F)c3)nc2c1=O] |
| 1.732 | 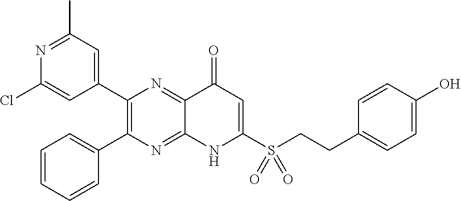 [SMILES: Cc1cc(-c2nc3c(=O)cc(S(=O)(=O)CCc4ccc(O)cc4)[nH]c3nc2-c2ccccc2)cc(Cl)n1] |
| 1.733 | 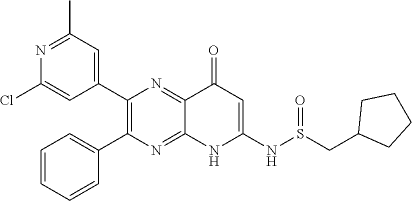 [SMILES: Cc1cc(-c2nc3c(=O)cc(NS(=O)CC4CCCC4)[nH]c3nc2-c2ccccc2)cc(Cl)n1] |
| 1.734 | 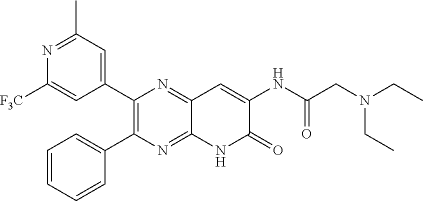 [SMILES: CCN(CC)CC(=O)Nc1cc2nc(-c3cc(C)nc(C(F)(F)F)c3)c(-c3ccccc3)nc2[nH]c1=O] |
| 1.735 | 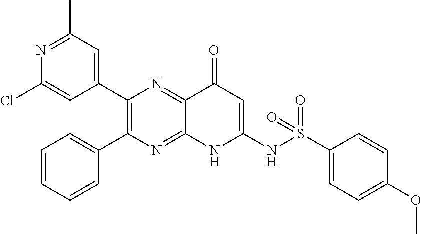 [SMILES: COc1ccc(S(=O)(=O)Nc2cc(=O)c3nc(-c4cc(C)nc(Cl)c4)c(-c4ccccc4)nc3[nH]2)cc1] |
| 1.736 | 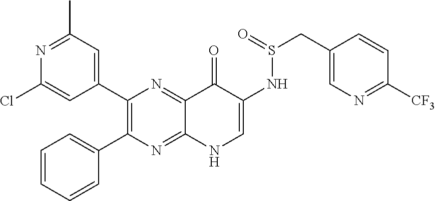 [SMILES: Cc1cc(-c2nc3c(=O)c(NS(=O)Cc4ccc(C(F)(F)F)nc4)c[nH]c3nc2-c2ccccc2)cc(Cl)n1] |
| 1.737 | 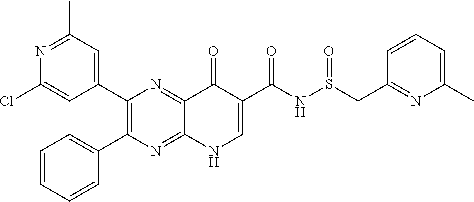 [SMILES: Cc1cc(-c2nc3c(=O)c(C(=O)NS(=O)Cc4cccc(C)n4)c[nH]c3nc2-c2ccccc2)cc(Cl)n1] |
| 1.738 | 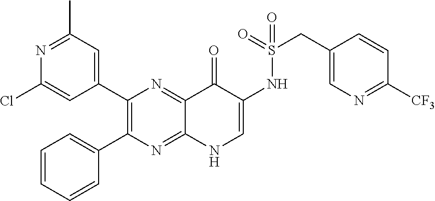 [SMILES: Cc1cc(-c2nc3c(=O)c(NS(=O)(=O)Cc4ccc(C(F)(F)F)nc4)c[nH]c3nc2-c2ccccc2)cc(Cl)n1] |
| 1.739 | 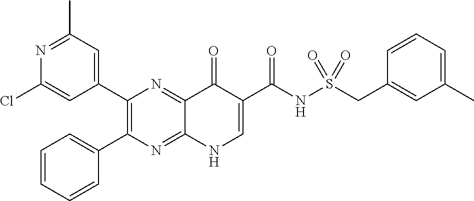 [SMILES: Cc1cccc(CS(=O)(=O)NC(=O)c2c[nH]c3nc(-c4ccccc4)c(-c4cc(C)nc(Cl)c4)nc3c2=O)c1] |
| 1.740 | 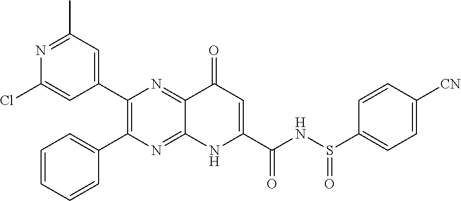 [SMILES: Cc1cc(-c2nc3c(=O)cc(C(=O)NS(=O)c4ccc(C#N)cc4)[nH]c3nc2-c2ccccc2)cc(Cl)n1] |
| 1.741 | 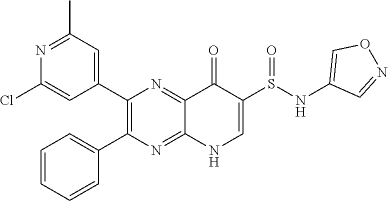 [SMILES: Cc1cc(-c2nc3c(=O)c(S(=O)Nc4cnoc4)c[nH]c3nc2-c2ccccc2)cc(Cl)n1] |
| 1.742 | 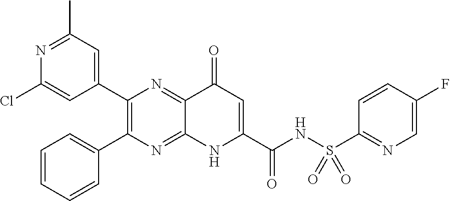 [SMILES: Cc1cc(-c2nc3c(=O)cc(C(=O)NS(=O)(=O)c4ccc(F)cn4)[nH]c3nc2-c2ccccc2)cc(Cl)n1] |
| 1.743 | 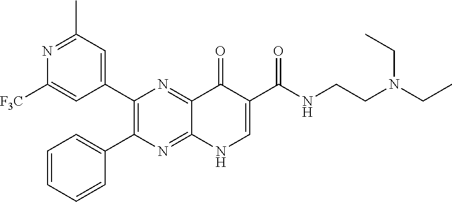 [SMILES: CCN(CC)CCNC(=O)c1c[nH]c2nc(-c3ccccc3)c(-c3cc(C)nc(C(F)(F)F)c3)nc2c1=O] |
| 1.744 | 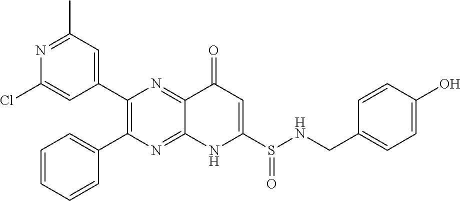 [SMILES: Cc1cc(-c2nc3c(=O)cc(S(=O)NCc4ccc(O)cc4)[nH]c3nc2-c2ccccc2)cc(Cl)n1] |
| 1.745 | 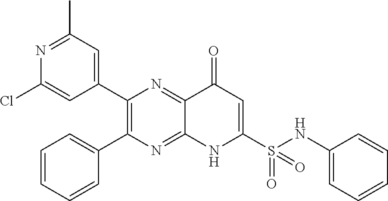 [SMILES: Cc1cc(-c2nc3c(=O)cc(S(=O)(=O)Nc4ccccc4)[nH]c3nc2-c2ccccc2)cc(Cl)n1] |
| 1.746 | 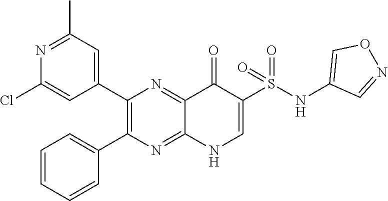 [SMILES: Cc1cc(-c2nc3c(=O)c(S(=O)(=O)Nc4cnoc4)c[nH]c3nc2-c2ccccc2)cc(Cl)n1] |
| 1.747 | 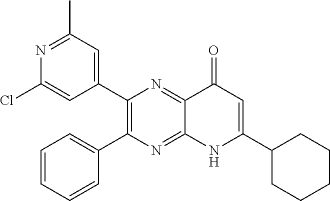 [SMILES: Cc1cc(-c2nc3c(=O)cc(C4CCCCC4)[nH]c3nc2-c2ccccc2)cc(Cl)n1] |
| 1.748 | 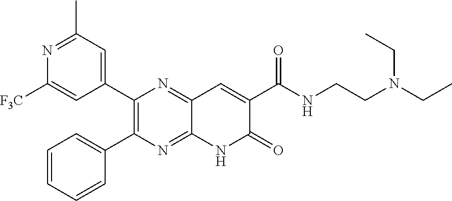 [SMILES: CCN(CC)CCNC(=O)c1cc2nc(-c3cc(C)nc(C(F)(F)F)c3)c(-c3ccccc3)nc2[nH]c1=O] |
| 1.749 | 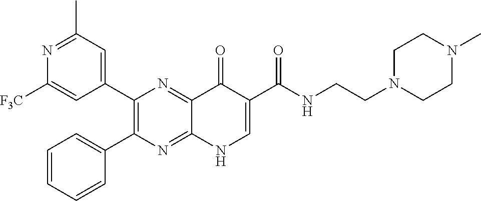 [SMILES: Cc1cc(-c2nc3c(=O)c(C(=O)NCCN4CCN(C)CC4)c[nH]c3nc2-c2ccccc2)cc(C(F)(F)F)n1] |
| 1.750 | 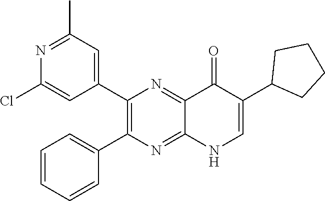 [SMILES: Cc1cc(-c2nc3c(=O)c(C4CCCC4)c[nH]c3nc2-c2ccccc2)cc(Cl)n1] |
| 1.751 | 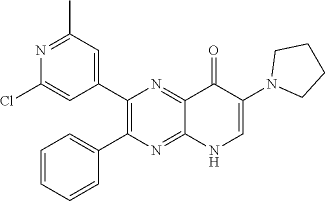 [SMILES: Cc1cc(-c2nc3c(=O)c(N4CCCC4)c[nH]c3nc2-c2ccccc2)cc(Cl)n1] |
| 1.752 | 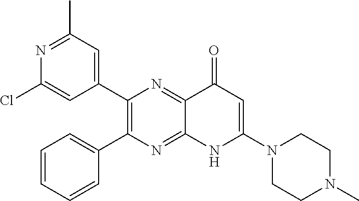 [SMILES: Cc1cc(-c2nc3c(=O)cc(N4CCN(C)CC4)[nH]c3nc2-c2ccccc2)cc(Cl)n1] |
| 1.753 | 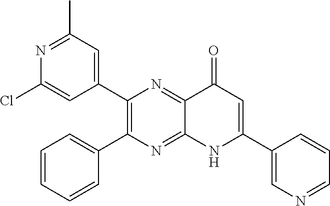 [SMILES: Cc1cc(-c2nc3c(=O)cc(-c4cccnc4)[nH]c3nc2-c2ccccc2)cc(Cl)n1] |
| 1.754 | 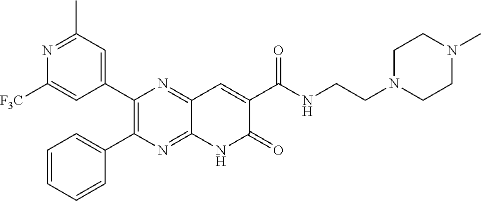 [SMILES: Cc1cc(-c2nc3cc(C(=O)NCCN4CCN(C)CC4)c(=O)[nH]c3nc2-c2ccccc2)cc(C(F)(F)F)n1] |
| 1.755 | 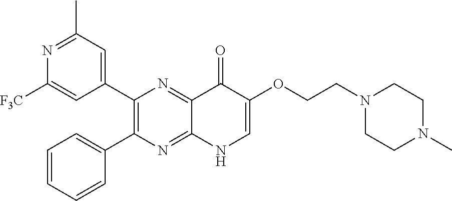 [SMILES: Cc1cc(-c2nc3c(=O)c(OCCN4CCN(C)CC4)c[nH]c3nc2-c2ccccc2)cc(C(F)(F)F)n1] |
| 1.756 | 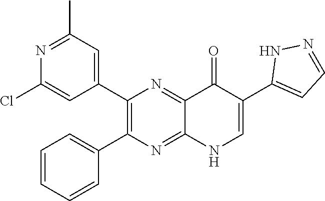 [SMILES: Cc1cc(-c2nc3c(=O)c(-c4ccn[nH]4)c[nH]c3nc2-c2ccccc2)cc(Cl)n1] |
| 1.757 | 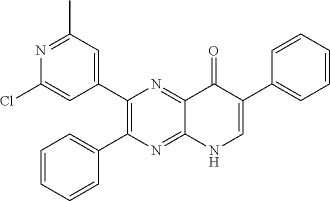 [SMILES: Cc1cc(-c2nc3c(=O)c(-c4ccccc4)c[nH]c3nc2-c2ccccc2)cc(Cl)n1] |
| 1.758 | 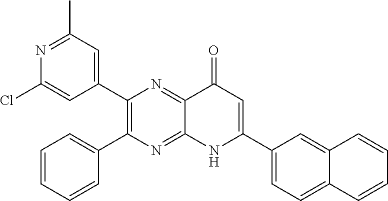 [SMILES: Cc1cc(-c2nc3c(=O)cc(-c4ccc5ccccc5c4)[nH]c3nc2-c2ccccc2)cc(Cl)n1] |
| 1.759 | 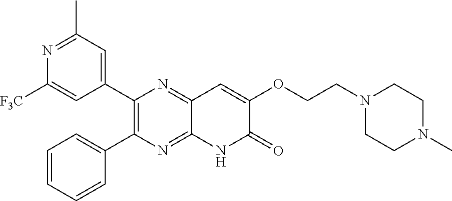 [SMILES: Cc1cc(-c2nc3cc(OCCN4CCN(C)CC4)c(=O)[nH]c3nc2-c2ccccc2)cc(C(F)(F)F)n1] |
| 1.760 | 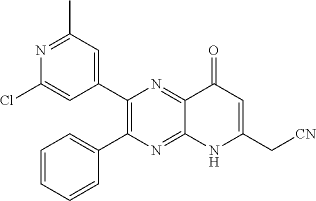 [SMILES: Cc1cc(-c2nc3c(=O)cc(CC#N)[nH]c3nc2-c2ccccc2)cc(Cl)n1] |
| 1.761 | 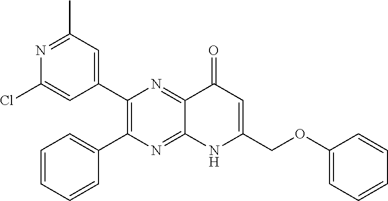 [SMILES: Cc1cc(-c2nc3c(=O)cc(COc4ccccc4)[nH]c3nc2-c2ccccc2)cc(Cl)n1] |
| 1.762 | 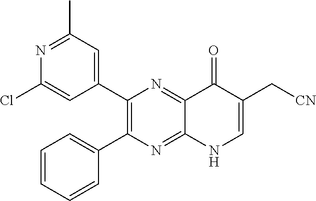 [SMILES: Cc1cc(-c2nc3c(=O)c(CC#N)c[nH]c3nc2-c2ccccc2)cc(Cl)n1] |
| 1.763 | 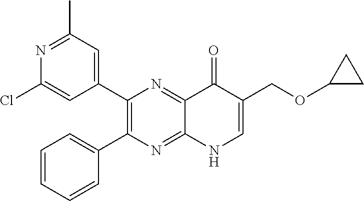 [SMILES: Cc1cc(-c2nc3c(=O)c(COC4CC4)c[nH]c3nc2-c2ccccc2)cc(Cl)n1] |
| 1.764 | 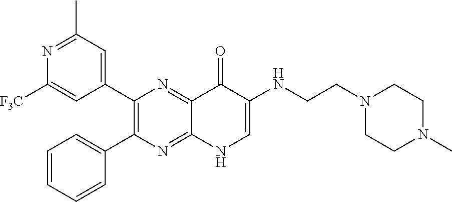 [SMILES: Cc1cc(-c2nc3c(=O)c(NCCN4CCN(C)CC4)c[nH]c3nc2-c2ccccc2)cc(C(F)(F)F)n1] |
| 1.765 | 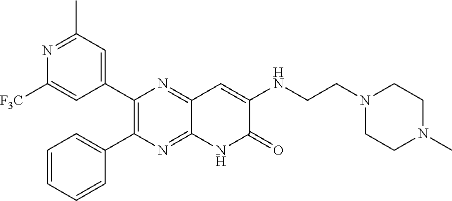 [SMILES: Cc1cc(-c2nc3cc(NCCN4CCN(C)CC4)c(=O)[nH]c3nc2-c2ccccc2)cc(C(F)(F)F)n1] |
| 1.766 | 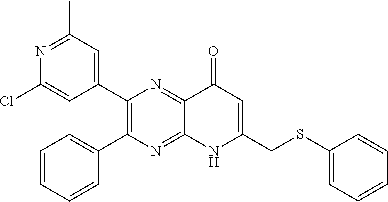 [SMILES: Cc1cc(-c2nc3c(=O)cc(CSc4ccccc4)[nH]c3nc2-c2ccccc2)cc(Cl)n1] |
| 1.767 | 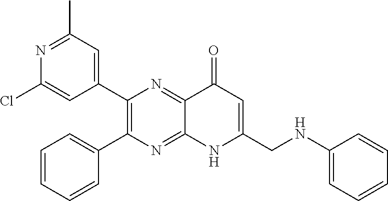 [SMILES: Cc1cc(-c2nc3c(=O)cc(CNc4ccccc4)[nH]c3nc2-c2ccccc2)cc(Cl)n1] |
| 1.768 | 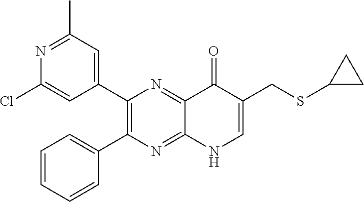 [SMILES: Cc1cc(-c2nc3c(=O)c(CSC4CC4)c[nH]c3nc2-c2ccccc2)cc(Cl)n1] |
| 1.769 | 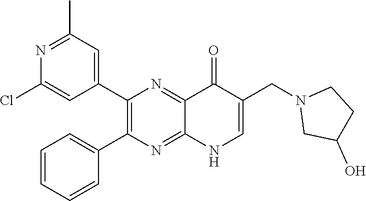 [SMILES: Cc1cc(-c2nc3c(=O)c(CN4CCC(O)C4)c[nH]c3nc2-c2ccccc2)cc(Cl)n1] |
| 1.770 | 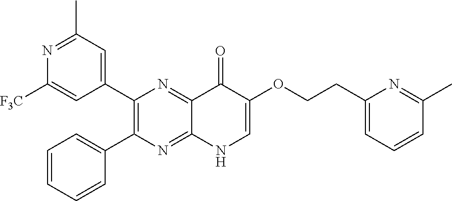 [SMILES: Cc1cccc(CCOc2c[nH]c3nc(-c4ccccc4)c(-c4cc(C)nc(C(F)(F)F)c4)nc3c2=O)n1] |
| 1.771 | 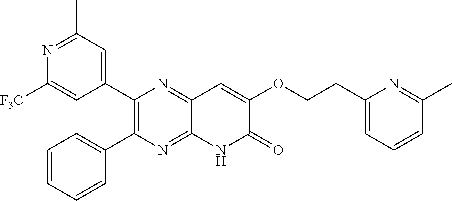 [SMILES: Cc1cccc(CCOc2cc3nc(-c4cc(C)nc(C(F)(F)F)c4)c(-c4ccccc4)nc3[nH]c2=O)n1] |
| 1.772 | 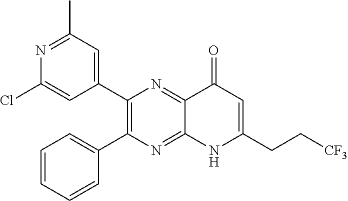 [SMILES: Cc1cc(-c2nc3c(=O)cc(CCC(F)(F)F)[nH]c3nc2-c2ccccc2)cc(Cl)n1] |
| 1.773 | 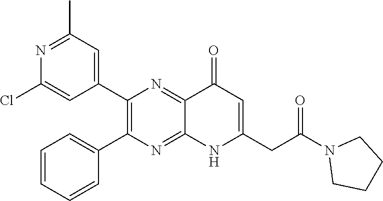 [SMILES: Cc1cc(-c2nc3c(=O)cc(CC(=O)N4CCCC4)[nH]c3nc2-c2ccccc2)cc(Cl)n1] |
| 1.774 | 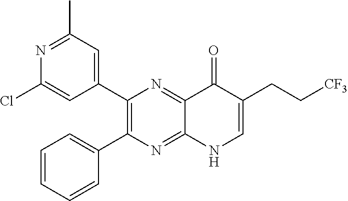 [SMILES: Cc1cc(-c2nc3c(=O)c(CCC(F)(F)F)c[nH]c3nc2-c2ccccc2)cc(Cl)n1] |
| 1.775 | 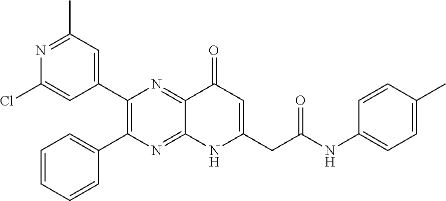 [SMILES: Cc1ccc(NC(=O)Cc2cc(=O)c3nc(-c4cc(C)nc(Cl)c4)c(-c4ccccc4)nc3[nH]2)cc1] |
| 1.776 | 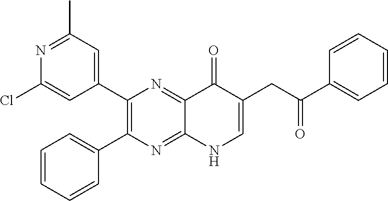 [SMILES: Cc1cc(-c2nc3c(=O)c(CC(=O)c4ccccc4)c[nH]c3nc2-c2ccccc2)cc(Cl)n1] |
| 1.777 | 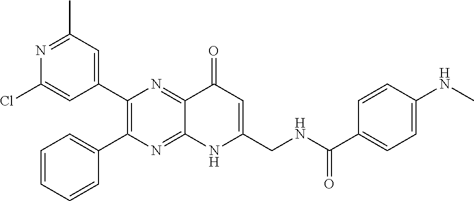 [SMILES: CNc1ccc(C(=O)NCc2cc(=O)c3nc(-c4cc(C)nc(Cl)c4)c(-c4ccccc4)nc3[nH]2)cc1] |
| 1.778 | 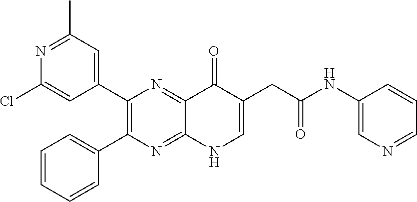 [SMILES: Cc1cc(-c2nc3c(=O)c(CC(=O)Nc4cccnc4)c[nH]c3nc2-c2ccccc2)cc(Cl)n1] |
| 1.779 | 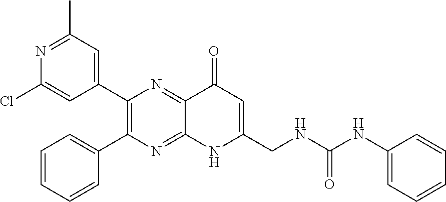 [SMILES: Cc1cc(-c2nc3c(=O)cc(CNC(=O)Nc4ccccc4)[nH]c3nc2-c2ccccc2)cc(Cl)n1] |
| 1.780 | 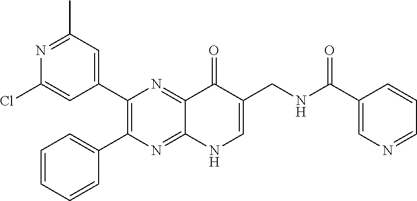 [SMILES: Cc1cc(-c2nc3c(=O)c(CNC(=O)c4cccnc4)c[nH]c3nc2-c2ccccc2)cc(Cl)n1] |
| 1.781 | 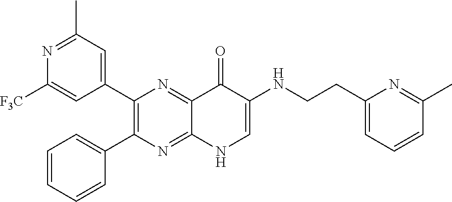 [SMILES: Cc1cccc(CCNc2c[nH]c3nc(-c4ccccc4)c(-c4cc(C)nc(C(F)(F)F)c4)nc3c2=O)n1] |
| 1.782 | 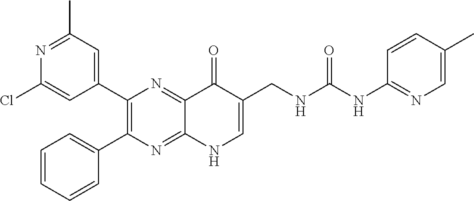 [SMILES: Cc1ccc(NC(=O)NCc2c[nH]c3nc(-c4ccccc4)c(-c4cc(C)nc(Cl)c4)nc3c2=O)nc1] |
| 1.783 | 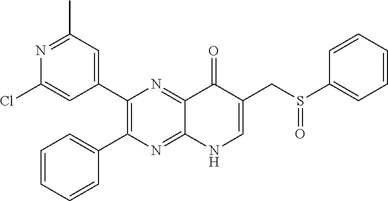 [SMILES: Cc1cc(-c2nc3c(=O)c(CS(=O)c4ccccc4)c[nH]c3nc2-c2ccccc2)cc(Cl)n1] |
| 1.784 | 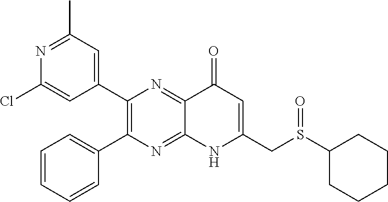 [SMILES: Cc1cc(-c2nc3c(=O)cc(CS(=O)C4CCCCC4)[nH]c3nc2-c2ccccc2)cc(Cl)n1] |
| 1.785 | 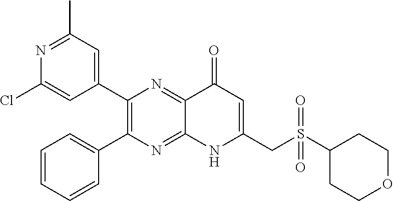 [SMILES: Cc1cc(-c2nc3c(=O)cc(CS(=O)(=O)C4CCOCC4)[nH]c3nc2-c2ccccc2)cc(Cl)n1] |
| 1.786 | 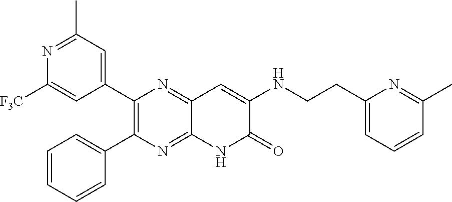 [SMILES: Cc1cccc(CCNc2cc3nc(-c4cc(C)nc(C(F)(F)F)c4)c(-c4ccccc4)nc3[nH]c2=O)n1] |
| 1.787 | 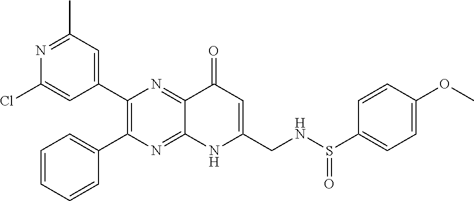 [SMILES: COc1ccc(S(=O)NCc2cc(=O)c3nc(-c4cc(C)nc(Cl)c4)c(-c4ccccc4)nc3[nH]2)cc1] |
| 1.788 | 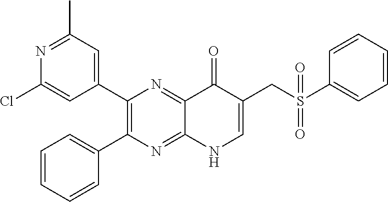 [SMILES: Cc1cc(-c2nc3c(=O)c(CS(=O)(=O)c4ccccc4)c[nH]c3nc2-c2ccccc2)cc(Cl)n1] |
| 1.789 | 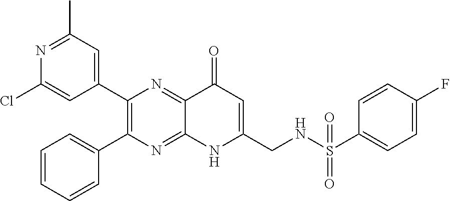 [SMILES: Cc1cc(-c2nc3c(=O)cc(CNS(=O)(=O)c4ccc(F)cc4)[nH]c3nc2-c2ccccc2)cc(Cl)n1] |
| 1.790 | 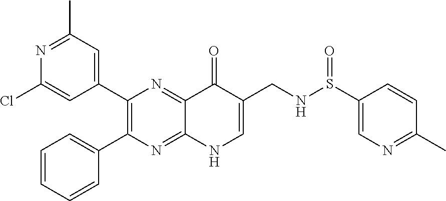 [SMILES: Cc1ccc(S(=O)NCc2c[nH]c3nc(-c4ccccc4)c(-c4cc(C)nc(Cl)c4)nc3c2=O)cn1] |
| 1.791 | 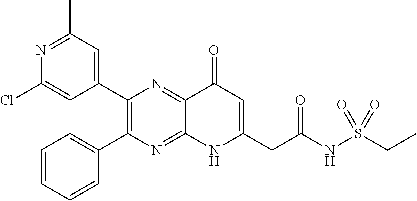 [SMILES: CCS(=O)(=O)NC(=O)Cc1cc(=O)c2nc(-c3cc(C)nc(Cl)c3)c(-c3ccccc3)nc2[nH]1] |
| 1.792 | 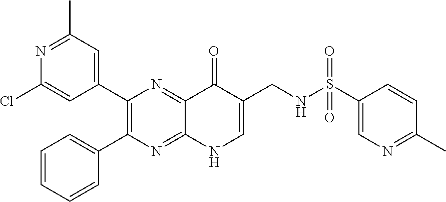 [SMILES: Cc1ccc(S(=O)(=O)NCc2c[nH]c3nc(-c4ccccc4)c(-c4cc(C)nc(Cl)c4)nc3c2=O)cn1] |
| 1.793 | 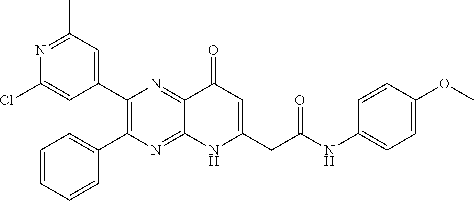 [SMILES: COc1ccc(NC(=O)Cc2cc(=O)c3nc(-c4cc(C)nc(Cl)c4)c(-c4ccccc4)nc3[nH]2)cc1] |
| 1.794 | 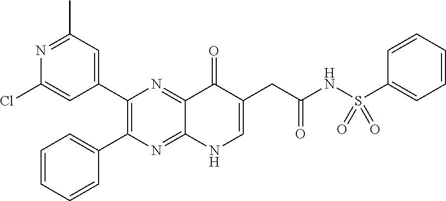 [SMILES: Cc1cc(-c2nc3c(=O)c(CC(=O)NS(=O)(=O)c4ccccc4)c[nH]c3nc2-c2ccccc2)cc(Cl)n1] |
| 1.795 | 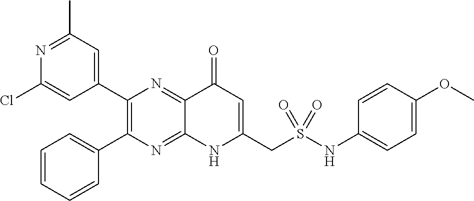 [SMILES: COc1ccc(NS(=O)(=O)Cc2cc(=O)c3nc(-c4cc(C)nc(Cl)c4)c(-c4ccccc4)nc3[nH]2)cc1] |
| 1.796 | 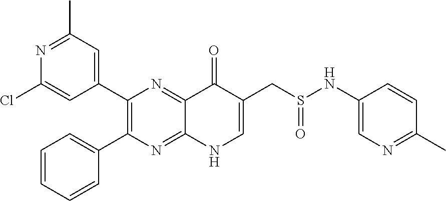 [SMILES: Cc1ccc(NS(=O)Cc2c[nH]c3nc(-c4ccccc4)c(-c4cc(C)nc(Cl)c4)nc3c2=O)cn1] |
| 1.797 | 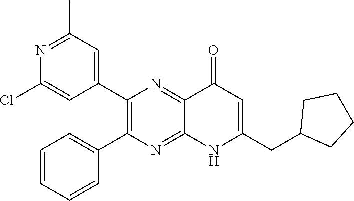 [SMILES: Cc1cc(-c2nc3c(=O)cc(CC4CCCC4)[nH]c3nc2-c2ccccc2)cc(Cl)n1] |
| 1.798 | 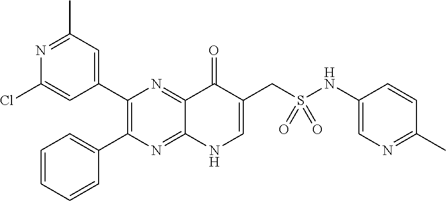 [SMILES: Cc1ccc(NS(=O)(=O)Cc2c[nH]c3nc(-c4ccccc4)c(-c4cc(C)nc(Cl)c4)nc3c2=O)cn1] |
| 1.799 | 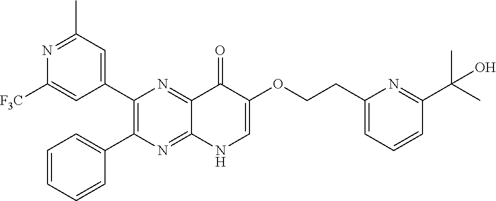 [SMILES: Cc1cc(-c2nc3c(=O)c(OCCc4cccc(C(C)(C)O)n4)c[nH]c3nc2-c2ccccc2)cc(C(F)(F)F)n1] |
| 1.800 | 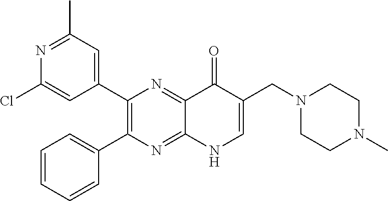 [SMILES: Cc1cc(-c2nc3c(=O)c(CN4CCN(C)CC4)c[nH]c3nc2-c2ccccc2)cc(Cl)n1] |
| 1.801 | 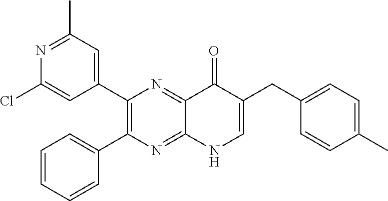 [SMILES: Cc1ccc(Cc2c[nH]c3nc(-c4ccccc4)c(-c4cc(C)nc(Cl)c4)nc3c2=O)cc1] |
| 1.802 | 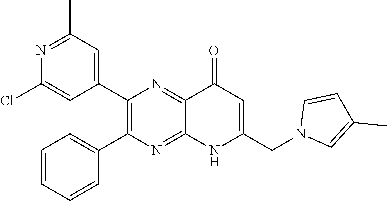 [SMILES: Cc1ccn(Cc2cc(=O)c3nc(-c4cc(C)nc(Cl)c4)c(-c4ccccc4)nc3[nH]2)c1] |
| 1.803 | 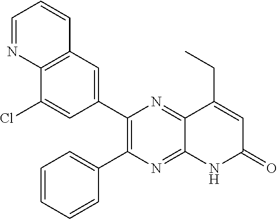 [SMILES: CCc1cc(=O)[nH]c2nc(-c3ccccc3)c(-c3cc(Cl)c4ncccc4c3)nc12] |
| 1.804 | 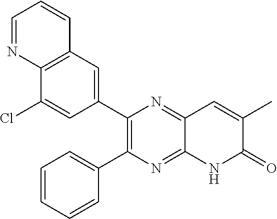 [SMILES: Cc1cc2nc(-c3cc(Cl)c4ncccc4c3)c(-c3ccccc3)nc2[nH]c1=O] |
| 1.805 | 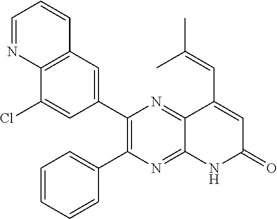 [SMILES: CC(C)=Cc1cc(=O)[nH]c2nc(-c3ccccc3)c(-c3cc(Cl)c4ncccc4c3)nc12] |
| 1.806 | 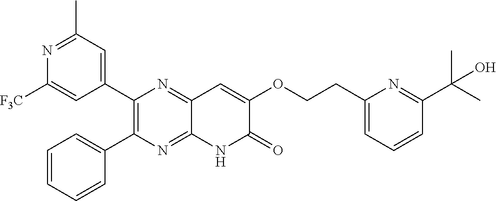 [SMILES: Cc1cc(-c2nc3cc(OCCc4cccc(C(C)(C)O)n4)c(=O)[nH]c3nc2-c2ccccc2)cc(C(F)(F)F)n1] |
| 1.807 | 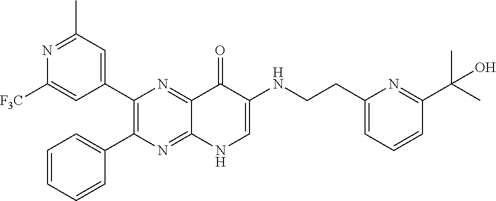 [SMILES: Cc1cc(-c2nc3c(=O)c(NCCc4cccc(C(C)(C)O)n4)c[nH]c3nc2-c2ccccc2)cc(C(F)(F)F)n1] |
| 1.808 | 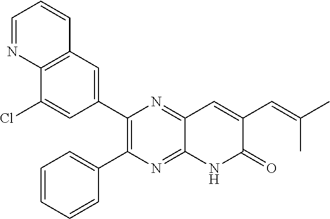 [SMILES: CC(C)=Cc1cc2nc(-c3cc(Cl)c4ncccc4c3)c(-c3ccccc3)nc2[nH]c1=O] |
| 1.809 | 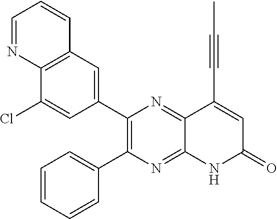 [SMILES: CC#Cc1cc(=O)[nH]c2nc(-c3ccccc3)c(-c3cc(Cl)c4ncccc4c3)nc12] |
| 1.810 | 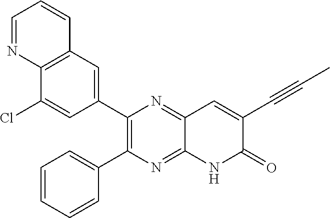 [SMILES: CC#Cc1cc2nc(-c3cc(Cl)c4ncccc4c3)c(-c3ccccc3)nc2[nH]c1=O] |
| 1.811 | 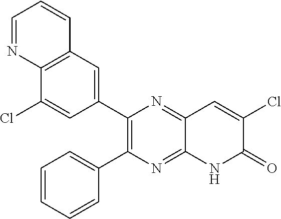 [SMILES: O=c1[nH]c2nc(-c3ccccc3)c(-c3cc(Cl)c4ncccc4c3)nc2cc1Cl] |
| 1.812 | 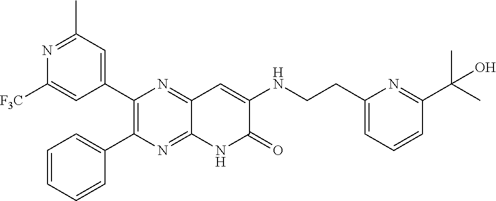 [SMILES: Cc1cc(-c2nc3cc(NCCc4cccc(C(C)(C)O)n4)c(=O)[nH]c3nc2-c2ccccc2)cc(C(F)(F)F)n1] |
| 1.813 | 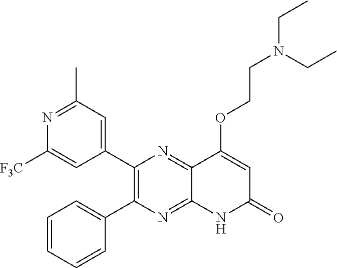 [SMILES: CCN(CC)CCOc1cc(=O)[nH]c2nc(-c3ccccc3)c(-c3cc(C)nc(C(F)(F)F)c3)nc12] |
| 1.814 | 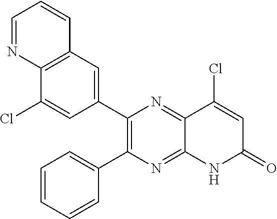 [SMILES: O=c1cc(Cl)c2nc(-c3cc(Cl)c4ncccc4c3)c(-c3ccccc3)nc2[nH]1] |
| 1.815 | 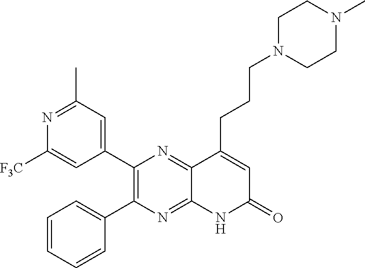 [SMILES: Cc1cc(-c2nc3c(CCCN4CCN(C)CC4)cc(=O)[nH]c3nc2-c2ccccc2)cc(C(F)(F)F)n1] |
| 1.816 | 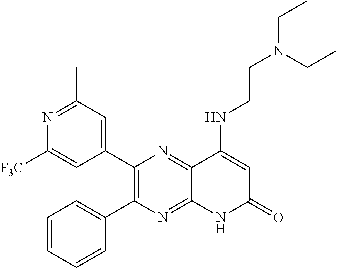 [SMILES: CCN(CC)CCNc1cc(=O)[nH]c2nc(-c3ccccc3)c(-c3cc(C)nc(C(F)(F)F)c3)nc12] |
| 1.817 | 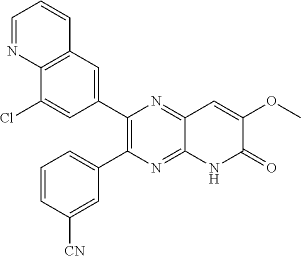 [SMILES: COc1cc2nc(-c3cc(Cl)c4ncccc4c3)c(-c3cccc(C#N)c3)nc2[nH]c1=O] |
| 1.818 | 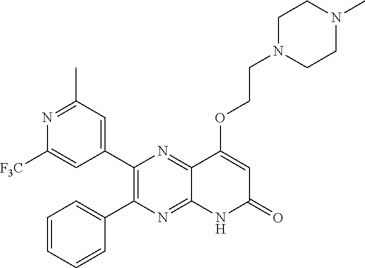 [SMILES: Cc1cc(-c2nc3c(OCCN4CCN(C)CC4)cc(=O)[nH]c3nc2-c2ccccc2)cc(C(F)(F)F)n1] |
| 1.819 | 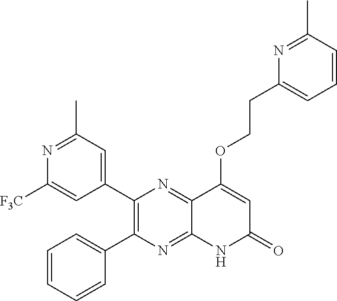 [SMILES: Cc1cccc(CCOc2cc(=O)[nH]c3nc(-c4ccccc4)c(-c4cc(C)nc(C(F)(F)F)c4)nc23)n1] |
| 1.820 | 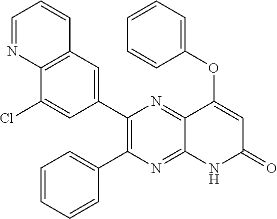 [SMILES: O=c1cc(Oc2ccccc2)c2nc(-c3cc(Cl)c4ncccc4c3)c(-c3ccccc3)nc2[nH]1] |
| 1.821 | 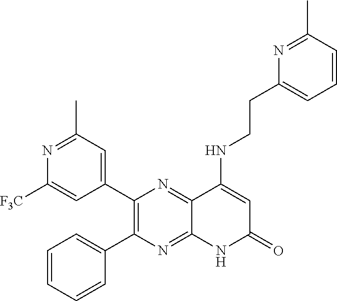 [SMILES: Cc1cccc(CCNc2cc(=O)[nH]c3nc(-c4ccccc4)c(-c4cc(C)nc(C(F)(F)F)c4)nc23)n1] |
| 1.822 | 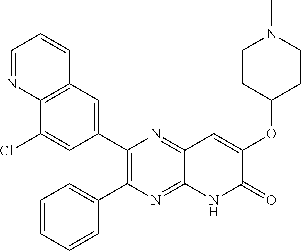 [SMILES: CN1CCC(Oc2cc3nc(-c4cc(Cl)c5ncccc5c4)c(-c4ccccc4)nc3[nH]c2=O)CC1] |
| 1.823 | 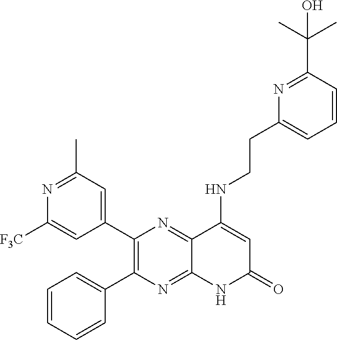 [SMILES: Cc1cc(-c2nc3c(NCCc4cccc(C(C)(C)O)n4)cc(=O)[nH]c3nc2-c2ccccc2)cc(C(F)(F)F)n1] |
| 1.824 | 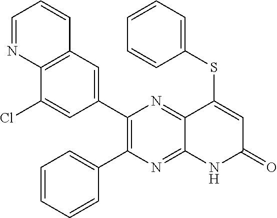 [SMILES: O=c1cc(Sc2ccccc2)c2nc(-c3cc(Cl)c4ncccc4c3)c(-c3ccccc3)nc2[nH]1] |
| 1.825 | 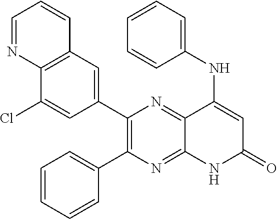 [SMILES: O=c1cc(Nc2ccccc2)c2nc(-c3cc(Cl)c4ncccc4c3)c(-c3ccccc3)nc2[nH]1] |
| 1.826 | 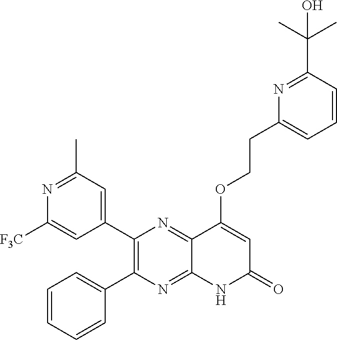 [SMILES: Cc1cc(-c2nc3c(OCCc4cccc(C(C)(C)O)n4)cc(=O)[nH]c3nc2-c2ccccc2)cc(C(F)(F)F)n1] |
| 1.827 | 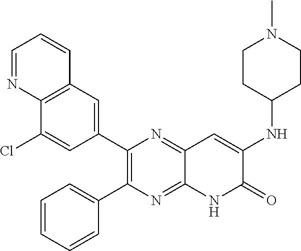 [SMILES: CN1CCC(Nc2cc3nc(-c4cc(Cl)c5ncccc5c4)c(-c4ccccc4)nc3[nH]c2=O)CC1] |
| 1.828 | 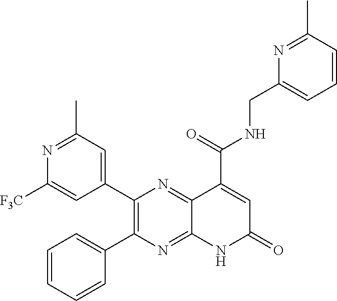 [SMILES: Cc1cccc(CNC(=O)c2cc(=O)[nH]c3nc(-c4ccccc4)c(-c4cc(C)nc(C(F)(F)F)c4)nc23)n1] |
| 1.829 | 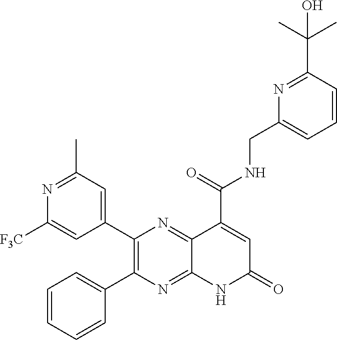 [SMILES: Cc1cc(-c2nc3c(C(=O)NCc4cccc(C(C)(C)O)n4)cc(=O)[nH]c3nc2-c2ccccc2)cc(C(F)(F)F)n1] |
| 1.830 | 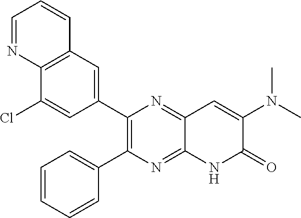 [SMILES: CN(C)c1cc2nc(-c3cc(Cl)c4ncccc4c3)c(-c3ccccc3)nc2[nH]c1=O] |
| 1.831 | 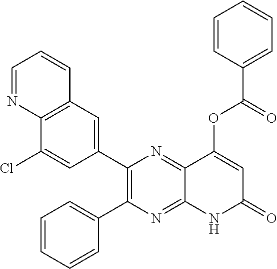 [SMILES: O=C(Oc1cc(=O)[nH]c2nc(-c3ccccc3)c(-c3cc(Cl)c4ncccc4c3)nc12)c1ccccc1] |
| 1.832 | 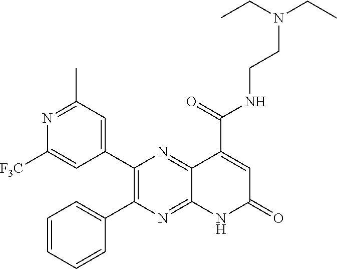 [SMILES: CCN(CC)CCNC(=O)c1cc(=O)[nH]c2nc(-c3ccccc3)c(-c3cc(C)nc(C(F)(F)F)c3)nc12] |
| 1.833 | 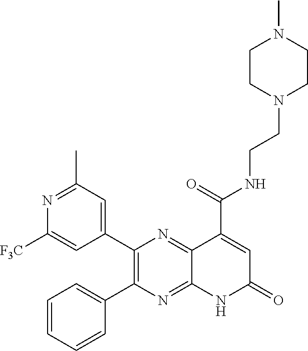 [SMILES: Cc1cc(-c2nc3c(C(=O)NCCN4CCN(C)CC4)cc(=O)[nH]c3nc2-c2ccccc2)cc(C(F)(F)F)n1] |
| 1.834 | 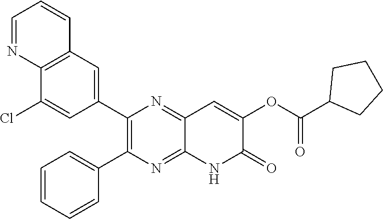 [SMILES: O=C(Oc1cc2nc(-c3cc(Cl)c4ncccc4c3)c(-c3ccccc3)nc2[nH]c1=O)C1CCCC1] |
| 1.835 | 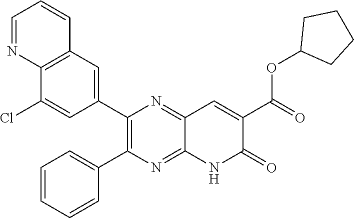 [SMILES: O=C(OC1CCCC1)c1cc2nc(-c3cc(Cl)c4ncccc4c3)c(-c3ccccc3)nc2[nH]c1=O] |
| 1.836 | 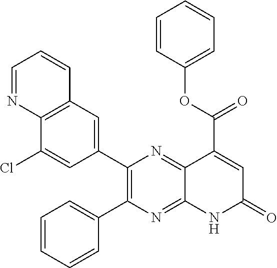 [SMILES: O=C(Oc1ccccc1)c1cc(=O)[nH]c2nc(-c3ccccc3)c(-c3cc(Cl)c4ncccc4c3)nc12] |
| 1.837 | 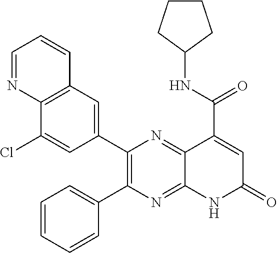 [SMILES: O=C(NC1CCCC1)c1cc(=O)[nH]c2nc(-c3ccccc3)c(-c3cc(Cl)c4ncccc4c3)nc12] |
| 1.838 | 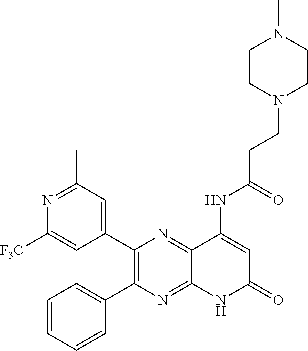 [SMILES: Cc1cc(-c2nc3c(NC(=O)CCN4CCN(C)CC4)cc(=O)[nH]c3nc2-c2ccccc2)cc(C(F)(F)F)n1] |
| 1.839 | 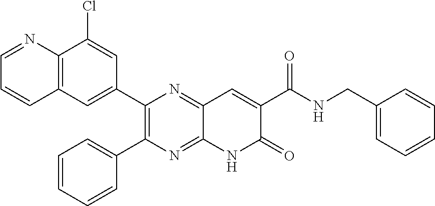 [SMILES: O=C(NCc1ccccc1)c1cc2nc(-c3cc(Cl)c4ncccc4c3)c(-c3ccccc3)nc2[nH]c1=O] |
| 1.840 | 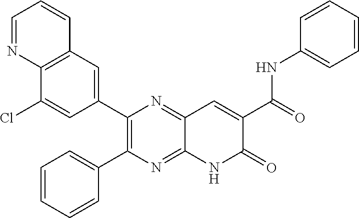 [SMILES: O=C(Nc1ccccc1)c1cc2nc(-c3cc(Cl)c4ncccc4c3)c(-c3ccccc3)nc2[nH]c1=O] |
| 1.841 | 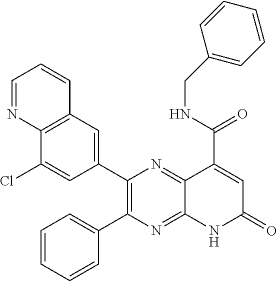 [SMILES: O=C(NCc1ccccc1)c1cc(=O)[nH]c2nc(-c3ccccc3)c(-c3cc(Cl)c4ncccc4c3)nc12] |
| 1.842 | 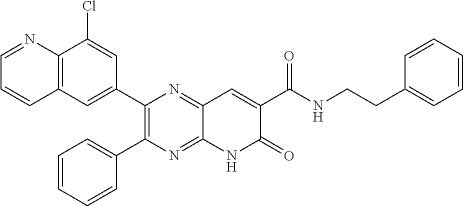 [SMILES: O=C(NCCc1ccccc1)c1cc2nc(-c3cc(Cl)c4ncccc4c3)c(-c3ccccc3)nc2[nH]c1=O] |
| 1.843 | 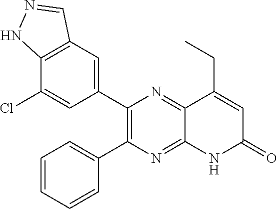 [SMILES: CCc1cc(=O)[nH]c2nc(-c3ccccc3)c(-c3cc(Cl)c4[nH]ncc4c3)nc12] |
| 1.844 | 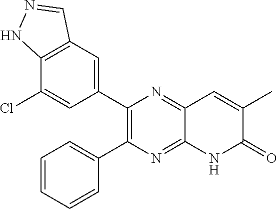 [SMILES: Cc1cc2nc(-c3cc(Cl)c4[nH]ncc4c3)c(-c3ccccc3)nc2[nH]c1=O] |
| 1.845 | 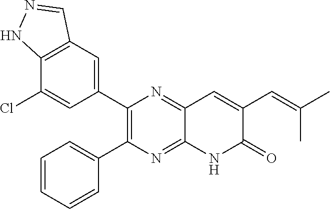 [SMILES: CC(C)=Cc1cc2nc(-c3cc(Cl)c4[nH]ncc4c3)c(-c3ccccc3)nc2[nH]c1=O] |
| 1.846 | 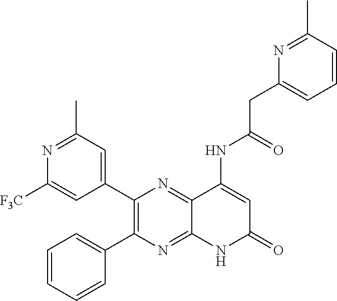 [SMILES: Cc1cccc(CC(=O)Nc2cc(=O)[nH]c3nc(-c4ccccc4)c(-c4cc(C)nc(C(F)(F)F)c4)nc23)n1] |
| 1.847 | 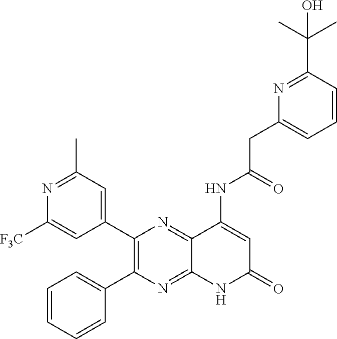 [SMILES: Cc1cc(-c2nc3c(NC(=O)Cc4cccc(C(C)(C)O)n4)cc(=O)[nH]c3nc2-c2ccccc2)cc(C(F)(F)F)n1] |
| 1.848 | 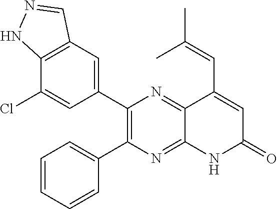 [SMILES: CC(C)=Cc1cc(=O)[nH]c2nc(-c3ccccc3)c(-c3cc(Cl)c4[nH]ncc4c3)nc12] |
| 1.849 | 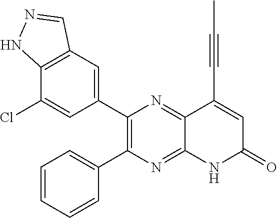 [SMILES: CC#Cc1cc(=O)[nH]c2nc(-c3ccccc3)c(-c3cc(Cl)c4[nH]ncc4c3)nc12] |
| 1.850 | 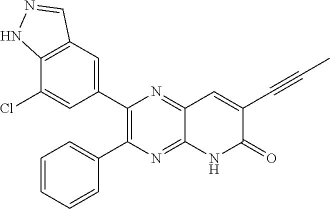 [SMILES: CC#Cc1cc2nc(-c3cc(Cl)c4[nH]ncc4c3)c(-c3ccccc3)nc2[nH]c1=O] |
| 1.851 | 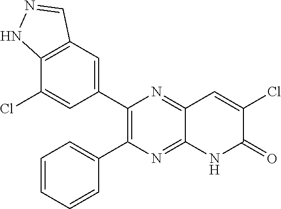 [SMILES: O=c1[nH]c2nc(-c3ccccc3)c(-c3cc(Cl)c4[nH]ncc4c3)nc2cc1Cl] |
| 1.852 | 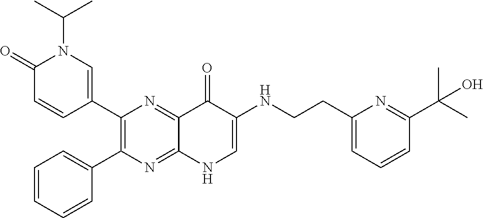 [SMILES: CC(C)n1cc(-c2nc3c(=O)c(NCCc4cccc(C(C)(C)O)n4)c[nH]c3nc2-c2ccccc2)ccc1=O] |
| 1.853 | 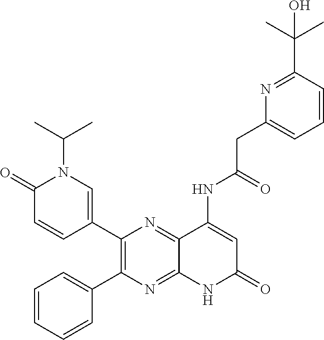 [SMILES: CC(C)n1cc(-c2nc3c(NC(=O)Cc4cccc(C(C)(C)O)n4)cc(=O)[nH]c3nc2-c2ccccc2)ccc1=O] |
| 1.854 | 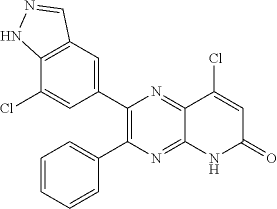 [SMILES: O=c1cc(Cl)c2nc(-c3cc(Cl)c4[nH]ncc4c3)c(-c3ccccc3)nc2[nH]1] |
| 1.855 | 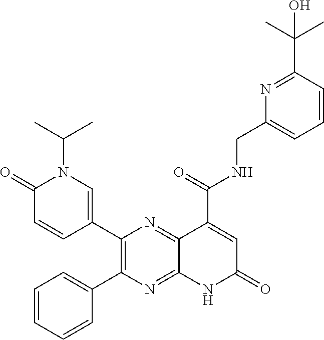 [SMILES: CC(C)n1cc(-c2nc3c(C(=O)NCc4cccc(C(C)(C)O)n4)cc(=O)[nH]c3nc2-c2ccccc2)ccc1=O] |
| 1.856 | 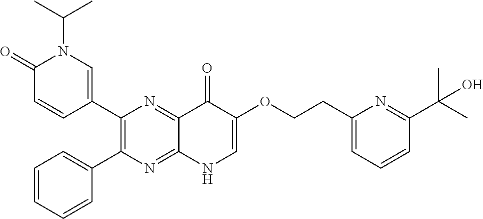 [SMILES: CC(C)n1cc(-c2nc3c(=O)c(OCCc4cccc(C(C)(C)O)n4)c[nH]c3nc2-c2ccccc2)ccc1=O] |
| 1.857 | 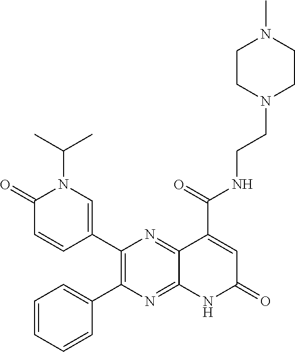 [SMILES: CC(C)n1cc(-c2nc3c(C(=O)NCCN4CCN(C)CC4)cc(=O)[nH]c3nc2-c2ccccc2)ccc1=O] |
| 1.858 | 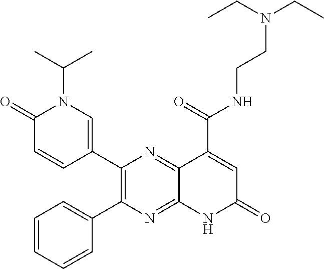 [SMILES: CCN(CC)CCNC(=O)c1cc(=O)[nH]c2nc(-c3ccccc3)c(-c3ccc(=O)n(C(C)C)c3)nc12] |
| 1.859 | 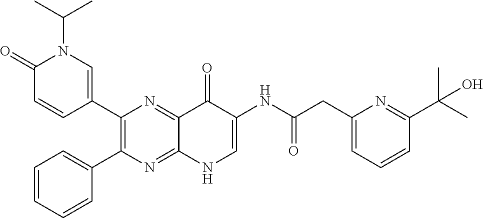 [SMILES: CC(C)n1cc(-c2nc3c(=O)c(NC(=O)Cc4cccc(C(C)(C)O)n4)c[nH]c3nc2-c2ccccc2)ccc1=O] |
| 1.860 | 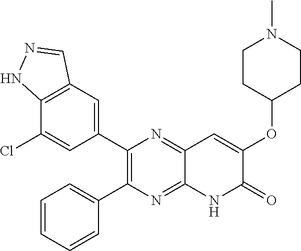 [SMILES: CN1CCC(Oc2cc3nc(-c4cc(Cl)c5[nH]ncc5c4)c(-c4ccccc4)nc3[nH]c2=O)CC1] |
| 1.861 | 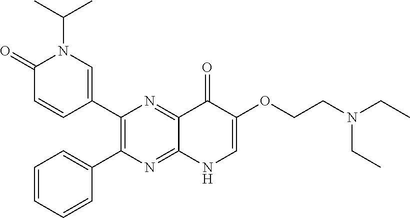 [SMILES: CCN(CC)CCOc1c[nH]c2nc(-c3ccccc3)c(-c3ccc(=O)n(C(C)C)c3)nc2c1=O] |
| 1.862 | 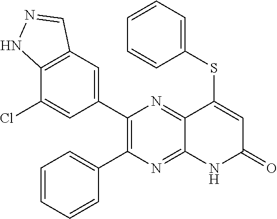 [SMILES: O=c1cc(Sc2ccccc2)c2nc(-c3cc(Cl)c4[nH]ncc4c3)c(-c3ccccc3)nc2[nH]1] |
| 1.863 | 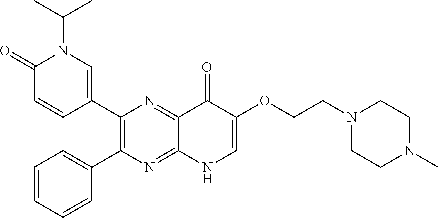 [SMILES: CC(C)n1cc(-c2nc3c(=O)c(OCCN4CCN(C)CC4)c[nH]c3nc2-c2ccccc2)ccc1=O] |
| 1.864 | 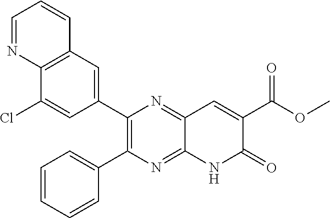 [SMILES: COC(=O)c1cc2nc(-c3cc(Cl)c4ncccc4c3)c(-c3ccccc3)nc2[nH]c1=O] |
| 1.865 | 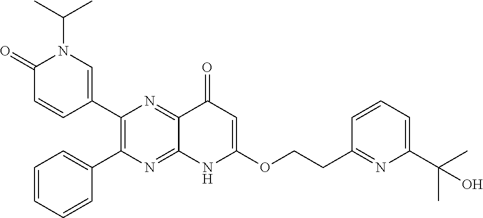 [SMILES: CC(C)n1cc(-c2nc3c(=O)cc(OCCc4cccc(C(C)(C)O)n4)[nH]c3nc2-c2ccccc2)ccc1=O] |
| 1.866 | 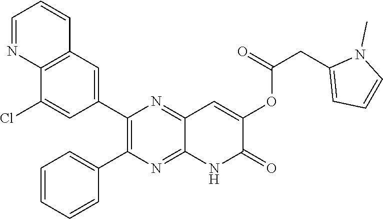 [SMILES: Cn1cccc1CC(=O)Oc1cc2nc(-c3cc(Cl)c4ncccc4c3)c(-c3ccccc3)nc2[nH]c1=O] |
| 1.867 | 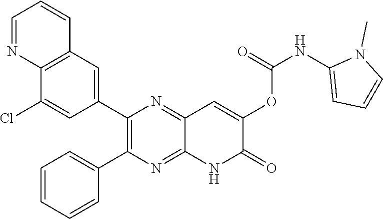 [SMILES: Cn1cccc1NC(=O)Oc1cc2nc(-c3cc(Cl)c4ncccc4c3)c(-c3ccccc3)nc2[nH]c1=O] |
| 1.868 | 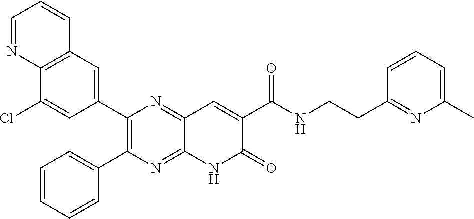 [SMILES: Cc1cccc(CCNC(=O)c2cc3nc(-c4cc(Cl)c5ncccc5c4)c(-c4ccccc4)nc3[nH]c2=O)n1] |
| 1.869 | 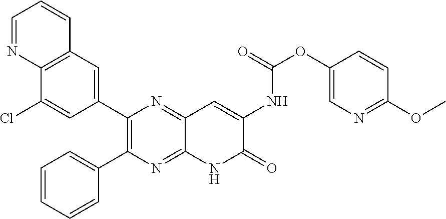 [SMILES: COc1ccc(OC(=O)Nc2cc3nc(-c4cc(Cl)c5ncccc5c4)c(-c4ccccc4)nc3[nH]c2=O)cn1] |
| 1.870 | 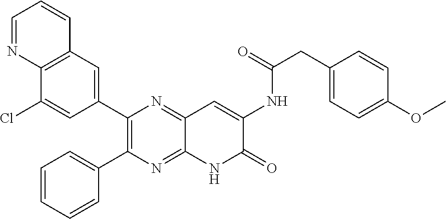 [SMILES: COc1ccc(CC(=O)Nc2cc3nc(-c4cc(Cl)c5ncccc5c4)c(-c4ccccc4)nc3[nH]c2=O)cc1] |
| 1.871 | 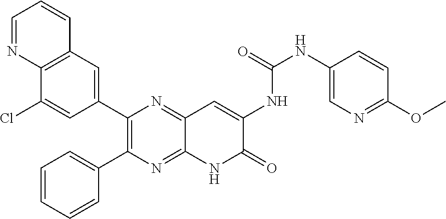 [SMILES: COc1ccc(NC(=O)Nc2cc3nc(-c4cc(Cl)c5ncccc5c4)c(-c4ccccc4)nc3[nH]c2=O)cn1] |
| 1.872 | 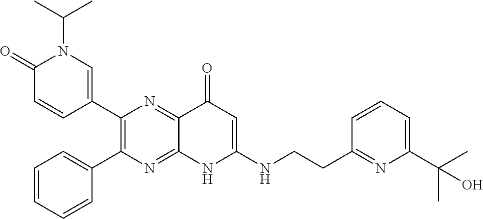 [SMILES: CC(C)n1cc(-c2nc3c(=O)cc(NCCc4cccc(C(C)(C)O)n4)[nH]c3nc2-c2ccccc2)ccc1=O] |
| 1.873 | 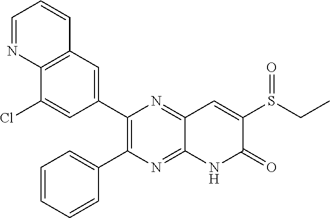 [SMILES: CCS(=O)c1cc2nc(-c3cc(Cl)c4ncccc4c3)c(-c3ccccc3)nc2[nH]c1=O] |
| 1.874 | 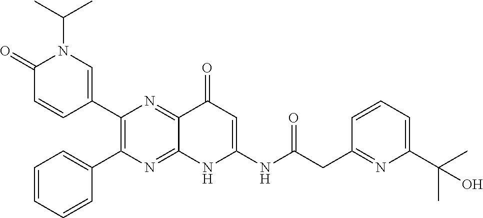 [SMILES: CC(C)n1cc(-c2nc3c(=O)cc(NC(=O)Cc4cccc(C(C)(C)O)n4)[nH]c3nc2-c2ccccc2)ccc1=O] |
| 1.875 | 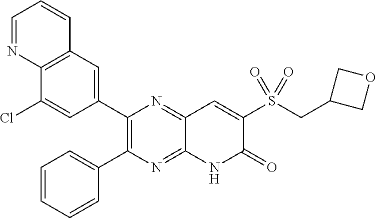 [SMILES: O=c1[nH]c2nc(-c3ccccc3)c(-c3cc(Cl)c4ncccc4c3)nc2cc1S(=O)(=O)CC1COC1] |
| 1.876 | 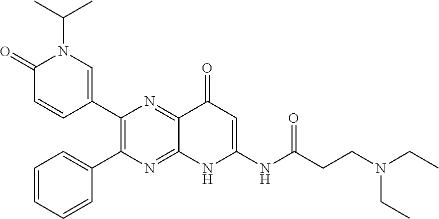 [SMILES: CCN(CC)CCC(=O)Nc1cc(=O)c2nc(-c3ccc(=O)n(C(C)C)c3)c(-c3ccccc3)nc2[nH]1] |
| 1.877 | 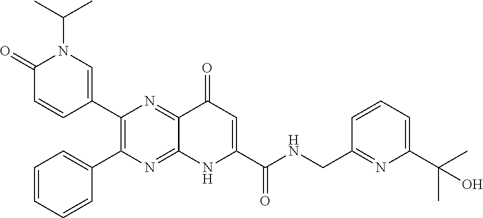 [SMILES: CC(C)n1cc(-c2nc3c(=O)cc(C(=O)NCc4cccc(C(C)(C)O)n4)[nH]c3nc2-c2ccccc2)ccc1=O] |
| 1.878 | 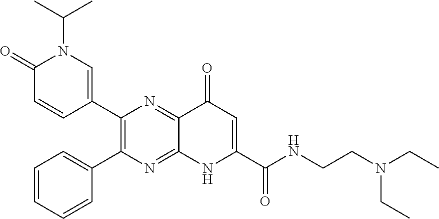 [SMILES: CCN(CC)CCNC(=O)c1cc(=O)c2nc(-c3ccc(=O)n(C(C)C)c3)c(-c3ccccc3)nc2[nH]1] |
| 1.879 | 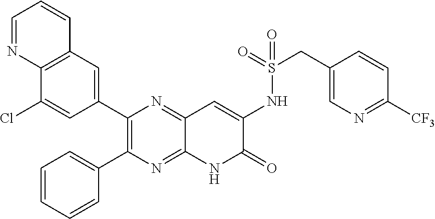 [SMILES: O=c1[nH]c2nc(-c3ccccc3)c(-c3cc(Cl)c4ncccc4c3)nc2cc1NS(=O)(=O)Cc1ccc(C(F)(F)F)nc1] |
| 1.880 | 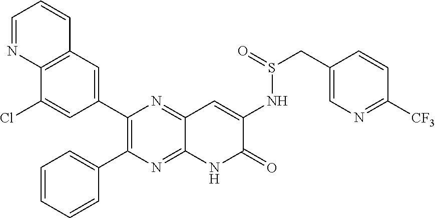 [SMILES: O=c1[nH]c2nc(-c3ccccc3)c(-c3cc(Cl)c4ncccc4c3)nc2cc1NS(=O)Cc1ccc(C(F)(F)F)nc1] |
| 1.881 | 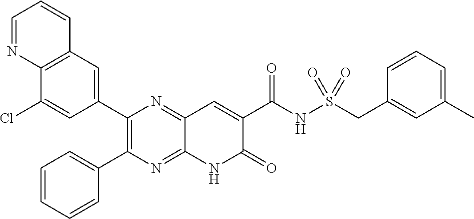 [SMILES: Cc1cccc(CS(=O)(=O)NC(=O)c2cc3nc(-c4cc(Cl)c5ncccc5c4)c(-c4ccccc4)nc3[nH]c2=O)c1] |
| 1.882 | 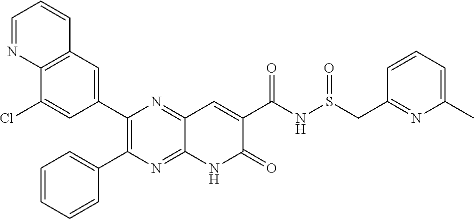 [SMILES: Cc1cccc(CS(=O)NC(=O)c2cc3nc(-c4cc(Cl)c5ncccc5c4)c(-c4ccccc4)nc3[nH]c2=O)n1] |
| 1.883 | 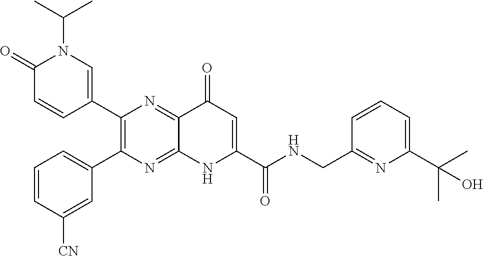 [SMILES: CC(C)n1cc(-c2nc3c(=O)cc(C(=O)NCc4cccc(C(C)(C)O)n4)[nH]c3nc2-c2cccc(C#N)c2)ccc1=O] |
| 1.884 | 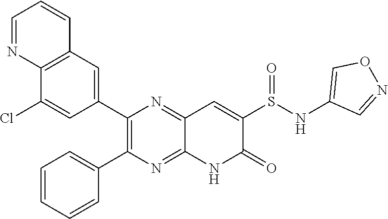 [SMILES: O=c1[nH]c2nc(-c3ccccc3)c(-c3cc(Cl)c4ncccc4c3)nc2cc1S(=O)Nc1cnoc1] |
| 1.885 | 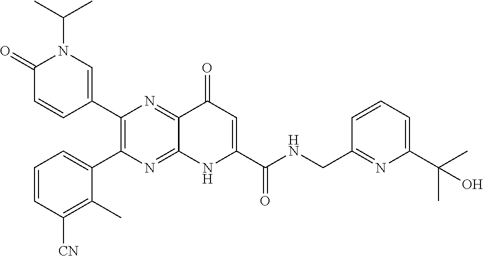 [SMILES: Cc1c(C#N)cccc1-c1nc2[nH]c(C(=O)NCc3cccc(C(C)(C)O)n3)cc(=O)c2nc1-c1ccc(=O)n(C(C)C)c1] |
| 1.886 | 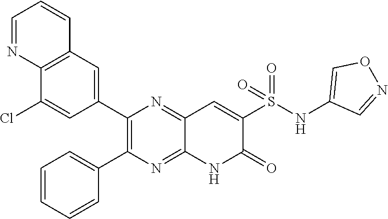 [SMILES: O=c1[nH]c2nc(-c3ccccc3)c(-c3cc(Cl)c4ncccc4c3)nc2cc1S(=O)(=O)Nc1cnoc1] |
| 1.887 | 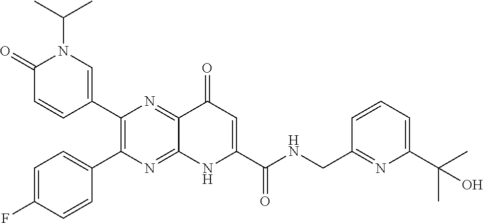 [SMILES: CC(C)n1cc(-c2nc3c(=O)cc(C(=O)NCc4cccc(C(C)(C)O)n4)[nH]c3nc2-c2ccc(F)cc2)ccc1=O] |
| 1.888 | 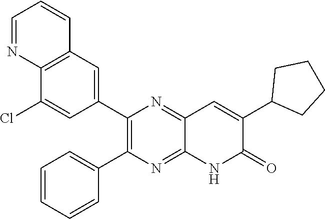 [SMILES: O=c1[nH]c2nc(-c3ccccc3)c(-c3cc(Cl)c4ncccc4c3)nc2cc1C1CCCC1] |
| 1.889 | 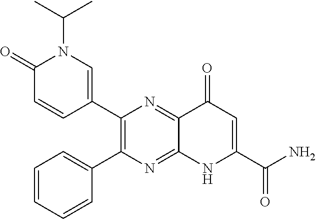 [SMILES: CC(C)n1cc(-c2nc3c(=O)cc(C(N)=O)[nH]c3nc2-c2ccccc2)ccc1=O] |
| 1.890 | 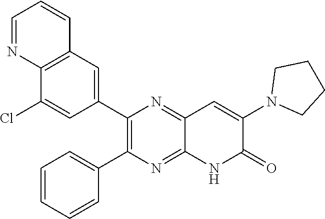 [SMILES: O=c1[nH]c2nc(-c3ccccc3)c(-c3cc(Cl)c4ncccc4c3)nc2cc1N1CCCC1] |
| 1.891 | 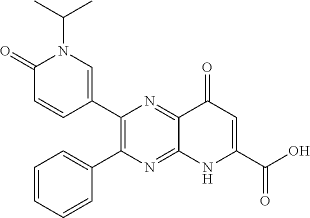 [SMILES: CC(C)n1cc(-c2nc3c(=O)cc(C(=O)O)[nH]c3nc2-c2ccccc2)ccc1=O] |
| 1.892 | 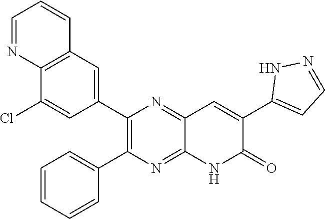 [SMILES: O=c1[nH]c2nc(-c3ccccc3)c(-c3cc(Cl)c4ncccc4c3)nc2cc1-c1ccn[nH]1] |
| 1.893 | 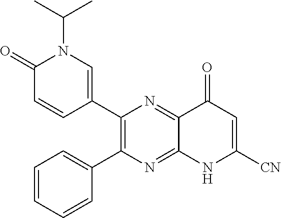 [SMILES: CC(C)n1cc(-c2nc3c(=O)cc(C#N)[nH]c3nc2-c2ccccc2)ccc1=O] |
| 1.894 | 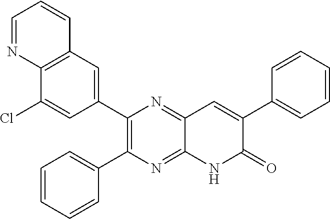 [SMILES: O=c1[nH]c2nc(-c3ccccc3)c(-c3cc(Cl)c4ncccc4c3)nc2cc1-c1ccccc1] |
| 1.895 | 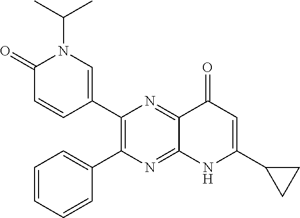 [SMILES: CC(C)n1cc(-c2nc3c(=O)cc(C4CC4)[nH]c3nc2-c2ccccc2)ccc1=O] |
| 1.896 | 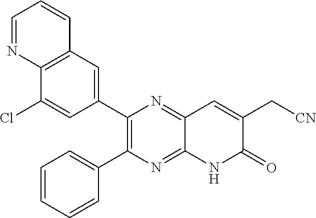 [SMILES: N#CCc1cc2nc(-c3cc(Cl)c4ncccc4c3)c(-c3ccccc3)nc2[nH]c1=O] |
| 1.897 | 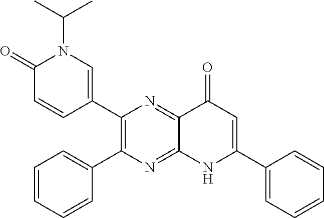 [SMILES: CC(C)n1cc(-c2nc3c(=O)cc(-c4ccccc4)[nH]c3nc2-c2ccccc2)ccc1=O] |
| 1.898 | 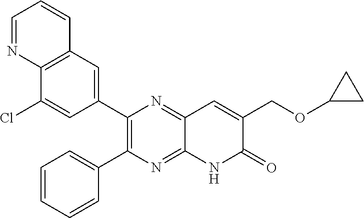 [SMILES: O=c1[nH]c2nc(-c3ccccc3)c(-c3cc(Cl)c4ncccc4c3)nc2cc1COC1CC1] |
| 1.899 | 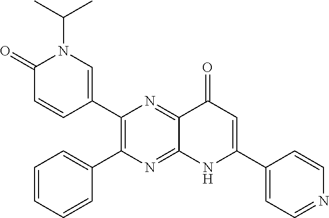 [SMILES: CC(C)n1cc(-c2nc3c(=O)cc(-c4ccncc4)[nH]c3nc2-c2ccccc2)ccc1=O] |
| 1.900 | 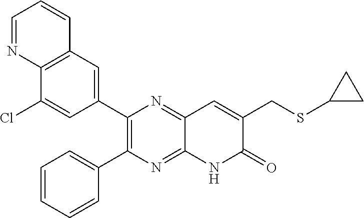 [SMILES: O=c1[nH]c2nc(-c3ccccc3)c(-c3cc(Cl)c4ncccc4c3)nc2cc1CSC1CC1] |
| 1.901 | 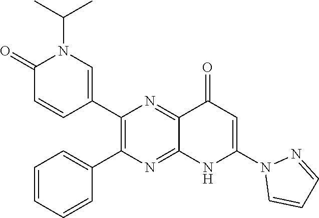 [SMILES: CC(C)n1cc(-c2nc3c(=O)cc(-n4cccn4)[nH]c3nc2-c2ccccc2)ccc1=O] |
| 1.902 | 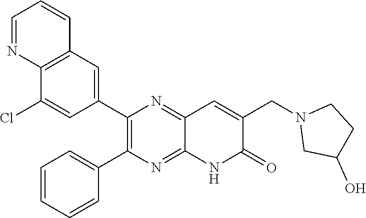 [SMILES: O=c1[nH]c2nc(-c3ccccc3)c(-c3cc(Cl)c4ncccc4c3)nc2cc1CN1CCC(O)C1] |
| 1.903 | 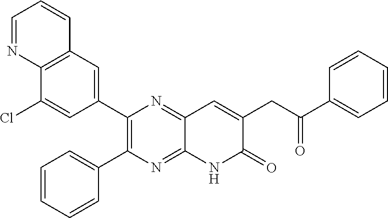 [SMILES: O=C(Cc1cc2nc(-c3cc(Cl)c4ncccc4c3)c(-c3ccccc3)nc2[nH]c1=O)c1ccccc1] |
| 1.904 | 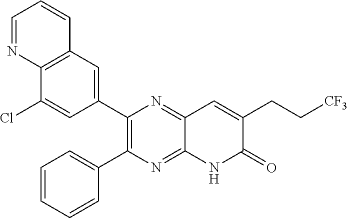 [SMILES: O=c1[nH]c2nc(-c3ccccc3)c(-c3cc(Cl)c4ncccc4c3)nc2cc1CCC(F)(F)F] |
| 1.905 | 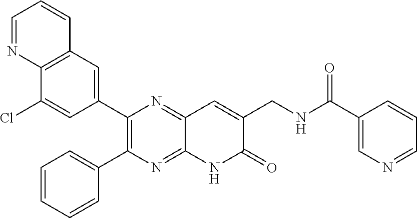 [SMILES: O=C(NCc1cc2nc(-c3cc(Cl)c4ncccc4c3)c(-c3ccccc3)nc2[nH]c1=O)c1cccnc1] |
| 1.906 | 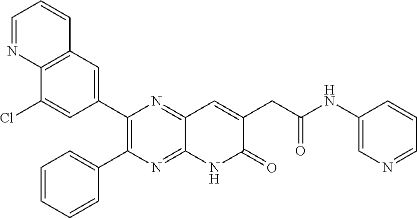 [SMILES: O=C(Cc1cc2nc(-c3cc(Cl)c4ncccc4c3)c(-c3ccccc3)nc2[nH]c1=O)Nc1cccnc1] |
| 1.907 | 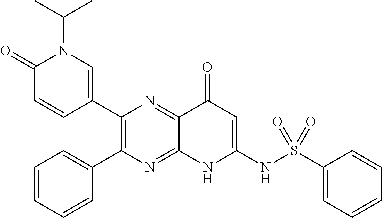 [SMILES: CC(C)n1cc(-c2nc3c(=O)cc(NS(=O)(=O)c4ccccc4)[nH]c3nc2-c2ccccc2)ccc1=O] |
| 1.908 | 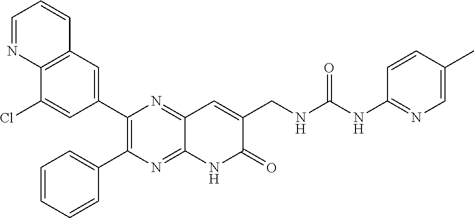 [SMILES: Cc1ccc(NC(=O)NCc2cc3nc(-c4cc(Cl)c5ncccc5c4)c(-c4ccccc4)nc3[nH]c2=O)nc1] |
| 1.909 | 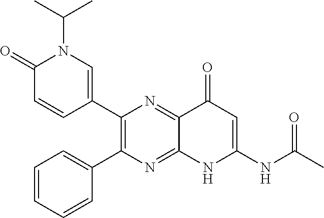 [SMILES: CC(=O)Nc1cc(=O)c2nc(-c3ccc(=O)n(C(C)C)c3)c(-c3ccccc3)nc2[nH]1] |
| 1.910 | 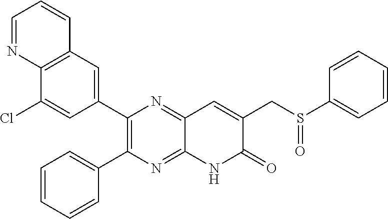 [SMILES: O=c1[nH]c2nc(-c3ccccc3)c(-c3cc(Cl)c4ncccc4c3)nc2cc1CS(=O)c1ccccc1] |
| 1.911 | 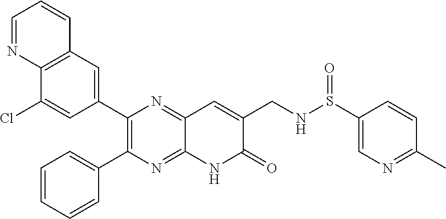 [SMILES: Cc1ccc(S(=O)NCc2cc3nc(-c4cc(Cl)c5ncccc5c4)c(-c4ccccc4)nc3[nH]c2=O)cn1] |
| 1.912 | 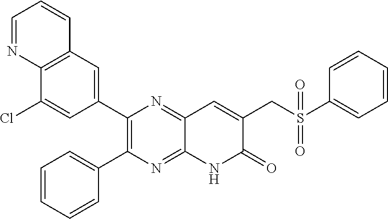 [SMILES: O=c1[nH]c2nc(-c3ccccc3)c(-c3cc(Cl)c4ncccc4c3)nc2cc1CS(=O)(=O)c1ccccc1] |
| 1.913 | 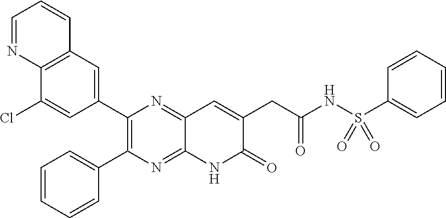 [SMILES: O=C(Cc1cc2nc(-c3cc(Cl)c4ncccc4c3)c(-c3ccccc3)nc2[nH]c1=O)NS(=O)(=O)c1ccccc1] |
| 1.914 | 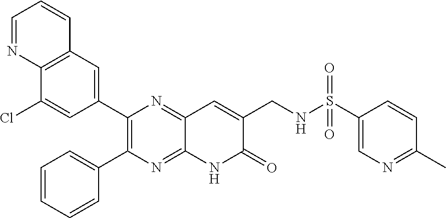 [SMILES: Cc1ccc(S(=O)(=O)NCc2cc3nc(-c4cc(Cl)c5ncccc5c4)c(-c4ccccc4)nc3[nH]c2=O)cn1] |
| 1.915 | 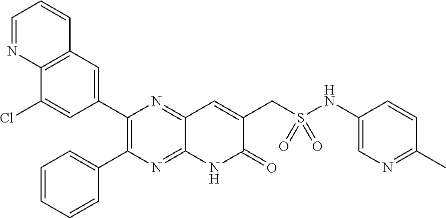 [SMILES: Cc1ccc(NS(=O)(=O)Cc2cc3nc(-c4cc(Cl)c5ncccc5c4)c(-c4ccccc4)nc3[nH]c2=O)cn1] |
| 1.916 | 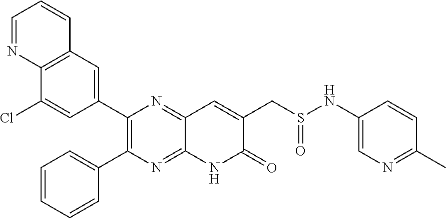 [SMILES: Cc1ccc(NS(=O)Cc2cc3nc(-c4cc(Cl)c5ncccc5c4)c(-c4ccccc4)nc3[nH]c2=O)cn1] |
| 1.917 | 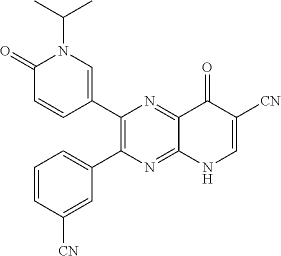 [SMILES: CC(C)n1cc(-c2nc3c(=O)c(C#N)c[nH]c3nc2-c2cccc(C#N)c2)ccc1=O] |
| 1.918 | 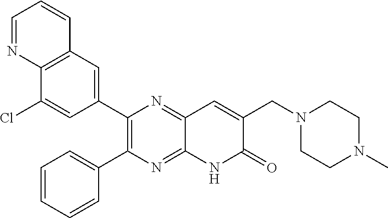 [SMILES: CN1CCN(Cc2cc3nc(-c4cc(Cl)c5ncccc5c4)c(-c4ccccc4)nc3[nH]c2=O)CC1] |
| 1.919 | 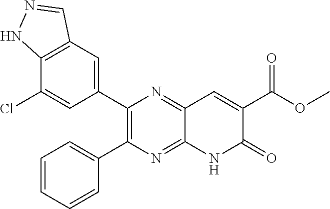 [SMILES: COC(=O)c1cc2nc(-c3cc(Cl)c4[nH]ncc4c3)c(-c3ccccc3)nc2[nH]c1=O] |
| 1.920 | 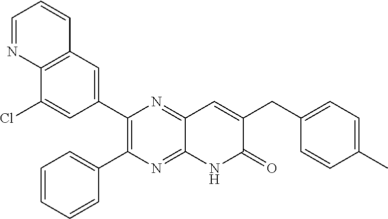 [SMILES: Cc1ccc(Cc2cc3nc(-c4cc(Cl)c5ncccc5c4)c(-c4ccccc4)nc3[nH]c2=O)cc1] |
| 1.921 | 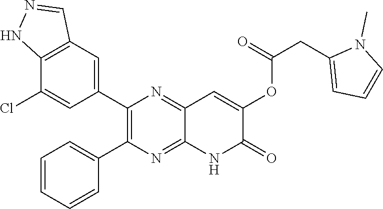 [SMILES: Cn1cccc1CC(=O)Oc1cc2nc(-c3cc(Cl)c4[nH]ncc4c3)c(-c3ccccc3)nc2[nH]c1=O] |
| 1.922 | 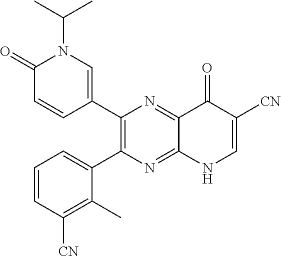 [SMILES: Cc1c(C#N)cccc1-c1nc2[nH]cc(C#N)c(=O)c2nc1-c1ccc(=O)n(C(C)C)c1] |
| 1.923 | 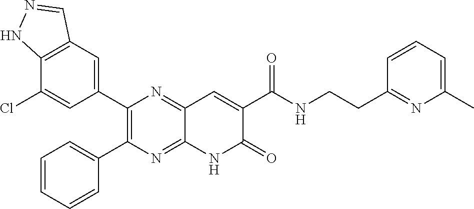 [SMILES: Cc1cccc(CCNC(=O)c2cc3nc(-c4cc(Cl)c5[nH]ncc5c4)c(-c4ccccc4)nc3[nH]c2=O)n1] |
| 1.924 | 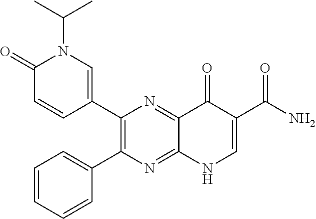 [SMILES: CC(C)n1cc(-c2nc3c(=O)c(C(N)=O)c[nH]c3nc2-c2ccccc2)ccc1=O] |
| 1.925 | 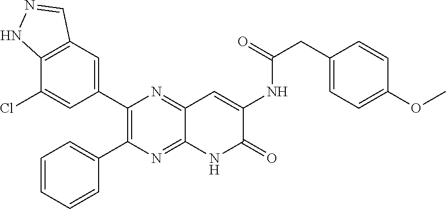 [SMILES: COc1ccc(CC(=O)Nc2cc3nc(-c4cc(Cl)c5[nH]ncc5c4)c(-c4ccccc4)nc3[nH]c2=O)cc1] |
| 1.926 | 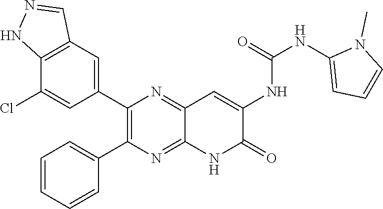 [SMILES: Cn1cccc1NC(=O)Nc1cc2nc(-c3cc(Cl)c4[nH]ncc4c3)c(-c3ccccc3)nc2[nH]c1=O] |
| 1.927 | 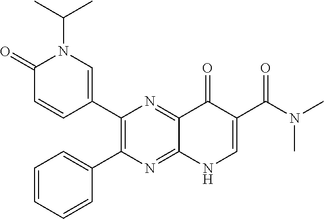 [SMILES: CC(C)n1cc(-c2nc3c(=O)c(C(=O)N(C)C)c[nH]c3nc2-c2ccccc2)ccc1=O] |
| 1.928 | 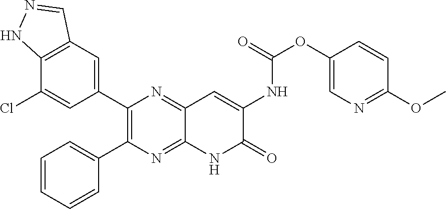 [SMILES: COc1ccc(OC(=O)Nc2cc3nc(-c4cc(Cl)c5[nH]ncc5c4)c(-c4ccccc4)nc3[nH]c2=O)cn1] |
| 1.929 | 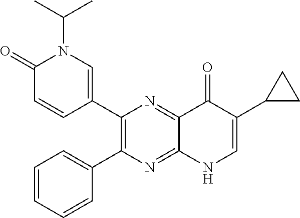 [SMILES: CC(C)n1cc(-c2nc3c(=O)c(C4CC4)c[nH]c3nc2-c2ccccc2)ccc1=O] |
| 1.930 | 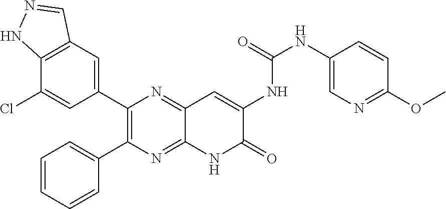 [SMILES: COc1ccc(NC(=O)Nc2cc3nc(-c4cc(Cl)c5[nH]ncc5c4)c(-c4ccccc4)nc3[nH]c2=O)cn1] |
| 1.931 | 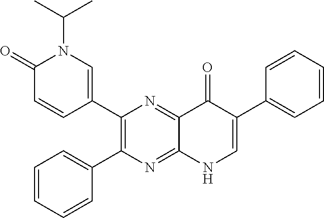 [SMILES: CC(C)n1cc(-c2nc3c(=O)c(-c4ccccc4)c[nH]c3nc2-c2ccccc2)ccc1=O] |
| 1.932 | 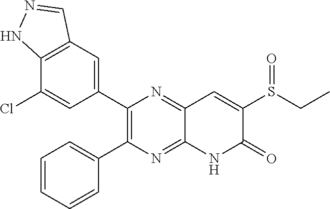 [SMILES: CCS(=O)c1cc2nc(-c3cc(Cl)c4[nH]ncc4c3)c(-c3ccccc3)nc2[nH]c1=O] |
| 1.933 | 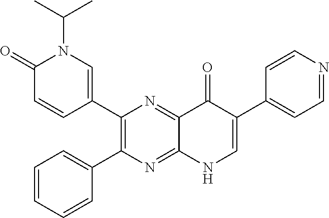 [SMILES: CC(C)n1cc(-c2nc3c(=O)c(-c4ccncc4)c[nH]c3nc2-c2ccccc2)ccc1=O] |
| 1.934 | 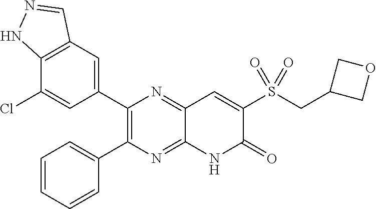 [SMILES: O=c1[nH]c2nc(-c3ccccc3)c(-c3cc(Cl)c4[nH]ncc4c3)nc2cc1S(=O)(=O)CC1COC1] |
| 1.935 | 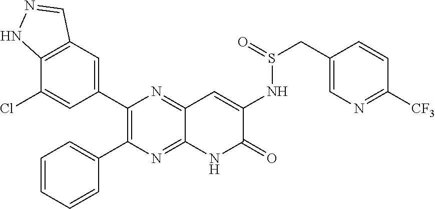 [SMILES: O=c1[nH]c2nc(-c3ccccc3)c(-c3cc(Cl)c4[nH]ncc4c3)nc2cc1NS(=O)Cc1ccc(C(F)(F)F)nc1] |
| 1.936 | 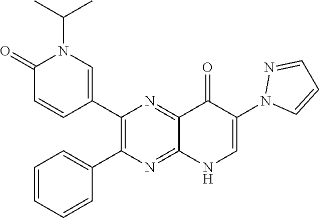 [SMILES: CC(C)n1cc(-c2nc3c(=O)c(-n4cccn4)c[nH]c3nc2-c2ccccc2)ccc1=O] |
| 1.937 | 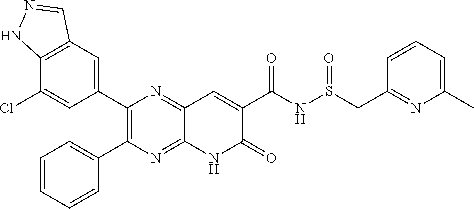 [SMILES: Cc1cccc(CS(=O)NC(=O)c2cc3nc(-c4cc(Cl)c5[nH]ncc5c4)c(-c4ccccc4)nc3[nH]c2=O)n1] |
| 1.938 | 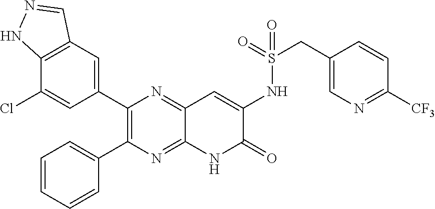 [SMILES: O=c1[nH]c2nc(-c3ccccc3)c(-c3cc(Cl)c4[nH]ncc4c3)nc2cc1NS(=O)(=O)Cc1ccc(C(F)(F)F)nc1] |
| 1.939 | 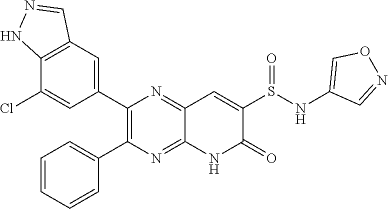 [SMILES: O=c1[nH]c2nc(-c3ccccc3)c(-c3cc(Cl)c4[nH]ncc4c3)nc2cc1S(=O)Nc1cnoc1] |
| 1.940 | 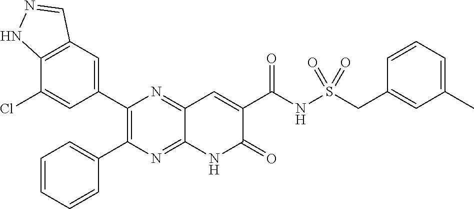 [SMILES: Cc1cccc(CS(=O)(=O)NC(=O)c2cc3nc(-c4cc(Cl)c5[nH]ncc5c4)c(-c4ccccc4)nc3[nH]c2=O)c1] |
| 1.941 | 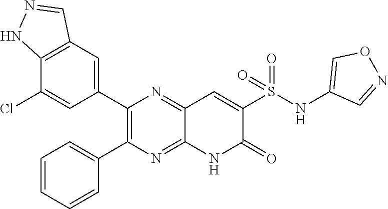 [SMILES: O=c1[nH]c2nc(-c3ccccc3)c(-c3cc(Cl)c4[nH]ncc4c3)nc2cc1S(=O)(=O)Nc1cnoc1] |
| 1.942 | 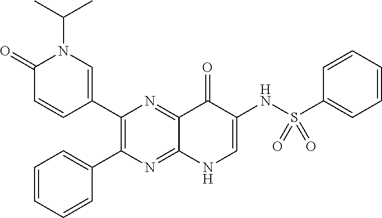 [SMILES: CC(C)n1cc(-c2nc3c(=O)c(NS(=O)(=O)c4ccccc4)c[nH]c3nc2-c2ccccc2)ccc1=O] |
| 1.943 | 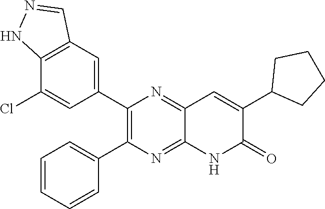 [SMILES: O=c1[nH]c2nc(-c3ccccc3)c(-c3cc(Cl)c4[nH]ncc4c3)nc2cc1C1CCCC1] |
| 1.944 | 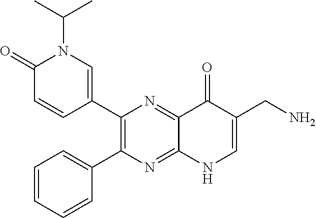 [SMILES: CC(C)n1cc(-c2nc3c(=O)c(CN)c[nH]c3nc2-c2ccccc2)ccc1=O] |
| 1.945 | 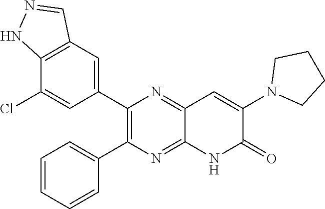 [SMILES: O=c1[nH]c2nc(-c3ccccc3)c(-c3cc(Cl)c4[nH]ncc4c3)nc2cc1N1CCCC1] |
| 1.946 | 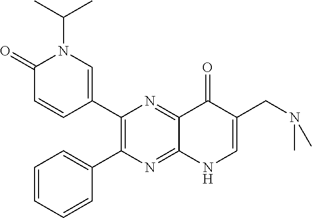 [SMILES: CC(C)n1cc(-c2nc3c(=O)c(CN(C)C)c[nH]c3nc2-c2ccccc2)ccc1=O] |
| 1.947 | 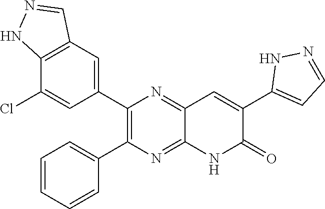 [SMILES: O=c1[nH]c2nc(-c3ccccc3)c(-c3cc(Cl)c4[nH]ncc4c3)nc2cc1-c1ccn[nH]1] |
| 1.948 | 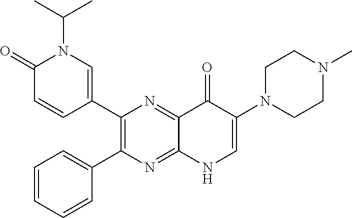 [SMILES: CC(C)n1cc(-c2nc3c(=O)c(N4CCN(C)CC4)c[nH]c3nc2-c2ccccc2)ccc1=O] |
| 1.949 | 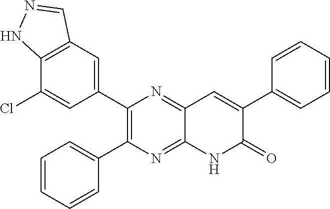 [SMILES: O=c1[nH]c2nc(-c3ccccc3)c(-c3cc(Cl)c4[nH]ncc4c3)nc2cc1-c1ccccc1] |
| 1.950 | 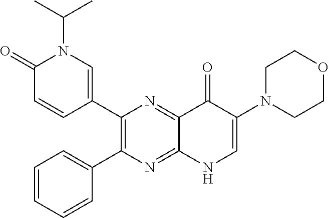 [SMILES: CC(C)n1cc(-c2nc3c(=O)c(N4CCOCC4)c[nH]c3nc2-c2ccccc2)ccc1=O] |
| 1.951 | 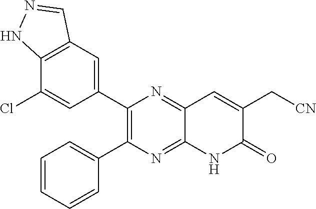 [SMILES: N#CCc1cc2nc(-c3cc(Cl)c4[nH]ncc4c3)c(-c3ccccc3)nc2[nH]c1=O] |
| 1.952 | 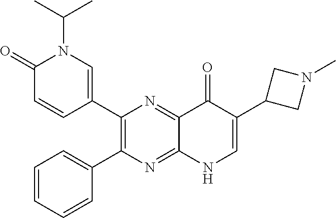 [SMILES: CC(C)n1cc(-c2nc3c(=O)c(C4CN(C)C4)c[nH]c3nc2-c2ccccc2)ccc1=O] |
| 1.953 | 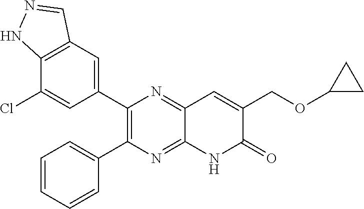 [SMILES: O=c1[nH]c2nc(-c3ccccc3)c(-c3cc(Cl)c4[nH]ncc4c3)nc2cc1COC1CC1] |
| 1.954 | 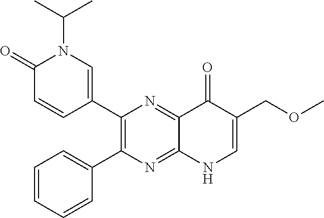 [SMILES: COCc1c[nH]c2nc(-c3ccccc3)c(-c3ccc(=O)n(C(C)C)c3)nc2c1=O] |
| 1.955 | 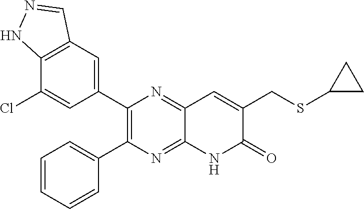 [SMILES: O=c1[nH]c2nc(-c3ccccc3)c(-c3cc(Cl)c4[nH]ncc4c3)nc2cc1CSC1CC1] |
| 1.956 | 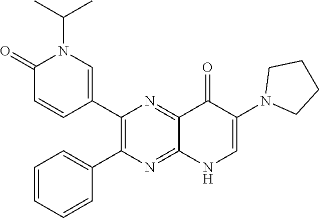 [SMILES: CC(C)n1cc(-c2nc3c(=O)c(N4CCCC4)c[nH]c3nc2-c2ccccc2)ccc1=O] |
| 1.957 | 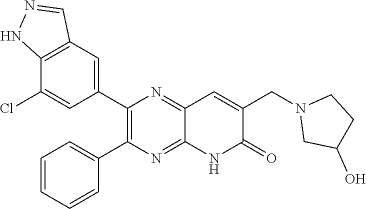 [SMILES: O=c1[nH]c2nc(-c3ccccc3)c(-c3cc(Cl)c4[nH]ncc4c3)nc2cc1CN1CCC(O)C1] |
| 1.958 | 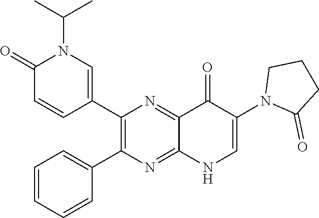 [SMILES: CC(C)n1cc(-c2nc3c(=O)c(N4CCCC4=O)c[nH]c3nc2-c2ccccc2)ccc1=O] |
| 1.959 | 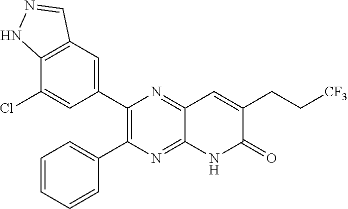 [SMILES: O=c1[nH]c2nc(-c3ccccc3)c(-c3cc(Cl)c4[nH]ncc4c3)nc2cc1CCC(F)(F)F] |
| 1.960 | 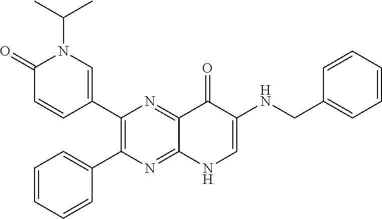 [SMILES: CC(C)n1cc(-c2nc3c(=O)c(NCc4ccccc4)c[nH]c3nc2-c2ccccc2)ccc1=O] |
| 1.961 | 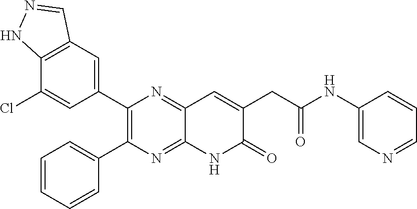 [SMILES: O=C(Cc1cc2nc(-c3cc(Cl)c4[nH]ncc4c3)c(-c3ccccc3)nc2[nH]c1=O)Nc1cccnc1] |
| 1.962 | 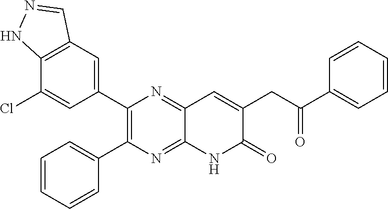 [SMILES: O=C(Cc1cc2nc(-c3cc(Cl)c4[nH]ncc4c3)c(-c3ccccc3)nc2[nH]c1=O)c1ccccc1] |
| 1.963 | 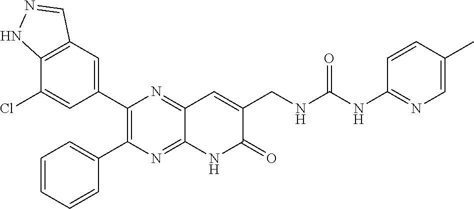 [SMILES: Cc1ccc(NC(=O)NCc2cc3nc(-c4cc(Cl)c5[nH]ncc5c4)c(-c4ccccc4)nc3[nH]c2=O)nc1] |
| 1.964 | 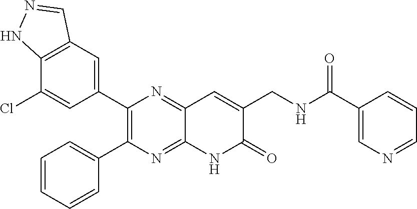 [SMILES: O=C(NCc1cc2nc(-c3cc(Cl)c4[nH]ncc4c3)c(-c3ccccc3)nc2[nH]c1=O)c1cccnc1] |
| 1.965 | 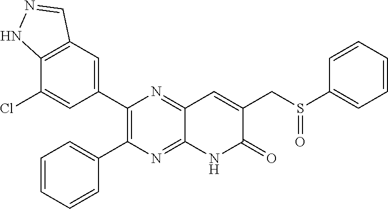 [SMILES: O=c1[nH]c2nc(-c3ccccc3)c(-c3cc(Cl)c4[nH]ncc4c3)nc2cc1CS(=O)c1ccccc1] |
| 1.966 | 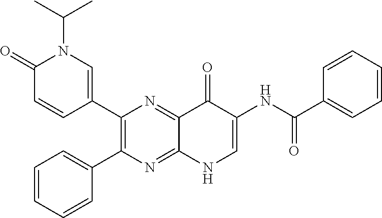 [SMILES: CC(C)n1cc(-c2nc3c(=O)c(NC(=O)c4ccccc4)c[nH]c3nc2-c2ccccc2)ccc1=O] |
| 1.967 | 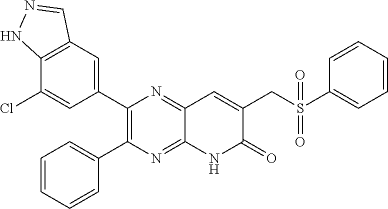 [SMILES: O=c1[nH]c2nc(-c3ccccc3)c(-c3cc(Cl)c4[nH]ncc4c3)nc2cc1CS(=O)(=O)c1ccccc1] |
| 1.968 | 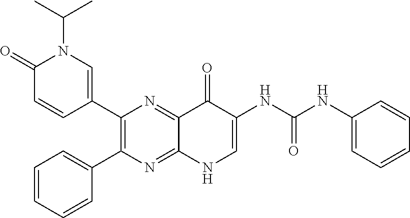 [SMILES: CC(C)n1cc(-c2nc3c(=O)c(NC(=O)Nc4ccccc4)c[nH]c3nc2-c2ccccc2)ccc1=O] |
| 1.969 | 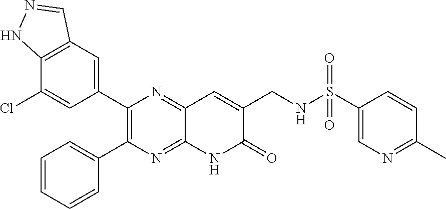 [SMILES: Cc1ccc(S(=O)(=O)NCc2cc3nc(-c4cc(Cl)c5[nH]ncc5c4)c(-c4ccccc4)nc3[nH]c2=O)cn1] |
| 1.970 | 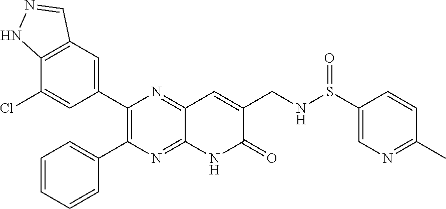 [SMILES: Cc1ccc(S(=O)NCc2cc3nc(-c4cc(Cl)c5[nH]ncc5c4)c(-c4ccccc4)nc3[nH]c2=O)cn1] |
| 1.971 | 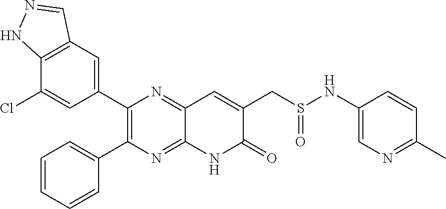 [SMILES: Cc1ccc(NS(=O)Cc2cc3nc(-c4cc(Cl)c5[nH]ncc5c4)c(-c4ccccc4)nc3[nH]c2=O)cn1] |
| 1.972 | 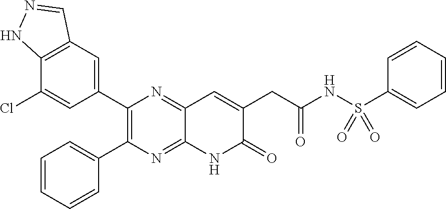 [SMILES: O=C(Cc1cc2nc(-c3cc(Cl)c4[nH]ncc4c3)c(-c3ccccc3)nc2[nH]c1=O)NS(=O)(=O)c1ccccc1] |
| 1.973 | 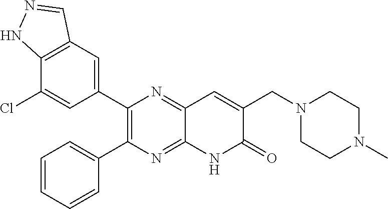 [SMILES: CN1CCN(Cc2cc3nc(-c4cc(Cl)c5[nH]ncc5c4)c(-c4ccccc4)nc3[nH]c2=O)CC1] |
| 1.974 | 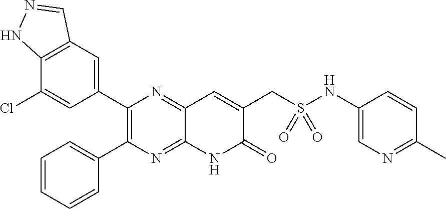 [SMILES: Cc1ccc(NS(=O)(=O)Cc2cc3nc(-c4cc(Cl)c5[nH]ncc5c4)c(-c4ccccc4)nc3[nH]c2=O)cn1] |
| 1.975 | 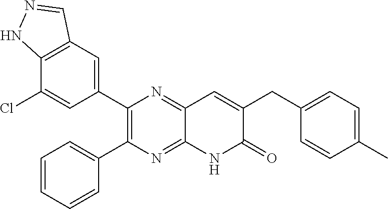 [SMILES: Cc1ccc(Cc2cc3nc(-c4cc(Cl)c5[nH]ncc5c4)c(-c4ccccc4)nc3[nH]c2=O)cc1] |
| 1.976 | 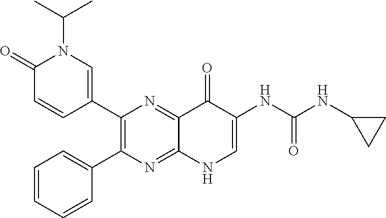 [SMILES: CC(C)n1cc(-c2nc3c(=O)c(NC(=O)NC4CC4)c[nH]c3nc2-c2ccccc2)ccc1=O] |
| 1.977 | 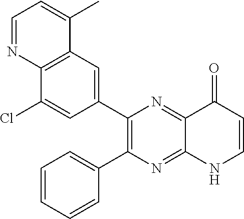 [SMILES: Cc1ccnc2c(Cl)cc(-c3nc4c(=O)cc[nH]c4nc3-c3ccccc3)cc12] |
| 1.978 | 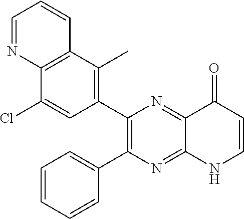 [SMILES: Cc1c(-c2nc3c(=O)cc[nH]c3nc2-c2ccccc2)cc(Cl)c2ncccc12] |
| 1.979 | 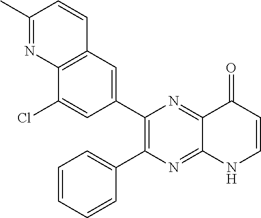 [SMILES: Cc1ccc2cc(-c3nc4c(=O)cc[nH]c4nc3-c3ccccc3)cc(Cl)c2n1] |
| 1.980 | 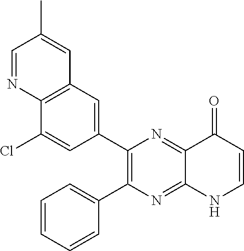 [SMILES: Cc1cnc2c(Cl)cc(-c3nc4c(=O)cc[nH]c4nc3-c3ccccc3)cc2c1] |
| 1.981 | 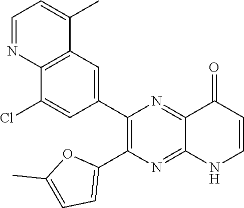 [SMILES: Cc1ccc(-c2nc3[nH]ccc(=O)c3nc2-c2cc(Cl)c3nccc(C)c3c2)o1] |
| 1.982 | 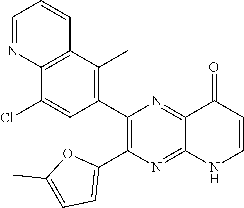 [SMILES: Cc1ccc(-c2nc3[nH]ccc(=O)c3nc2-c2cc(Cl)c3ncccc3c2C)o1] |
| 1.983 | 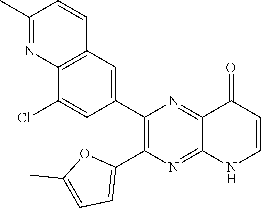 [SMILES: Cc1ccc2cc(-c3nc4c(=O)cc[nH]c4nc3-c3ccc(C)o3)cc(Cl)c2n1] |
| 1.984 | 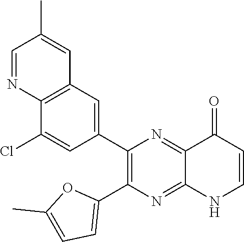 [SMILES: Cc1cnc2c(Cl)cc(-c3nc4c(=O)cc[nH]c4nc3-c3ccc(C)o3)cc2c1] |
| 1.985 | 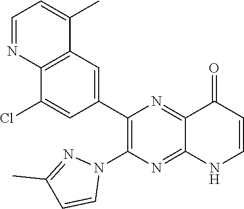 [SMILES: Cc1ccn(-c2nc3[nH]ccc(=O)c3nc2-c2cc(Cl)c3nccc(C)c3c2)n1] |
| 1.986 | 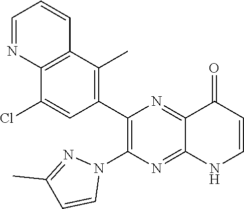 [SMILES: Cc1ccn(-c2nc3[nH]ccc(=O)c3nc2-c2cc(Cl)c3ncccc3c2C)n1] |
| 1.987 | 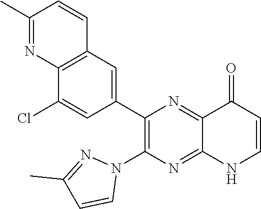 [SMILES: Cc1ccc2cc(-c3nc4c(=O)cc[nH]c4nc3-n3ccc(C)n3)cc(Cl)c2n1] |
| 1.988 | 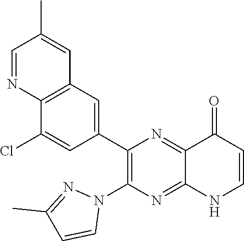 [SMILES: Cc1cnc2c(Cl)cc(-c3nc4c(=O)cc[nH]c4nc3-n3ccc(C)n3)cc2c1] |
| 1.989 | 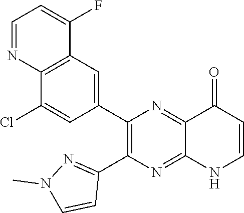 [SMILES: Cn1ccc(-c2nc3[nH]ccc(=O)c3nc2-c2cc(Cl)c3nccc(F)c3c2)n1] |
| 1.990 | 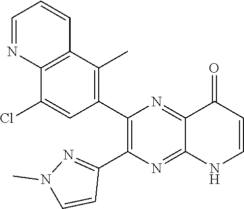 [SMILES: Cc1c(-c2nc3c(=O)cc[nH]c3nc2-c2ccn(C)n2)cc(Cl)c2ncccc12] |
| 1.991 | 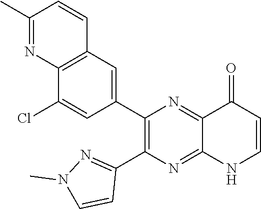 [SMILES: Cc1ccc2cc(-c3nc4c(=O)cc[nH]c4nc3-c3ccn(C)n3)cc(Cl)c2n1] |
| 1.992 | 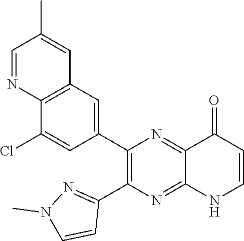 [SMILES: Cc1cnc2c(Cl)cc(-c3nc4c(=O)cc[nH]c4nc3-c3ccn(C)n3)cc2c1] |
| 1.993 | 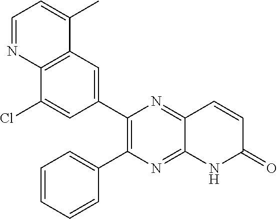 [SMILES: Cc1ccnc2c(Cl)cc(-c3nc4ccc(=O)[nH]c4nc3-c3ccccc3)cc12] |
| 1.994 | 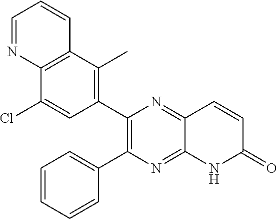 [SMILES: Cc1c(-c2nc3ccc(=O)[nH]c3nc2-c2ccccc2)cc(Cl)c2ncccc12] |
| 1.995 | 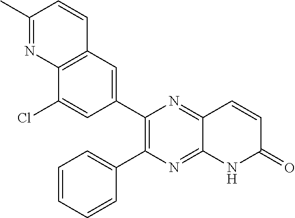 [SMILES: Cc1ccc2cc(-c3nc4ccc(=O)[nH]c4nc3-c3ccccc3)cc(Cl)c2n1] |
| 1.996 | 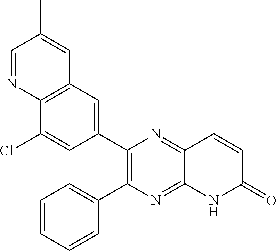 [SMILES: Cc1cnc2c(Cl)cc(-c3nc4ccc(=O)[nH]c4nc3-c3ccccc3)cc2c1] |
| 1.997 | 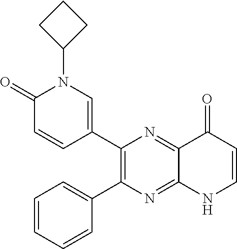 [SMILES: O=c1cc[nH]c2nc(-c3ccccc3)c(-c3ccc(=O)n(C4CCC4)c3)nc12] |
| 1.998 | 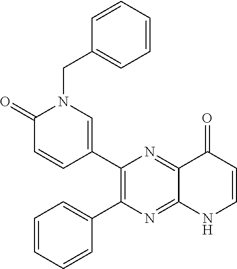 [SMILES: O=c1cc[nH]c2nc(-c3ccccc3)c(-c3ccc(=O)n(Cc4ccccc4)c3)nc12] |
| 1.999 | 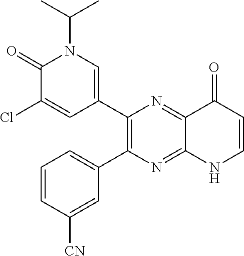 [SMILES: CC(C)n1cc(-c2nc3c(=O)cc[nH]c3nc2-c2cccc(C#N)c2)cc(Cl)c1=O] |
| 2.000 | 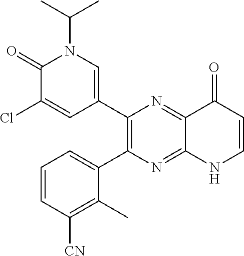 [SMILES: Cc1c(C#N)cccc1-c1nc2[nH]ccc(=O)c2nc1-c1cc(Cl)c(=O)n(C(C)C)c1] |
| 2.001 | 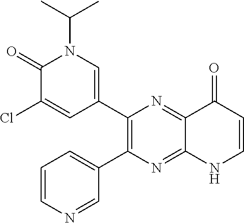 [SMILES: CC(C)n1cc(-c2nc3c(=O)cc[nH]c3nc2-c2cccnc2)cc(Cl)c1=O] |
| 2.002 | 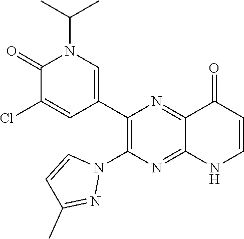 [SMILES: Cc1ccn(-c2nc3[nH]ccc(=O)c3nc2-c2cc(Cl)c(=O)n(C(C)C)c2)n1] |
| 2.003 | 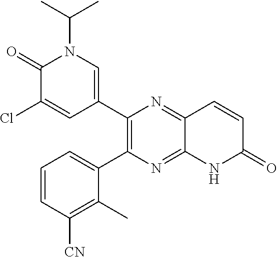 [SMILES: Cc1c(C#N)cccc1-c1nc2[nH]c(=O)ccc2nc1-c1cc(Cl)c(=O)n(C(C)C)c1] |
| 2.004 | 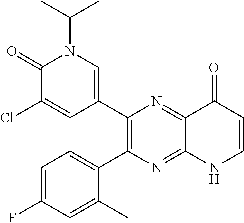 [SMILES: Cc1cc(F)ccc1-c1nc2[nH]ccc(=O)c2nc1-c1cc(Cl)c(=O)n(C(C)C)c1] |
| 2.005 | 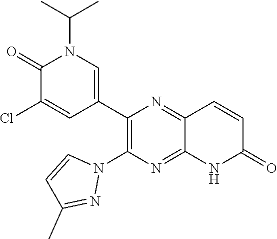 [SMILES: Cc1ccn(-c2nc3[nH]c(=O)ccc3nc2-c2cc(Cl)c(=O)n(C(C)C)c2)n1] |
| 2.006 | 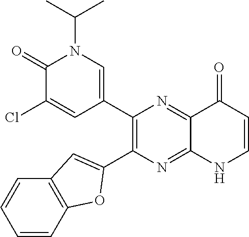 [SMILES: CC(C)n1cc(-c2nc3c(=O)cc[nH]c3nc2-c2cc3ccccc3o2)cc(Cl)c1=O] |
| 2.007 | 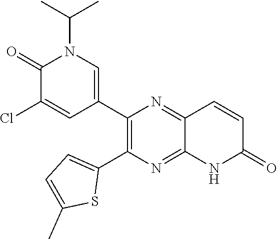 [SMILES: Cc1ccc(-c2nc3[nH]c(=O)ccc3nc2-c2cc(Cl)c(=O)n(C(C)C)c2)s1] |
| 2.008 | 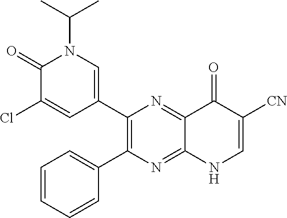 [SMILES: CC(C)n1cc(-c2nc3c(=O)c(C#N)c[nH]c3nc2-c2ccccc2)cc(Cl)c1=O] |
| 2.009 | 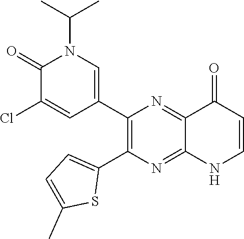 [SMILES: Cc1ccc(-c2nc3[nH]ccc(=O)c3nc2-c2cc(Cl)c(=O)n(C(C)C)c2)s1] |
| 2.010 | 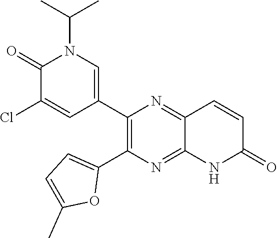 [SMILES: Cc1ccc(-c2nc3[nH]c(=O)ccc3nc2-c2cc(Cl)c(=O)n(C(C)C)c2)o1] |
| 2.011 | 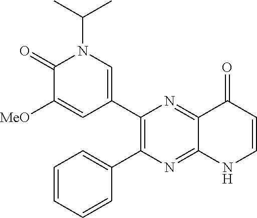 [SMILES: COc1cc(-c2nc3c(=O)cc[nH]c3nc2-c2ccccc2)cn(C(C)C)c1=O] |
| 2.012 | 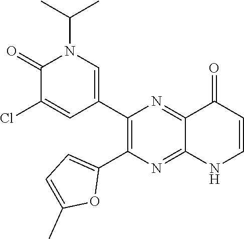 [SMILES: Cc1ccc(-c2nc3[nH]ccc(=O)c3nc2-c2cc(Cl)c(=O)n(C(C)C)c2)o1] |
| 2.013 | 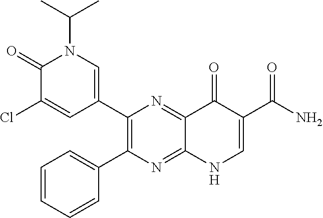 [SMILES: CC(C)n1cc(-c2nc3c(=O)c(C(N)=O)c[nH]c3nc2-c2ccccc2)cc(Cl)c1=O] |
| 2.014 | 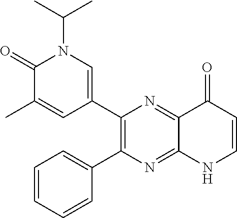 [SMILES: Cc1cc(-c2nc3c(=O)cc[nH]c3nc2-c2ccccc2)cn(C(C)C)c1=O] |
| 2.015 | 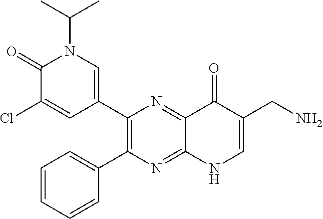 [SMILES: CC(C)n1cc(-c2nc3c(=O)c(CN)c[nH]c3nc2-c2ccccc2)cc(Cl)c1=O] |
| 2.016 | 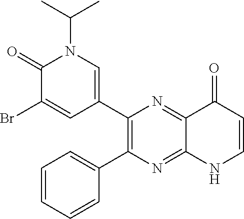 [SMILES: CC(C)n1cc(-c2nc3c(=O)cc[nH]c3nc2-c2ccccc2)cc(Br)c1=O] |
| 2.017 | 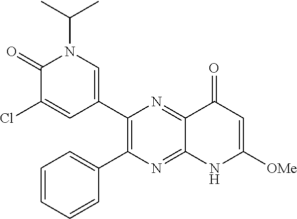 [SMILES: COc1cc(=O)c2nc(-c3cc(Cl)c(=O)n(C(C)C)c3)c(-c3ccccc3)nc2[nH]1] |
| 2.018 | 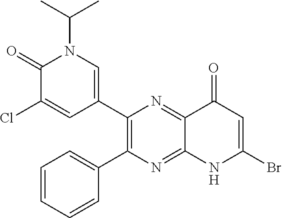 [SMILES: CC(C)n1cc(-c2nc3c(=O)cc(Br)[nH]c3nc2-c2ccccc2)cc(Cl)c1=O] |
| 2.019 | 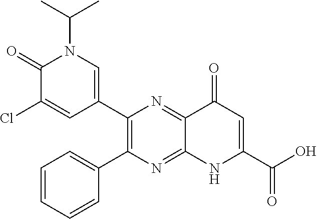 [SMILES: CC(C)n1cc(-c2nc3c(=O)cc(C(=O)O)[nH]c3nc2-c2ccccc2)cc(Cl)c1=O] |
| 2.020 | 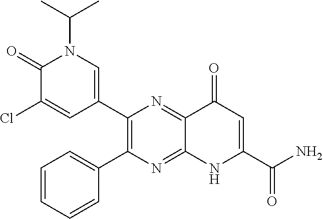 [SMILES: CC(C)n1cc(-c2nc3c(=O)cc(C(N)=O)[nH]c3nc2-c2ccccc2)cc(Cl)c1=O] |
| 2.021 | 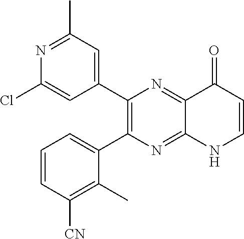 [SMILES: Cc1cc(-c2nc3c(=O)cc[nH]c3nc2-c2cccc(C#N)c2C)cc(Cl)n1] |
| 2.022 | 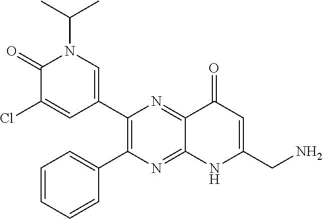 [SMILES: CC(C)n1cc(-c2nc3c(=O)cc(CN)[nH]c3nc2-c2ccccc2)cc(Cl)c1=O] |
| 2.023 | 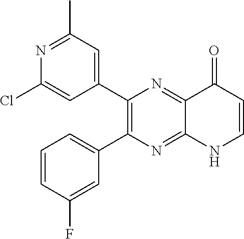 [SMILES: Cc1cc(-c2nc3c(=O)cc[nH]c3nc2-c2cccc(F)c2)cc(Cl)n1] |
| 2.024 | 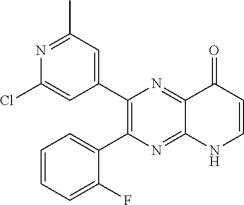 [SMILES: Cc1cc(-c2nc3c(=O)cc[nH]c3nc2-c2ccccc2F)cc(Cl)n1] |
| 2.025 | 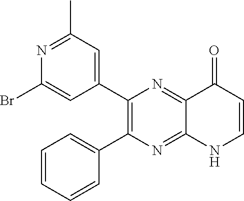 [SMILES: Cc1cc(-c2nc3c(=O)cc[nH]c3nc2-c2ccccc2)cc(Br)n1] |
| 2.026 | 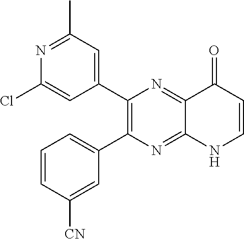 [SMILES: Cc1cc(-c2nc3c(=O)cc[nH]c3nc2-c2cccc(C#N)c2)cc(Cl)n1] |
| 2.027 | 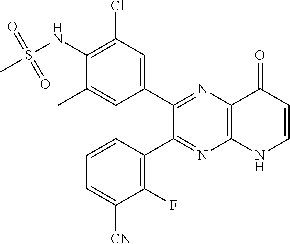 [SMILES: Cc1cc(-c2nc3c(=O)cc[nH]c3nc2-c2cccc(C#N)c2F)cc(Cl)c1NS(C)(=O)=O] |
| 2.028 | 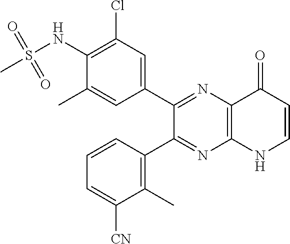 [SMILES: Cc1cc(-c2nc3c(=O)cc[nH]c3nc2-c2cccc(C#N)c2C)cc(Cl)c1NS(C)(=O)=O] |
| 2.029 | 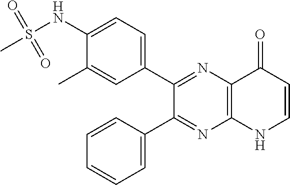 [SMILES: Cc1cc(-c2nc3c(=O)cc[nH]c3nc2-c2ccccc2)ccc1NS(C)(=O)=O] |
| 2.030 | 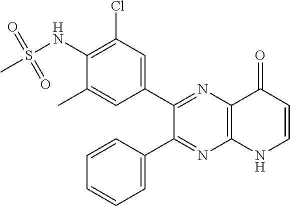 [SMILES: Cc1cc(-c2nc3c(=O)cc[nH]c3nc2-c2ccccc2)cc(Cl)c1NS(C)(=O)=O] |
| 2.031 | 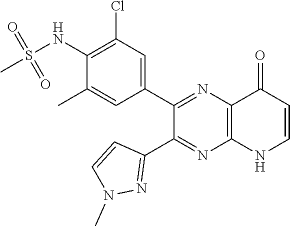 [SMILES: Cc1cc(-c2nc3c(=O)cc[nH]c3nc2-c2ccn(C)n2)cc(Cl)c1NS(C)(=O)=O] |
| 2.032 | 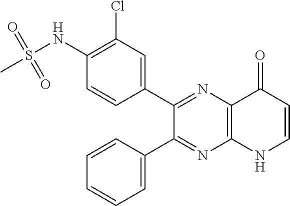 [SMILES: CS(=O)(=O)Nc1ccc(-c2nc3c(=O)cc[nH]c3nc2-c2ccccc2)cc1Cl] |
| 2.033 | 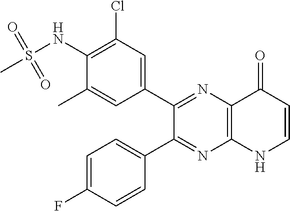 [SMILES: Cc1cc(-c2nc3c(=O)cc[nH]c3nc2-c2ccc(F)cc2)cc(Cl)c1NS(C)(=O)=O] |
| 2.034 | 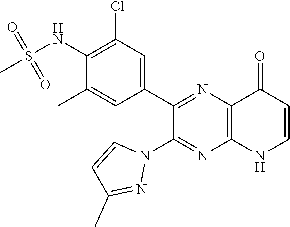 [SMILES: Cc1ccn(-c2nc3[nH]ccc(=O)c3nc2-c2cc(C)c(NS(C)(=O)=O)c(Cl)c2)n1] |
| 2.035 | 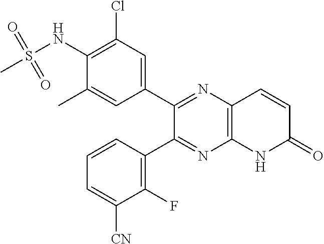 [SMILES: Cc1cc(-c2nc3ccc(=O)[nH]c3nc2-c2cccc(C#N)c2F)cc(Cl)c1NS(C)(=O)=O] |
| 2.036 | 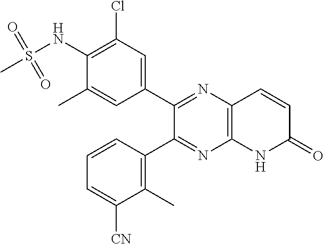 [SMILES: Cc1cc(-c2nc3ccc(=O)[nH]c3nc2-c2cccc(C#N)c2C)cc(Cl)c1NS(C)(=O)=O] |
| 2.037 | 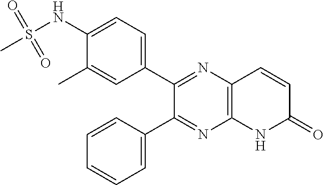 [SMILES: Cc1cc(-c2nc3ccc(=O)[nH]c3nc2-c2ccccc2)ccc1NS(C)(=O)=O] |
| 2.038 | 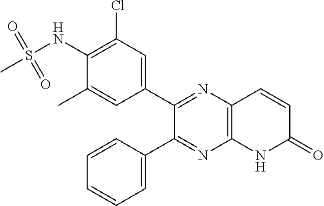 [SMILES: Cc1cc(-c2nc3ccc(=O)[nH]c3nc2-c2ccccc2)cc(Cl)c1NS(C)(=O)=O] |
| 2.039 | 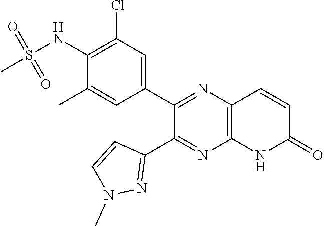 [SMILES: Cc1cc(-c2nc3ccc(=O)[nH]c3nc2-c2ccn(C)n2)cc(Cl)c1NS(C)(=O)=O] |
| 2.040 | 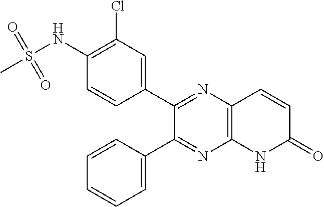 [SMILES: CS(=O)(=O)Nc1ccc(-c2nc3ccc(=O)[nH]c3nc2-c2ccccc2)cc1Cl] |
| 2.041 | 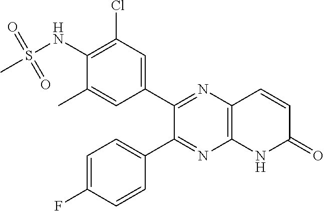 [SMILES: Cc1cc(-c2nc3ccc(=O)[nH]c3nc2-c2ccc(F)cc2)cc(Cl)c1NS(C)(=O)=O] |
| 2.042 | 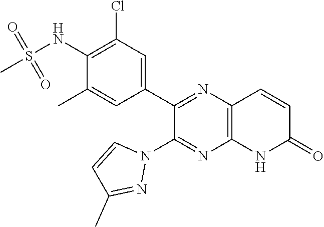 [SMILES: Cc1ccn(-c2nc3[nH]c(=O)ccc3nc2-c2cc(C)c(NS(C)(=O)=O)c(Cl)c2)n1] |
| 2.043 | 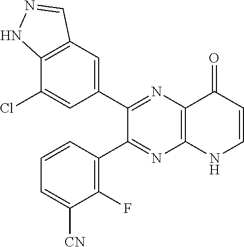 [SMILES: N#Cc1cccc(-c2nc3[nH]ccc(=O)c3nc2-c2cc(Cl)c3[nH]ncc3c2)c1F] |
| 2.044 | 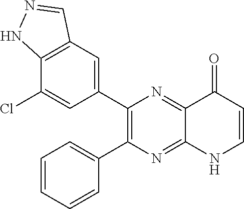 [SMILES: O=c1cc[nH]c2nc(-c3ccccc3)c(-c3cc(Cl)c4[nH]ncc4c3)nc12] |
| 2.045 | 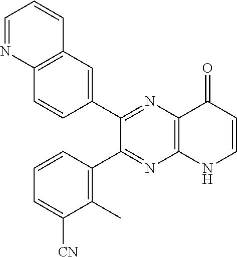 [SMILES: Cc1c(C#N)cccc1-c1nc2[nH]ccc(=O)c2nc1-c1ccc2ncccc2c1] |
| 2.046 | 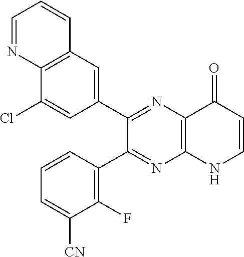 [SMILES: N#Cc1cccc(-c2nc3[nH]ccc(=O)c3nc2-c2cc(Cl)c3ncccc3c2)c1F] |
| 2.047 | 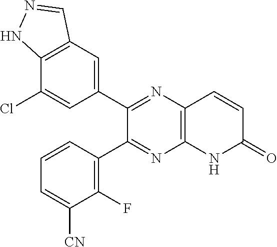 [SMILES: N#Cc1cccc(-c2nc3[nH]c(=O)ccc3nc2-c2cc(Cl)c3[nH]ncc3c2)c1F] |
| 2.048 | 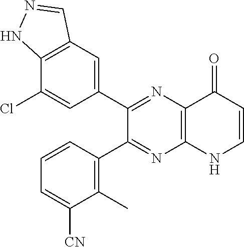 [SMILES: Cc1c(C#N)cccc1-c1nc2[nH]ccc(=O)c2nc1-c1cc(Cl)c2[nH]ncc2c1] |
| 2.049 | 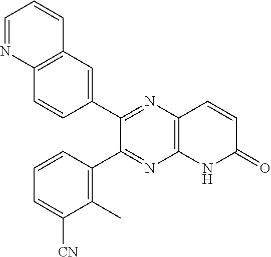 [SMILES: Cc1c(C#N)cccc1-c1nc2[nH]c(=O)ccc2nc1-c1ccc2ncccc2c1] |
| 2.050 | 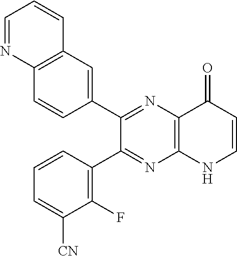 [SMILES: N#Cc1cccc(-c2nc3[nH]ccc(=O)c3nc2-c2ccc3ncccc3c2)c1F] |
| 2.051 | 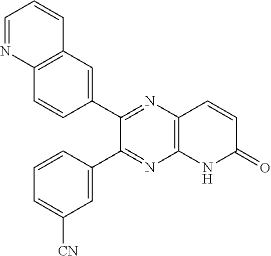 [SMILES: N#Cc1cccc(-c2nc3[nH]c(=O)ccc3nc2-c2ccc3ncccc3c2)c1] |
| 2.052 | 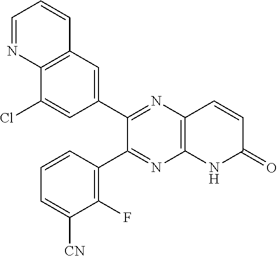 [SMILES: N#Cc1cccc(-c2nc3[nH]c(=O)ccc3nc2-c2cc(Cl)c3ncccc3c2)c1F] |
| 2.053 | 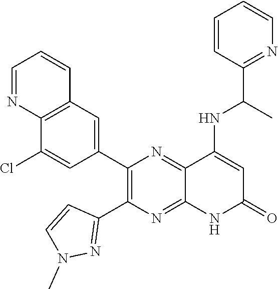 [SMILES: CC(Nc1cc(=O)[nH]c2nc(-c3ccn(C)n3)c(-c3cc(Cl)c4ncccc4c3)nc12)c1ccccn1] |
| 2.054 | 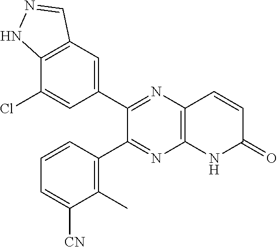 [SMILES: Cc1c(C#N)cccc1-c1nc2[nH]c(=O)ccc2nc1-c1cc(Cl)c2[nH]ncc2c1] |
| 2.055 | 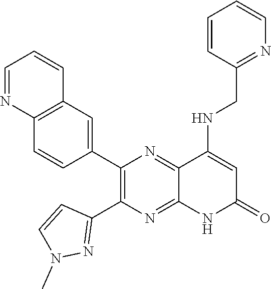 [SMILES: Cn1ccc(-c2nc3[nH]c(=O)cc(NCc4ccccn4)c3nc2-c2ccc3ncccc3c2)n1] |
| 2.056 | 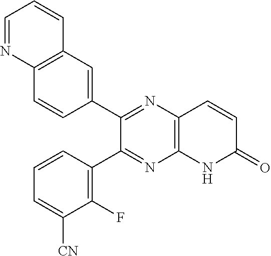 [SMILES: N#Cc1cccc(-c2nc3[nH]c(=O)ccc3nc2-c2ccc3ncccc3c2)c1F] |
| 2.057 | 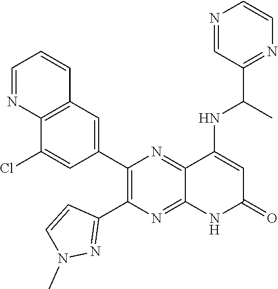 [SMILES: CC(Nc1cc(=O)[nH]c2nc(-c3ccn(C)n3)c(-c3cc(Cl)c4ncccc4c3)nc12)c1cnccn1] |
| 2.058 | 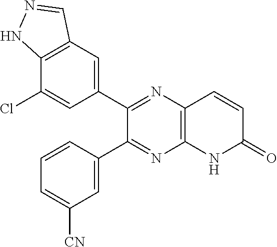 [SMILES: N#Cc1cccc(-c2nc3[nH]c(=O)ccc3nc2-c2cc(Cl)c3[nH]ncc3c2)c1] |
| 2.059 | 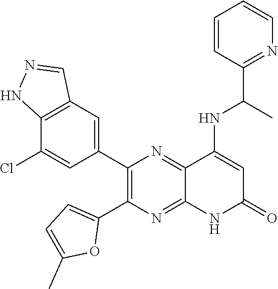 [SMILES: Cc1ccc(-c2nc3[nH]c(=O)cc(NC(C)c4ccccn4)c3nc2-c2cc(Cl)c3[nH]ncc3c2)o1] |
| 2.060 | 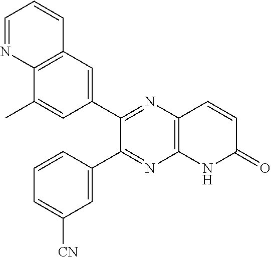 [SMILES: Cc1cc(-c2nc3ccc(=O)[nH]c3nc2-c2cccc(C#N)c2)cc2cccnc12] |
| 2.061 | 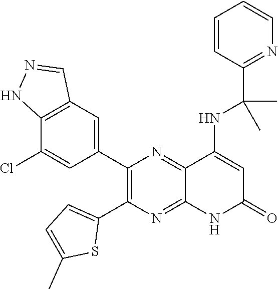 [SMILES: Cc1ccc(-c2nc3[nH]c(=O)cc(NC(C)(C)c4ccccn4)c3nc2-c2cc(Cl)c3[nH]ncc3c2)s1] |
| 2.062 | 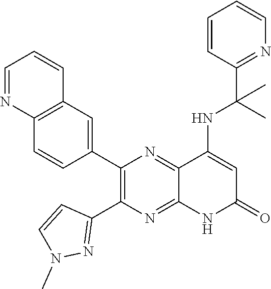 [SMILES: Cn1ccc(-c2nc3[nH]c(=O)cc(NC(C)(C)c4ccccn4)c3nc2-c2ccc3ncccc3c2)n1] |
| 2.063 | 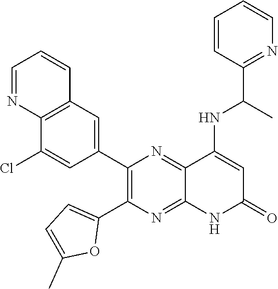 [SMILES: Cc1ccc(-c2nc3[nH]c(=O)cc(NC(C)c4ccccn4)c3nc2-c2cc(Cl)c3ncccc3c2)o1] |
| 2.064 | 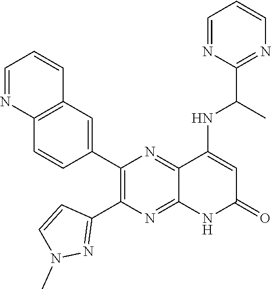 [SMILES: CC(Nc1cc(=O)[nH]c2nc(-c3ccn(C)n3)c(-c3ccc4ncccc4c3)nc12)c1ncccn1] |
| 2.065 | 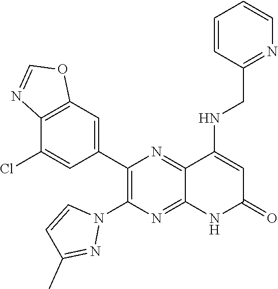 [SMILES: Cc1ccn(-c2nc3[nH]c(=O)cc(NCc4ccccn4)c3nc2-c2cc(Cl)c3ncoc3c2)n1] |
| 2.066 | 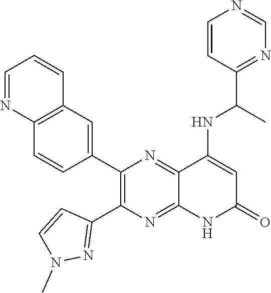 [SMILES: CC(Nc1cc(=O)[nH]c2nc(-c3ccn(C)n3)c(-c3ccc4ncccc4c3)nc12)c1ccncn1] |
| 2.067 | 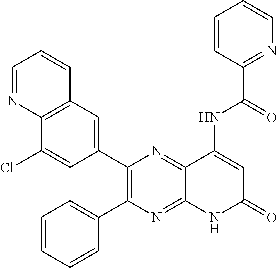 [SMILES: O=C(Nc1cc(=O)[nH]c2nc(-c3ccccc3)c(-c3cc(Cl)c4ncccc4c3)nc12)c1ccccn1] |
| 2.068 | 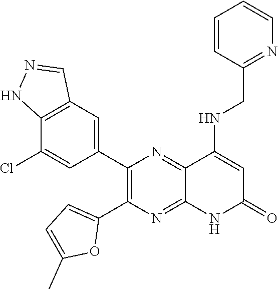 [SMILES: Cc1ccc(-c2nc3[nH]c(=O)cc(NCc4ccccn4)c3nc2-c2cc(Cl)c3[nH]ncc3c2)o1] |
| 2.069 | 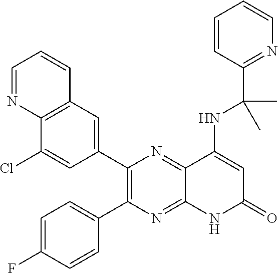 [SMILES: CC(C)(Nc1cc(=O)[nH]c2nc(-c3ccc(F)cc3)c(-c3cc(Cl)c4ncccc4c3)nc12)c1ccccn1] |
| 2.070 | 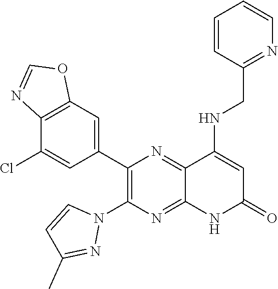 [SMILES: Cc1ccn(-c2nc3[nH]c(=O)cc(NCc4ccccn4)c3nc2-c2cc(Cl)c3ncoc3c2)n1] |
| 2.071 | 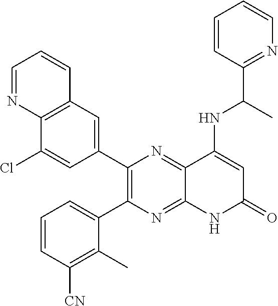 [SMILES: Cc1c(C#N)cccc1-c1nc2[nH]c(=O)cc(NC(C)c3ccccn3)c2nc1-c1cc(Cl)c2ncccc2c1] |
| 2.072 | 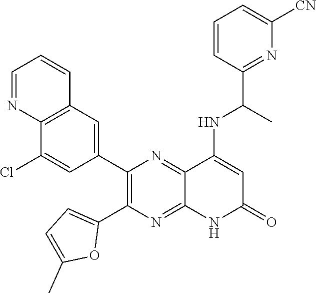 [SMILES: Cc1ccc(-c2nc3[nH]c(=O)cc(NC(C)c4cccc(C#N)n4)c3nc2-c2cc(Cl)c3ncccc3c2)o1] |
| 2.073 | 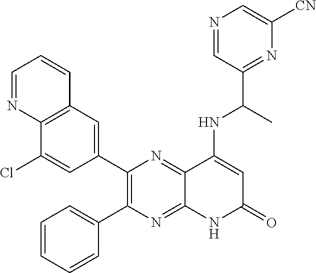 [SMILES: CC(Nc1cc(=O)[nH]c2nc(-c3ccccc3)c(-c3cc(Cl)c4ncccc4c3)nc12)c1cncc(C#N)n1] |
| 2.074 | 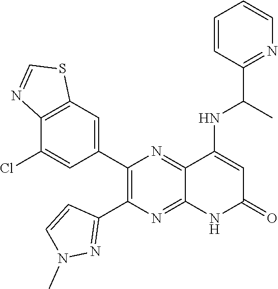 [SMILES: CC(Nc1cc(=O)[nH]c2nc(-c3ccn(C)n3)c(-c3cc(Cl)c4ncsc4c3)nc12)c1ccccn1] |
| 2.075 | 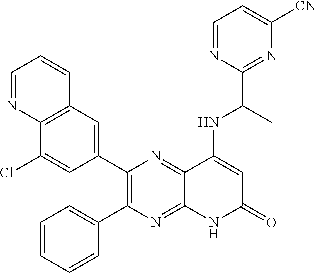 [SMILES: CC(Nc1cc(=O)[nH]c2nc(-c3ccccc3)c(-c3cc(Cl)c4ncccc4c3)nc12)c1nccc(C#N)n1] |
| 2.076 | 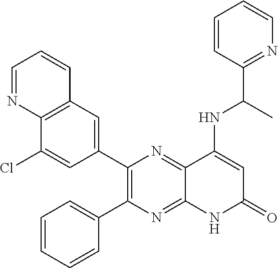 [SMILES: CC(Nc1cc(=O)[nH]c2nc(-c3ccccc3)c(-c3cc(Cl)c4ncccc4c3)nc12)c1ccccn1] |
| 2.077 | 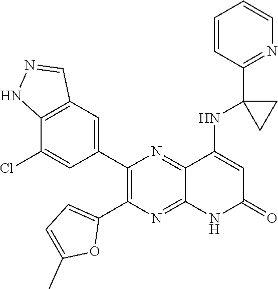 [SMILES: Cc1ccc(-c2nc3[nH]c(=O)cc(NC4(c5ccccn5)CC4)c3nc2-c2cc(Cl)c3[nH]ncc3c2)o1] |
| 2.078 | 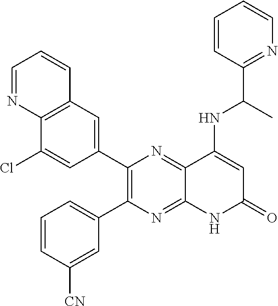 [SMILES: CC(Nc1cc(=O)[nH]c2nc(-c3cccc(C#N)c3)c(-c3cc(Cl)c4ncccc4c3)nc12)c1ccccn1] |
| 2.079 | 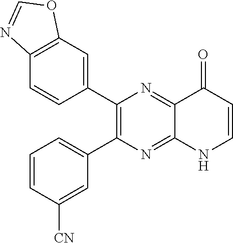 [SMILES: N#Cc1cccc(-c2nc3[nH]ccc(=O)c3nc2-c2ccc3ncoc3c2)c1] |
| 2.080 | 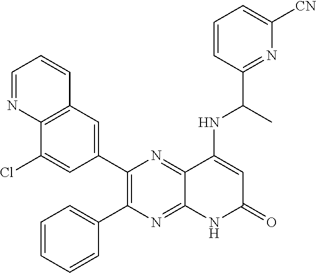 [SMILES: CC(Nc1cc(=O)[nH]c2nc(-c3ccccc3)c(-c3cc(Cl)c4ncccc4c3)nc12)c1cccc(C#N)n1] |
| 2.081 | 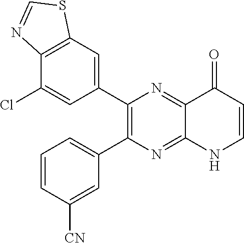 [SMILES: N#Cc1cccc(-c2nc3[nH]ccc(=O)c3nc2-c2cc(Cl)c3ncsc3c2)c1] |
| 2.082 | 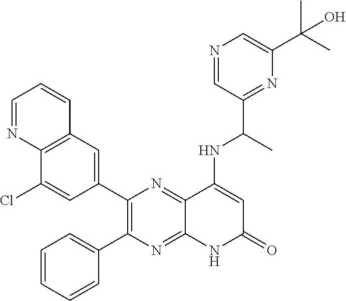 [SMILES: CC(Nc1cc(=O)[nH]c2nc(-c3ccccc3)c(-c3cc(Cl)c4ncccc4c3)nc12)c1cncc(C(C)(C)O)n1] |
| 2.083 | 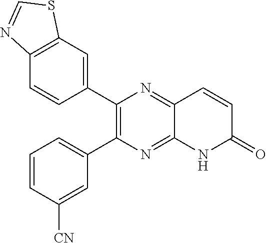 [SMILES: N#Cc1cccc(-c2nc3[nH]c(=O)ccc3nc2-c2ccc3ncsc3c2)c1] |
| 2.084 | 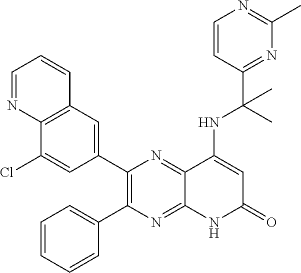 [SMILES: Cc1nccc(C(C)(C)Nc2cc(=O)[nH]c3nc(-c4ccccc4)c(-c4cc(Cl)c5ncccc5c4)nc23)n1] |
| 2.085 | 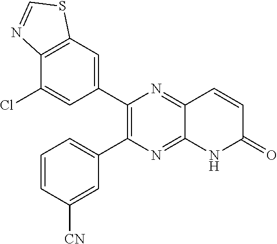 [SMILES: N#Cc1cccc(-c2nc3[nH]c(=O)ccc3nc2-c2cc(Cl)c3ncsc3c2)c1] |
| 2.086 | 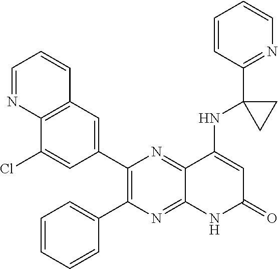 [SMILES: O=c1cc(NC2(c3ccccn3)CC2)c2nc(-c3cc(Cl)c4ncccc4c3)c(-c3ccccc3)nc2[nH]1] |
| 2.087 | 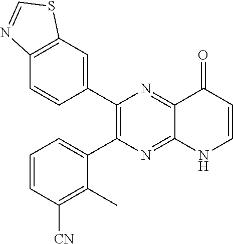 [SMILES: Cc1c(C#N)cccc1-c1nc2[nH]ccc(=O)c2nc1-c1ccc2ncsc2c1] |
| 2.088 | 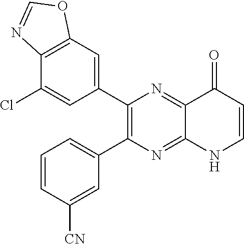 [SMILES: N#Cc1cccc(-c2nc3[nH]ccc(=O)c3nc2-c2cc(Cl)c3ncoc3c2)c1] |
| 2.089 | 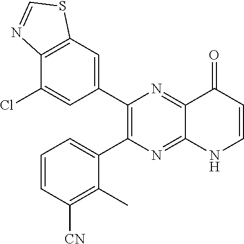 [SMILES: Cc1c(C#N)cccc1-c1nc2[nH]ccc(=O)c2nc1-c1cc(Cl)c2ncsc2c1] |
| 2.090 | 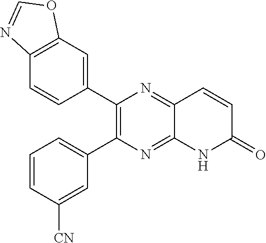 [SMILES: N#Cc1cccc(-c2nc3[nH]c(=O)ccc3nc2-c2ccc3ncoc3c2)c1] |
| 2.091 | 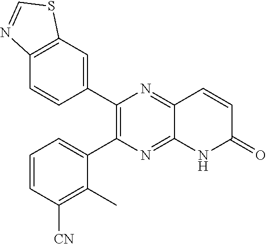 [SMILES: Cc1c(C#N)cccc1-c1nc2[nH]c(=O)ccc2nc1-c1ccc2ncsc2c1] |
| 2.092 | 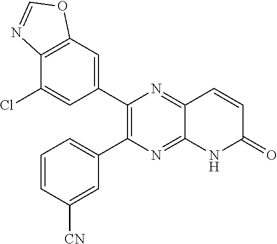 [SMILES: N#Cc1cccc(-c2nc3[nH]c(=O)ccc3nc2-c2cc(Cl)c3ncoc3c2)c1] |
| 2.093 | 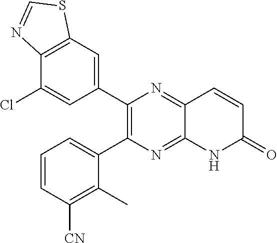 [SMILES: Cc1c(C#N)cccc1-c1nc2[nH]c(=O)ccc2nc1-c1cc(Cl)c2ncsc2c1] |
| 2.094 | 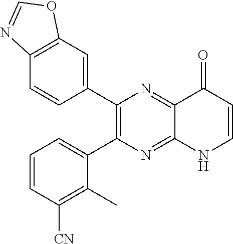 [SMILES: Cc1c(C#N)cccc1-c1nc2[nH]ccc(=O)c2nc1-c1ccc2ncoc2c1] |
| 2.095 | 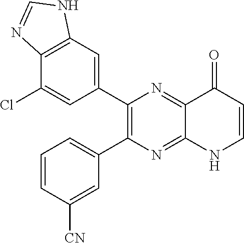 [SMILES: N#Cc1cccc(-c2nc3[nH]ccc(=O)c3nc2-c2cc(Cl)c3nc[nH]c3c2)c1] |
| 2.096 | 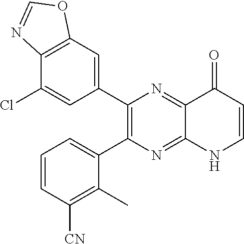 [SMILES: Cc1c(C#N)cccc1-c1nc2[nH]ccc(=O)c2nc1-c1cc(Cl)c2ncoc2c1] |
| 2.097 | 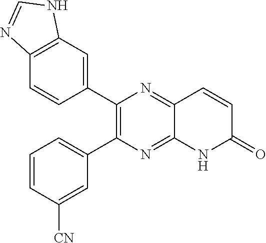 [SMILES: N#Cc1cccc(-c2nc3[nH]c(=O)ccc3nc2-c2ccc3nc[nH]c3c2)c1] |
| 2.098 | 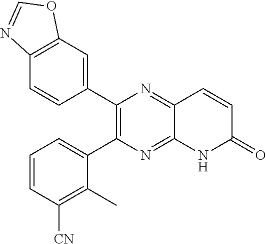 [SMILES: Cc1c(C#N)cccc1-c1nc2[nH]c(=O)ccc2nc1-c1ccc2ncoc2c1] |
| 2.099 | 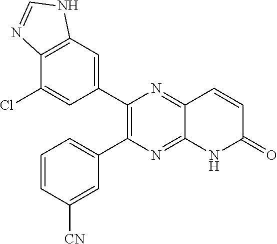 [SMILES: N#Cc1cccc(-c2nc3[nH]c(=O)ccc3nc2-c2cc(Cl)c3nc[nH]c3c2)c1] |
| 2.100 | 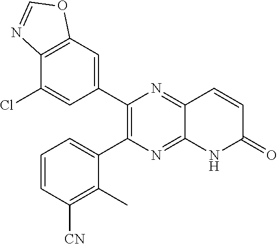 [SMILES: Cc1c(C#N)cccc1-c1nc2[nH]c(=O)ccc2nc1-c1cc(Cl)c2ncoc2c1] |
| 2.101 | 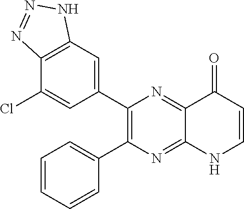 [SMILES: O=c1cc[nH]c2nc(-c3ccccc3)c(-c3cc(Cl)c4nn[nH]c4c3)nc12] |
| 2.102 | 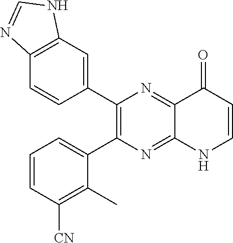 [SMILES: Cc1c(C#N)cccc1-c1nc2[nH]ccc(=O)c2nc1-c1ccc2nc[nH]c2c1] |
| 2.103 | 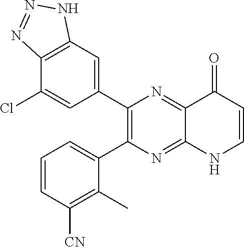 [SMILES: Cc1c(C#N)cccc1-c1nc2[nH]ccc(=O)c2nc1-c1cc(Cl)c2nn[nH]c2c1] |
| 2.104 | 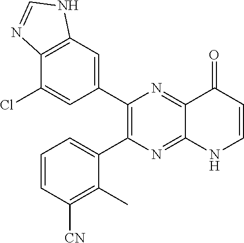 [SMILES: Cc1c(C#N)cccc1-c1nc2[nH]ccc(=O)c2nc1-c1cc(Cl)c2nc[nH]c2c1] |
| 2.105 | 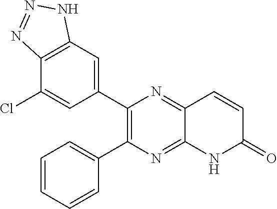 [SMILES: O=c1ccc2nc(-c3cc(Cl)c4nn[nH]c4c3)c(-c3ccccc3)nc2[nH]1] |
| 2.106 | 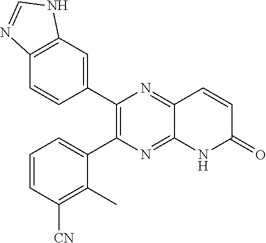 [SMILES: Cc1c(C#N)cccc1-c1nc2[nH]c(=O)ccc2nc1-c1ccc2nc[nH]c2c1] |
| 2.107 | 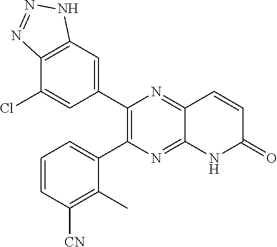 [SMILES: Cc1c(C#N)cccc1-c1nc2[nH]c(=O)ccc2nc1-c1cc(Cl)c2nn[nH]c2c1] |
| 2.108 | 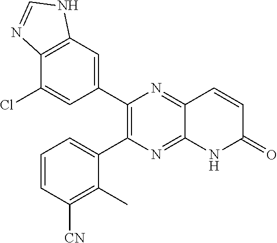 [SMILES: Cc1c(C#N)cccc1-c1nc2[nH]c(=O)ccc2nc1-c1cc(Cl)c2nc[nH]c2c1] |
| 2.109 | 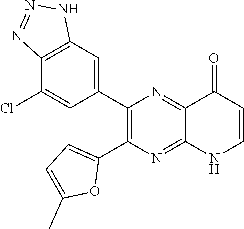 [SMILES: Cc1ccc(-c2nc3[nH]ccc(=O)c3nc2-c2cc(Cl)c3nn[nH]c3c2)o1] |
| 2.110 | 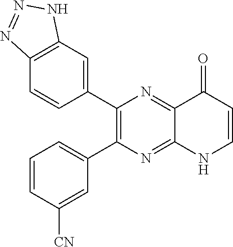 [SMILES: N#Cc1cccc(-c2nc3[nH]ccc(=O)c3nc2-c2ccc3nn[nH]c3c2)c1] |
| 2.111 | 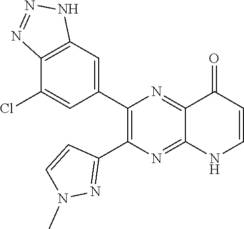 [SMILES: Cn1ccc(-c2nc3[nH]ccc(=O)c3nc2-c2cc(Cl)c3nn[nH]c3c2)n1] |
| 2.112 | 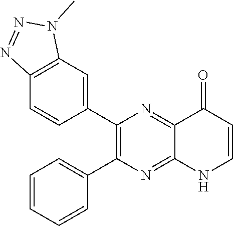 [SMILES: Cn1nnc2ccc(-c3nc4c(=O)cc[nH]c4nc3-c3ccccc3)cc21] |
| 2.113 | 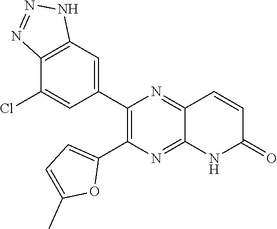 [SMILES: Cc1ccc(-c2nc3[nH]c(=O)ccc3nc2-c2cc(Cl)c3nn[nH]c3c2)o1] |
| 2.114 | 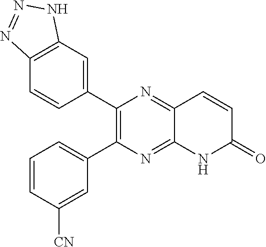 [SMILES: N#Cc1cccc(-c2nc3[nH]c(=O)ccc3nc2-c2ccc3nn[nH]c3c2)c1] |
| 2.115 | 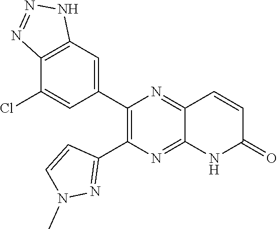 [SMILES: Cn1ccc(-c2nc3[nH]c(=O)ccc3nc2-c2cc(Cl)c3nn[nH]c3c2)n1] |
| 2.116 | 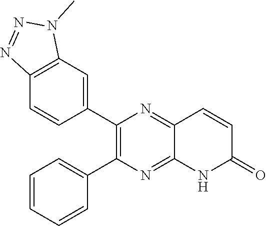 [SMILES: Cn1nnc2ccc(-c3nc4ccc(=O)[nH]c4nc3-c3ccccc3)cc21] |
| 2.117 | 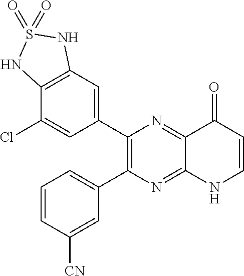 [SMILES: N#Cc1cccc(-c2nc3[nH]ccc(=O)c3nc2-c2cc(Cl)c3c(c2)NS(=O)(=O)N3)c1] |
| 2.118 | 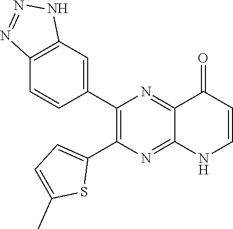 [SMILES: Cc1ccc(-c2nc3[nH]ccc(=O)c3nc2-c2ccc3nn[nH]c3c2)s1] |
| 2.119 | 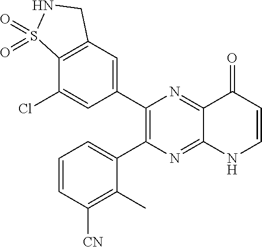 [SMILES: Cc1c(C#N)cccc1-c1nc2[nH]ccc(=O)c2nc1-c1cc(Cl)c2c(c1)CNS2(=O)=O] |
| 2.120 | 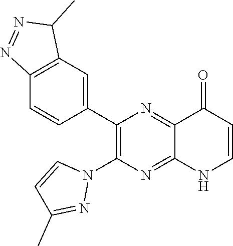 [SMILES: Cc1ccn(-c2nc3[nH]ccc(=O)c3nc2-c2ccc3c(c2)C(C)N=N3)n1] |
| 2.121 | 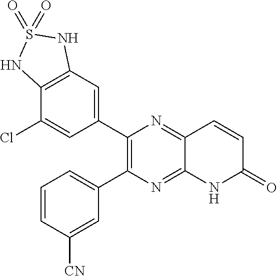 [SMILES: N#Cc1cccc(-c2nc3[nH]c(=O)ccc3nc2-c2cc(Cl)c3c(c2)NS(=O)(=O)N3)c1] |
| 2.122 | 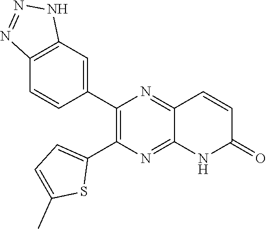 [SMILES: Cc1ccc(-c2nc3[nH]c(=O)ccc3nc2-c2ccc3nn[nH]c3c2)s1] |
| 2.123 | 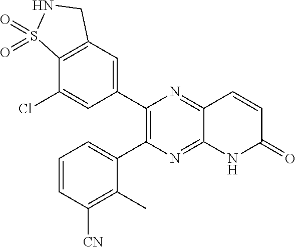 [SMILES: Cc1c(C#N)cccc1-c1nc2[nH]c(=O)ccc2nc1-c1cc(Cl)c2c(c1)CNS2(=O)=O] |
| 2.124 | 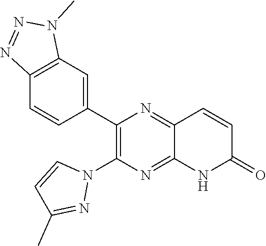 [SMILES: Cc1ccn(-c2nc3[nH]c(=O)ccc3nc2-c2ccc3nnn(C)c3c2)n1] |
| 2.125 | 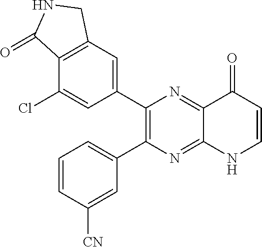 [SMILES: N#Cc1cccc(-c2nc3[nH]ccc(=O)c3nc2-c2cc(Cl)c3c(c2)CNC3=O)c1] |
| 2.126 | 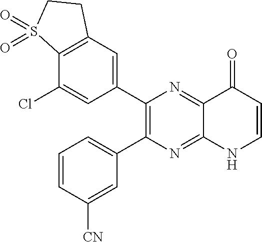 [SMILES: N#Cc1cccc(-c2nc3[nH]ccc(=O)c3nc2-c2cc(Cl)c3c(c2)CCS3(=O)=O)c1] |
| 2.127 | 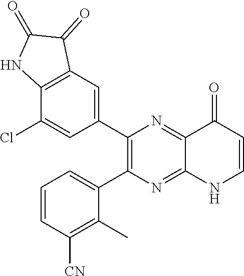 [SMILES: Cc1c(C#N)cccc1-c1nc2[nH]ccc(=O)c2nc1-c1cc(Cl)c2c(c1)C(=O)C(=O)N2] |
| 2.128 | 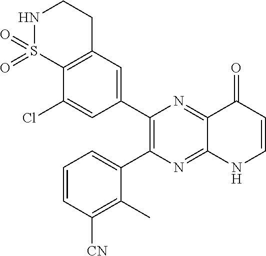 [SMILES: Cc1c(C#N)cccc1-c1nc2[nH]ccc(=O)c2nc1-c1cc(Cl)c2c(c1)CCNS2(=O)=O] |
| 2.129 | 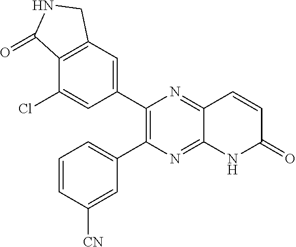 [SMILES: N#Cc1cccc(-c2nc3[nH]c(=O)ccc3nc2-c2cc(Cl)c3c(c2)CNC3=O)c1] |
| 2.130 | 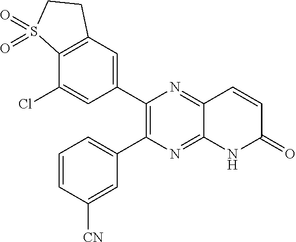 [SMILES: N#Cc1cccc(-c2nc3[nH]c(=O)ccc3nc2-c2cc(Cl)c3c(c2)CCS3(=O)=O)c1] |
| 2.131 | 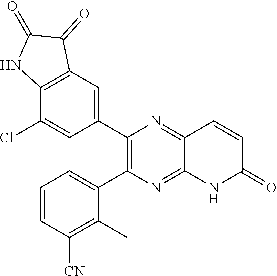 [SMILES: Cc1c(C#N)cccc1-c1nc2[nH]c(=O)ccc2nc1-c1cc(Cl)c2c(c1)C(=O)C(=O)N2] |
| 2.132 | 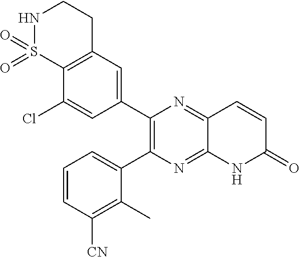 [SMILES: Cc1c(C#N)cccc1-c1nc2[nH]c(=O)ccc2nc1-c1cc(Cl)c2c(c1)CCNS2(=O)=O] |
| 2.133 | 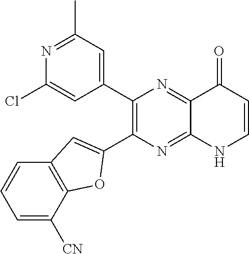 [SMILES: Cc1cc(-c2nc3c(=O)cc[nH]c3nc2-c2cc3cccc(C#N)c3o2)cc(Cl)n1] |
| 2.134 | 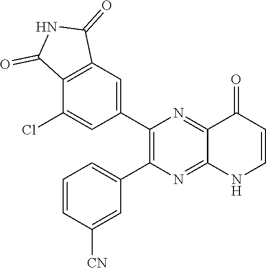 [SMILES: N#Cc1cccc(-c2nc3[nH]ccc(=O)c3nc2-c2cc(Cl)c3c(c2)C(=O)NC3=O)c1] |
| 2.135 | 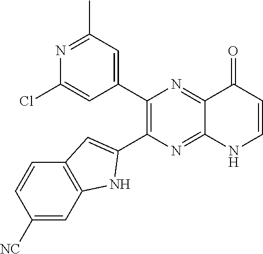 [SMILES: Cc1cc(-c2nc3c(=O)cc[nH]c3nc2-c2cc3ccc(C#N)cc3[nH]2)cc(Cl)n1] |
| 2.136 | 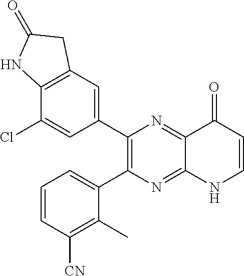 [SMILES: Cc1c(C#N)cccc1-c1nc2[nH]ccc(=O)c2nc1-c1cc(Cl)c2c(c1)CC(=O)N2] |
| 2.137 | 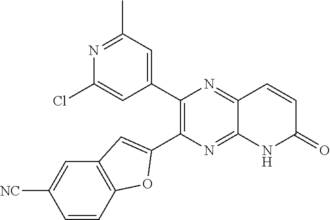 [SMILES: Cc1cc(-c2nc3ccc(=O)[nH]c3nc2-c2cc3cc(C#N)ccc3o2)cc(Cl)n1] |
| 2.138 | 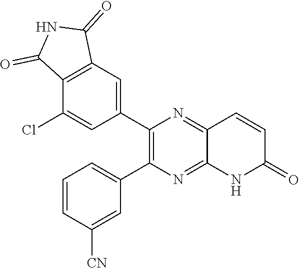 [SMILES: N#Cc1cccc(-c2nc3[nH]c(=O)ccc3nc2-c2cc(Cl)c3c(c2)C(=O)NC3=O)c1] |
| 2.139 | 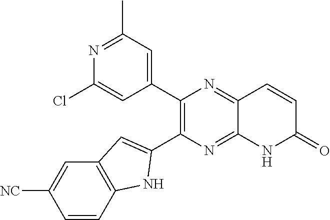 [SMILES: Cc1cc(-c2nc3ccc(=O)[nH]c3nc2-c2cc3cc(C#N)ccc3[nH]2)cc(Cl)n1] |
| 2.140 | 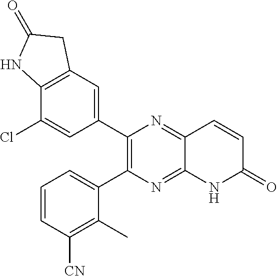 [SMILES: Cc1c(C#N)cccc1-c1nc2[nH]c(=O)ccc2nc1-c1cc(Cl)c2c(c1)CC(=O)N2] |
| 2.141 | 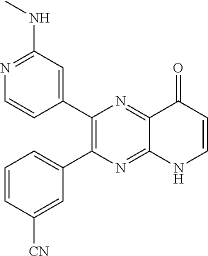 [SMILES: CNc1cc(-c2nc3c(=O)cc[nH]c3nc2-c2cccc(C#N)c2)ccn1] |
| 2.142 | 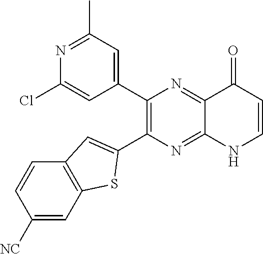 [SMILES: Cc1cc(-c2nc3c(=O)cc[nH]c3nc2-c2cc3ccc(C#N)cc3s2)cc(Cl)n1] |
| 2.143 | 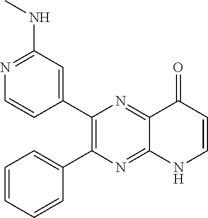 [SMILES: CNc1cc(-c2nc3c(=O)cc[nH]c3nc2-c2ccccc2)ccn1] |
| 2.144 | 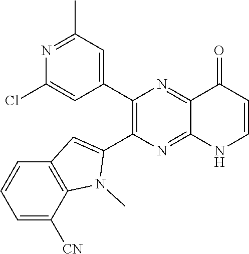 [SMILES: Cc1cc(-c2nc3c(=O)cc[nH]c3nc2-c2cc3cccc(C#N)c3n2C)cc(Cl)n1] |
| 2.145 | 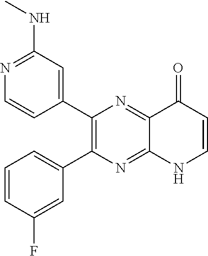 [SMILES: CNc1cc(-c2nc3c(=O)cc[nH]c3nc2-c2cccc(F)c2)ccn1] |
| 2.146 | 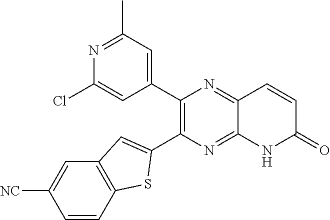 [SMILES: Cc1cc(-c2nc3ccc(=O)[nH]c3nc2-c2cc3cc(C#N)ccc3s2)cc(Cl)n1] |
| 2.147 | 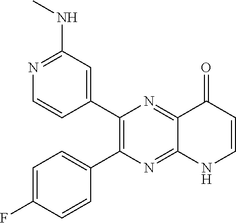 [SMILES: CNc1cc(-c2nc3c(=O)cc[nH]c3nc2-c2ccc(F)cc2)ccn1] |
| 2.148 | 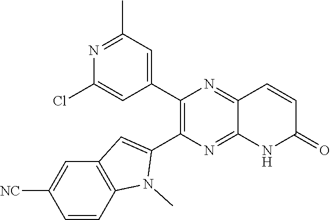 [SMILES: Cc1cc(-c2nc3ccc(=O)[nH]c3nc2-c2cc3cc(C#N)ccc3n2C)cc(Cl)n1] |
| 2.149 | 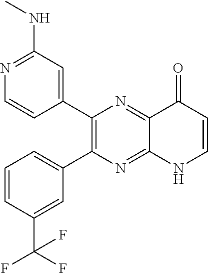 [SMILES: CNc1cc(-c2nc3c(=O)cc[nH]c3nc2-c2cccc(C(F)(F)F)c2)ccn1] |
| 2.150 | 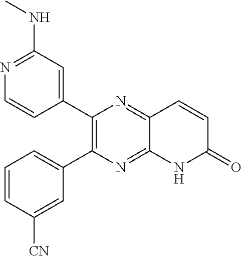 [SMILES: CNc1cc(-c2nc3ccc(=O)[nH]c3nc2-c2cccc(C#N)c2)ccn1] |
| 2.151 | 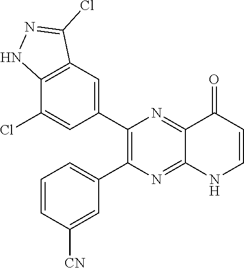 [SMILES: N#Cc1cccc(-c2nc3[nH]ccc(=O)c3nc2-c2cc(Cl)c3[nH]nc(Cl)c3c2)c1] |
| 2.152 | 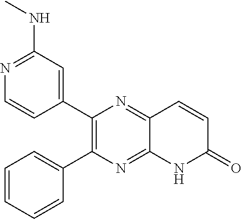 [SMILES: CNc1cc(-c2nc3ccc(=O)[nH]c3nc2-c2ccccc2)ccn1] |
| 2.153 | 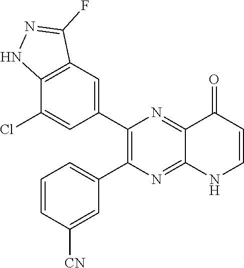 [SMILES: N#Cc1cccc(-c2nc3[nH]ccc(=O)c3nc2-c2cc(Cl)c3[nH]nc(F)c3c2)c1] |
| 2.154 | 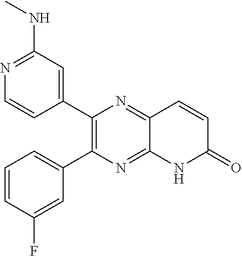 [SMILES: CNc1cc(-c2nc3ccc(=O)[nH]c3nc2-c2cccc(F)c2)ccn1] |
| 2.155 | 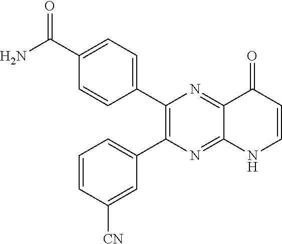 [SMILES: N#Cc1cccc(-c2nc3[nH]ccc(=O)c3nc2-c2ccc(C(N)=O)cc2)c1] |
| 2.156 | 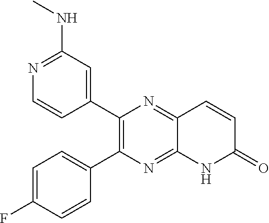 [SMILES: CNc1cc(-c2nc3ccc(=O)[nH]c3nc2-c2ccc(F)cc2)ccn1] |
| 2.157 | 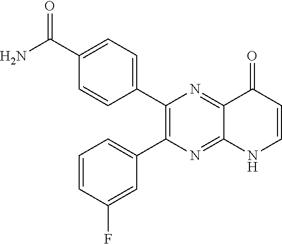 [SMILES: NC(=O)c1ccc(-c2nc3c(=O)cc[nH]c3nc2-c2cccc(F)c2)cc1] |
| 2.158 | 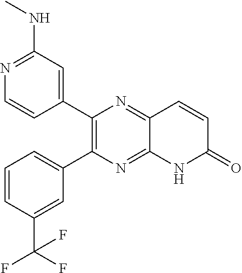 [SMILES: CNc1cc(-c2nc3ccc(=O)[nH]c3nc2-c2cccc(C(F)(F)F)c2)ccn1] |
| 2.159 | 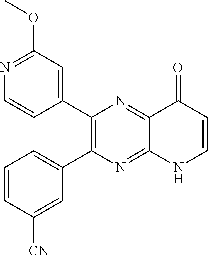 [SMILES: COc1cc(-c2nc3c(=O)cc[nH]c3nc2-c2cccc(C#N)c2)ccn1] |
| 2.160 | 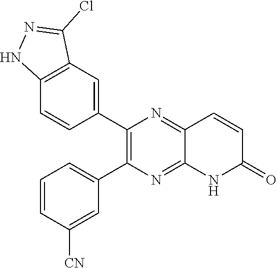 [SMILES: N#Cc1cccc(-c2nc3[nH]c(=O)ccc3nc2-c2ccc3[nH]nc(Cl)c3c2)c1] |
| 2.161 | 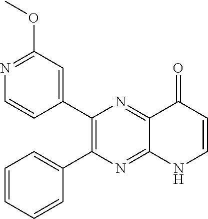 [SMILES: COc1cc(-c2nc3c(=O)cc[nH]c3nc2-c2ccccc2)ccn1] |
| 2.162 | 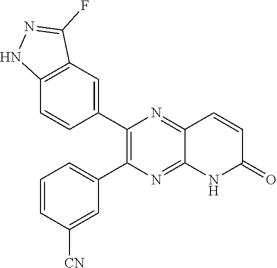 [SMILES: N#Cc1cccc(-c2nc3[nH]c(=O)ccc3nc2-c2ccc3[nH]nc(F)c3c2)c1] |
| 2.163 | 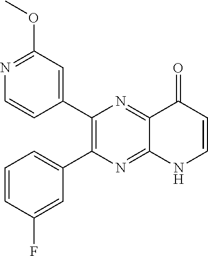 [SMILES: COc1cc(-c2nc3c(=O)cc[nH]c3nc2-c2cccc(F)c2)ccn1] |
| 2.164 | 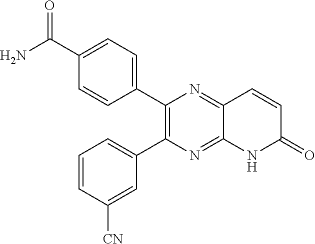 [SMILES: N#Cc1cccc(-c2nc3[nH]c(=O)ccc3nc2-c2ccc(C(N)=O)cc2)c1] |
| 2.165 | 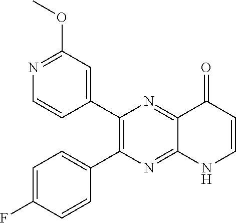 [SMILES: COc1cc(-c2nc3c(=O)cc[nH]c3nc2-c2ccc(F)cc2)ccn1] |
| 2.166 | 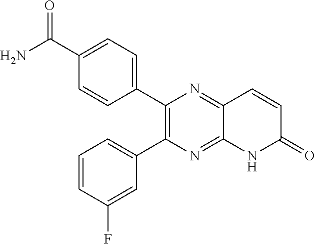 [SMILES: NC(=O)c1ccc(-c2nc3ccc(=O)[nH]c3nc2-c2cccc(F)c2)cc1] |
| 2.167 | 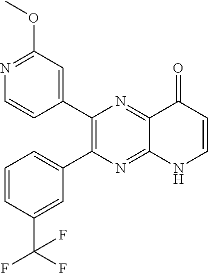 [SMILES: COc1cc(-c2nc3c(=O)cc[nH]c3nc2-c2cccc(C(F)(F)F)c2)ccn1] |
| 2.168 | 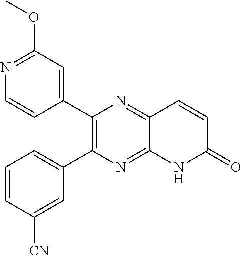 [SMILES: COc1cc(-c2nc3ccc(=O)[nH]c3nc2-c2cccc(C#N)c2)ccn1] |
| 2.169 | 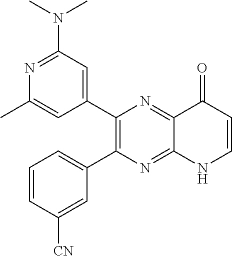 [SMILES: Cc1cc(-c2nc3c(=O)cc[nH]c3nc2-c2cccc(C#N)c2)cc(N(C)C)n1] |
| 2.170 | 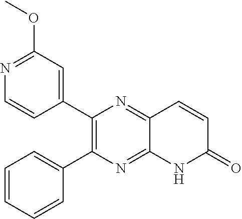 [SMILES: COc1cc(-c2nc3ccc(=O)[nH]c3nc2-c2ccccc2)ccn1] |
| 2.171 | 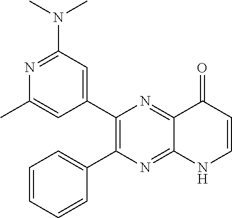 [SMILES: Cc1cc(-c2nc3c(=O)cc[nH]c3nc2-c2ccccc2)cc(N(C)C)n1] |
| 2.172 | 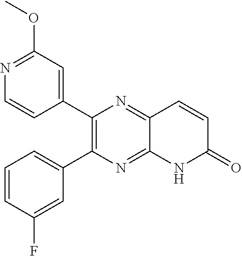 [SMILES: COc1cc(-c2nc3ccc(=O)[nH]c3nc2-c2cccc(F)c2)ccn1] |
| 2.173 | 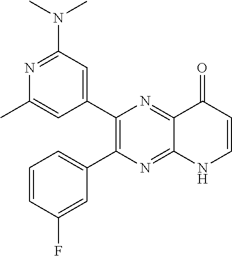 [SMILES: Cc1cc(-c2nc3c(=O)cc[nH]c3nc2-c2cccc(F)c2)cc(N(C)C)n1] |
| 2.174 | 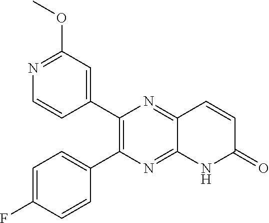 [SMILES: COc1cc(-c2nc3ccc(=O)[nH]c3nc2-c2ccc(F)cc2)ccn1] |
| 2.175 | 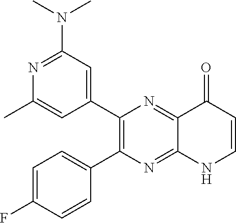 [SMILES: Cc1cc(-c2nc3c(=O)cc[nH]c3nc2-c2ccc(F)cc2)cc(N(C)C)n1] |
| 2.176 | 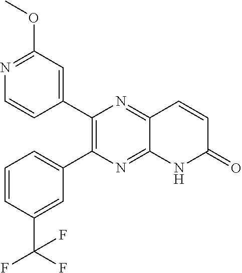 [SMILES: COc1cc(-c2nc3ccc(=O)[nH]c3nc2-c2cccc(C(F)(F)F)c2)ccn1] |
| 2.177 | 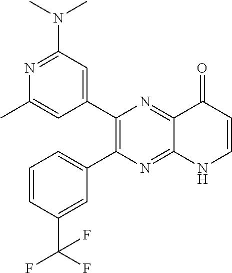 [SMILES: Cc1cc(-c2nc3c(=O)cc[nH]c3nc2-c2cccc(C(F)(F)F)c2)cc(N(C)C)n1] |
| 2.178 | 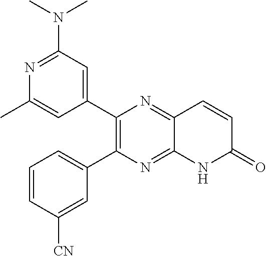 [SMILES: Cc1cc(-c2nc3ccc(=O)[nH]c3nc2-c2cccc(C#N)c2)cc(N(C)C)n1] |
| 2.179 | 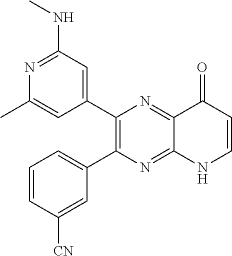 [SMILES: CNc1cc(-c2nc3c(=O)cc[nH]c3nc2-c2cccc(C#N)c2)cc(C)n1] |
| 2.180 | 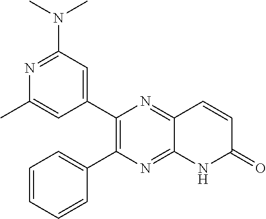 [SMILES: Cc1cc(-c2nc3ccc(=O)[nH]c3nc2-c2ccccc2)cc(N(C)C)n1] |
| 2.181 | 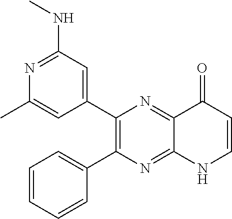 [SMILES: CNc1cc(-c2nc3c(=O)cc[nH]c3nc2-c2ccccc2)cc(C)n1] |
| 2.182 | 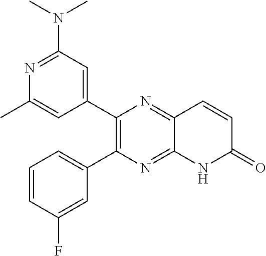 [SMILES: Cc1cc(-c2nc3ccc(=O)[nH]c3nc2-c2cccc(F)c2)cc(N(C)C)n1] |
| 2.183 | 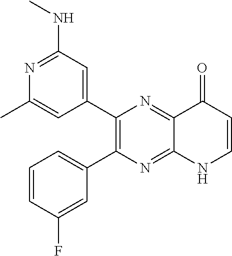 [SMILES: CNc1cc(-c2nc3c(=O)cc[nH]c3nc2-c2cccc(F)c2)cc(C)n1] |
| 2.184 | 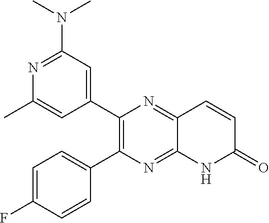 [SMILES: Cc1cc(-c2nc3ccc(=O)[nH]c3nc2-c2ccc(F)cc2)cc(N(C)C)n1] |
| 2.185 | 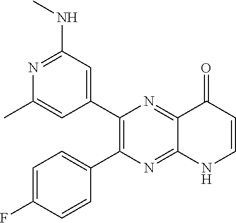 [SMILES: CNc1cc(-c2nc3c(=O)cc[nH]c3nc2-c2ccc(F)cc2)cc(C)n1] |
| 2.186 | 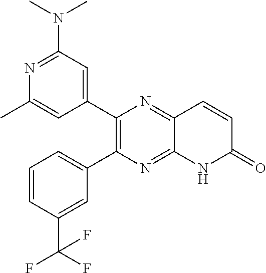 [SMILES: Cc1cc(-c2nc3ccc(=O)[nH]c3nc2-c2cccc(C(F)(F)F)c2)cc(N(C)C)n1] |
| 2.187 | 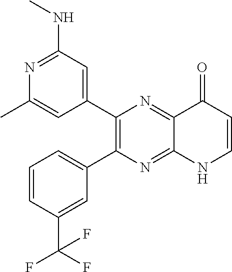 [SMILES: CNc1cc(-c2nc3c(=O)cc[nH]c3nc2-c2cccc(C(F)(F)F)c2)cc(C)n1] |
| 2.188 | 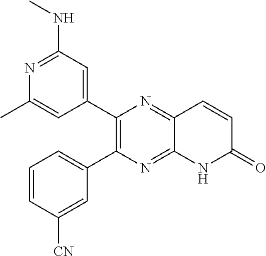 [SMILES: CNc1cc(-c2nc3ccc(=O)[nH]c3nc2-c2cccc(C#N)c2)cc(C)n1] |
| 2.189 | 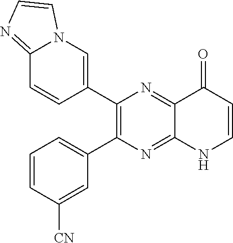 [SMILES: N#Cc1cccc(-c2nc3[nH]ccc(=O)c3nc2-c2ccc3nccn3c2)c1] |
| 2.190 | 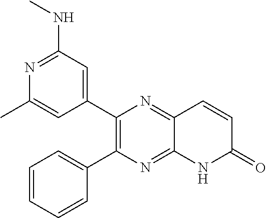 [SMILES: CNc1cc(-c2nc3ccc(=O)[nH]c3nc2-c2ccccc2)cc(C)n1] |
| 2.191 | 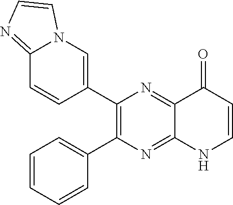 [SMILES: O=c1cc[nH]c2nc(-c3ccccc3)c(-c3ccc4nccn4c3)nc12] |
| 2.192 | 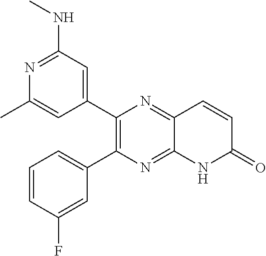 [SMILES: CNc1cc(-c2nc3ccc(=O)[nH]c3nc2-c2cccc(F)c2)cc(C)n1] |
| 2.193 | 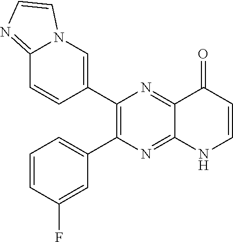 [SMILES: O=c1cc[nH]c2nc(-c3cccc(F)c3)c(-c3ccc4nccn4c3)nc12] |
| 2.194 | 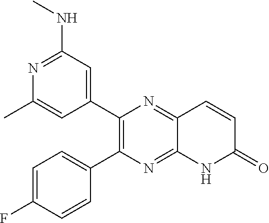 [SMILES: CNc1cc(-c2nc3ccc(=O)[nH]c3nc2-c2ccc(F)cc2)cc(C)n1] |
| 2.195 | 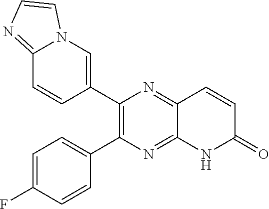 [SMILES: O=c1ccc2nc(-c3ccc4nccn4c3)c(-c3ccc(F)cc3)nc2[nH]1] |
| 2.196 | 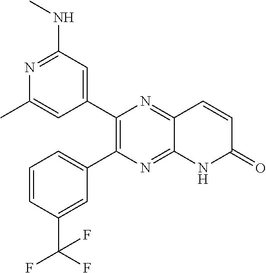 [SMILES: CNc1cc(-c2nc3ccc(=O)[nH]c3nc2-c2cccc(C(F)(F)F)c2)cc(C)n1] |
| 2.197 | 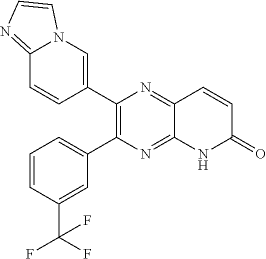 [SMILES: O=c1ccc2nc(-c3ccc4nccn4c3)c(-c3cccc(C(F)(F)F)c3)nc2[nH]1] |
| 2.198 | 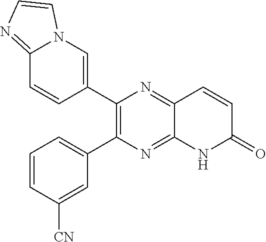 [SMILES: N#Cc1cccc(-c2nc3[nH]c(=O)ccc3nc2-c2ccc3nccn3c2)c1] |
| 2.199 | 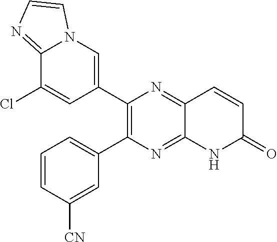 [SMILES: N#Cc1cccc(-c2nc3[nH]c(=O)ccc3nc2-c2cc(Cl)c3nccn3c2)c1] |
| 2.200 | 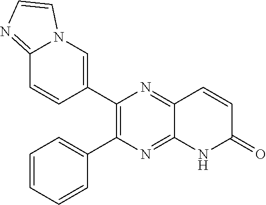 [SMILES: O=c1ccc2nc(-c3ccc4nccn4c3)c(-c3ccccc3)nc2[nH]1] |
| 2.201 | 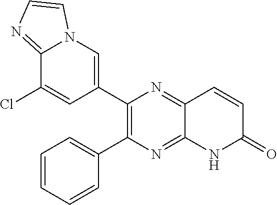 [SMILES: O=c1ccc2nc(-c3cc(Cl)c4nccn4c3)c(-c3ccccc3)nc2[nH]1] |
| 2.202 | 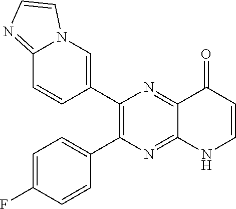 [SMILES: O=c1cc[nH]c2nc(-c3ccc(F)cc3)c(-c3ccc4nccn4c3)nc12] |
| 2.203 | 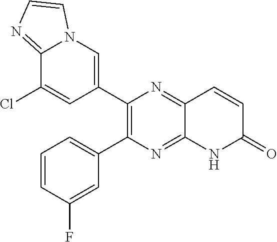 [SMILES: O=c1ccc2nc(-c3cc(Cl)c4nccn4c3)c(-c3cccc(F)c3)nc2[nH]1] |
| 2.204 | 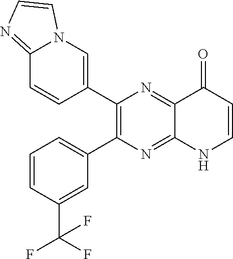 [SMILES: O=c1cc[nH]c2nc(-c3cccc(C(F)(F)F)c3)c(-c3ccc4nccn4c3)nc12] |
| 2.205 | 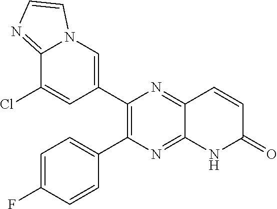 [SMILES: O=c1ccc2nc(-c3cc(Cl)c4nccn4c3)c(-c3ccc(F)cc3)nc2[nH]1] |
| 2.206 | 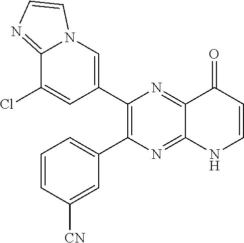 [SMILES: N#Cc1cccc(-c2nc3[nH]ccc(=O)c3nc2-c2cc(Cl)c3nccn3c2)c1] |
| 2.207 | 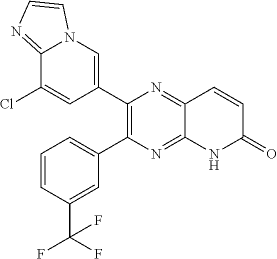 [SMILES: O=c1ccc2nc(-c3cc(Cl)c4nccn4c3)c(-c3cccc(C(F)(F)F)c3)nc2[nH]1] |
| 2.208 | 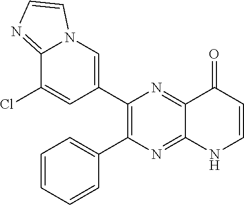 [SMILES: O=c1cc[nH]c2nc(-c3ccccc3)c(-c3cc(Cl)c4nccn4c3)nc12] |
| 2.209 | 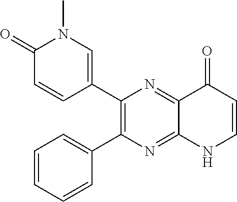 [SMILES: Cn1cc(-c2nc3c(=O)cc[nH]c3nc2-c2ccccc2)ccc1=O] |
| 2.210 | 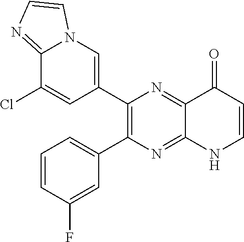 [SMILES: O=c1cc[nH]c2nc(-c3cccc(F)c3)c(-c3cc(Cl)c4nccn4c3)nc12] |
| 2.211 | 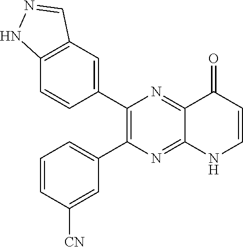 [SMILES: N#Cc1cccc(-c2nc3[nH]ccc(=O)c3nc2-c2ccc3[nH]ncc3c2)c1] |
| 2.212 | 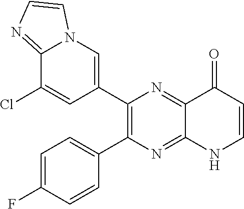 [SMILES: O=c1cc[nH]c2nc(-c3ccc(F)cc3)c(-c3cc(Cl)c4nccn4c3)nc12] |
| 2.213 | 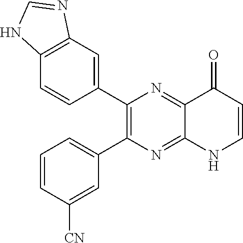 [SMILES: N#Cc1cccc(-c2nc3[nH]ccc(=O)c3nc2-c2ccc3[nH]cnc3c2)c1] |
| 2.214 | 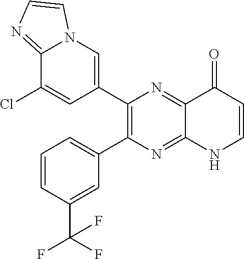 [SMILES: O=c1cc[nH]c2nc(-c3cccc(C(F)(F)F)c3)c(-c3cc(Cl)c4nccn4c3)nc12] |
| 2.215 | 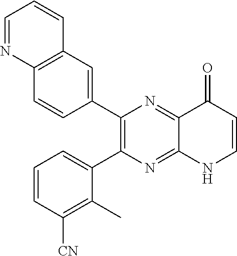 [SMILES: Cc1c(C#N)cccc1-c1nc2[nH]ccc(=O)c2nc1-c1ccc2ncccc2c1] |
| 2.216 | 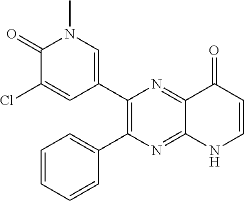 [SMILES: Cn1cc(-c2nc3c(=O)cc[nH]c3nc2-c2ccccc2)cc(Cl)c1=O] |
| 2.217 | 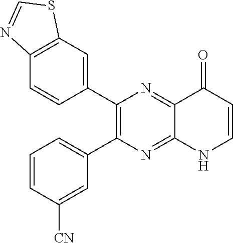 [SMILES: N#Cc1cccc(-c2nc3[nH]ccc(=O)c3nc2-c2ccc3ncsc3c2)c1] |
| 2.218 | 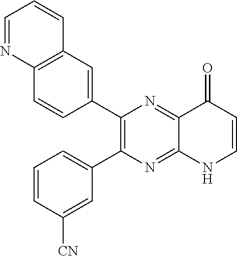 [SMILES: N#Cc1cccc(-c2nc3[nH]ccc(=O)c3nc2-c2ccc3ncccc3c2)c1] |
| 2.219 | 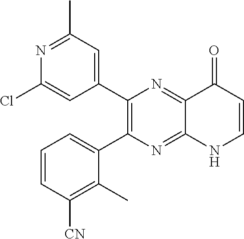 [SMILES: Cc1cc(-c2nc3c(=O)cc[nH]c3nc2-c2cccc(C#N)c2C)cc(Cl)n1] |

In some embodiments, provided herein are compounds described in Table 1, or a tautomer or isomer thereof, or a pharmaceutically acceptable salt of any of the foregoing, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible isomers of a compound depicted.

Additionally, the structure or name is intended to embrace tautomeric forms of the compounds described herein. For example, when R¹ is hydrogen, the tautomer of Formula (II) is Formula (IIa):

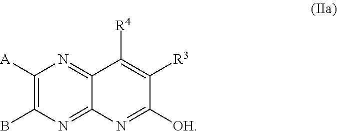

Similarly, when R¹ is hydrogen, the tautomer of Formula (III) is Formula (IIIa):

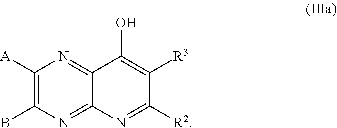

All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution study. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate

In some embodiments, compounds of the formula (I) may be synthesized according to Scheme 1, 2, 3, or 4.

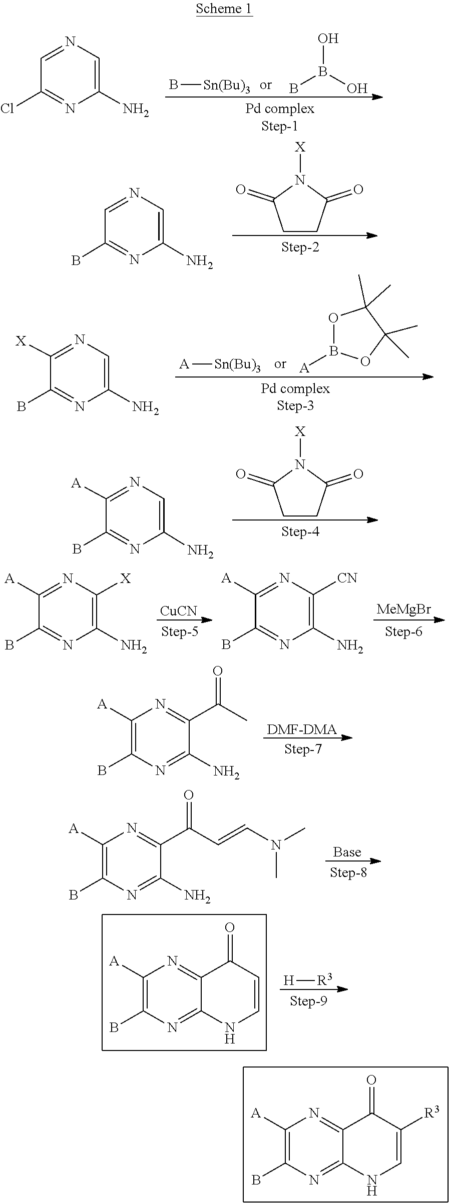

wherein A, B and R³ are as defined for formula (I), or any variation thereof detailed herein. It is understood that modifications of Scheme 1 can be made, such as further substitution of the structures depicted. Particular examples are provided in the Example section below.

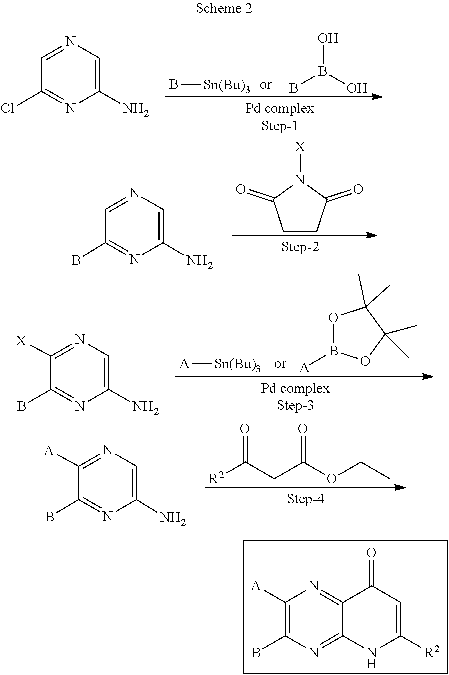

wherein A, B and R² are as defined for formula (I), or any variation thereof detailed herein. It is understood that modifications of Scheme 2 can be made, such as further substitution of the structures depicted. Particular examples are provided in the Example section below.

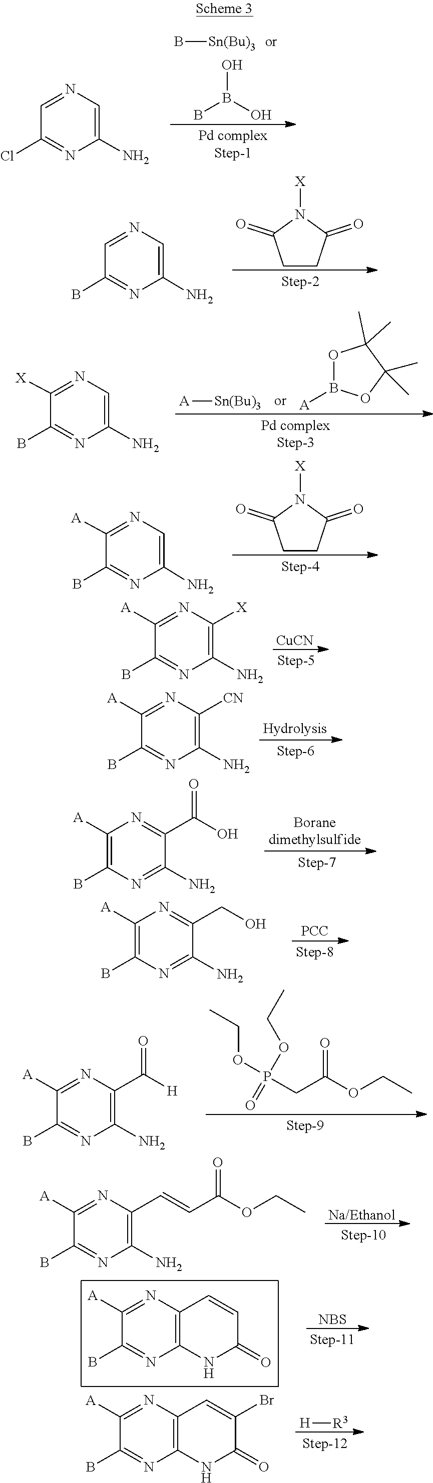

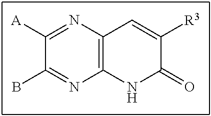

wherein A, B and R³ are as defined for formula (I), or any variation thereof detailed herein. It is understood that modifications of Scheme 3 can be made, such as further substitution of the structures depicted. Particular examples are provided in the Example section below.

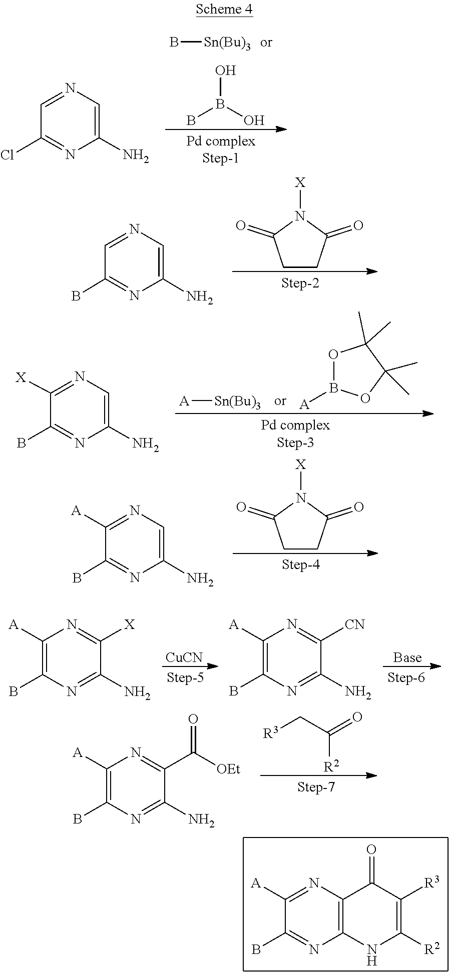

wherein A, B, R² and R³ are as defined for formula (I), or any variation thereof detailed herein. It is understood that modifications of Scheme 4 can be made, such as further substitution of the structures depicted. Particular examples are provided in the Example section below.

It is understood that General Synthetic Scheme 1, Scheme 2, Scheme 3 and Scheme 4 present synthetic routes involving steps clearly familiar to those skilled in the art, wherein the substituents described in compounds of formula (I) herein can be varied with a choice of appropriate starting materials and reagents utilized in the steps presented.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a pharmaceutically acceptable salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease in an individual comprising administering an effective amount of a compound of formula (I) or any embodiment, variation or aspect thereof (collectively, a compound of formula (I) or the present compounds or the compounds detailed or described herein) or a pharmaceutically acceptable salt thereof, to the individual. In some embodiments, provided herein is a method of treating a disease mediated by a G protein coupled receptor signaling pathway in an individual comprising administering an effective amount of a compound of formula (I), or a pharmaceutically acceptable salt thereof, to the individual. In some embodiments, the disease is mediated by a class A G protein coupled receptor. In some embodiments, the disease is mediated by a class B G protein coupled receptor. In some embodiments, the disease is mediated by a class C G protein coupled receptor. In some embodiments, the G protein coupled receptor is a purinergic G protein receptor. In some embodiments, the G protein coupled receptor is an adenosine receptor, such as any of the A₁, A_{2A}, A_{2B}, and A₃ receptors.

The present compounds or salts thereof are believed to be effective for treating a variety of diseases and disorders. For example, in some embodiments, the present compositions may be used to treat a proliferative disease, such as cancer. In some embodiments the cancer is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer, including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In some embodiments, the present compounds or salts thereof are used in treatment of tumors which produce high levels of ATP and/or adenosine. For example, in some embodiments the extracellular concentration of adenosine is 10-20 times higher in the tumor compared to adjacent tissue. In some embodiments, the present compounds or salts thereof are used in treatment of tumors that express high levels of an ectonucleotidase. In some embodiments, the ectonucleotidase is CD39. In some embodiments, the ectonucleotidase is CD73.

Also provided herein is a method of enhancing an immune response in an individual in need thereof comprising administering an effective amount of a compound of formula (I), or a pharmaceutically acceptable salt thereof, to the individual. Adenosine receptors are known to play an immunosuppressive role in cancer biology. High levels of adenosine present in the tumor microenvironment bind to adenosine receptors on immune cells to provide an immunosuppressive microenvironment. Specifically, binding of adenosine to the A_{2A} receptor provides an immunosuppressive signal that inhibits T cell proliferation, cytokine production and cytotoxicity. The A_{2A} receptor signaling has been implicated in adenosine-mediated inhibition of NK cell cytotoxicity, NKT cell cytokine production and CD40L upregulation. Therefore, use of an A_{2A} receptor antagonist, such as those provided herein, may reverse the immunosuppressive effect of adenosine on immune cells. In some embodiments, the immune response is enhanced by a compound of formula (I) or a salt thereof enhancing activity of natural killer (NK) cells. In some embodiments, the present compounds or salts thereof increase NK cell-meditated cytotoxicity. In some embodiments, the immune response is enhanced by enhancing the activity of CD8⁺ T cells. In some embodiments, the present compounds or salts thereof cause an inflammatory response in the tumor microenvironment.

The present disclosure further provides a method of increasing the activity of a natural killer cell in an individual comprising administering an effective amount of a compound of formula (I), or a pharmaceutically acceptable salt thereof, to the individual. In some of these embodiments, the present compounds or salts thereof increase NK cell-meditated cytotoxicity. In some embodiments, a compound of formula (I) or a salt thereof increases the number of NK cells.

A compound of formula (I) or a salt thereof may be useful for modulating the activity of G protein receptor coupled signaling pathway proteins. In some embodiments, a compound of formula (I) or a salt thereof activates a G protein receptor coupled signaling pathway protein (i.e. is an agonist of a G protein receptor). In some embodiments, a compound of formula (I) or a salt thereof inhibits a G protein receptor coupled signaling pathway protein (i.e., is a G protein receptor antagonist). In some embodiments, a compound of formula (I) or a salt thereof is an adenosine receptor antagonist. In some embodiments, a compound of formula (I) or a salt thereof is an antagonist of any of the A₁, A_{2A}, A_{2B}, and A₃ receptors.

Accordingly, also provided herein is a method of modulating the activity of an A_{2A} receptor in an individual comprising administering an effective amount of a compound of formula (I), or a pharmaceutically acceptable salt thereof to an individual. In some embodiments a compound of formula (I) or a salt thereof is an A_{2A} receptor antagonist. In some embodiments, a compound of formula (I) or a salt thereof reduces A_{2A} receptor signaling by at least 10%, 20%, 30%, 40%, 50%, 60%, 70%, 80%, 90%, 91%, 92%, 93%, 94%, 95%, 96%, 97%, 98%, or 99%. In some embodiments, a compound of formula (I) or a salt thereof reduces A_{2A} receptor signaling by 40-99%, 50-99%, 60-99%, 70-99%, 80-99%, 90-99%, or 95-99%. In some of these embodiments, a compound of formula (I) or a salt thereof binds to the A_{2A} receptor with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 10 nM, less than 1 nM or less than 100 pM. In some embodiments, [compound x] binds to the A_{2A} receptor with an IC₅₀ of 500 nM to 100 pM, 400 nM to 100 pM, 300 nM to 100 pM, 200 nM to 100 pM, or 100 nM to 100 pM.

Also provided herein is a method of modulating the activity of an A_{2B} receptor in an individual comprising administering an effective amount of a compound of formula (I), or a pharmaceutically acceptable salt thereof to an individual. In some embodiments a compound of formula (I) or a salt thereof is an A_{2B} receptor antagonist. In some embodiments, a compound of formula (I) or a salt thereof reduces A_{2B} receptor signaling by at least 10%, 20%, 30%, 40%, 50%, 60%, 70%, 80%, 90%, 91%, 92%, 93%, 94%, 95%, 96%, 97%, 98%, or 99%. In some embodiments, a compound of formula (I) or a salt thereof reduces A_{2B} receptor signaling by 40-99%, 50-99%, 60-99%, 70-99%, 80-99%, 90-99%, or 95-99%. In some of these embodiments, a compound of formula (I) or a salt thereof binds to the A_{2B} receptor with an IC₅₀ of less than 1 pM, less than 900 nM, less than 800 nM, less than 700 nM, less than 600 nM, less than 500 nM, less than 400 nM, less than 300 nM, less than 200 nM, less than 100 nM, less than 10 nM, less than 1 nM or less than 100 pM. In some embodiments, a compound of formula (I) or a salt thereof binds to the A_{2B} receptor with an IC₅₀ of 500 nM to 100 pM, 400 nM to 100 pM, 300 nM to 100 pM, 200 nM to 100 pM, or 100 nM to 100 pM.

Also provided herein is a method of modulating the activity of an A₃ receptor in an individual comprising administering an effective amount of a compound of formula (I), or a pharmaceutically acceptable salt thereof to an individual. In some embodiments a compound of formula (I) or a salt thereof is an A₃ receptor antagonist. In some embodiments, a compound of formula (I) or a salt thereof reduces A₃ receptor signaling by at least 10%, 20%, 30%, 40%, 50%, 60%, 70%, 80%, 90%, 91%, 92%, 93%, 94%, 95%, 96%, 97%, 98%, or 99%. In some embodiments, a compound of formula (I) or a salt thereof reduces A₃ receptor signaling by 40-99%, 50-99%, 60-99%, 70-99%, 80-99%, 90-99%, or 95-99%. In some of these embodiments, a compound of formula (I) or a salt thereof binds to the A₃ receptor with an IC₅₀ of less than 1 pM, less than 900 nM, less than 800 nM, less than 700 nM, less than 600 nM, less than 500 nM, less than 400 nM, less than 300 nM, less than 200 nM, less than 100 nM, less than 10 nM, less than 1 nM or less than 100 pM. In some embodiments, a compound of formula (I) or a salt thereof binds to the A₃ receptor with an IC₅₀ of 500 nM to 100 pM, 400 nM to 100 pM, 300 nM to 100 pM, 200 nM to 100 pM, or 100 nM to 100 pM.

In some embodiments, the present invention comprises a method of inhibiting tumor metastasis in an individual in need thereof comprising administering a compound of formula (I), or a pharmaceutically acceptable salt thereof, to the individual. In some embodiments, the metastasis is to the lung, liver, lymph node, bone, adrenal gland, brain, peritoneum, muscle, or vagina. In some embodiments, a compound of formula (I) or a salt thereof inhibits metastasis of melanoma cells. In some embodiments, the present disclosure includes a method of delaying tumor metastasis comprising administering a compound of formula (I), or a pharmaceutically acceptable salt thereof, to the individual. In some of these embodiments, the time to metastasis is delayed by 1 month, 2 months 3 months, 4 months, 5 months, 6 months, 12 months, or more, upon treatment with the compounds of the present invention.

In some embodiments, a compound of formula (I) or a salt thereof is used to treat an individual having a proliferative disease, such as cancer as described herein. In some embodiments, the individual is at risk of developing a proliferative disease, such as cancer. In some of these embodiments, the individual is determined to be at risk of developing cancer based upon one or more risk factors. In some of these embodiments, the risk factor is a family history and/or gene associated with cancer. In some embodiments, the individual has a cancer that expresses a high level of a nucleotide metabolizing enzyme. In some embodiments, the nucleotide metabolizing enzyme is a nucleotidase, such as CD73 (ecto-5′-nucleotidase, Ecto5′NTase). In some of these embodiments, the individual has a cancer that expresses a high level of a nucleotidase, such as CD73. In any of these embodiments, the nucleotide metabolizing enzyme is an ecto-nucleotidase. In some embodiments, the ecto-nucleotidase degrades adenosine monophosphate. In some embodiments, the nucleotide metabolizing enzyme is CD39 (ecto-nucleoside triphosphate diphosphohydrolase 1, E-NTPDase1). In some of these embodiments, the individual has a cancer that expresses a high level of CD39. In some embodiments, the individual has a cancer that expresses a high level of an adenosine receptor, such as the A_{2A} receptor.

### Combination Therapy

As provided herein, the presently disclosed compounds or a salt thereof may activate the immune system by modulating the activity of a G protein coupled receptor signaling pathway, for example acting as an A_{2A} receptor antagonist, which results in significant anti-tumor effects. Accordingly, the present compounds or a salt thereof may be used in combination with other anti-cancer agents to enhance tumor immunotherapy. In some embodiments, provided herein is a method of treating a disease mediated by a G protein coupled receptor signaling pathway in an individual comprising administering an effective amount of a compound of formula (I), or a pharmaceutically acceptable salt thereof, and an additional therapeutic agent to the individual. In some embodiments, the disease mediated by a G protein coupled receptor signaling pathway is a proliferative disease such as cancer.

In some embodiments, the additional therapeutic agent is a cancer immunotherapy. In some embodiments, the additional therapeutic agent is an immunostimulatory agent. In some embodiments, the additional therapeutic agent targets a checkpoint protein. In some embodiments, the additional therapeutic agent is effective to stimulate, enhance or improve an immune response against a tumor.

In another aspect, provided herein is a combination therapy in which a compound of formula (I) is coadministered (which may be separately or simultaneously) with one or more additional agents that are effective in stimulating immune responses to thereby further enhance, stimulate or upregulate immune responses in a subject. For example, provided is a method for stimulating an immune response in a subject comprising administering to the subject a compound of formula (I) or a salt thereof and one or more immunostimulatory antibodies, such as an anti-PD-1 antibody, an anti-PD-L1 antibody and/or an anti-CTLA-4 antibody, such that an immune response is stimulated in the subject, for example to inhibit tumor growth. As another example, provided is a method for stimulating an immune response in a subject comprising administering to the subject a compound of formula (I) or a salt thereof and one or more immunostimulatory antibodies or immunotherapy like Chimeric antigen receptor (CAR) T-cell therapy; immunostimulatory antibodies, such as an anti-PD-1 antibody, an anti-PD-L1 antibody and/or an anti-CTLA-4 antibody, such that an immune response is stimulated in the subject, for example to inhibit tumor growth. In one embodiment, the subject is administered a compound of formula (I) or a salt thereof and an anti-PD-1 antibody. In another embodiment, the subject is administered a compound of formula (I) or a salt thereof and an anti-PD-L1 antibody. In yet another embodiment, the subject is administered a compound of formula (I) or a salt thereof and an anti-CTLA-4 antibody. In another embodiment, the immunostimulatory antibody (e.g., anti-PD-1, anti-PD-L1 and/or anti-CTLA-4 antibody) is a human antibody. Alternatively, the immunostimulatory antibody can be, for example, a chimeric or humanized antibody (e.g., prepared from a mouse anti-PD-1, anti-PD-L1 and/or anti-CTLA-4 antibody). In another embodiment, the subject is administered a compound of formula (I) or a salt thereof and CART-cells (genetically modified T cells).

In one embodiment, the present disclosure provides a method for treating a proliferative disease (e.g., cancer), comprising administering a compound of formula (I) or a salt thereof and an anti-PD-1 antibody to a subject. In further embodiments, a compound of formula (I) or a salt thereof is administered at a subtherapeutic dose, the anti-PD-1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a compound of formula (I) or a salt thereof and a subtherapeutic dose of anti-PD-1 antibody to a subject. In certain embodiments, the subject is human. In certain embodiments, the anti-PD-1 antibody is a human sequence monoclonal antibody

In one embodiment, the present invention provides a method for treating a hyperproliferative disease (e.g., cancer), comprising administering a compound of formula (I) or a salt thereof and an anti-PD-L1 antibody to a subject. In further embodiments, a compound of formula (I) or a salt thereof is administered at a subtherapeutic dose, the anti-PD-L1 antibody is administered at a subtherapeutic dose, or both are administered at a subtherapeutic dose. In another embodiment, the present invention provides a method for altering an adverse event associated with treatment of a hyperproliferative disease with an immunostimulatory agent, comprising administering a compound of formula (I) or a salt thereof and a subtherapeutic dose of anti-PD-L1 antibody to a subject. In certain embodiments, the subject is human. In certain embodiments, the anti-PD-L1 antibody is a human sequence monoclonal antibody.

In certain embodiments, the combination of therapeutic agents discussed herein can be administered concurrently as a single composition in a pharmaceutically acceptable carrier, or concurrently as separate compositions each in a pharmaceutically acceptable carrier. In another embodiment, the combination of therapeutic agents can be administered sequentially. For example, an anti-CTLA-4 antibody and a compound of formula (I) or a salt thereof can be administered sequentially, such as anti-CTLA-4 antibody being administered first and a compound of formula (I) or a salt thereof second, or a compound of formula (I) or a salt thereof being administered first and anti-CTLA-4 antibody second. Additionally or alternatively, an anti-PD-1 antibody and a compound of formula (I) or a salt thereof can be administered sequentially, such as anti-PD-1 antibody being administered first and a compound of formula (I) or a salt thereof second, or a compound of formula (I) or a salt thereof being administered first and anti-PD-1 antibody second. Additionally or alternatively, an anti-PD-L1 antibody and a compound of formula (I) or a salt thereof can be administered sequentially, such as anti-PD-L1 antibody being administered first and a compound of formula (I) or a salt thereof second, or a compound of formula (I) or a salt thereof being administered first and anti-PD-L1 antibody second.

Furthermore, if more than one dose of the combination therapy is administered sequentially, the order of the sequential administration can be reversed or kept in the same order at each time point of administration, sequential administrations can be combined with concurrent administrations, or any combination thereof.

Optionally, the combination of a compound of formula (I) or a salt thereof can be further combined with an immunogenic agent, such as cancerous cells, purified tumor antigens (including recombinant proteins, peptides, and carbohydrate molecules), cells, and cells transfected with genes encoding immune stimulating cytokines.

A compound of formula (I) or a salt thereof can also be further combined with standard cancer treatments. For example, a compound of formula (I) or a salt thereof can be effectively combined with chemotherapeutic regimes. In these instances, it is possible to reduce the dose of other chemotherapeutic reagent administered with the combination of the instant disclosure (Mokyr et al. (1998) Cancer Research 58: 5301-5304). Other combination therapies with a compound of formula (I) or a salt thereof include radiation, surgery, or hormone deprivation. Angiogenesis inhibitors can also be combined with a compound of formula (I) or a salt thereof. Inhibition of angiogenesis leads to tumor cell death, which can be a source of tumor antigen fed into host antigen presentation pathways.

In another example, a compound of formula (I) or a salt thereof can be used in conjunction with anti-neoplastic antibodies. By way of example and not wishing to be bound by theory, treatment with an anti-cancer antibody or an anti-cancer antibody conjugated to a toxin can lead to cancer cell death (e.g., tumor cells) which would potentiate an immune response mediated by CTLA-4, PD-1, PD-L1 or a compound of formula (I) or a salt thereof. In an exemplary embodiment, a treatment of a hyperproliferative disease (e.g., a cancer tumor) can include an anti-cancer antibody in combination with a compound of formula (I) or a salt thereof and anti-CTLA-4 and/or anti-PD-1 and/or anti-PD-L1 antibodies, concurrently or sequentially or any combination thereof, which can potentiate anti-tumor immune responses by the host. Other antibodies that can be used to activate host immune responsiveness can be further used in combination with a compound of formula (I) or a salt thereof.

In some embodiments, a compound of formula (I) or a salt thereof can be combined with an anti-CD73 therapy, such as an anti-CD73 antibody.

In some embodiments, a compound of formula (I) or a salt thereof can be combined with an anti-CD39 therapy, such as an anti-CD39 antibody.

In yet further embodiments, a compound of formula (I) or a salt thereof is administered in combination another G protein receptor antagonist, such as an adenosine A₁ and/or A₃ antagonist.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

Also provided are articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein (e.g., hypertension) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

### Synthetic Examples

### Example S1: Synthesis of 3-phenyl-2-(quinolin-6-yl)pyrido[2,3-b]pyrazin-8(5H)-one (Compound No 1.5)

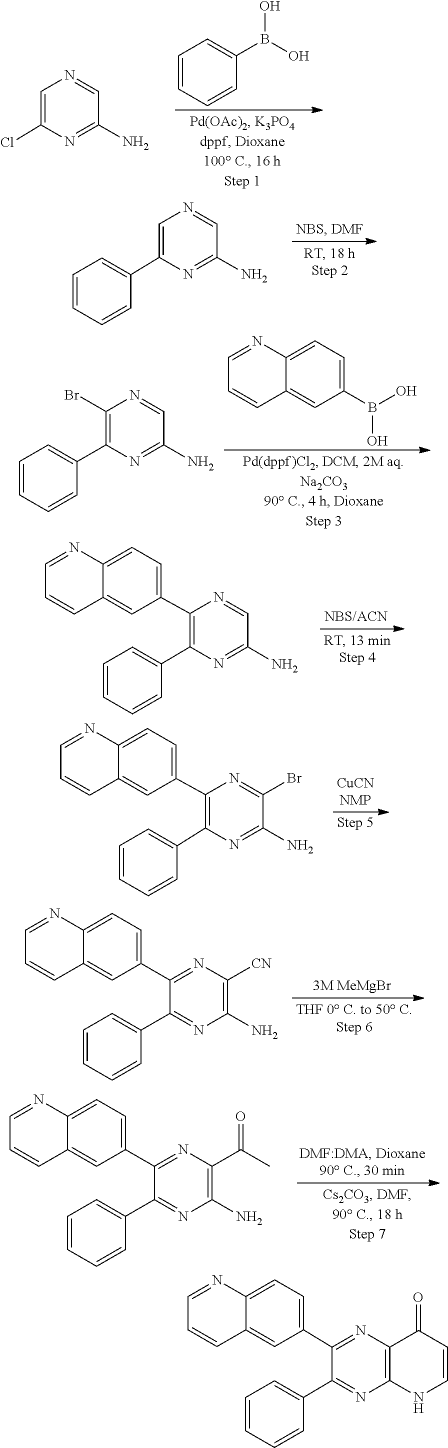

Step-1: Synthesis of 6-phenylpyrazin-2-amine: To a solution of 6-chloropyrazin-2-amine (1.00 g, 7.75 mmol, 1 eq) in 1,4-dioxane (30 mL) was added phenylboronic acid (1.42 g, 11.62 mmol, 1.5 eq), K₃PO₄ (3.286 g, 15.50 mmol, 2 eq), Pd(OAc)₂ (0.086 g, 0.38 mmol, 0.05 eq), 1,1′-bis(diphenylphosphino)ferrocene (0.214 g, 0.38 mmol, 0.05 eq). The reaction mixture was deoxygenated using N₂ atmosphere and the reaction mixture was heated at 100° C. overnight. The reaction was monitored by TLC and LCMS and found to be complete after 18 h. The reaction mixture was cooled to RT, filtered through ciliate-bed and washed with ethyl acetate (2×20 mL). The reaction mixture was diluted with water (50 mL) and extracted with ethyl acetate (2×50 mL). The separated organic layer was dried over sodium sulfate and concentrated under reduced pressure. The crude product was purified by CombiFlasb on silica gel using CH₃OH—CH₂Cl₂ system as eluent to afford 1.10 g (63%) of 6-phenylpyrazin-2-amine. LCMS: 172 [M+1]⁺

Step-2: Synthesis of 5-bromo-6-phenylpyrazin-2-amine: To a solution of 6-phenylpyrazin-2-amine (0.150 g, 0.877 mmol, 1 eq) in DMF (3 mL) was added N-bromosuccinimide (0.156 g, 0.877 mmol, 1 eq) and the reaction mixture was stirred at RT for 1 h. The reaction was monitored by TLC and NMR. After completion, the reaction mixture was diluted with water (50 mL) and extracted by ethyl acetate (2×20 mL). Combined organic layer was washed with water (5×20 mL) followed by brine and dried over anhydrous sodium sulfate. The solvent was evaporated under reduced pressure to get the crude product which was purified by CombiFalsh on silica gel using EtOAc-Hexane system as eluent to afford 100 mg (46%) of 5-bromo-6-phenylpyrazin-2-amine. LCMS: 251 [M+1]⁺

Step-3: Synthesis of 6-phenyl-5-(quinolin-6-yl)pyrazin-2-amine: To a stirred solution of quinolin-6-ylboronic acid (0.100 g, 0.57 mmol, 1.2 eq) and 5-bromo-6-phenylpyrazin-2-amine (0.120 g, 0.48 mmol, 1.0 eq) in dioxane (3 mL) was added 2M aqueous Na₂CO₃ (0.101 g, 0.96 mmol, 2.0 eq 0.5 mL). The reaction was purged with N₂ for 5 min. To this reaction mixture was added Pd(dppf)Cl₂.DCM (0.020 g, 5 mol %) and N₂ was purged again for 5 more mins. The reaction mixture was heated at 90° C. for 4h. The reaction mixture was allowed to cool to RT and extracted using ethyl acetate (2×35 mL). The combined organic layers were washed (brine), dried (anhydrous Na₂SO₄) and concentrated under vacuum to get the solid residue which was purified by normal phase silica gel flash column chromatography to get the desired product as off white solid (0.030 g, 21%). LCMS: 299 [M+1]⁺

Step-4: Synthesis of 3-bromo-6-phenyl-5-(quinolin-6-yl)pyrazin-2-amine: To a solution of 6-phenyl-5-(quinolin-6-yl)pyrazin-2-amine (20 mg, 0.068 mmol, 1 eq) in acetonitrile (12 mL) at room temperature was added N-bromosuccinimide (12 mg, 0.068 mmol, 1 eq) portion wise and the reaction mixture was allowed to stir at room temperature. Progress of reaction was monitored by TLC and was found to be complete after 13 minutes. Reaction mixture was diluted with water and extracted with ethyl acetate (3×20 mL). Combined organic layer was washed with water (3×20 mL) and dried over anhydrous sodium sulfate. Removal of solvent gave crude which was purified by reversed phase HPLC to give 10 mg (40%) of 3-bromo-6-phenyl-5-(quinolin-6-yl)pyrazin-2-amine. LCMS: 377 [M+1]⁺

Step-5: Synthesis of 3-amino-5-phenyl-6-(quinolin-6-yl)pyrazine-2-carbonitrile: To a stirred solution of 6-phenyl-5-(quinolin-6-yl)pyrazin-2-amine (0.220 g, 0.58 mmol, 1.0 eq) in NMP (1.5 mL) was added cuprous cyanide (0.155 g, 1.74 mmol, 3.0 eq). The reaction mixture was allowed to stir at 170° C. for 1h. The progress of the reaction was monitored by LCMS. The reaction mixture was allowed to cool to RT and extracted using ethyl acetate (3×50 mL). The combined organic layers were washed (brine), dried (anhydrous Na₂SO₄) and concentrated under vacuum to get the solid which was purified by normal phase column chromatography to get the desired product as an off white solid (0.020 g, 10%). LCMS: 324 [M+1]⁺

Step-6: Synthesis of 1-(3-amino-5-phenyl-6-(quinolin-6-yl)pyrazin-2-yl)ethan-1-one: To a stirred solution of 3-amino-5-phenyl-6-(quinolin-6-yl)pyrazine-2-carbonitrile (0.100 g, 0.32 mmol, 1.0 eq) in THF (5 mL) was added 3M MeMgBr in diethyl ether (1 mL, 0.360 g, 10.0 eq 3.0 mmol) at 0° C. The resulting reaction mixture was stirred at 50° C. for 16h. Reaction mixture was then cool to RT and acidified slowly with dilute HCl. The acidified reaction mixture was stirred for 1h at 50° C. The reaction mixture was again allowed to cool to RT and extracted by using ethyl acetate (2×25 mL). The combined organic layers were washed (brine), dried (anhydrous Na₂SO₄) and concentrated under vacuum to get the desired product as light yellow solid (0.090 g, 97%) LCMS: 291 [M+1]⁺

Step-7: Synthesis of 3-phenyl-2-(quinolin-6-yl)pyrido[2,3-b]pyrazin-8(5H)-one: To a solution of 1-(6-amino-3-(quinolin-6-yl)-2,3′-bipyridin-5-yl)ethanone (0.090 g, 0.31 mmol, 1.00 eq), in 1,4 dioxane (5 mL), was added DMF:DMA (0.044 g, 0.37 mmol, 1.2 eq). The reaction mixture was heated at 90° C. for 30 minutes. The reaction was monitored by TLC and LCMS. The reaction solvent was evaporated under reduced pressure. The semisolid crude material obtained was redissolved in DMF (2 mL) and Cs₂CO₃ (0.150 g, 0.46 mmol, 1.5 eq) was added. The reaction mixture was again heated at 90° C. for 18 h. The progress of the reaction was monitored by LCMS. The reaction mixture was diluted with ice cold water (25 mL) and extracted by using ethyl acetate (3×25 mL). The residue was purified by reverse phase column chromatography to afford the desired product (0.010 g, 10%). LCMS: 351 [M+1]⁺; ¹H NMR (400 MHz, DMSO-d₆) δ 8.92 (d, J=3.07 Hz, 1H), 8.35 (d, J=7.45 Hz, 1H), 8.14 (br s, 1H), 8.06 (d, J=5.26 Hz, 1H), 7.91 (d, J=8.77 Hz, 1H), 7.65 (d, J=7.02 Hz, 1H), 7.45-7.59 (m, 3H), 7.29-7.44 (m, 3H), 6.33 (d, J=7.02 Hz, 1H).

### Example S2: Synthesis of 5-methyl-3-phenyl-2-(quinolin-6-yl)pyrido[2,3-b]pyrazin-8(5H)-one (Compound No 1.8)

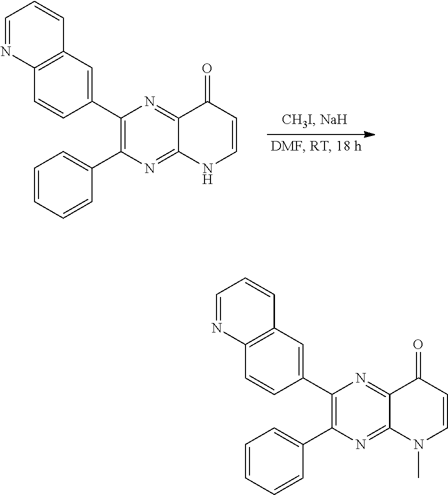 [SMILES: O=c1cc[nH]c2nc(-c3ccccc3)c(-c3ccc4ncccc4c3)nc12 ; Cn1ccc(=O)c2nc(-c3ccc4ncccc4c3)c(-c3ccccc3)nc21]

To a solution of 3-phenyl-2-(quinolin-6-yl)pyrido[2,3-b]pyrazin-8(5H)-one (10 mg, 0.02 mmol, 1.00 eq) in DMF (5 mL) was added cesium carbonate (18.5 mg, 0.05 mmol, 2.0 eq). After 10 minute methyl iodide (3 mg, 0.02 mmol, 1.2 eq) was added and reaction mixture was stirred at room temperature for 1 h. The reaction mixture was diluted with ice cold water (20 mL) and extracted with ethyl acetate (2×20 ml). The combined organic layer was wash with water (5×20 mL). The organic layer was dried over sodium sulfate and concentrated under reduced pressure to afford the title compound (0.004 g, 40%). LCMS: 351 [M+1]⁺. ¹H NMR (DMSO-d₆, 400 MHz) δ 8.94 (s, 1H), 8.39 (d, 1H), 8.24 (d, 1H), 8.19 (s, 1H), 7.94 (d, 1H), 7.66 (d, 1H), 7.55-7.61 (m, 2H), 7.43 (d, 1H), 7.34-7.40 (m, 3H), 6.37 (d, 1H), 3.94 (s, 3H).

### Example S3: Synthesis of 2-(8-chloroquinolin-6-yl)-3-phenylpyrido[2,3-b]pyrazin-6(5H)-one (Compound No 1.12)

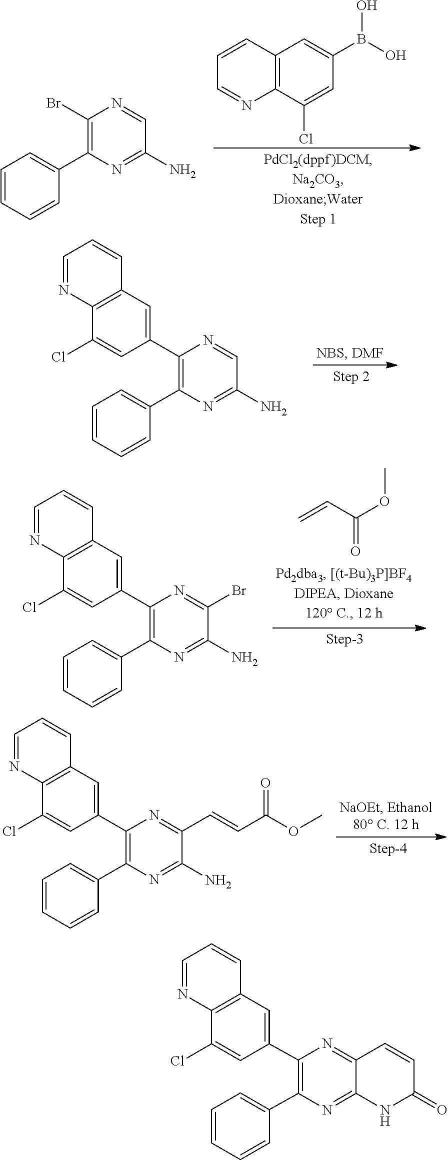

Step 1: Synthesis of 5-(8-chloroquinolin-6-yl)-6-phenylpyrazin-2-amine: To a solution of 5-bromo-6-phenylpyrazin-2-amine (1 g, 4 mmol, 1 eq) in 1,4 dioxane (50 mL): water (10 mL) was added 8-chloroquinolin-6-ylboronic acid (990 mg, 4.8 mmol, 1.2 eq), Na₂CO₃ (840 mg, 8.0 mmol, 2 eq), PdCl₂(dppf)DCM complex (160 mg, 0.2 mmol, 0.05 eq). The reaction mixture was deoxygenated using N₂ atmosphere and the reaction mixture was heated at 80° C. for 18 h. The reaction was monitored by NMR and LCMS. The reaction mixture was diluted with water (150 mL) and extracted using ethyl acetate (2×250 mL). The separated organic layer was dried over sodium sulfate and concentrated under reduced pressure. The crude product was purified by normal phase silica-gel column chromatography to afford the tide compound (150 mg, 12%) LCMS: 333 [M+1]⁺

Step-2: Synthesis of 3-bromo-5-(8-chloroquinolin-6-yl)-6-phenylpyrazin-2-amine: To a solution of 5-(8-chloroquinolin-6-yl)-6-phenylpyrazin-2-anine (1.6 g, 4.81 mmol, 1 eq) in DMF (20 mL) was added N-bromosuccinimide (0.85 g, 4.81 mmol, 1.0 eq) at 0° C. The reaction mixture was stirred at same temperature for 2 h. The reaction was monitored by TLC. The reaction was added with water and the solid precipitates out. The solid was filtered and dried to use for next step without further purification (1.1 g, 55%). LCMS: 412 [M+1]⁺

Step-3: Synthesis of methyl (2E)-3-[3-amino-6-(8-chloroquinolin-6-yl)-5-phenylpyrazin-2-yl]prop-2-enoate: To a stirred solution 3-bromo-5-(8-chloroquinolin-6-yl)-6-phenylpyrazin-2-amine (0.50 g, 1.21 mmol, 1.0 eq) and methyl prop-2-enoate (0.207 g, 2.43 mmol, 2.0 eq) in dioxane (10 mL) was added DIPEA (0.3 mL, 1.81 mmol, 1.5 eq), The reaction was purged with N₂ for 5 min. Following this Pd₂dba₃ (0.018 g, 0.02 mmol, 2 mol %) and tri-tert-butylphosphonium tetrafluoroborate (0.017 g, 0.06 mmol, 5 mol %) was added and N₂ was purged again for 5 min. The reaction was then heated at 120° C. for 12h. The reaction was allowed to cool to RT and extracted using ethyl acetate (2×30 mL). The combined organic layers were washed (brine), dried (anhydrous Na₂SO₄) and concentrated under vacuum to get the solid which was purified by normal phase silica-gel column chromatography to get the title compound (0.20 g, 40%). LCMS: 417 [M+1]⁺.

Step 4: Synthesis of 2-(8-chloroquinolin-6-yl)-3-phenylpyrido[2,3-b]pyrazin-6(5H)-one: Sodium metal (0.044 g, 4.0 eq 1.92 mmol) was added to ethanol (2 mL) at 0° C. The resulting mixture was stirred at this temperature for 15 min. Solution of methyl (2E)-3-[3-amino-6-(8-chloroquinolin-6-yl)-5-phenylpyrazin-2-yl]prop-2-enoate (0.200 g, 0.48 mmol, 1.0 equiv) in ethanol (3 mL) was added to the above reaction mixture at 0° C. and the resulting reaction mixture was heated at 80° C. for 12h. The reaction mixture was cooled to RT. The solvent was removed under reduced pressure and the crude was directly purified by normal phase silica-gel column chromatography to get the title compound (0.007 g, 3.0%). LCMS: 384 [M+1]+. ¹H NMR (DMSO-d₆, 400 MHz) δ 12.71 (s, 1H), 9.03 (dd, 1H), 8.39 (dd, 1H), 8.13 (1H), 8.04 (d, 1H), 7.85 (d, 1H), 7.65 (dd, 1H), 7.44-7.52 (m, 2H), 7.34-7.43 (m, 3H), 6.92 (dd, 1H).

### Example S4: Synthesis of 3-phenyl-2-(quinolin-6-yl)pyrido[2,3-b]pyrazin-6(5H)-one (Compound No 1.4)

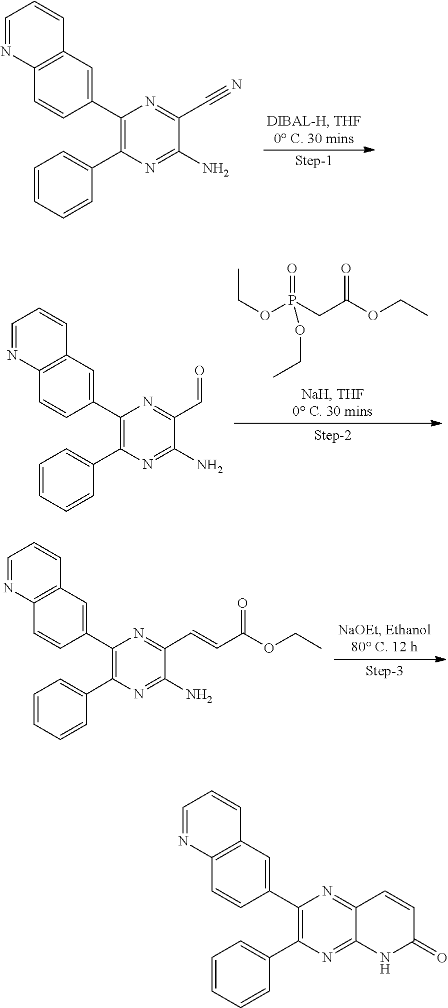 [SMILES: N#Cc1nc(-c2ccc3ncccc3c2)c(-c2ccccc2)nc1N ; Nc1nc(-c2ccccc2)c(-c2ccc3ncccc3c2)nc1C=O ; CCOC(=O)CP(=O)(OCC)OCC ; CCOC(=O)/C=C/c1nc(-c2ccc3ncccc3c2)c(-c2ccccc2)nc1N ; O=c1ccc2nc(-c3ccc4ncccc4c3)c(-c3ccccc3)nc2[nH]1]

Step-1: Synthesis of 3-amino-S-phenyl-6-(quinolin-6-yl)pyrazine-2-carbaldehyde: To a stirred solution of 3-amino-5-phenyl-6-(quinolin-6-yl)pyrazine-2-carbonitrile (0.20 g, 0.619 mmol, 1.0 equiv) in THF (10 mL) was added 1M solution of DIBAL-H in toluene (2.1 mL, 2.1 mmol, 3.5 eq) and the reaction mixture was allowed to stir at 0° C. for 30 min. Progress of the reaction was monitored by TLC and to the reaction mixture was added 2M HCl in water (16 mL) drop-wise at 0° C. and the reaction mixture was allowed to stir at the same temperature for 10 min. The reaction mixture was basified with saturated sodium carbonate solution (20 mL) and extracted with ethyl acetate (3×75 mL). Combined organic layers were washed (brine), dried (anhydrous Na₂SO₄) and concentrated under vacuum to get the yellow solid which was used as such for next step without further purification (0.27 g). LCMS: 327 [M+1]⁺.

Step-2: Synthesis of ethyl (2E)-3-[3-amino-5-phenyl-6-(quinolin-6-yl)pyrazin-2-yl]prop-2-enoate: To a solution of ethyl 2-diethoxyphosphorylacetate (0.18 g, 0.83 mmol, 1.0 eq) in THF (10 mL) was added NaH (0.037 g 0.91 mmol, 1.1 equiv) at 0° C. To this mixture was added 3-amino-5-phenyl-6-(quinolin-6-yl)pyrazine-2-carbaldehyde (0.27 g, 0.83 mmol, 1.0 eq). Progress of the reaction was monitored by TLC. The reaction mixture was quenched by adding cold water and extracted by using ethyl acetate. The combined organic layers were washed (brine), dried (anhydrous Na₂SO₄) and concentrated under vacuum to get the desired product as yellow solid which was purified by normal phase silica-gel column chromatography to get the title compound (0.221 g, 67%). LCMS: 397 [M+1]⁺.

Step-3: Synthesis of 3-phenyl-2-(quinolin-6-yl)pyrido[2,3-b]pyrazin-6(5H)-one: Sodium metal (0.053, 4.0 equiv, 2.23 mmol) was added to ethanol (2 mL) at 0° C. The resulting mixture was stirred at this temperature for 15 min. Solution of ethyl (2E)-3-[3-amino-5-phenyl-6-(quinolin-6-yl)pyrazin-2-yl]prop-2-enoate (0.221 g, 0.48 mmol, 1.0 eq) in ethanol (3 mL) was added to the above reaction mixture at 0° C. and the resulting reaction mixture was heated at 80° C. for 12h. The reaction mixture was cooled to RT. The solvent was removed under reduced pressure and directly purified by normal phase silica-gel column chromatography to get the title compound (0.015 g, 7.0%). LCMS: 351[M+1]⁺; ¹H NMR (DMSO-d₆ 400 MHz) δ 12.66 (s, 1H), 8.90-8.93 (m, 1H), 8.32 (d, 1H), 8.08-8.13 (m, 2H), 7.90 (d, 1H), 7.63 (dd, 1H), 7.54 (dd, 1H), 7.45 (d, 2H), 7.30-7.42 (m, 3H), 6.90 (d, 1H).

### Example S5: Synthesis of 2-(8-chloroquinolin-6-yl)-3-(3-methyl-1H-pyrazol-1-yl)pyrido[2,3-b]pyrazin-6(5H)-one (Compound No 1.185)

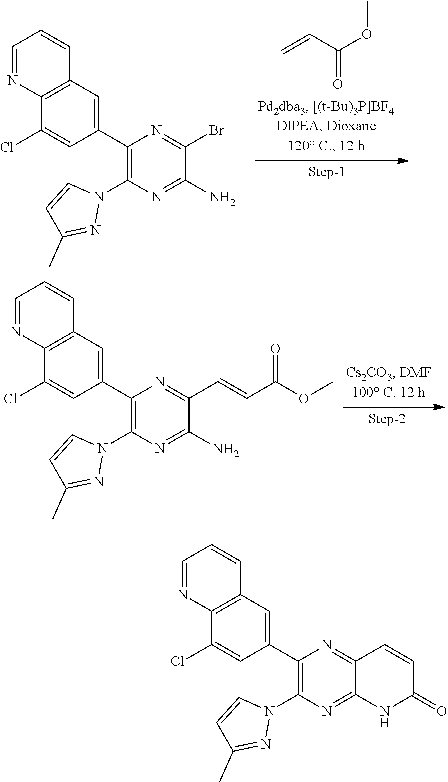

Step 1: Synthesis of methyl 3-[3-amino-6-(8-chloroquinolin-6-yl)-5-(3-methyl-1H-pyrazol-1-yl)pyrazin-2-yl]prop-2-enoate: To a stirred solution of 3-bromo-5-(8-chloroquinolin-6-yl)-6-(3-methyl-1H-pyrazol-1-yl)pyrazin-2-amine (1.00 g, 2.41 mmol, 1.0 eq) and methyl prop-2-enoate (0.412 g, 4.83 mmol, 2.0 eq) in dioxane (10 mL) was added DIPEA (0.7 mL, 3.61 mmol, 1.5 eq), The reaction was purged with N₂ for 5 min. Following this Pd₂dba₃ (0.044 g, 0.04 mmol, 2 mol %) and tri-tert-butylphosphonium tetrafluoroborate (0.035 g, 0.12 mmol, 5 mol %) was added and N₂ was purged again for 5 minute. The reaction was then heated at 120° C. for 12 h. The reaction was allowed to cool to RT and extracted using ethyl acetate (2×30 mL). The combined organic layers were washed (brine), dried (anhydrous Na₂SO₄) and concentrated under vacuum to get the solid which was purified by normal phase silica-gel column chromatography to get the title compound (0.700 g, 70%). LCMS: 421 [M+1]⁺

Step 2: Synthesis of 2-(8-chloroquinolin-6-yl)-3-(3-methyl-1H-pyrazol-1-yl)pyrido[2,3-b]pyrazin-6(5H)-one: To the stirred solution of methyl 3-[3-amino-6-(8-chloroquinolin-6-yl)-5-(3-methyl-1H-pyrazol-1-yl)pyrazin-2-yl]prop-2-enoate in DMF (10 mL) was added cesium carbonate (0.384 g, 1.19 mmol) The resulting mixture was stirred at this temperature for 15 min and then heated at 100° C. for 12 h. The reaction mixture was cooled to RT and extracted using ethyl acetate (2×30 mL). The combined organic layers were washed (brine), dried (anhydrous Na₂SO₄) and concentrated under vacuum to obtain the crude, the crude was purified by RP-HPLC to afford the title compound (20 mg, 11%). LCMS: 389[M+1]⁺; ¹H NMR (DMSO-d₆, 400 MHz) δ 12.80 (br s, 1H), 9.04 (dd, J=1.75, 3.95 Hz, 1H), 8.48 (dd, J=1.53, 8.55 Hz, 1H), 8.09-8.15 (m, 2H), 8.04 (d, J=1.75 Hz, 1H), 7.64-7.71 (m, 2H), 6.91 (d, J=9.65 Hz, 1H), 6.40 (d, J=2.63 Hz, 1H), 1.23 (s, 3H).

### Example S6: Synthesis of 2-(8-Chloroquinolin-6-yl)-3-(1-methyl-1H-pyrazol-3-yl)pyrido[2,3-b]pyrazin-6(5H)-one (Compound No 1.184)

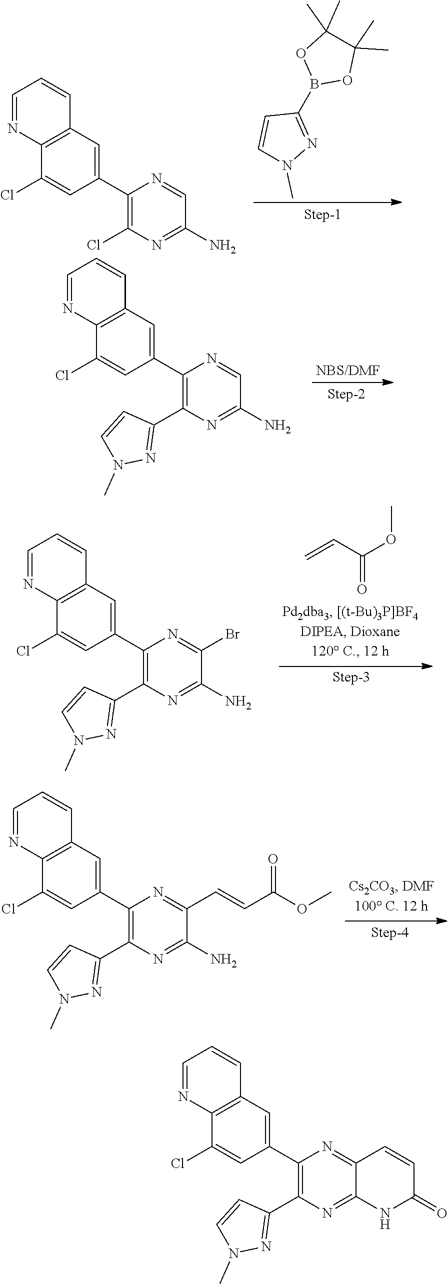

Step-1: Synthesis of 5-(8-chloroquinolin-6-yl)-6-(1-methyl-1H-pyrazol-3-yl)pyrazin-2-amine: To a stirred solution of 6-chloro-5-(quinolin-6-yl)pyrazin-2-amine (1.0 g, 3.44 mmol, 1.0 eq.) in dioxane:water (16 mL: 4 mL) was added 1-methyl-3-(4,4,5,5-tetramethyl-1,3,2-dioxaborolan-2-yl)-1H-pyrazole (0.860 g, 4.12 mmol, 1.2 eq.). The reaction mixture was purged with nitrogen for 5 min then charged with Na₂CO₃ (0.73 g, 6.88 mmol, 2.0 eq.) and Pd(dppf)Cl₂.DCM complex (0.080 g, 10 mol %). The reaction mixture was again purged with nitrogen. The reaction mixture was allowed to heat at 100° C. for 16 h. The reaction was monitored by TLC and LCMS. The reaction mixture was filtered through celite and distilled. The reaction was diluted with water and extracted with ethyl acetate (3×200 mL). The combined organic layers were washed (brine), dried (anhydrous Na₂SO₄) and concentrated under vacuum, to get the crude which was purified by normal phase silica-gel column chromatography to get the title compound (0.400 g, 34%). LCMS: 337[M+1].⁺

Step-2: Synthesis of 3-bromo-5-(8-chloroquinolin-6-yl)-6-(1-methyl-1H-pyrazol-3-yl)pyrazin-2-amine: To a stirred solution 5-(8-chloroquinolin-6-yl)-6-(1-methyl-1H-pyrazol-3-yl)pyrazin-2-amine (0.400 g, 1.18 mmol, 1 eq.) in DMF (5 ml) was added NBS (210 mg, 1.18 mmol, 1.0 eq.) at 0° C. Reaction mixture was stirred at 0° C. for 10 min. The reaction was monitored by TLC and LCMS and found to be complete after 10 min. The reaction mixture was quenched with cold water 10 mL and was extracted with EtOAc (3×20 mL). The resulting solution was concentrated under reduced pressure. The crude product was purified by normal phase column chromatography to get the title compound (0.300 g, 61%). LCMS: 415 [M+1]⁺.

Step-3: Synthesis of Methyl 3-(3-amino-6-(8-chloroquinolin-6-yl)-5-(1-methyl-1H-pyrazol-3-yl)pyrazin-2-yl)acrylate: To a stirred solution of 3-bromo-5-(8-chloroquinolin-6-yl)-6-(1-methyl-1H-pyrazol-3-yl)pyrazin-2-amine (0.132 g, 0.31 mmol, 1.0 eq) and methyl prop-2-enoate (0.058 g, 0.63 mmol, 2.0 eq) in dioxane (5 mL) was added DIPEA (0.1 mL, 0.46 mmol, 1.5 eq), The reaction was purged with N₂ for 5 min. Following this Pd₂dba₃ (0.006 g, 0.006 mmol, 2 mol %) and tri-tert-butylphosphonium tetrafluoroborate (0.005 g, 0.01 mmol, 5 mol %) was added and N₂ was purged again for 5 minute. The reaction was then heated at 120° C. for 12 h. The reaction was allowed to cool to RT and extracted using ethyl acetate (2×30 mL). The combined organic layers were washed (brine), dried (anhydrous Na₂SO₄) and concentrated under vacuum to get the solid which was purified by normal phase silica-gel column chromatography to get the title compound (0.80 g, 60%). LCMS: 421 [M+1]⁺

Step-4: Synthesis of 2-(8-Chloroquinolin-6-yl)-3-(1-methyl-1H-pyrazol-3-yl)pyrido[2,3-b]pyrazin-6(5H)-one: To the stirred solution of Methyl 3-(3-amino-6-(8-chloroquinolin-6-yl)-5-(1-methyl-1H-pyrazol-3-yl)pyrazin-2-yl)acrylate (0.080 g, 0.19 mmol, 1.0 eq.) in DMF (5 mL) was added cesium carbonate (0.123 g, 0.38 mmol, 2.0 eq). The resulting mixture was stirred at this temperature for 15 min and then heated at 120° C. for 12 h. The reaction mixture was cooled to RT and extracted using ethyl acetate (2×30 mL). The combined organic layers were washed (brine), dried (anhydrous Na₂SO₄) and concentrated under vacuum to obtain the crude, the crude was purified by normal phase silica-gel column chromatography to afford the title compound (10 mg, 13%). LCMS: 389[M+1]⁺; ¹H NMR (DMSO-d6, 400 MHz) δ 12.64 (br. s., 1H), 9.05 (br. s., 1H), 8.49 (d, 1H), 8.13 (br. s., 1H), 8.06 (d, 1H), 7.93 (br. s., 1H), 7.71 (br. s., 2H), 6.87 (d, 1H), 6.37 (br. s., 1H), 3.72 (s, 3H).

It is understood that compounds from Table 1 (1.1-1.3, 1.6-1.7, 1.9-1.11, 1.13-1.183, 1.186-2.219) are synthesized by the General Synthetic Scheme 1, Scheme 2, Scheme 3, Scheme 4 or present routes involving steps clearly familiar to those skilled in the art, wherein the substituents described in compounds of formula (I) herein can be varied with a choice of appropriate starting materials and reagents utilized in the steps presented.

### BIOLOGICAL EXAMPLES

### Example B1. Radioligand Binding Competition Assay

### Example B1(a)

Binding of selected compounds to the adenosine A2A, A1, A2B, and A3 receptors is tested using a binding competition assay.

The general protocol for the radioligand binding competition assay is as follows. Competition binding is performed in duplicate in the wells of a 96 well plate (Master Block, Greiner, 786201) containing binding buffer (optimized for each receptor), membrane extracts (amount of protein/well optimized for each receptor), radiotracer (final concentration optimized for each receptor), and test compound. Nonspecific binding is determined by co-incubation with 200-fold excess of cold competitor. The samples are incubated in a final volume of 0.1 mL at 25° C. for 60 minutes and then filtered over filter plates. Filters are washed six times with 0.5 mL of ice-cold washing buffer (optimized for each receptor) and 50 pL of Microscint 20 (Packard) are added on each filter. The filter plates are sealed, incubated 15 min on an orbital shaker and scintillation counted with a TopCount for 30 sec/filter.

For the A_{2A} adenosine receptor radioligand binding assay, the following modifications are made to the general protocol. GF/C filters (Perkin Elmer, 6005174), presoaked in 0.01% Brij for 2h at room temperature are used. Filters are washed six times with 0.5 mL of ice-cold washing buffer (50 mM Tris pH 7.4) and 50 pL of Microscint 20 (Packard) are added in each well. The plates are then incubated for 15 min on an orbital shaker and then counted with a TopCount™ for 1 min/well.

Another radioligand binding assay used to evaluate the binding affinity for the adenosine A_{2A} receptor assay is performed in duplicate in the wells of a 384 plate. Assay buffer contains DPBS 500 mM, MgCl₂ 0.1 mM, and 1% DMSO. Membrane-bead suspension is prepared by mixing 25.98 pL of human adenosine A_{2A} membrane preparation (Perkin Elmer, RBHA2AM400UA) at 33.4 pg/mL, 28 pL of ADA at 20 pg/mL, and 932 pL of SPA beads at 3.33 mg/mL) and the mixture is incubated for 20 min at room temperature. 20 pL of radiotracer (³H-SCH 58261) at 15 nM is mixed into each well containing test articles at various concentrations and the plate is centrifuged at 1000 rpm for 1 minute. 30 pL of the membrane-bead suspension is added to each well. The plates are sealed and incubated for 1 hr at room temperature with vigorous mixing on a plate mixer. Plates are read on Microbeta² (Perkin Elmer, 2450-0010).

For the adenosine A₁ radioligand binding competition assay, a similar procedure is used except that the following reagents are used: CHO-KI-A1 cell membranes; binding buffer comprising HEPES 25 mM pH 7.4, MgCl₂ 5 mM, CaCl₂) 1 mM, NaCl 100 mM, saponin 10 pg/mL; wash buffer comprising HEPES 25 mM pH 7.4, MgCl₂ 5 mM, CaCl₂) 1 mM, NaCl 100 mM; a Unifilter GF/B—treated for 2h with 0.5% PEI; and 1.6 nM of ³H-DPCPX as the tracer.

Similarly, the following reagents are used for the adenosine A_{2B} radioligand binding competition assay: HEK-293-A_{2B} cell membranes, 20 pg/well, preincubated 30 min at RT with 25 μg/mL Adenosine Deaminase; a binding buffer comprising HEPES 10 mM pH 7.4, EDTA 1 mM, 0.5% BSA; a wash buffer comprising HEPES 10 mM pH 7.4, EDTA 1 mM; a Unifilter GF/C—treated for 2h with 0.5% PEI; and 10 nM ³H-DPCPX as the tracer.

For the adenosine A₃ radioligand binding competition assay, the following reagents are used: CHO-KI-A3 cell membranes, 1.5 μg/well; a binding buffer comprising HEPES 25 mM pH 7.4, MgCl₂ 5 mM, CaCl₂) 1 mM, 0.5% BSA; a wash buffer comprising HEPES 25 mM pH 7.4, MgCl₂ 5 mM, CaCl₂) 1 mM; a Unifilter GF/C—treated for 2h with 0.5% BS; and 0.4 nM of ¹²⁵I-AB-MECA as the tracer.

The results of the binding assay are given as percent residual binding at a given concentration. Percent of residual binding means binding of a compound in the presence of competitor normalized to the amount of binding in the absence of competitor.

### Example B1(b)

A second A_{2A} adenosine receptor radioligand binding assay protocol was used. The protocol used adenosine A2a (human) membrane (PerkinElmer RBHA2AM400UA) at a concentration of 5 pg/well/100 μl and the radioligand [3H] CGS-21680 (Cat No. PerkinElmer-NET1021250UC) at a final concentration of 6 nM. Testing compounds were diluted with DMSO to make 8-point 4-fold serial dilution, starting at 0.2 mM. CGS-15943 was the reference compound. 1 μl of compounds/high control/low control was transferred to the assay plate according to a plate map, followed by 100 μl of membrane stocks and 100 μl of radioligand, in assay buffer (50 mM Tris-HCl, 10 mM MgCl₂, 1 mM EDTA, pH 7.4). The plate was sealed and incubated at RT for 2 hours. Unifilter-96 GF/C filter plates (Perkin Elmer Cat #6005174) were soaked with 50 μl of 0.3% PEI per well for at least 0.5 hour at room temperature. When the binding assays were completed, the reaction mixtures were filtered through GF/C plates using Perkin Elmer Filtermate Harvester, and each plate washed 4 times with cold wash buffer (50 mM Tris-HCl, 154 mM NaCl, pH 7.4). The filter plates were dried for 1 hour at 50 degrees. After drying, the bottom of the filter plate wells was sealed, 50 μl of Perkin Elmer Microscint 20 cocktail was added, and the top of the filter plate was sealed. ³H trapped on the filter was counted using Perkin Elmer MicroBeta2 Reader. The data were analyzed with GraphPad Prism 5 to obtain binding IC₅₀ values. The “Inhibition [% Control]” was calculated using the equation: % Inh=(1−Background subtracted Assay value/Background subtracted HC value)*100, where HC is high control. A2a binding IC₅₀ values are shown in Table B1.

A second A₁ adenosine receptor radioligand binding assay protocol is used. The protocol uses adenosine A1 (human) membrane (PerkinElmer ES-010-M400UA) at a concentration of 2.5 pg/well/100 μl and the radioligand [3H] DPCPX (Cat No. PerkinElmer-NET974250UC) at a final concentration of 1 nM. Testing compounds are tested at a final concentration of 200 nM. CGS-15943, the reference compound, is tested in an 8-point 4-fold serial dilution, starting at a top concentration of 1 μM. 1 μl of compounds/high control/low control is transferred to the assay plate according to a plate map, followed by 100 μl of membrane stocks and 100 μl of radioligand, in assay buffer (25 mM HEPES, 5 mM MgCl₂, 1 mM CaCl₂), 100 mM NaCl, pH 7.4). The plate is sealed and incubated at RT for 1 hour. Unifilter-96 GF/C filter plates (Perkin Elmer Cat #6005174) are soaked with 50 μl of 0.3% PEI per well for at least 0.5 hour at room temperature. When the binding assays are completed, the reaction mixtures are filtered through GF/C plates using Perkin Elmer Filtermate Harvester, and each plate washed 4 times with cold wash buffer (25 mM HEPES, 5 mM MgCl₂, 1 mM CaCl₂), 100 mM NaCl, pH 7.4). The filter plates are dried for 1 hour at 50 degrees. After drying, the bottom of the filter plate wells is sealed, 50 μl of Perkin Elmer Microscint 20 cocktail is added, and the top of the filter plate is sealed. ³H trapped on the filter is counted using Perkin Elmer MicroBeta2 Reader. The data are analyzed with GraphPad Prism 5 to obtain binding IC₅₀ values for the reference compound. The “Inhibition [% Control]” is calculated using the equation: % Inh=(1−Background subtracted Assay value/Background subtracted HC value)*100, where HC is high control.

TABLE B1

| Compound No. | A2a binding IC50 (nM) |
|---|---|
| 1.4 | 5.5 |
| 1.5 | 18 |
| 1.8 | 958 |
| 1.12 | 6.6 |

### Example B2. cAMP Assay

The functional activity of compound 1.5 was tested using Assay 2 below, to detect the presence of cAMP. Assay 1 is an alternative assay for this purpose. Activation of G-protein coupled receptors (such as A_{2A}) results in activation of adenylyl cyclase which converts ATP into cAMP which is used as a downstream signaling molecule. Molecules which act as GPCR (or specifically A_{2A} receptor) antagonists cause a decrease in intracellular cAMP concentration.

Assay 1: This assay uses HEK-293 cells expressing human recombinant adenosine A_{2A} receptor that are grown prior to the test in media without antibiotic. The cells are detached by gentle flushing with PBS-EDTA (5 mM EDTA), recovered by centrifugation and suspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂), 0.5 g/L BSA, supplemented with Rolipram).

12 pL of cells are mixed with 6 pL of the test compound at increasing concentrations and then incubated for 10 min. Thereafter 6 pL of the reference agonist is added at a final concentration corresponding to the historical EC₈₀. The plates are then incubated for 30 min at room temperature. After addition of the lysis buffer and 1 hour incubation, cAMP concentrations are estimated, according to the manufacturer specification, with the HTRF® kit.

Assay 2 (Table B2): This assay used HEK-293 cells expressing human recombinant adenosine A_{2A} receptor that were grown prior to the test in media without antibiotic. 100 nL of test articles at 100× of final concentration were transferred to assay plate by Echo. Cells were washed twice with 5 mL of PBS and 10 pL of cells were mixed with 5 mL PBS. After aspirating the PBS and adding 1.5 mL versine, cells were incubated at 37° C. for 2-5 min. After centrifugation, 4 mL of medium was added and adjusted cell density to 5,000 cells/well with Stimulation Buffer. 10 pL of cells were aliquoted to the assay plate, centrifuged at 1000 rpm for 1 minute, and incubated for 60 minutes at room temperature. 5 pL 4× Eu-cAMP tracer solution and 5 pL 4× Ulight™-anti-cAMP solution were added to assay plate, followed by centrifugation and 60-minute incubation at room temperature. Plates were read on EnVision. The IC₅₀ of compound 1.5 for reducing A_{2A} cAMP levels is shown in Table B2. A similar assay using HEK-293 cells expressing human recombinant adenosine A₁ receptor may also be performed to detect inhibition of the activation of adenosine A₁ receptor, for example.

TABLE B2

| Compound No. | A2a cAMP IC50 (nM) |
|---|---|
| 1.5 | 324.8 |

### Example B3: GTP γ³⁵S Scintillation Proximity Assay for A_{2A} Receptor

A scintillation proximity assay (SPA) is used to determine the kinetic profile of the binding of test compound to the A_{2A} receptor.

For antagonist testing, membrane extracts are prepared from HEK-293 cells expressing recombinant human A_{2A} receptor, are mixed with GDP (volume:volume) and are incubated in assay buffer comprising 20 mM HEPES pH 7.4; 100 mM NaCl, 10 μg/mL saponin, 5 mM MgCl₂ for at least 15 min on ice. In parallel, GTPγ[³⁵S] is mixed with the beads (volume:volume) just before starting the reaction. The following reagents are successively added in the wells of an Optiplate (Perkin Elmer): 25 pL of test compound or reference ligand, 25 pL of the membranes: GDP mix, 25 pL of reference agonist at historical EC₈₀ and 25 pL of GTPγ[³⁵S] (PerkinElmer NEG030X), diluted in assay buffer to give 0.1 nM. The plate is incubated at room temperature for 1 hour. Then, 20 pL of IGEPAL is added for 30 minutes at room temperature. Following this incubation, 20 pL of beads (PVT-anti rabbit (PerkinElmer, RPNQ0016)), diluted in assay buffer at 50 mg/mL (0.5 mg/10 μL) and 20 pL of an Anti-GαS/olf antibody are added for a final incubation of 3 hours at room temperature. Then, the plates are centrifuged for 10 min at 2000 rpm, incubated at room temperature for 1 hour and counted for 1 min/well with a PerkinElmer TopCount reader.

### Example B4: Functional T Cell Assay

Human T Cell Activation Assay: Fresh human blood is diluted with the same volume of PBS and the buffy coat containing peripheral blood mononuclear cells (PBMCs) is prepared and resuspended in culture medium at a density of 2×10⁶/mL. 2×10⁵ PBMCs (in 100 pL) are plated to each well of a 96-well flat bottom plate. 25 pL of 8× final concentration of 10-fold serial diluted or single concentration compounds are added to indicated wells and incubated for 30 mins in 37° C./5% CO₂. 25 pL of 8× final concentration of NECA (1 μM) is added to indicated wells and incubated for 30 min in 37° C./5% CO₂. Beads included in T cell activation/expansion kit (Miltenyi biotec Cat #130-091-441) at a bead-to-cell ratio of 1:6 in 50 pL is added to all wells with the final concentration of DMSO at 0.1% and final volume at 200 μL. 60 pL of supernatant post 24 hr and 48 hr incubation is collected for TNF-α and IFN-γ concentration evaluation using TNF-α ELISA ready-set-go kit (eBioscience, Cat #88-7346-77) and IFN-γ ELISA ready-set-go kit (eBioscience, Cat #88-7316-77), respectively.

### Example B5: cAMP Assay

CD8+ T-cells are isolated from peripheral blood mononuclear cells (PBMC) from normal donors using CD8+T lymphocyte enrichment kit.

In a 96-well plate coated with anti-CD3 antibody, CD8⁺ T-cells (1×10⁵) are cultured alone, with 3 μM of NECA, or in the presence of 1 μM of the compound of interest with or without 3 μM of NECA. The cells are incubated for 30 min at 37° C. and 5% CO₂, and the reaction is stopped by addition of 200 pL, 0.1 M hydrochloric acid. cAMP levels are determined by an ELISA kit.

### Example B6: Anti-Tumor Activities in Immuno-Oncology Mouse Models

The anti-tumor activities of test articles are evaluated in selective mouse models (e.g., syngeneic model, xenograft model, or PDX) as a monotherapy or combination therapies. Using MC-38 syngeneic model as an example: female C57BL/6 mice are inoculated subcutaneously at right flank with MC-38 cells for tumor development. Five days after tumor inoculation, mice with tumor size ranging from 40-85 mm³ are selected and assigned into sub-groups using stratified randomization with 10 mice per group based upon their tumor volumes. Mice receive pre-defined treatments include vehicle, test article at various doses alone, test article at various doses plus other anti-cancer therapy, and other anti-cancer therapy control. Body weight and tumor sizes are measured three times per week during the treatment. Tumor volume is expressed in mm³ using the formula: V=0.5 a×b² where a and b are the long and short diameters of the tumor, respectively. The tumor sizes are used for the calculations of both tumor growth inhibition (TGI) and T/C values. When an individual animal reaches to the termination endpoint (e.g., with TV >1000 mm³), the mouse is euthanized. The time from inoculation to the termination are deemed as its survival time. Survival curves are plotted by the Kaplan-Meier method. At the end of study, plasma and tumor samples are collected to explore biomarkers.

### Example B7: Mouse Splenocyte Assay

IC₅₀ values of compounds for reversal of NECA suppression of mIFNγ release are determined in mouse splenocytes isolated from Balb/c mice. The mIFNγ release is CD3e/CD28-induced release. Mouse splenocytes (2×10⁵ cells/well) are activated with Anti-mouse CD3e (2.5 μg/ml, coated overnight at 4° C.; Cat #14-0032-82, eBioscience) and then incubated with serial dilutions of compounds (3 fold, 8 point dose response starting at 1 μM) in the presence of NECA (at a concentration such as 0.1, 3.0, or 6.0 μM; Cat #E2387, Sigma) for 30 min at 37° C., 5% CO₂ in an incubator (cell culture conditions) prior to treating them with Anti-mouse CD28 (0.1 pg/ml soluble; Cat #16-0289-81, eBiosciences). Splenocytes are further incubated under cell culture conditions for 72 hr; the supernatant is then harvested and diluted to 1:100, and ELISA is performed as per the manufacturer's protocol (mIFN-γ kit; Cat #555138 and 550534, BD Biosciences). Plates are read in a plate reader by measuring absorbance at 450 nm. Values for the reversal of NECA suppressed mIFN-γ release by compounds are calculated by the following formula:

$$Normalized mIFN - γ release = \frac{( {[ mIFN - γ ]}_{test} - {[ mIFN - γ ]}_{blank} )}{( {[ mIFN - γ ]}_{NECA} - {[ mIFN - γ ]}_{blank} )}$$

where [mIFN-γ]ₜₑₛₜ is the test reading, [mIFN-γ]_{blank} is the average reading obtained rom blank wells, and [mIFN-γ]_{NECA} is the average reading obtained from NECA treated, activated cells. The IC₅₀ values are calculated by fitting the curve to the “four-parameter variable slope logistic model” using Graph Pad Prism.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

All references throughout, such as publications, patents, and published patent applications, are incorporated herein by reference in their entireties.

What is claimed is:

1. A compound of the Formula (I): 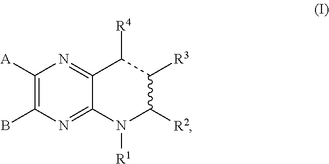
or a tautomer or isomer thereof, or a pharmaceutically acceptable salt of any of the foregoing, wherein:
R¹ is H or C₁-C₆ alkyl wherein the C₁-C₆ alkyl of R¹ is optionally substituted with oxo or R^{a};
R² and R⁴ are each independently H, R^{b} or oxo, provided that when R² is —NR⁹R¹⁰, then at least one of R⁹ and R¹⁰ is not H;
R³ is H or R^{c};
each R^{a}, R^{b}, and R^{c} is independently C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁸, —OC(O)R⁸, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁹S(O)R⁹, —NR⁹S(O)₂R⁹, —C(O)NR⁹S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁹, —(C₁-C₃ alkylene)OC(O)R⁹, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁹, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R^{a}, R^{b}, and R^{c} is independently optionally substituted by halogen, oxo, —OR¹¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O))(OR¹¹)(OR¹²), C₃-C₅ cycloalkyl, or C₁-C₆ alkyl optionally substituted by oxo, —OH or halogen; [symbol]
is a single bond or a double bond, wherein when [symbol] is a double bond, R² is oxo; [symbol]
is a single bond or a double bond, wherein when [symbol] is a double bond, R⁴ is oxo;
one of [symbol] and [symbol] is a double bond and the other is a single bond;
A is C₆-C₁₂ aryl, 5- to 10-membered heteroaryl, 9- to 10-membered carbocycle, or 9- to 10-membered heterocycle, wherein the C₆-C₁₂ aryl, 5- to 10-membered heteroaryl, 9- to 10-membered carbocycle, or 9- to 10-membered heterocycle of A is optionally further substituted with R⁶;
B is phenyl, 5- to 6-membered heteroaryl, 5- to 6-membered carbocycle, 5- to 6-membered heterocycle, or 9- to 10-membered heteroaryl, wherein the phenyl, 5- to 6-membered heteroaryl, 5- to 6-membered carbocycle, 5- to 6-membered heterocycle, or 9- to 10-membered heteroaryl of B is optionally further substituted with R¹;
each R⁶ and R⁷ is independently oxo, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl), wherein each R⁶ and R⁷ is independently optionally substituted by halogen, oxo, —OR¹¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen,
provided that when R¹ is C₁-C₆ alkyl, then R⁶ is oxo, C₁-C₆ alkyl, C₂-C₆ alkenyl, C₂-C₆ alkynyl, halogen, —CN, —OR⁸, —SR⁸, —NR⁹R¹⁰, —NO₂, —C═NH(OR⁸), —C(O)R⁹, —OC(O)R⁹, —C(O)OR⁸, —C(O)NR⁹R¹⁰, —OC(O)NR⁹R¹⁰, —NR⁸C(O)R⁹, —NR⁸C(O)OR⁹, —NR⁸C(O)NR⁹R¹⁰, —S(O)R⁸, —S(O)₂R⁸, —NR⁸S(O)R⁹, —C(O)NR⁸S(O)R⁹, —NR⁸S(O)₂R⁹, —C(O)NR⁸S(O)₂R⁹, —S(O)NR⁹R¹⁰, —S(O)₂NR⁹R¹⁰, —P(O)(OR⁹)(OR¹⁰), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁸, —(C₁-C₃ alkylene)SR⁸, —(C₁-C₃ alkylene)NR⁹R¹⁰, —(C₁-C₃ alkylene)CF₃, —(C₁-C₃ alkylene)NO₂, —C═NH(OR⁸), —(C₁-C₃ alkylene)C(O)R⁸, —(C₁-C₃ alkylene)OC(O)R⁸, —(C₁-C₃ alkylene)C(O)OR⁸, —(C₁-C₃ alkylene)C(O)NR⁹R¹⁰, —(C₁-C₃ alkylene)OC(O)NR⁹R¹⁰, —(C₁-C₃ alkylene)NR⁸C(O)R⁹, —(C₁-C₃ alkylene)NR⁸C(O)OR⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —C(O)(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)P(O)(OR⁹)(OR¹⁰), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) or —(C₁-C₃ alkylene)(C₆-C₁₄ aryl);
each R⁸ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl, 3-6-membered heterocyclyl, —(C₁-C₃ alkylene)(C₃-C₆ cycloalkyl), —(C₁-C₃ alkylene)(C₆-C₁₄ aryl), —(C₁-C₃ alkylene)(5-6-membered heteroaryl), or —(C₁-C₃ alkylene)(3-6-membered heterocyclyl), wherein the C₁-C₆ alkyl, C₂-C₆ alkenyl, C₂-C₆ alkynyl, C₃-C₆ cycloalkyl, C₆-C₁₄ aryl, 5-6-membered heteroaryl, 3-6-membered heterocyclyl, —(C₁-C₃ alkylene)(C₃-C₆ cycloalkyl), —(C₁-C₃ alkylene)(C₆-C₁₄ aryl), —(C₁-C₃ alkylene)(5-6-membered heteroaryl), and —(C₁-C₃ alkylene)(3-6-membered heterocyclyl) of R⁸ are independently optionally substituted by halogen, oxo, —CN, —OR¹³, —NR¹³R¹⁴, —P(O)(OR¹³)(OR¹⁴), phenyl optionally substituted by halogen, or C₁-C₆ alkyl optionally substituted by halogen, —OH or oxo;
R⁹ and R¹⁰ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl, 3-6 membered heterocyclyl, —(C₁-C₃ alkylene)NR¹¹R¹², —(C₁-C₃ alkylene)(C₃-C₆ cycloalkyl), —(C₁-C₃ alkylene)(3-6-membered heterocyclyl), —(C₁-C₃ alkylene)(5-6-membered heteroaryl) or —(C₁-C₃ alkylene)(C aryl), wherein the C₁-C₆ alkyl, C₂-C₆ alkenyl, C₂-C₆ alkynyl, C₃-C₆ cycloalkyl, C₆-C₁₄ aryl, 5-6-membered heteroaryl, 3-6 membered heterocyclyl, —(C₁-C₃ alkylene)(C₃-C₆ cycloalkyl), —(C₁-C₃ alkylene)(3-6-membered heterocyclyl), —(C₁-C₃ alkylene)(5-6-membered heteroaryl) and —(C₁-C₃ alkylene)(C6 aryl) of R⁹ and R¹⁰ are independently optionally substituted by halogen, oxo, —CN, —OR¹³, —NR¹³R¹⁴ or C₁-C₆ alkyl optionally substituted by halogen, —OH or oxo;
or R⁹ and R¹⁰ are taken together with the atom to which they attached to form a 3-6 membered heterocyclyl optionally substituted by halogen, oxo, —OR¹³, —NR¹³R¹⁴ or C₁-C₆ alkyl optionally substituted by halogen, oxo or —OH;
R¹¹ and R¹² are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹ and R¹² are taken together with the atom to which they attached to form a 3-6 membered heterocyclyl optionally substituted by halogen, oxo or C₁-C₆ alkyl optionally substituted by halogen or oxo; and
R¹³ and R¹⁴ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹³ and R¹⁴ are taken together with the atom to which they attached to form a 3-6 membered heterocyclyl optionally substituted by halogen, oxo or C₁-C₆ alkyl optionally substituted by oxo or halogen.

2. The compound of claim 1, or a tautomer or isomer thereof, or a pharmaceutically acceptable salt of any of the foregoing, wherein R² is H, oxo, C₁-C₆ alkyl, C₂-C₆ alkenyl, C₂-C₆ alkynyl, C₃-C₆ cycloalkyl, 3-12-membered heterocyclyl, 5- to 10-membered heteroaryl, C₆-C₁₄ aryl, halogen, —CN, —OR⁸, —C(O)NR⁹R¹⁰, —C(O)R⁸, —C(O)OR⁸, —OC(O)R⁸, —OC(O)NR⁹R¹⁰, —NR⁸C(O)R⁹, —NR⁸C(O)OR⁹, —NR⁸C(O)NR⁹R¹⁰, —S(O)₂R⁸, —NR⁸S(O)R⁹, —NR⁸S (O)₂R⁹, —C(O)NR⁸S(O)R⁹, —C(O)NR⁸S(O)₂R⁹, —S(O)NR⁹R¹⁰, —S(O)₂NR⁹R¹⁰, —(C₁-C₃ alkylene)CN, —(C₁-C₃ alkylene)SR⁸, —(C₁-C₃ alkylene)NR⁹R¹⁰, —(C₁-C₃ alkylene)C(O)R⁸, —(C₁-C₃ alkylene)C(O)NR⁹R¹⁰, —(C₁-C₃ alkylene)NR⁸C(O)R⁹, —(C₁-C₃ alkylene)NR⁸C(O)NR⁹R¹⁰, —(C₁-C₃ alkylene)S(O)R⁸, —(C₁-C₃ alkylene)S(O)₂R⁸, —(C₁-C₃ alkylene)NR⁸S(O)R⁹, —(C₁-C₃ alkylene)NR⁸S(O)₂R⁹, —(C₁-C₃ alkylene)C(O)NR⁸S(O)₂R⁹, —(C₁-C₃ alkylene)S(O)NR⁹R¹⁰, —(C₁-C₃ alkylene)S(O)₂NR⁹R¹⁰, —(C₁-C₃ alkylene)(C₃-C₆ cycloalkyl), —S(O)R⁸, —(C₁-C₃ alkylene)OR⁸, —(C₁-C₃ alkylene)(5-10-membered heteroaryl), each of which is optionally substituted by halogen, oxo, —OR¹, —NR¹¹R¹², —C(O)R¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹)(OR¹²), C₃-C₈ cycloalkyl, or C₁-C₆ alkyl optionally substituted by oxo, —OH or halogen.

3. The compound of claim 1 or 2, or a tautomer or isomer thereof, or a pharmaceutically acceptable salt of any of the foregoing, wherein R² is selected from the group consisting of: H, oxo, methyl, phenyl, cyclopropyl, fluoro, chloro, bromo, —CN, methoxy,

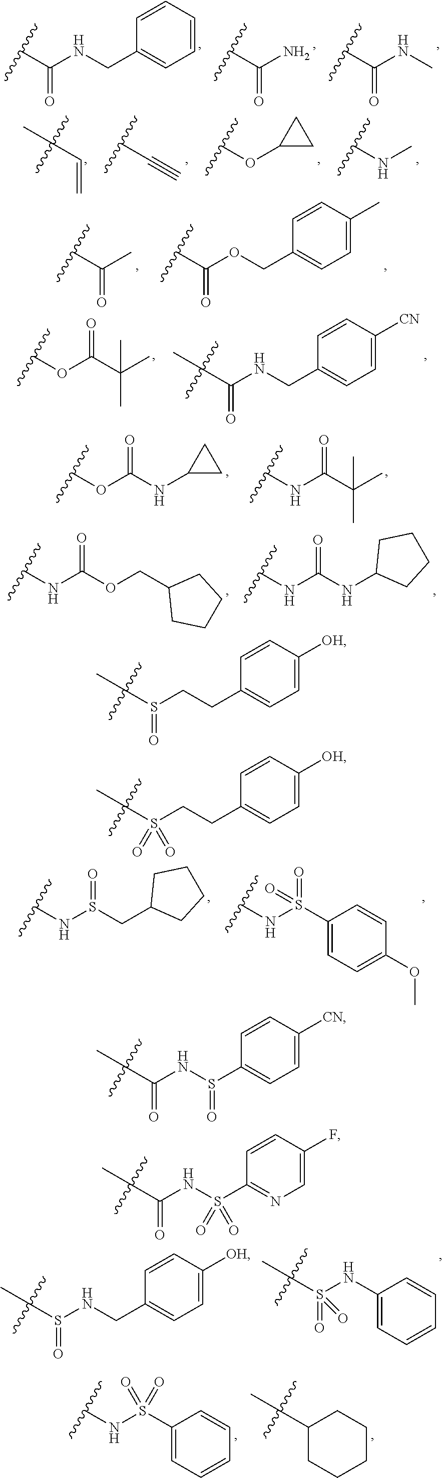

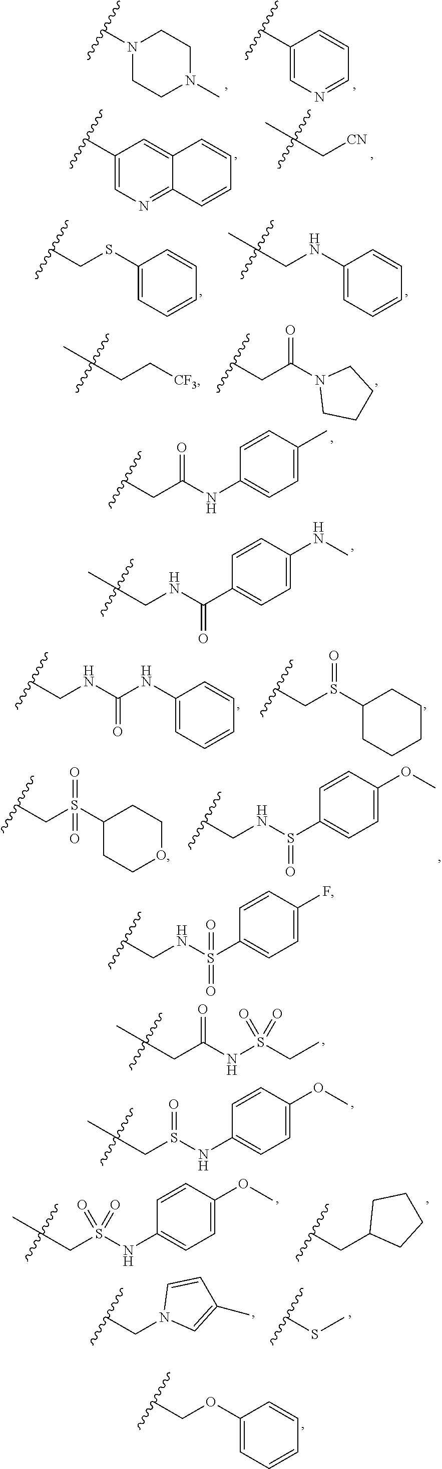

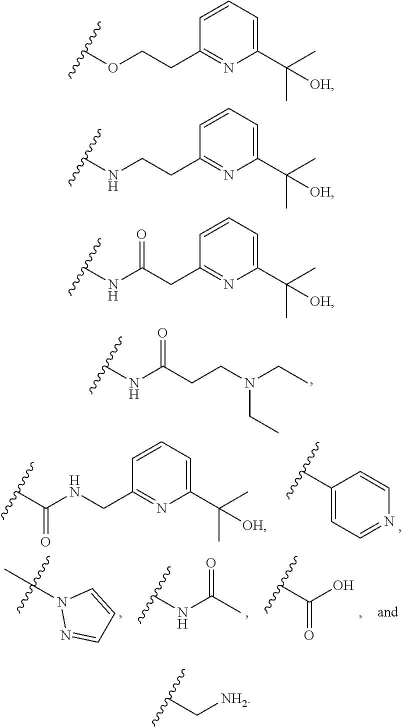

4. The compound of any one of claims 1-3, or a tautomer or isomer thereof, or a pharmaceutically acceptable salt of any of the foregoing, wherein R² is H.

5. The compound of claim 1, or a tautomer or isomer thereof, or a pharmaceutically acceptable salt of any of the foregoing, wherein the compound is of the Formula (II):

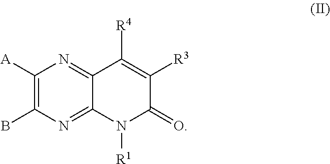

6. The compound of any one of claims 1-5, or a tautomer or isomer thereof, or a pharmaceutically acceptable salt of any of the foregoing, wherein R⁴ is H, oxo, C₁-C₆ alkyl, C₂-C₆ alkenyl, C₂-C₆ alkynyl, —CN, —NR⁹R¹⁰, halogen, 3-12-membered heterocyclyl, 5- to 10-membered heteroaryl, C₆-C₁₄ aryl, —C(O)NR⁹R¹⁰, —OR⁸, —SR⁸, —C(O)OR⁸, or —NR⁸C(O)R⁹, each of which is independently optionally substituted by halogen, oxo, —OR¹¹, —NR¹¹R¹², —C(O)R¹¹, —CN, —S(O)R¹¹, —S(O)₂R¹¹, —P(O)(OR¹¹)(OR¹²), —(C₁-C₃ alkylene)OR¹¹, —(C₁-C₃ alkylene)NR¹¹R¹², —(C₁-C₃ alkylene)C(O)R¹¹, —(C₁-C₃ alkylene)S(O)R¹¹, —(C₁-C₃ alkylene)S(O)₂R¹¹, —(C₁-C₃ alkylene)P(O)(OR¹¹)(OR¹²), C₃-C₈ cycloalkyl, or C₁-C₆ alkyl optionally substituted by oxo, —OH or halogen.

7. The compound of any one of claims 1-6, or a tautomer or isomer thereof, or a pharmaceutically acceptable salt of an of the foregoing, wherein R⁴ is selected from the group consisting of: H, oxo, methyl, ethyl, 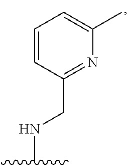 [SMILES: *NCc1cccc(C)n1]
—CN, phenyl, cyclopropyl, bromo, chloro, methoxy,

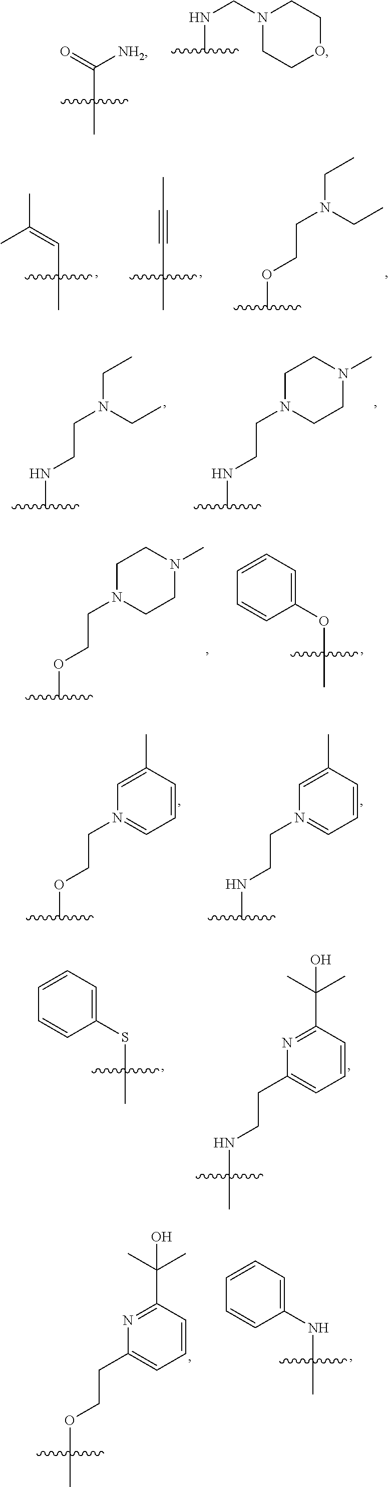

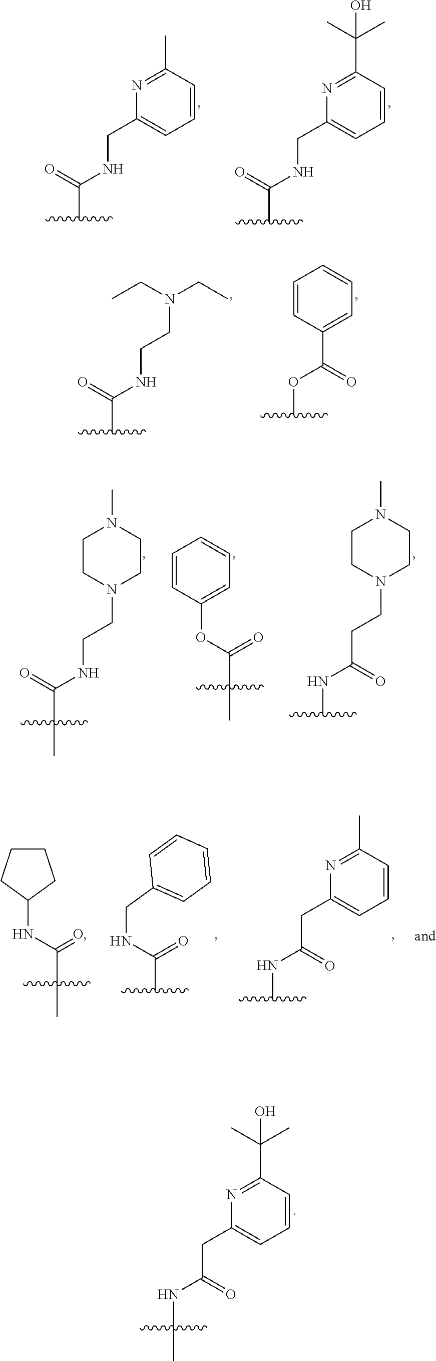

8. The compound of any one of claims 1-7, or a tautomer or isomer thereof, or a pharmaceutically acceptable salt of any of the foregoing, wherein R⁴ is H.

9. The compound of any one of claims 1-4, or a tautomer or isomer thereof, or a pharmaceutically acceptable salt of any of the foregoing, wherein the compound is of the Formula (III):

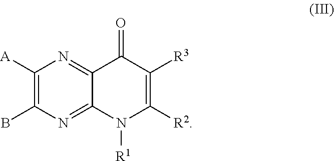

10. The compound of any one of claims 1-9, or a tautomer or isomer thereof, or a pharmaceutically acceptable salt of any of the foregoing, wherein R¹ is H or methyl.

11. The compound of any one of claims 1-10, or a tautomer or isomer thereof, or a pharmaceutically acceptable salt of any of the foregoing, wherein R¹ is H.

12. The compound of any one of claims 1-11, or a tautomer or isomer thereof, or a pharmaceutically acceptable salt of any of the foregoing, wherein A is C₆-C₁₂ aryl optionally further substituted with R⁶.

13. The compound of any one of claims 1-12, or a tautomer or isomer thereof, or a pharmaceutically acceptable salt of any of the foregoing, wherein A is phenyl or naphthyl, optionally substituted with R⁶.

14. The compound of any one of claims 1-11, or a tautomer or isomer thereof, or a pharmaceutically acceptable salt of any of the foregoing, wherein A is 5- to 10-membered heteroaryl optionally further substituted with R⁶.

15. The compound of any one of claims 1-11, or a tautomer or isomer thereof, or a pharmaceutically acceptable salt of any of the foregoing, wherein A is 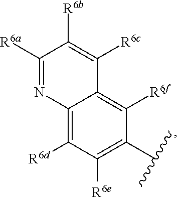
wherein R^{6a}, R^{6b}, R^{6c}, R^{6d}, R^{6e}, and R^{6f} are each independently H, C₁-C₆ alkyl, halogen, —CN, or —OC₁-C₆ alkyl.

16. The compound of any one of claims 1-11, or a tautomer or isomer thereof, or a pharmaceutically acceptable salt of any of the foregoing, wherein A is 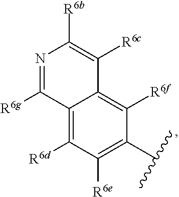
wherein R^{6b}, R^{6c}, R^{6d}, R^{6e}, R^{6f}, and R^{6g} are each independently H, C₁-C₆ alkyl, halogen, —CN, or —OC₁-C₆ alkyl.

17. The compound of any one of claims 1-11, or a tautomer or isomer thereof, or a pharmaceutically acceptable salt of any of the foregoing, wherein A is 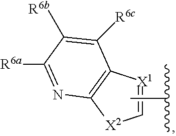
wherein X¹ is selected from the group consisting of N, C, and CH; X² is selected from the group consisting of NH, O, and S; and R^{6a}, R^{6b}, and R^{6c} are each independently H, C₁-C₆ alkyl, halogen, —CN, or —OC₁-C₆ alkyl.

18. The compound of any one of claims 1-11, or a tautomer or isomer thereof, or a pharmaceutically acceptable salt of any of the foregoing, wherein A is 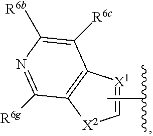
wherein X¹ is selected from the group consisting of N, C, and CH; X² is selected from the group consisting of NH, O, and S; and R^{6g}, R^{6b}, and R^{6c} are each independently H, C₁-C₆ alkyl, halogen, —CN, or —OC₁-C₆ alkyl.

19. The compound of any one of claims 1-11, or a tautomer or isomer thereof, or a pharmaceutically acceptable salt of any of the foregoing, wherein A is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl and imidazothiazolyl, each optionally substituted with R⁶.

20. The compound of any one of claims 1-10, or a tautomer or isomer thereof, or a pharmaceutically acceptable salt of any of the foregoing, wherein A is selected from the group consisting of: 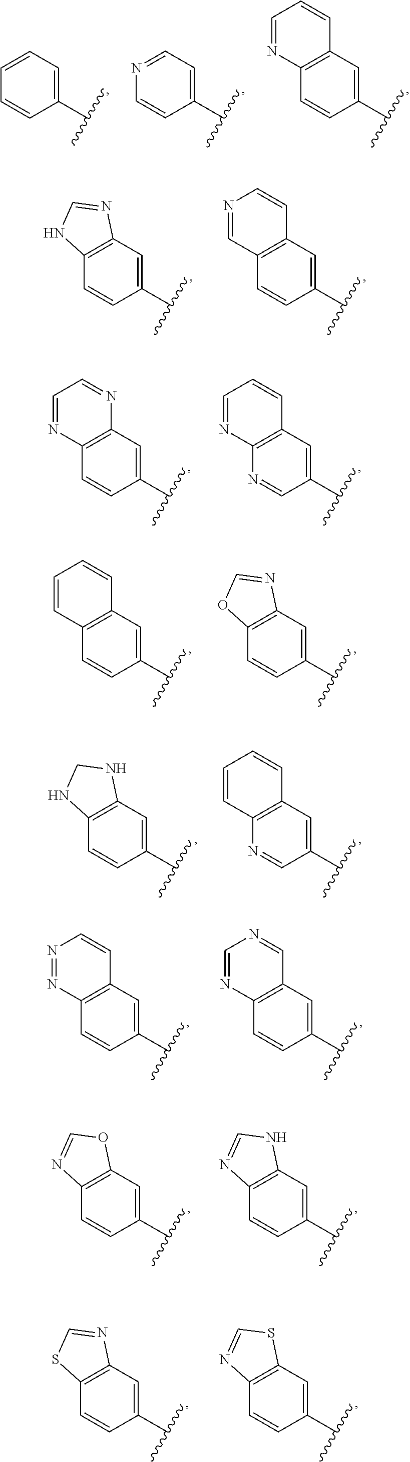 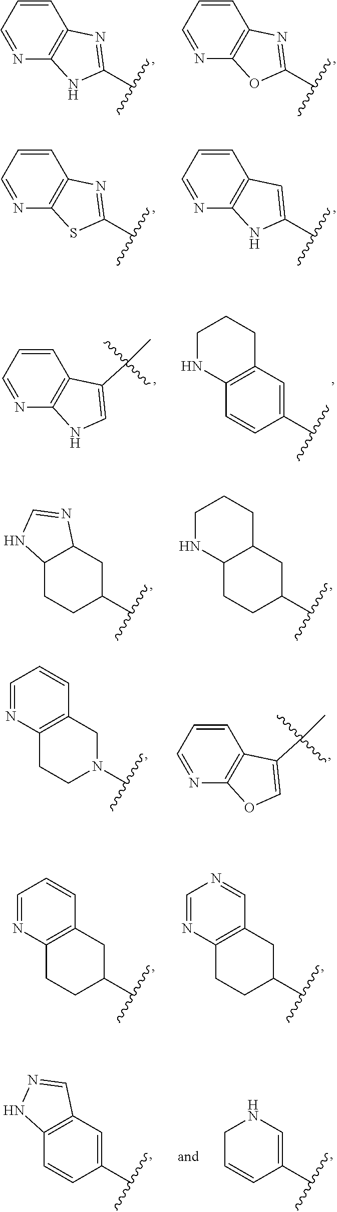
each of which is optionally substituted with R⁶.

21. The compound of any one of claims 1-11, or a tautomer or isomer thereof, or a pharmaceutically acceptable salt of any of the foregoing, wherein A is a 9- to 10-membered carbocycle optionally further substituted with R⁶.

22. The compound of any one of claims 1-11, or a tautomer or isomer thereof, or a pharmaceutically acceptable salt of any of the foregoing, wherein A is selected from the group consisting of decahydronaphthalenyl, octahydroindenyl, 1,2,3,4-tetrahydronaphthalenyl, and 2,3-dihydroindenyl, each optionally substituted with R⁶.

23. The compound of any one of claims 1-11, or a tautomer or isomer thereof, or a pharmaceutically acceptable salt of any of the foregoing, wherein A is a 9- to 10-membered heterocycle optionally further substituted with R⁶.

24. The compound of any one of claims 1-11, or a tautomer or isomer thereof, or a pharmaceutically acceptable salt of any of the foregoing, wherein A is selected from the group consisting of tetrahydroquinolinyl, tetrahydroisoquinolinyl, decahydroquinolinyl, decahydroisoquinolinyl, indolinyl, isoindolinyl, tetrahydronaphthyridinyl and hexahydrobenzoimidazolyl, each optionally further substituted with R⁶.

25. The compound of any one of claims 1-11, or a tautomer or isomer thereof, or a pharmaceutically acceptable salt of any of the foregoing, wherein A is selected from the group consisting of 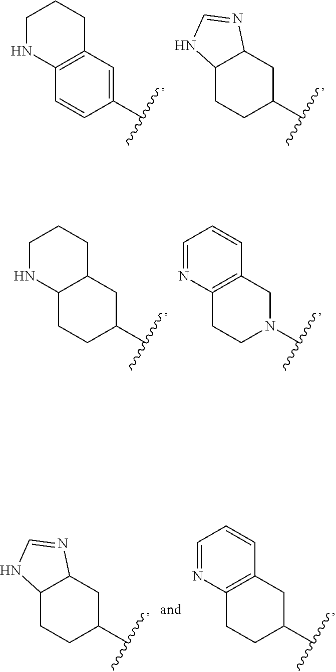 [SMILES: *c1ccc2c(c1)CCCN2 ; *C1CCC2NC=NC2C1 ; *C1CCC2NCCCC2C1 ; *N1CCc2ncccc2C1 ; *C1CCC2NC=NC2C1 ; *C1CCc2ncccc2C1]
each of which is optionally substituted with R⁶.

26. The compound of any one of claims 1-25, or a tautomer or isomer thereof, or a pharmaceutically acceptable salt of any of the foregoing, wherein each R⁶ is independently C₁-C₆ alkyl optionally substituted with halogen, halogen, oxo, C₃-C₆ cycloalkyl, —(C₁-C₃ alkylene)(C₆-C₁₄ aryl), —CN, —OR⁸, —NR⁹R¹⁰, or —NR⁸S(O)₂R⁹.

27. The compound of any one of claims 1-11, or a tautomer or isomer thereof, or a pharmaceutically acceptable salt of any of the foregoing, wherein A is selected from the group consisting of:

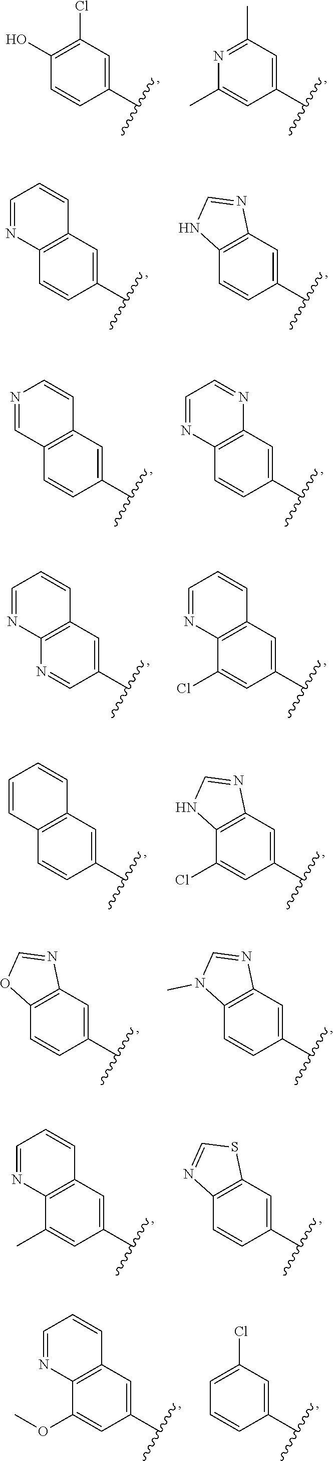

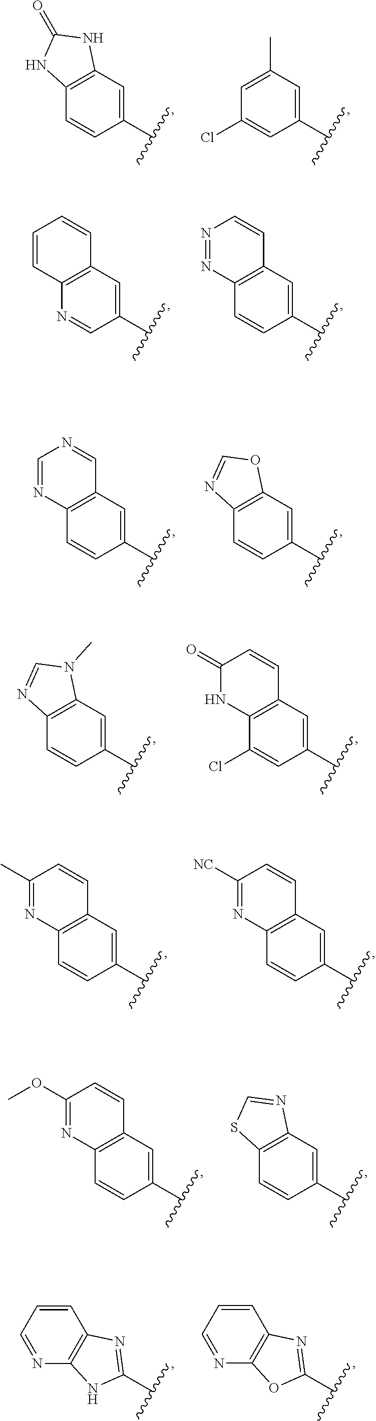

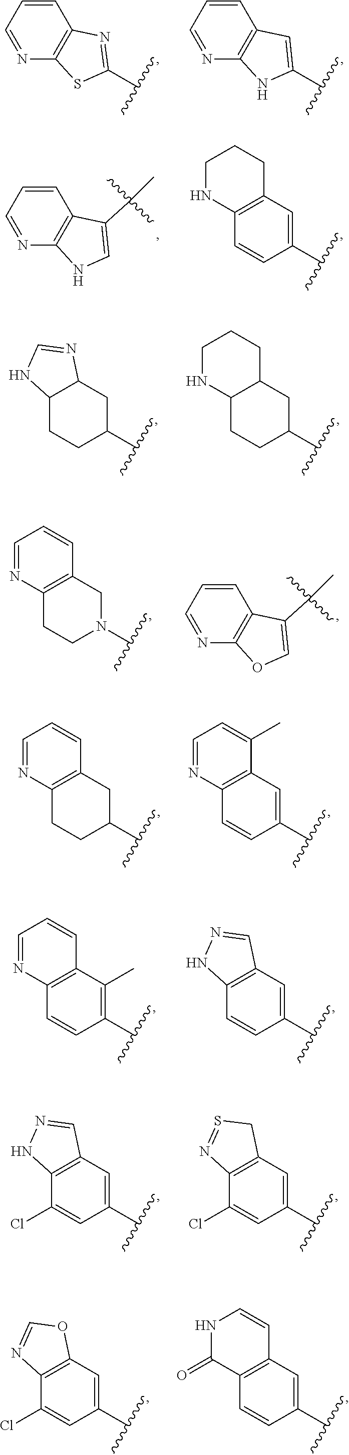

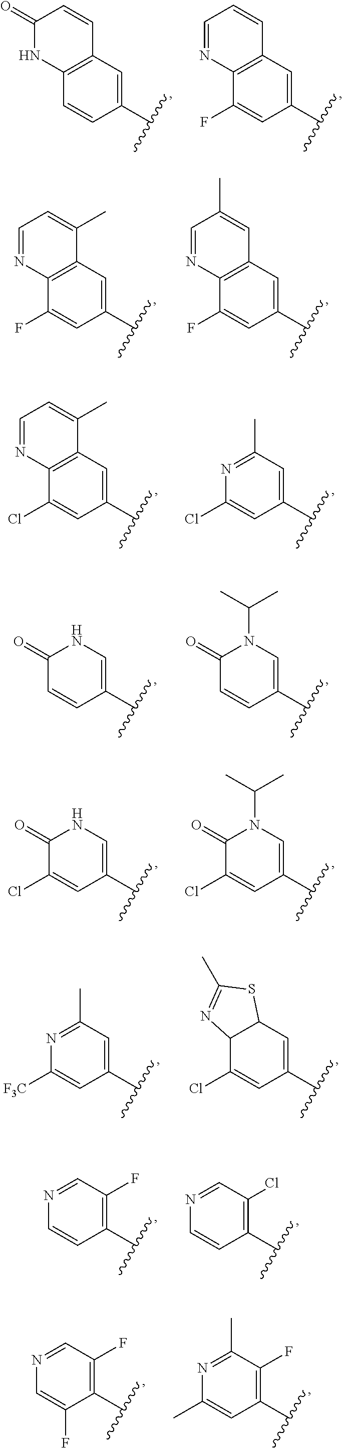

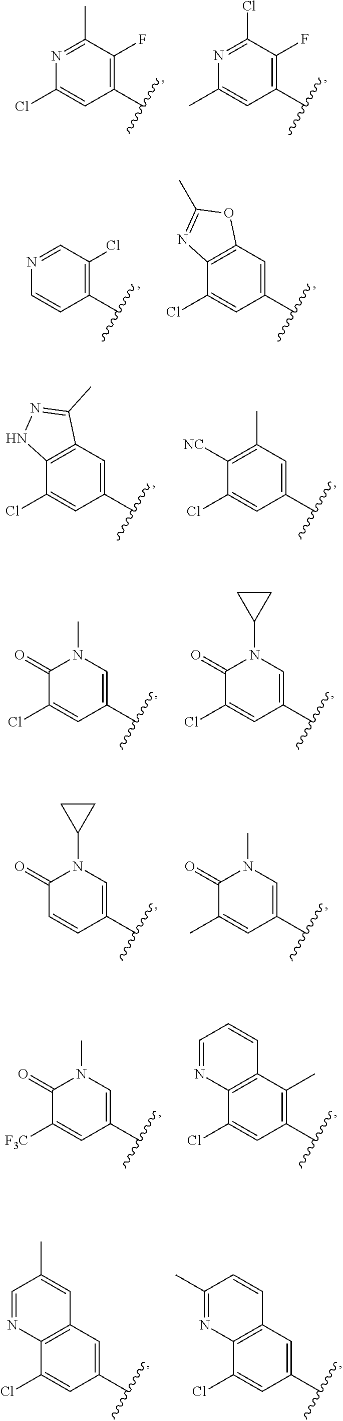

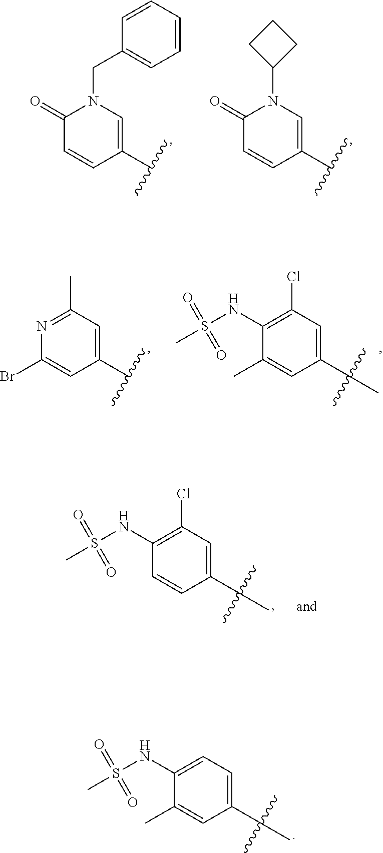

28. The compound of any one of claims 1-27, or a tautomer or isomer thereof, or a pharmaceutically acceptable salt of any of the foregoing, wherein B is phenyl, optionally further substituted with R⁷.

29. The compound of any one of claims 1-27, or a tautomer or isomer thereof, or a pharmaceutically acceptable salt of any of the foregoing, wherein B is 5- to 6-membered heteroaryl optionally further substituted with R⁷.

30. The compound of any one of claims 1-27, or a tautomer or isomer thereof, or a pharmaceutically acceptable salt of any of the foregoing, wherein B is pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridyl, pyridazinyl, pyrimidinyl, pyrazinyl, triazinyl, or tetrazinyl, each optionally substituted with R⁷.

31. The compound of any one of claims 1-27, or a tautomer or isomer thereof, or a pharmaceutically acceptable salt of any of the foregoing, wherein B is furanyl, pyridinyl, oxazoyl, or oxadiazoyl, each optionally substituted with R⁷.

32. The compound of any one of claims 1-27, or a tautomer or isomer thereof, or a pharmaceutically acceptable salt of any of the foregoing, wherein B is a 5- to 6-membered fully saturated carbocycle optionally further substituted with R⁷.

33. The compound of any one of claims 1-27, or a tautomer or isomer thereof, or a pharmaceutically acceptable salt of any of the foregoing, wherein B is cyclopentyl or cyclohexyl, optionally further substituted with R⁷.

34. The compound of any one of claims 1-27, or a tautomer or isomer thereof, or a pharmaceutically acceptable salt of any of the foregoing, wherein B is a 9- to 10-membered heteroaryl optionally further substituted with R⁷.

35. The compound of any one of claims 1-27, or a tautomer or isomer thereof, or a pharmaceutically acceptable salt of any of the foregoing, wherein B is selected from the group consisting of pyridyl, quinolinyl, isoquinolinyl, quinoxalinyl, cinnolinyl, quinazolinyl, naphthyridinyl, benzoxazolyl, benzothiazolyl, benzoimidazoyl, pyrrolyl, pyrazolyl, imidazolyl, triazolyl, tetrazolyl, furanyl, isoxazolyl, oxazolyl, oxadiazolyl, thiophenyl, isothiazolyl, thiazolyl, thiadiazolyl, pyridazinyl, pyrimidinyl, pyrazinyl, triazinyl, tetrazinyl, indolyl, isoindolyl, indazolyl, benzotriazolyl, benzofuranyl, benzoisoxazolyl, benzoxadiazolyl, benzothiophenyl, benzoisothiazolyl, benzothiadiazolyl, pyrrolopyridinyl, pyrazolopyridinyl, imidazopyridinyl, triazolopyridinyl, furopyridinyl, oxazolopyridinyl, isoxazolopyridinyl, oxadiazolopyridinyl, thienopyridinyl, thiazolopyridinyl, isothiazolopyridinyl, thiadiazolopyridinyl, thienopyridinyl, phthalazinyl, pyrazolothiazolyl, pyrazolothiazolyl and imidazothiazolyl, each optionally substituted with R⁷.

36. The compounds of any one of claims 1-27, or a tautomer or isomer thereof, or a pharmaceutically acceptable salt of any of the foregoing, wherein B is selected from the group consisting of: 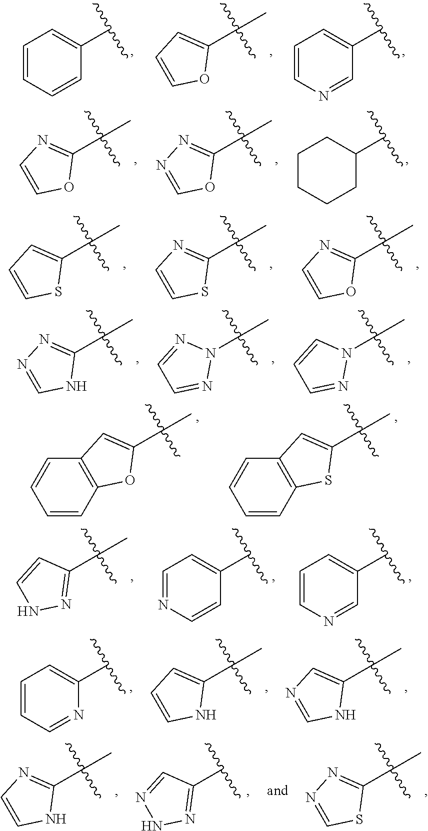
each of which is optionally substituted with R⁷.

37. The compound of any one of claims 1-36, or a tautomer or isomer thereof, or a pharmaceutically acceptable salt of any of the foregoing, wherein each R⁷ is independently C₁-C₆ alkyl, halogen, or —CN.

38. The compound of any one of claims 1-27, or a tautomer or isomer thereof, or a pharmaceutically acceptable salt of any of the foregoing, wherein B is selected from the group consisting of:

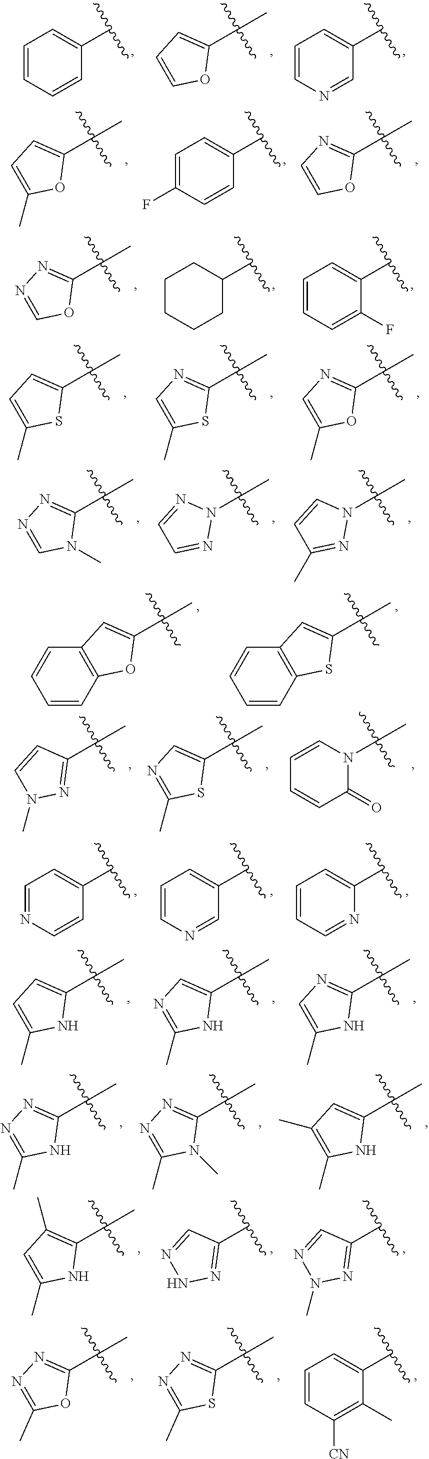

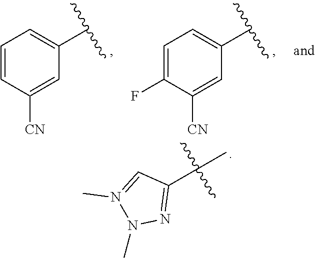

39. The compound of any one of claims 1-38, or a tautomer or isomer thereof, or a pharmaceutically acceptable salt of any of the foregoing, wherein R³ is H.

40. A compound selected from the group consisting of the compounds in Table 1, or a tautomer or isomer thereof, or a pharmaceutically acceptable salt of any of the foregoing.

41. A pharmaceutical composition comprising a compound of any one of claims 1-40, or a tautomer or isomer thereof, or a pharmaceutically acceptable salt of any of the foregoing, and a pharmaceutically acceptable carrier.

42. A method of treating disease mediated by an adenosine signaling pathway in an individual in need thereof comprising administering to the individual a therapeutically effective amount of a compound of any one of claims 1-40, or a tautomer or isomer thereof, or a pharmaceutically acceptable salt of any of the foregoing.

43. A method of treating cancer in an individual in need thereof comprising administering to the individual a therapeutically effective amount of a compound of any one of claims 1-40, or a tautomer or isomer thereof, or a pharmaceutically acceptable salt of any of the foregoing.

44. A method of inhibiting adenosine receptors of subtypes A₂ₐ, A_{2b} or A₃ in a cell, comprising administering a compound of any one of claims 1-40, or a tautomer or isomer thereof, or a pharmaceutically acceptable salt of any of the foregoing, to the cells.

45. The method of claim 44, wherein the adenosine receptors are of subtype A₂ₐ.

46. Use of a compound of any one of claims 1-40, or a pharmaceutically acceptable salt or solvate thereof, in the manufacture of a medicament for treatment of a disease mediated by an adenosine signaling pathway.

47. A kit comprising a compound of any one of claims 1-40, or a tautomer or isomer thereof, or a pharmaceutically acceptable salt of any of the foregoing.
